(19) United States
(12) Patent Application Publication
(10) Pub. No.: US 2022/0062240 A1
(43) Pub. Date: Mar. 3, 2022

(54) METHODS OF TREATING CANCER USING A CLK INHIBITOR

(71) Applicant: BioSplice Therapeutics, Inc., San Diego, CA (US)

(72) Inventors: Betty Tam, San Diego, CA (US); Carine Bossard, San Diego, CA (US); Kevin Tseng Chiu, San Diego, CA (US); Heekyung Chung, San Diego, CA (US); Luis A. Dellamary, San Marcos, CA (US); Chi Ching Mak, San Diego, CA (US); Long Hoang Do, Solana Beach, CA (US); Seong Yeon Cho, La Jolla, CA (US); Luke Jervis, Del Mar, CA (US)

(21) Appl. No.: 17/254,352

(22) PCT Filed: Jun. 26, 2019
(86) PCT No.: PCT/US2019/039301

§ 371 Date: Dec. 21, 2020

Related U.S. Application Data
(60) Provisional application No. 62/846,335, filed on May 10, 2019.
(60) Provisional application No. 62/690,146, filed on Jun. 26, 2018.

(51) Int. Cl. A61K 31/4162; G01N 33/574; A61K 31/4184; A61K 31/437; A61K 31/4725; A61K 31/519; A61P 35/00

(52) U.S. Cl. CPC A61K 31/4162; G01N 33/57496; A61K 31/4184; A61P 35/00; A61K 31/4725; A61K 31/519; A61K 31/437

(57) ABSTRACT

Provided herein are methods of treating a cancer in a subject using a CLK inhibitor or pharmaceutically acceptable salt or solvate thereof.

### RELATED APPLICATION

This application claims the benefit of U.S. Provisional Application Nos. 62/690,146, filed Jun. 26, 2018 and 62/846,335, filed May 10, 2019, which are incorporated herein by reference in their entirety.

### TECHNICAL FIELD

This present disclosure relates to the fields of cancer biology and molecular biology, and more specifically, to methods of treating cancer using CDC-like kinase (CLK) inhibitor.

### BACKGROUND

Carcinogenesis is a multistep transformation of a normal cell into a cancerous cell, which is characterized by unchecked growth. These steps enable a cancer cell's “hallmark capabilities,” including chronic proliferation, resistance to apoptosis, metastatic and angiogenic potential, immune evasion, and replicative immortality (Hanahan and Weinberg, Cell 100:57-70, 2000). Motility, cytostasis and differentiation, proliferation, and viability are the intracellular signaling networks or circuits contributing to the development of these hallmark capabilities of a cancer cell (Hanahan and Weinberg, Cell 144:646-674, 2011). There is robust crosstalk among these pathways which support cancer cell growth. The nexus of these biological processes is changes in gene expression, which can fundamentally inhibit or promote cancer cell hallmark capabilities. One pathway which can directly modulate genes important in multiple cancer signaling networks is the Wnt/β-catenin signaling pathway.

Wnt signaling is an evolutionary conserved pathway which plays an important role in embryonic development, cell viability, and regeneration (Clevers et al., Cell 149:1192-1205, 2012; Clevers, Cell 127:469-480, 2006). Signaling is activated upon Wnt ligand binding to a Frizzled family cell receptor and is transmitted via canonical (β-catenin dependent) or non-canonical (β-catenin-independent) pathways (Clevers, Cell 127(3):469-480, 2006). Activation of canonical Wnt signaling releases β-catenin from the protein complex of GSK3-β, AXIN, and adenomatous polyposis coli (APC), and promotes the proteosomal degradation of the freed β-catenin (Nusse et al., EMBO J. 31:2670-2684, 2012). Upon subsequent translocation into the nucleus, β-catenin interacts with TCF/LEF transcription factors to activate expression of target genes important not only in cell fate, but in cell proliferation and survival (Moon et al., Nat. Rev. Genet. 5:691-701, 2004). Approximately 90% of colorectal cancers (CRC) are characterized by somatic mutations in the WNT/β-catenin signaling pathway; with 80% of those resulting from loss-of-function mutation of the APC gene and to a smaller extent CTNNB1 (Kwong et al., Adv. Exp. Med. Biol. 656:85-106, 2009; Nature 487:330-337, 2012). Loss of APC function causes abnormal activation of the canonical pathway resulting in higher levels of β-catenin which contributes to tumorigenesis. The aberrant activation of Wnt/β-catenin pathway is implicated in other cancer types such as, gastric cancer, breast cancer, liver cancer, pancreatic cancer, and lung cancer (Clevers, Cell 127(3):469-480, 2006; Moon et al., Nat. Rev. Genet. 5:691-701, 2004). There are no approved therapeutic agents targeting Wnt signaling to date (Kahn, Nature Rev. Drug Discov. 13:513-532, 2014).

### SUMMARY

The present disclosure is based on the discovery that CLK inhibitors can decrease the level of Wnt/β-catenin signaling activity in a mammalian cell and can modulate mRNA splicing in a mammalian cell. In view of these discoveries, provided herein are methods of treating a cancer in a subject, methods of selecting a treatment for a subject, methods of selecting a subject for treatment, and methods of selecting a subject for participation in a clinical trial, that each include identifying a subject having a cancer cell (e.g., any of the types of cancer cell described herein) that has an elevated level of Wnt pathway activity as compared to a reference level. Also provided herein are methods of determining the efficacy of a CLK inhibitor in a subject that include detecting a level of Wnt/β-catenin signaling activity in a cancer cell obtained from the subject. Also provided are methods of decreasing the activity of one or more of CLK1, CLK2, CLK3, and CLK4 (e.g., in vitro or in a mammalian cell) that include the use of any of the CLK inhibitors or pharmaceutically acceptable salts or solvates thereof described herein. Also provided herein are methods of altering mRNA splicing in a mammalian cell having aberrant mRNA splicing activity that include the use of any of the CLK inhibitors or pharmaceutically acceptable salts or solvates thereof described herein. Also provided herein are methods of treating a cancer using a CLK inhibitor, methods of selecting a treatment including a CLK inhibitor for a subject, methods of selecting a subject for treatment with a CLK inhibitor, and methods of selecting a subject for participation in a clinical trial, that each include the use of a CLK inhibitor, that include a step of identifying a subject having aberrant mRNA splicing activity.

Also provided herein are methods of treating a cancer in a subject that include: identifying a subject having a cancer cell that has an elevated level of Wnt pathway activity as compared to a reference level; and administering to the identified subject a therapeutically effective amount of a CLK inhibitor or a pharmaceutically acceptable salt or solvate thereof.

Also provided herein are methods of treating a cancer in a subject that include administering a therapeutically effective amount of a CLK inhibitor or a pharmaceutically acceptable salt or solvate thereof to a subject identified as having a cancer cell that has an elevated level of Wnt pathway activity as compared to a reference level.

Also provided herein are methods of selecting a treatment for a subject that include: identifying a subject having a cancer cell that has an elevated level of Wnt pathway activity as compared to a reference level; and selecting for the identified subject a treatment including a therapeutically effective amount of a CLK inhibitor or a pharmaceutically acceptable salt or solvate thereof.

Also provided herein are methods of selecting a treatment for a subject that include selecting a treatment including a therapeutically effective amount of a CLK inhibitor or a pharmaceutically acceptable salt or solvate thereof for a subject identified as having a cancer cell that has an elevated level of Wnt pathway activity as compared to a reference level.

Also provided herein are methods of selecting a subject for treatment that include: identifying a subject having a cancer cell that has an elevated level of Wnt pathway activity as compared to a reference level; and selecting an identified subject for treatment with a therapeutically effective amount of a CLK inhibitor or a pharmaceutically acceptable salt or solvate thereof.

Also provided herein are methods of selecting a subject for treatment that include selecting a subject identified as having a cancer cell that has an elevated level of Wnt pathway activity as compared to a reference level, for treatment with a therapeutically effective amount of a CLK inhibitor or a pharmaceutically acceptable salt or solvate thereof.

Also provided herein are methods of selecting a subject for participation in a clinical trial that include: identifying a subject having a cancer cell that has an elevated level of Wnt pathway activity as compared to a reference level; and selecting the identified subject for participation in a clinical trial that includes administration of a therapeutically effective amount of a CLK inhibitor or a pharmaceutically acceptable salt or solvate thereof.

Also provided herein are methods of selecting a subject for participation in a clinical trial that include selecting a subject identified as having a cancer cell that has an elevated level of Wnt pathway activity as compared to a reference level for participation in a clinical trial that includes administration of a therapeutically effective amount of a CLK inhibitor or a pharmaceutically acceptable salt or solvate thereof.

Also provided herein are methods of treating a subject having a cancer that include: (a) administering to the subject a therapeutic agent; (b) after (a), identifying the subject as having a cancer cell that has an elevated level of Wnt pathway activity as compared to a reference level; and (c) administering to the identified subject a treatment including a therapeutically effective amount of a CLK inhibitor or a pharmaceutically acceptable salt or solvate thereof.

Also provided herein are methods of treating a subject having a cancer that include: identifying a subject previously administered a therapeutic agent, as having a cancer cell that has an elevated level of Wnt pathway activity as compared to a reference level; and administering to the identified subject a treatment including a therapeutically effective amount of a CLK inhibitor or a pharmaceutically acceptable salt or solvate thereof.

Also provided herein are methods of treating a subject having a cancer that include administering to a subject previously administered a therapeutic agent and later identified as having an elevated level of Wnt pathway activity as compared to a reference level, a therapeutically effective amount of a CLK inhibitor or a pharmaceutically acceptable salt or solvate thereof.

Also provided herein are methods of determining the efficacy of a CLK inhibitor or a pharmaceutically acceptable salt or solvate thereof in a subject that include: (a) determining a first level of Wnt pathway activity in a cancer cell obtained from a subject at a first time point; (b) administering to the subject after the first time a CLK inhibitor or a pharmaceutically acceptable salt or solvate thereof; (c) determining a second level of Wnt pathway activity in a cancer cell obtained from the subject at a second time point; and (d) determining that the CLK inhibitor is effective in a subject having a second level of Wnt pathway activity that is decreased as compared to the first level of Wnt pathway activity. Some embodiments of any of the methods described herein further include: (e) after (d), administering one or more additional doses of the CLK inhibitor to the subject.

In some embodiments of any of the methods described herein, the level of Wnt pathway activity is the level of CLK1, CLK2, CLK3, CLK4, or β-catenin expression. In some embodiments of any of the methods described herein, the level of CLK1, CLK2, CLK3, CLK4, or β-catenin expression is the level of CLK1, CLK2, CLK3, CLK4, or β-catenin protein. In some embodiments of any of the methods described herein, the level of Wnt pathway activity is the level of β-catenin in the nucleus.

In some embodiments of any of the methods described herein, the Wnt pathway activity is detection of a mutation in a Wnt pathway gene selected from the group of: gain-of-function mutation in a β-catenin gene, a loss-of-function mutation in an AXIN gene, a loss-of-function mutation in an AXIN2 gene, a loss-of-function mutation in a APC gene, a loss-of-function mutation in a CTNNB1 gene, a loss-of-function mutation in a Tsc1 gene, a loss-of-function mutation in a Tsc2 gene, and a loss-of-function mutation GSK3D gene.

In some embodiments of any of the methods described herein, the Wnt pathway activity is detection of an elevated level of expression of one or more Wnt-upregulated genes. In some embodiments of any of the methods described herein, the one or more Wnt-upregulated genes are selected from the group of: CCND1, CSNK2A1, CXCL12, LRP5, MMP7, MMP9, LEF1, AXIN2, MYC, TCF7L2, TCF7, LRP6, DVL2, BIRC, ERRB2, MAPK8, PKN1, AXIN2, ABCB1, ADAM10, ALEX1, ASCL2, BAMBI, BCL2L2, BIRC5, BMI1, BMP4, CCND1, CD44, CDKN2A, CDX1, CEBPD, CLDN1, COX2, DNMT1, EDN1, EFNB1, ENC1, EPHB2, EPHB3, FGF18, FGFBP, FRA1, FSCN1, FZD6, FZD7, FZD8, GAST, HDAC3, HEF1, HES1, ID2, ITF2, JAG1, JUN, LlCAM, LAMC2, LGR5, MENA, MET, MMP14, MYB, MYCBP, NOS2, NOTCH2, NRCAM, PLCB4, PLAU, PLAUR, PPARD, RUVBL1, S100A4, S100A6, SGK1, SMC3, SOX9, SP5, SRSF3, SUZ12, TCF1, TIAM1, TIMP-1. TN-C, VEGF, WNT-5a, WNT-5b, WNT11, and YAP.

In some embodiments of any of the methods described herein, the Wnt pathway activity is detection of a decreased level of expression of one or more of APC, FRZB, CTGF, and GSK3B.

In some embodiments of any of the methods described herein, the cancer is a small cell lung cancer, colorectal cancer, head and neck cancer, ovarian cancer, melanoma, renal cell carcinoma, pancreatic cancer, or non-small cell lung cancer.

Also provided herein are methods of decreasing the activity of one or more of CLK1, CLK2, CLK3, and CLK4, the method includes contacting one or more of CLK1, CLK2, CLK3 and CLK4 with an effective amount of a CLK inhibitor or a pharmaceutically acceptable salt or solvate thereof. In some embodiments of any of the methods described herein, the method includes contacting one or both of CLK2 and CLK3 with an effective amount of a CLK inhibitor or a pharmaceutically acceptable salt or solvate thereof.

Also provided herein are methods of decreasing the activity of one or more of CLK1, CLK2, CLK3 and CLK4 in a mammalian cell that include contacting the mammalian cell with an effective amount of a CLK inhibitor or a pharmaceutically acceptable salt or solvate thereof. In some embodiments of any of the methods described herein, the mammalian cell is a cancer cell. In some embodiments of any of the methods described herein, the cancer cell has been identified as having an elevated level of Wnt pathway activity as compared to a reference level. In some embodiments of any of the methods described herein, the contacting results in a decrease in the activity of one or both of CLK2 and CLK3 in the mammalian cell.

Also provided herein are methods of altering mRNA splicing in a mammalian cell having aberrant mRNA splicing activity that include contacting the mammalian cell with an effective amount of a CLK inhibitor or a pharmaceutically acceptable salt or solvate thereof. In some embodiments of any of the methods described herein, the mammalian cell is a cancer cell. In some embodiments of any of the methods described herein, the cancer cell having aberrant mRNA spicing activity has one or more of: an increased level of phosphorylated SRSF6 as compared to a reference level; an increased level of phosphorylated SRSF5 as compared to a reference level; a mutation in a SF3B1 gene, a SRSF1 gene, a SRSF2 gene, a U2AF1 gene, or a ZRSR2 gene; and an increased level of SRSF1, SRSF2, SRSF3, SRSF4, SRSF5, SRSF6, and SRSF10 as compared to a reference level.

Also provided herein are methods of treating a cancer in a subject that include: identifying a subject having a cancer cell that has aberrant mRNA splicing activity as compared to a reference level; and administering to the identified subject a therapeutically effective amount of a CLK inhibitor or a pharmaceutically acceptable salt or solvate thereof.

Also provided herein are methods of treating a cancer in a subject that include administering a therapeutically effective amount of a CLK inhibitor or a pharmaceutically acceptable salt or solvate thereof to a subject identified as having a cancer cell that has aberrant mRNA splicing activity as compared to a reference level.

Also provided herein are methods of selecting a treatment for a subject that include: identifying a subject having a cancer cell that has aberrant mRNA splicing activity as compared to a reference level; and selecting for the identified subject a treatment including a therapeutically effective amount of a CLK inhibitor or a pharmaceutically acceptable salt or solvate thereof.

Also provided herein are methods of selecting a treatment for a subject that include selecting a treatment including a therapeutically effective amount of a CLK inhibitor or a pharmaceutically acceptable salt or solvate thereof for a subject identified as having a cancer cell that has aberrant mRNA splicing activity as compared to a reference level.

Also provided herein are methods of selecting a subject for treatment that include: identifying a subject having a cancer cell that has aberrant mRNA splicing activity as compared to a reference level; and selecting an identified subject for treatment with a therapeutically effective amount of a CLK inhibitor or a pharmaceutically acceptable salt or solvate thereof.

Also provided herein are methods of selecting a subject for treatment that include selecting a subject identified as having a cancer cell that has aberrant mRNA splicing activity as compared to a reference level, for treatment with a therapeutically effective amount of a CLK inhibitor or a pharmaceutically acceptable salt or solvate thereof.

Also provided herein are methods of selecting a subject for participation in a clinical trial that include: identifying a subject having a cancer cell that has aberrant mRNA splicing activity as compared to a reference level; and selecting the identified subject for participation in a clinical trial that comprises administration of a therapeutically effective amount of a CLK inhibitor or a pharmaceutically acceptable salt or solvate thereof.

Also provided herein are methods of selecting a subject for participation in a clinical trial that include selecting a subject identified as having a cancer cell that has aberrant mRNA splicing activity as compared to a reference level for participation in a clinical trial that includes administration of a therapeutically effective amount of a CLK inhibitor or a pharmaceutically acceptable salt or solvate thereof.

Also provided herein are methods of treating a subject having a cancer that include: (a) administering to the subject a therapeutic agent; (b) after (a), identifying the subject as having a cancer cell that has aberrant mRNA splicing activity as compared to a reference level; and (c) administering to the identified subject a treatment including a therapeutically effective amount of a CLK inhibitor or a pharmaceutically acceptable salt or solvate thereof.

Also provided herein are methods of treating a subject having a cancer that include: identifying a subject previously administered a therapeutic agent, as having a cancer cell that has aberrant mRNA splicing activity as compared to a reference level; and administering to the identified subject a treatment comprising a therapeutically effective amount of a CLK inhibitor or a pharmaceutically acceptable salt or solvate thereof.

Also provided herein are methods of treating a subject having a cancer that include administering to a subject previously administered a therapeutic agent and later identified as having aberrant mRNA splicing activity as compared to a reference level, a therapeutically effective amount of a CLK inhibitor or a pharmaceutically acceptable salt or solvate thereof.

In some embodiments of any of the methods described herein, the level of aberrant mRNA splicing is determined by detecting: the level of SRSF6 phosphorylation in the cell; the level of SRSF5 phosphorylation in the cell; the level of a ˜55 kDa isoform of SRSF6 in the cell; or the level of ˜35 kDa isoform of SRSF1 in the cell.

Also provided herein are methods of determining the efficacy of a CLK inhibitor in a subject that include: (a) determining a first level of SRSF6 phosphorylation and/or SRSF5 phosphorylation in a cancer cell obtained from a subject at a first time point; (b) administering to the subject after the first time a CLK inhibitor or a pharmaceutically acceptable salt or solvate thereof, (c) determining a second level of SRSF6 phosphorylation and/or SRSF5 phosphorylation in a cancer cell obtained from the subject at a second time point; and (d) determining that the CLK inhibitor is effective in a subject having a second level that is decreased as compared to the first level.

Also provided herein are methods of determining the efficacy of a CLK inhibitor or a pharmaceutically acceptable salt or solvate thereof in a subject that include: (a) determining a first level of a ˜55 kDa isoform of SRSF6 in a cancer cell obtained from a subject at a first time point; (b) administering to the subject after the first time a CLK inhibitor or a pharmaceutically acceptable salt or solvate thereof, (c) determining a second level of the ˜55 kDa isoform of SRSF6 in a cancer cell obtained from the subject at a second time point; and (d) determining that the CLK inhibitor is effective in a subject having a second level of the ˜55 kDa isoform of SRSF6 that is increased as compared to the first level of the ˜55 kDa isoform of SRSF6.

Also provided herein are methods of determining the efficacy of a compound of any one of Formulas III-XI or a pharmaceutically acceptable salt or solvate thereof in a subject that include: (a) determining a first level of a ˜35 kDa isoform of SRSF1 in a cancer cell obtained from a subject at a first time point; (b) administering to the subject after the first time point a compound of any one of Formulas (I)-(XII) or a pharmaceutically acceptable salt or solvate thereof, (c) determining a second level of the ˜35 kDa isoform of SRSF1 in a cancer cell obtained from the subject at a second time point; and (d) determining that the CLK inhibitor is effective in a subject having a second level of the ˜35 kDa isoform of SRSF1 that is increased as compared to the first level of the ˜35 kDa isoform of SRSF1.

Some embodiments of any of the methods described herein further includes: (e) after (d), administering one or more additional doses of the CLK inhibitor to the subject.

In some embodiments of any of the methods described herein, the CLK inhibitor is a multi-isoform CLK inhibitor. In some embodiments of any of the methods described herein, the multi-isoform CLK inhibitor has an IC₅₀ of between about 1 nM and about 10 μM for each of CLK2 and CLK3. In some embodiments of any of the methods described herein, the multi-isoform CLK inhibitor has an IC₅₀ of between about 1 nM and about 1 μM for each of CLK2 and CLK3. In some embodiments of any of the methods described herein, the multi-isoform CLK inhibitor has an IC₅₀ of between about 1 nM and about 100 nM for each of CLK2 and CLK3.

In some embodiments of any of the methods described herein, the CLK inhibitor is a compound of any one of Formulas (I)-(XII) or a pharmaceutically acceptable salt or solvate thereof.

In some embodiments of any of the methods described herein, the multi-isoform CLK inhibitor has an IC₅₀ of between about 2 nM and about 10 μM for each of CLK1, CLK2, and CLK3. In some embodiments of any of the methods described herein, the multi-isoform CLK inhibitor has an IC₅₀ of between about 2 nM and about 1 μM for each of CLK1, CLK2, and CLK3. In some embodiments of any of the methods described herein, the multi-isoform CLK inhibitor has an IC₅₀ of between about 2 nM and about 10 μM for each of CLK1, CLK2, CLK3, and CLK4. In some embodiments of any of the methods described herein, the multi-isoform CLK inhibitor has an IC₅₀ of between about 2 nM and about 1 μM for each of CLK1, CLK2, CLK3, and CLK4.

In some embodiments of any of the methods described herein, the CLK inhibitor is a compound of Formula (I)

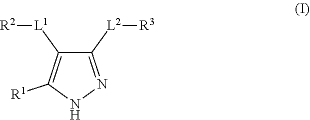

as well as prodrugs and pharmaceutically acceptable salt or solvate thereof, wherein:

R¹ is selected from the group consisting of H, halide, and unsubstituted —(C₁₋₃ alkyl);

R² is selected from the group consisting of unsubstituted —(C₁₋₃ alkyl), unsubstituted —(C₂₋₉ alkenyl), unsubstituted —(C₁₋₉ haloalkyl), —(C₁₋₂ alkylene)ₚ(C₃₋₆ carbocyclyl) optionally substituted with 1-12 R⁴, -monocyclic heterocyclyl optionally substituted with 1-10 R⁵, -phenyl substituted with 1-5 R⁶, -heteroaryl optionally substituted with 1-4 R⁷, —CO₂R, —OR⁹, and —(C═O)R¹⁰; wherein heteroaryl selected from the group consisting of pyridinyl, pyrimidinyl, pyrazinyl, oxazolyl, oxadiazolyl, thiazolyl, 2,3-dihydrobenzo[b]dioxinyl, 5,6,7,8-tetrahydroimidazo[1,2-a]pyrazinyl, 4,5,6,7-tetrahydro-1H-imidazo[4,5-c]pyridinyl, isoquinolinyl, and quinolinyl; wherein —(C₁₋₄ alkylene) is optionally substituted with one or more substituents as defined anywhere herein; with the proviso that when L¹ is a bond, R² is selected from the group consisting of -phenyl substituted with 1-5 R⁶ and -heteroaryl optionally substituted with 1-4 R⁷; wherein heteroaryl selected from the group consisting of pyridinyl, oxazolyl, oxadiazolyl, thiazolyl, 2,3-dihydrobenzo[b]dioxinyl, 5,6,7,8-tetrahydroimidazo[1,2-a]pyrazinyl, 4,5,6,7-tetrahydro-1H-imidazo[4,5-c]pyridinyl, isoquinolinyl, and quinolinyl;

R³ is selected from the group consisting of -heterocyclyl substituted with 1-10 R¹, —(C₁₋₄ alkylene)ₚphenyl substituted with 1-5 R¹², -heteroaryl optionally substituted with 1-4 R¹³, and —(C₁₋₄ alkylene)OR¹⁴; wherein heteroaryl selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein

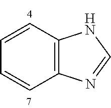

is only substituted at positions 4 and 7; wherein each —(C₁₋₄ alkylene) is, independently, optionally substituted with one or more substituents as defined anywhere herein;

with the proviso that when L² is a bond, R³ is selected from -heteroaryl optionally substituted with 1-4 R¹³; wherein heteroaryl selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein

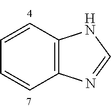

is only substituted at positions 4 and 7;

each R⁴ is halide;

each R⁵ is independently selected from the group consisting of halide, Me, and Et;

each R⁶ is independently selected from the group consisting of methyl, —CH₂F, —CHF₂, —CF₃, —OR^{15a}, and —(C₁₋₄ alkylene)ₚN(R^{16a})(R^{16b}); wherein —(C₁₋₄ alkylene) is optionally substituted with one or more substituents as defined anywhere herein;

each R⁷ is independently selected from the group consisting of F, methyl, —CH₂F, —CHF₂, —CF₃, —CF₂CH₃, —OR^{15a}, —CO₂R¹⁷, —NR¹⁸(C═O)R¹⁹, —(C₁₋₄ alkylene)ₚheterocyclyl optionally substituted with 1-10 R^{20b}, and —(C₁₋₄ alkylene)ₚN(R^{16a})(R^{16b}); wherein each —(C₁₋₄ alkylene) is, independently, optionally substituted with one or more substituents as defined anywhere herein;

R⁸ is unsubstituted —(C₁₋₉ alkyl);

R⁹ is unsubstituted —(C₁₋₉ alkyl);

R¹⁰ is -aryl optionally substituted with 1-5 R²¹;

each R¹¹ is independently selected from the group consisting of halide, methyl, and ethyl;

each R¹² is independently selected from the group consisting of —(C₁₋₄ alkylene)ₚheterocyclyl optionally substituted with 1-10 R^{20a}, -aryl optionally substituted with 1-5 R²², —(C₁₋₄ alkylene)N(R^{16a})(R^{16b}), and —OR^{23a}; wherein heterocyclyl selected from the group consisting of azetidinyl, pyrrolidinyl, piperidinyl, and piperazinyl; wherein each —(C₁₋₄ alkylene) is, independently, optionally substituted with one or more substituents as defined anywhere herein;

each R¹³ is independently selected from the group consisting of F, methyl, —CH₂F, —CHF₂, —CF₃, —(C₁₋₄ alkylene)ₚN(R^{16a})₂, —OR^{23b}, —(C₁₋₄ alkylene)ₚheterocyclyl optionally substituted with 1-10 R^{20b}, -aryl optionally substituted with 1-5 R²², and -heteroaryl substituted with 1-4 R²⁴; wherein —(C₁₋₄ alkylene) is optionally substituted with one or more substituents as defined anywhere herein;

R¹⁴ is selected from the group consisting of unsubstituted —(C₁₋₄ alkyl) and -aryl optionally substituted with 1-5 R²²;

each R^{15a} is independently selected from the group consisting of unsubstituted —(C₂₋₃ alkyl), and -heterocyclyl optionally substituted with 1-10 R^{20b};

each R^{15b} is independently selected from the group consisting of H, unsubstituted —(C₂₋₉ alkyl), and -heterocyclyl optionally substituted with 1-10 R^{20b};

each R^{16a} is independently selected from the group consisting of H and unsubstituted —(C₁₋₂ alkyl);

each R^{16b} is unsubstituted —(C₁₋₂ alkyl);

each R¹⁷ is unsubstituted —(C₁₋₉ alkyl);

each R¹⁸ is independently selected from the group consisting of H and Me;

each R¹⁹ is unsubstituted —(C₁₋₉ alkyl);

each R^{20a} is independently selected from the group consisting of halide and unsubstituted —(C₂₋₉ alkyl);

each R^{20b} is independently selected from the group consisting of halide and unsubstituted —(C₁₋₉ alkyl);

each R²¹ is independently selected from the group consisting of halide and unsubstituted —(C₁₋₉ alkyl);

each R²² is independently selected from the group consisting of halide and unsubstituted —(C₁₋₉ alkyl);

each R^{23a} is independently selected from the group consisting of unsubstituted —(C₂₋₉ alkyl), —(C₁₋₄ alkylene)OR²⁵, and —(C₁₋₄ alkylene)ₚheterocyclyl optionally substituted with 1-10 R^{20b}; wherein each —(C₁₋₄ alkylene) is, independently, optionally substituted with one or more substituents as defined anywhere herein;

each R^{23b} is independently selected from the group consisting of unsubstituted —(C₁₋₉ alkyl), —(C₁₋₄ alkylene)OR²⁵, and —(C₁₋₄ alkylene)ₚheterocyclyl optionally substituted with 1-10 R^{20b}; wherein each —(C₁₋₄ alkylene) is, independently, optionally substituted with one or more substituents as defined anywhere herein;

each R²⁴ is independently selected from the group consisting of halide and unsubstituted —(C₁₋₉ alkyl);

each R²⁵ is independently selected from the group consisting of H and unsubstituted —(C₁₋₉ alkyl);

L¹ is selected from the group consisting of a bond, —CH═CH—, —C≡C—, —(CH₂)ₚNR¹⁸(C═O)—, —(C═O)NR¹⁸(CH₂)ₚ—, —NR¹⁸(C═O)NR¹⁸—, —NH(CH₂)ₚ—, and —(CH₂)ₚNH—;

L² is selected from the group consisting of a bond, —(C═O)NR¹⁸—, —NR¹⁸(C═O)—, —NHCH₂—, and —CH₂NH—; and

each p is independently an integer of 0 or 1.

In some embodiments of any of the methods described herein, the CLK inhibitor is a compound of Formula (II)

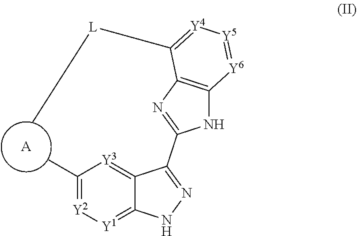

as well as prodrugs and pharmaceutically acceptable salt or solvate thereof, wherein:

Ring A is a 5-6-membered heteroaryl optionally substituted with 1-4 R¹;

L is -L¹-L²-L³-L⁴-;

L¹ is selected from the group consisting of unsubstituted —(C₁₋₃ alkylene)-, —NR²—, —NR³(C═O)—, —(C═O)NR³—, and —O—;

L² is selected from the group consisting of unsubstituted —(C₁₋₆ alkylene)- and —NR²—;

L³ is selected from the group consisting of unsubstituted —(C₁₋₆ alkylene)-, —O—, and -carbocyclylene- optionally substituted with one or more halides;

L⁴ is selected from the group consisting of unsubstituted —(C₁₋₆ alkylene)-, —O—, —NR²—, —NR³(C═O)—, —(C═O)NR³—, -arylene- optionally substituted with 1-5 R⁴, and -heteroarylene-optionally substituted with 1-4 R⁵;

with the proviso that —NR²— and —O— are not adjacent to each other;

with the proviso that two —NR³(C═O)— and/or —(C═O)NR³—, are not adjacent to each other;

each R¹ is selected from the group consisting of halide, unsubstituted —(C₁₋₃ alkyl), unsubstituted —(C₁₋₃ haloalkyl), and —CN;

each R² is selected from the group consisting of H and unsubstituted —(C₁₋₆ alkyl);

each R³ is selected from the group consisting of H and unsubstituted —(C₁₋₆ alkyl);

each R⁴ is selected from the group consisting of halide, unsubstituted —(C₁₋₆ alkyl), unsubstituted —(C₁₋₆ haloalkyl), and —CN;

each R⁵ is selected from the group consisting of halide, unsubstituted —(C₁₋₆ alkyl), unsubstituted —(C₁₋₆ haloalkyl), and —CN;

Y¹, Y², Y³, Y⁴, Y⁵, and Y⁶ are independently selected from the group consisting of carbon and nitrogen; wherein

if Y¹ is nitrogen then Y² and Y³ are CH;

if Y² is nitrogen then Y¹ and Y³ are CH;

if Y³ is nitrogen then Y¹ and Y² are CH;

if Y⁴ is nitrogen then Y⁵ and Y⁶ are CH;

if Y⁵ is nitrogen then Y⁴ and Y⁶ are CH; and

if Y⁶ is nitrogen then Y⁴ and Y⁵ are CH.

In some embodiments of any of the methods described herein, the CLK inhibitor is a compound of Formula (III)

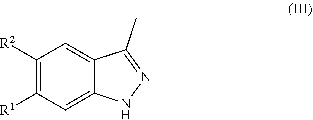

as well as prodrugs and pharmaceutically acceptable salt or solvate thereof, wherein:

R¹ is selected from the group of H and halide;

R² is a 6-membered -heteroaryl substituted with 1-4 R³;

each R³ is selected from the group of —OR⁴, —NHR⁵, and —(C₁₋₄ alkylene)ₚheterocyclyl optionally substituted with 1-10 R⁶; wherein —(C₁₋₄ alkylene) is optionally substituted with one or more substituents as defined anywhere herein;

each R⁴ is independently selected from the group of -heterocyclyl optionally substituted with 1-10 R⁷ and —CH₂CH(R)NH₂;

each R is independently selected from the group of —(C₁₋₄ alkylene)ₚheterocyclyl optionally substituted with 1-10 R⁹ and -carbocyclyl optionally substituted with 1-12 R¹⁰; wherein —(C₁₋₄ alkylene) is optionally substituted with one or more substituents as defined anywhere herein;

each R⁶ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁷ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁸ is independently selected from the group of —(C₁₋₄ alkylene)aryl optionally substituted with 1-5 R¹ and —(C₁₋₄ alkylene)heteroaryl optionally substituted with 1-4 R¹²; wherein

each —(C₁₋₄ alkylene) is, independently, optionally substituted with one or more substituents as defined anywhere herein;

each R⁹ is independently selected from the group of halide, —OH, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹⁰ is independently selected from the group of halide, —OH, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹¹ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹² is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); and

each p is independently 0 or 1.

In some embodiments of any of the methods described herein, the CLK inhibitor is a compound of Formula (IV)

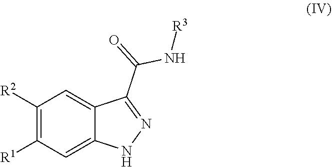

as well as prodrugs and pharmaceutically acceptable salt or solvate thereof, wherein:

R¹ is selected from the group of H and halide;

R² is a -heteroaryl optionally substituted with 1-4 R⁴;

R³ is selected from the group of -aryl optionally substituted with 1-5 R⁵ and -heteroaryl optionally substituted with 1-4 R⁶;

each R⁴ is independently selected from the group of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚN(R⁷)(R⁸), —NHC(═O)R⁹, —(C₁₋₄ alkylene)ₚOR¹⁰, unsubstituted -carbocyclyl, —(C₁₋₄ alkylene)ₚheterocyclyl optionally substituted with 1-10 R¹⁴, —(C₁₋₄ alkylene)ₚaryl optionally substituted with 1-5 R¹¹, and —(C₁₋₄ alkylene)ₚheteroaryl optionally substituted with 1-4 R¹²; wherein each —(C₁₋₄ alkylene) is, independently, optionally substituted with one or more substituents as defined anywhere herein;

each R⁵ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;

each R⁶ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R¹⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;

each R⁷ is independently selected from the group of H, unsubstituted —(C₁₋₆ alkyl), unsubstituted —(C₂₋₆ alkenyl), and unsubstituted —(C₂₋₆ alkynyl);

each R⁸ is independently selected from the group of H, unsubstituted —(C₁₋₆ alkyl), unsubstituted —(C₂₋₆ alkenyl), unsubstituted —(C₂₋₆ alkynyl), and -heterocyclyl optionally substituted with 1-10 R²¹;

alternatively, R⁷ and R⁸ are taken together to form a -heterocyclyl ring optionally substituted with 1-10 R²¹;

each R⁹ is independently selected from the group of —N(R²²)₂, -carbocyclyl optionally substituted with 1-12 R²³, -heterocyclyl optionally substituted with 1-10 R²¹, and -aryl optionally substituted with 1-5 R²⁴;

each R¹⁰ is independently selected from the group of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R²¹;

each R¹¹ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); each R¹² is independently selected from the group of halide, —(C₁₋₄ alkylene)pOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;

each R¹³ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹⁴ is independently selected from the group of halide, —(C₁₋₄ alkylene)pOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;

each R⁵ is independently selected from the group of H, unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;

alternatively, two adjacent R¹⁵ are taken together to form a -heterocyclyl ring optionally substituted with 1-10 R²¹;

each R¹⁶ is independently selected from the group of unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;

each R¹⁷ is independently selected from the group of H, unsubstituted —(C₁₋₆ alkyl), unsubstituted —(C₂₋₆ alkenyl), and unsubstituted —(C₂₋₆ alkynyl);

each R¹⁸ is independently selected from the group of H, unsubstituted —(C₁₋₆ alkyl), unsubstituted —(C₂₋₆ alkenyl), unsubstituted —(C₂₋₆ alkynyl), —(C₁₋₄ alkylene)NMe₂, and -heterocyclyl ring optionally substituted with 1-10 R²¹; wherein —(C₁₋₄ alkylene) is optionally substituted with one or more substituents as defined anywhere herein;

each R¹⁹ is independently selected from the group of unsubstituted —(C₁₋₆ alkyl), unsubstituted —(C₂₋₆ alkenyl), and unsubstituted —(C₂₋₆ alkynyl).

each R²⁰ is independently selected from the group of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —CH(CH₂OH)₂, —(C₁₋₄ alkylene)ₚheterocyclyl ring optionally substituted with 1-10 R²¹, and -aryl optionally substituted with 1-5 R²⁴; wherein —(C₁₋₄ alkylene) is optionally substituted with one or more substituents as defined anywhere herein;

each R²¹ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²² is independently selected from the group of unsubstituted —(C₁₋₆ alkyl), unsubstituted —(C₂₋₆ alkenyl), and unsubstituted —(C₂₋₆ alkynyl);

each R²³ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²⁴ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); and

each p is independently 0 or 1.

In some embodiments of any of the methods described herein, the CLK inhibitor is a compound of Formula (V)

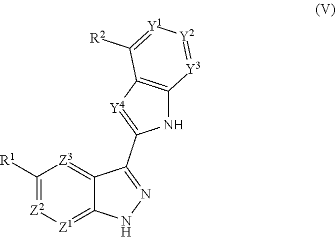

or a pharmaceutically acceptable salt or solvate thereof, wherein:

R¹ is a -heteroaryl optionally substituted with 1-2 R³;

R² is selected from the group of H, halide, -aryl optionally substituted with 1-5 R⁴-heteroaryl optionally substituted with 1-4 R⁵, and -heterocyclyl ring optionally substituted with 1-10 R⁶;

each R³ is independently selected from the group of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R⁷, —C(═O)N(R⁸)₂, —NHC(═O)R⁹, —(C₁₋₄ alkylene)ₚN(R¹⁰)(R¹), —(C₁₋₄ alkylene)ₚOR¹², and -carbocyclyl optionally substituted with 1-12 R¹³; wherein each —(C₁₋₄ alkylene) is, independently, optionally substituted with one or more substituents as defined anywhere herein;

each R⁴ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚNHSO₂R¹⁴, —NR⁵(C₁₋₄ alkylene)NR¹⁵R¹⁶, —(C₁₋₄ alkylene)ₚNR¹⁵R¹⁶, —OR¹⁷, and -heterocyclyl optionally substituted with 1-10 R¹⁹; wherein each —(C₁₋₄ alkylene) is, independently, optionally substituted with one or more substituents as defined anywhere herein;

each R⁵ is independently selected from the group of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and —C(═O)R¹⁸;

each R⁶ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁷ is independently selected from the group of halide, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁸ is independently selected from the group of H, unsubstituted —(C₁₋₆ alkyl), unsubstituted —(C₂₋₆ alkenyl), unsubstituted —(C₂₋₆ alkynyl), -heterocyclyl optionally substituted with 1-10 R¹⁹, —(C₁₋₄ alkylene)ₚcarbocyclyl optionally substituted with 1-12 R²⁰; wherein —(C₁₋₄ alkylene) is optionally substituted with one or more substituents as defined anywhere herein;

each R⁹ is independently selected from the group of unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²²)₂; wherein each —(C₁₋₄ alkylene) is, independently, optionally substituted with one or more substituents as defined anywhere herein;

each R¹⁰ is independently selected from the group of H, unsubstituted —(C₁₋₆ alkyl), unsubstituted —(C₂₋₆ alkenyl), and unsubstituted —(C₂₋₆ alkynyl);

each R¹¹ is independently selected from the group of H, unsubstituted —(C₁₋₆ alkyl), unsubstituted —(C₂₋₆ alkenyl), unsubstituted —(C₂₋₆ alkynyl), —(C₁₋₄ alkylene)ₚcarbocyclyl optionally substituted with 1-12 R²⁰; and —(C₁₋₄ alkylene)ₚaryl optionally substituted with 1-5 R²¹; wherein each —(C₁₋₄ alkylene) is, independently, optionally substituted with one or more substituents as defined anywhere herein;

each R¹² is independently selected from the group of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²²)₂; wherein each —(C₁₋₄ alkylene) is, independently, optionally substituted with one or more substituents as defined anywhere herein;

each R¹³ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); each R¹⁴ is independently selected from the group of unsubstituted —(C₁₋₆ alkyl), unsubstituted —(C₂₋₆ alkenyl), and unsubstituted —(C₂₋₆ alkynyl);

each R⁵ is independently selected from the group of H, unsubstituted —(C₁₋₆ alkyl), unsubstituted —(C₂₋₆ alkenyl), and unsubstituted —(C₂₋₆ alkynyl);

each R¹⁶ is independently selected from the group of H, unsubstituted —(C₁₋₆ alkyl), unsubstituted —(C₂₋₆ alkenyl), and unsubstituted —(C₂₋₆ alkynyl);

each R¹⁷ is independently selected from the group of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, and, —(C₁₋₄ alkylene)ₚN(R²²)₂; wherein each —(C₁₋₄ alkylene) is, independently, optionally substituted with one or more substituents as defined anywhere herein;

each R¹⁸ is independently selected from the group of unsubstituted —(C₁₋₆ alkyl), unsubstituted —(C₂₋₆ alkenyl), and unsubstituted —(C₂₋₆ alkynyl);

each R¹⁹ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²⁰ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²¹ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R²² is independently selected from the group of H, unsubstituted —(C₁₋₆ alkyl), unsubstituted —(C₂₋₆ alkenyl), and unsubstituted —(C₂₋₆ alkynyl);

each R²³ is independently selected from the group of H and halide;

Y¹, Y², and Y³ are independently selected from the group of —CR²³═ and —N═;

Y⁴ is selected from the group of —CH═ and —N═;

Z¹, Z², and Z³ are independently selected from the group of —CR²³═ and —N═; and

each p is independently 0 or 1.

In some embodiments of any of the methods described herein, the CLK inhibitor is a compound of Formula (VI)

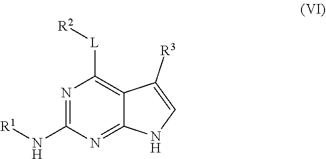

as well as prodrugs and pharmaceutically acceptable salt or solvate thereof, wherein:

R¹ is selected from the group of H, unsubstituted —(C₁₋₆ alkyl), unsubstituted —(C₂₋₆ alkenyl), unsubstituted —(C₂₋₆ alkynyl), and -heteroaryl optionally substituted with 1-4 R⁴, -aryl optionally substituted with 1-5 R;

R² is selected from the group of H, —(C₁₋₄ alkylene)ₚheteroaryl optionally substituted with 1-4 R⁶, —(C₁₋₄ alkylene)ₚheterocyclyl optionally substituted with 1-10 R⁷, and —(C₁₋₄ alkylene)ₚcarbocyclyl optionally substituted with 1-12 R; wherein each —(C₁₋₄ alkylene) is, independently, optionally substituted with one or more substituents as defined anywhere herein;

R³ is selected from the group of -heteroaryl optionally substituted with 1-4 R⁹ and -aryl optionally substituted with 1-5 R¹⁰;

each R⁴ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R¹⁴, and —(C₁₋₄ alkylene)ₚcarbocyclyl optionally substituted with 1-12 R¹⁵; wherein each —(C₁₋₄ alkylene) is, independently, optionally substituted with one or more substituents as defined anywhere herein;

each R⁵ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R¹⁴, and —(C₁₋₄ alkylene)ₚcarbocyclyl optionally substituted with 1-12 R¹⁵; wherein each —(C₁₋₄ alkylene) is, independently, optionally substituted with one or more substituents as defined anywhere herein;

each R⁶ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;

each R⁷ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R⁸ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); each R⁹ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;

each R¹⁰ is independently selected from the group of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;

each R¹¹ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

each R¹² is independently selected from the group of H, halide, unsubstituted —(C₁₋₆ alkyl), unsubstituted —(C₂₋₆ alkenyl), and unsubstituted —(C₂₋₆ alkynyl);

each R¹³ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); each R¹⁴ is independently selected from the group of halide, unsubstituted —(C₁₋₆ alkyl), unsubstituted —(C₂₋₆ alkenyl), and unsubstituted —(C₂₋₆ alkynyl);

each R⁵ is independently selected from the group of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);

L is selected from the group of a bond, —O—, and —NH—; and each p is independently 0 or 1.

In some embodiments of any of the methods described herein, the CLK inhibitor is a compound of Formula (VII)

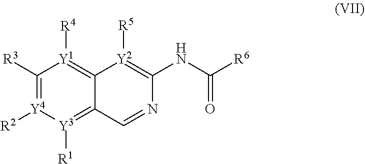

as well as prodrugs and pharmaceutically acceptable salt or solvate thereof, wherein:

R¹, R², R⁴, and R⁵ are independently absent or selected from the group of H and halide;

R³ is selected from the group of -heteroaryl optionally substituted with 1-4 R⁸ and -Xheterocyclyl optionally substituted with one or more halides or one or more unsubstituted —(C₁₋₅ alkyl);

R⁶ is selected from the group of -aryl substituted with 1-5 R⁹, —(C₂₋₄ alkenylene)aryl substituted with 1-5 R⁹, —(C₁₋₄ alkylene)ₚheteroaryl optionally substituted with 1-6 R¹⁰; -heterocyclyl optionally substituted with 1-10 R¹¹, -carbocyclyl optionally substituted with 1-12 R¹², and —(C₂₋₉ alkynyl) optionally substituted with one or more halides; wherein each —(C₁₋₄ alkylene) is, independently, optionally substituted with one or more substituents as defined anywhere herein; wherein —(C₁₋₄ alkenylene) is, optionally substituted with one or more substituents as defined anywhere herein;

with the proviso that R⁶ is heterocyclyl only when R³ is a 6-membered heteroaryl;

each R⁸ is independently selected from the group of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —N(R¹⁵)(R¹⁸), —(C₁₋₄ alkylene)ₚXR¹⁹, —C(═O)N(R¹⁵)₂, —(C₁₋₄ alkylene)ₚheterocyclyl optionally substituted with 1-10 R²⁰, and -carbocyclyl optionally substituted with 1-12 R²¹; wherein each —(C₁₋₄ alkylene) is, independently, optionally substituted with one or more substituents as defined anywhere herein;

alternatively, two adjacent R⁸ are taken together to form a ring which is selected from the group of -heterocyclyl optionally substituted with 1-10 R²² and -carbocyclyl optionally substituted with 1-12 R²¹;

each R⁹ is independently selected from the group of D, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —XR²³, —(C₁₋₄ alkylene)ₚN(R²⁴)₂, —(C₁₋₄ alkylene)ₚheterocyclyl optionally substituted with 1-10 R²²; wherein each —(C₁₋₄ alkylene) is, independently, optionally substituted with one or more substituents as defined anywhere herein;

each R¹⁰ is independently selected from the group of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —XR²³, —C(═O)N(R¹⁵)₂, —(C₁₋₄ alkylene)ₚN(R²⁴)₂, -heterocyclyl optionally substituted with 1-10 R²², and -carbocyclyl optionally substituted with 1-12 R²¹; wherein —(C₁₋₄ alkylene) is optionally substituted with one or more substituents as defined anywhere herein;

each R¹¹ is independently selected from the group of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group of halide, —(C₁₋₄ alkylene)ₚOR¹⁹; wherein —(C₁₋₄ alkylene) is optionally substituted with one or more substituents as defined anywhere herein; each R⁵ is selected from the group of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

R¹⁸ is independently selected from the group of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —(C₁₋₄ alkylene)ₚheterocyclyl optionally substituted with one or more halides or one or more unsubstituted —(C₁₋₅ alkyl); wherein —(C₁₋₄ alkylene) is, independently, optionally substituted with one or more substituents as defined anywhere herein;

each R¹⁹ is independently selected from the group of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl); wherein —(C₁₋₄ alkylene) is optionally substituted with one or more substituents as defined anywhere herein;

each R²⁰ independently is selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —OH;

each R²¹ is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN;

each R²² is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —OH, —N(R⁵)₂, —C(═O)R³⁴, and -carbocyclyl optionally substituted with 1-12 R²¹;

each R²³ is independently selected from the group of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)N(R¹⁵)₂, -heterocyclyl optionally substituted with 1-10 R³¹, and -carbocyclyl optionally substituted with 1-12 R²¹; wherein —(C₁₋₄ alkylene) is optionally substituted with one or more substituents as defined anywhere herein;

each R²⁴ is independently selected from the group of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)N(R⁵)₂; wherein each —(C₁₋₄ alkylene) is, independently, optionally substituted with one or more substituents as defined anywhere herein;

each R³¹ is independently selected from the group of halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

each R³⁴ is independently selected from the group of —O(C₁₋₅ alkyl) and a heteroaryl optionally substituted with 1-6 R³⁵;

each R³⁵ is a -heterocyclyl optionally substituted with one or more halides or one or more unsubstituted —(C₁₋₅ alkyl);

each X is selected from the group of O and S;

Y¹, Y², Y³, and Y⁴ are independently selected from the group consisting of carbon and nitrogen; wherein

if Y¹ is nitrogen then Y², Y³, and Y⁴ are carbon, and R⁴ is absent;

if Y² is nitrogen then Y¹, Y³, and Y⁴ are carbon, and R⁵ is absent;

if Y³ is nitrogen then Y¹, Y², and Y⁴ are carbon, and R¹ is absent;

if Y⁴ is nitrogen then Y¹, Y², and Y³ are carbon, and R² is absent; and

each p is independently 0 or 1.

In some embodiments of any of the methods described herein, the CLK inhibitor is a compound of Formula (VIII)

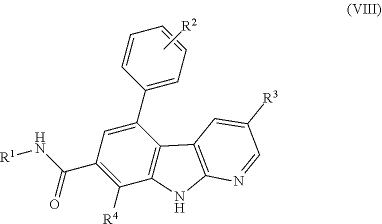

as well as prodrugs and pharmaceutically acceptable salt or solvate thereof, wherein:

R¹ is selected from the group of —(C₁₋₄ alkylene)N(R⁵)₂, —(C₁₋₄ alkylene)ₚheterocyclyl optionally substituted with 1-10 R⁶, and —(C₁₋₄ alkylene)ₚcarbocyclyl optionally substituted with 1-12 R⁷; wherein each —(C₁₋₄ alkylene) is, independently, optionally substituted with one or more substituents as defined anywhere herein;

R² is selected from the group of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —CN, —OR, —C(═O)NHR⁹, —NHC(═O)(R¹⁰), —SO₂R¹⁰, —NHSO₂R¹⁰, and —SO₂NHR⁹;

R³ is selected from the group of H, halide, unsubstituted —(C₁₋₅ alkyl), unsubstituted —(C₂₋₅ alkenyl), unsubstituted —(C₂₋₅ alkynyl), and unsubstituted —(C_{L}_5 haloalkyl);

R⁴ is selected from the group of H, halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

each R⁵ is independently selected from the group of H, unsubstituted —(C₁₋₅ alkyl), unsubstituted —(C₂_s alkenyl), and unsubstituted —(C₂_s alkynyl);

each R⁶ is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —OH, and —CN;

each R⁷ is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —OH, and —CN;

R⁸ is selected from the group of H, unsubstituted —(C₁₋₆ alkyl), unsubstituted —(C₂₋₆ alkenyl), unsubstituted —(C₂₋₆ alkynyl), and —(C₁₋₄ alkylene)ₚheterocyclyl optionally substituted with 1-10 R⁶; wherein —(C₁₋₄ alkylene) is optionally substituted with one or more substituents as defined anywhere herein;

each R⁹ is independently selected from the group of H, unsubstituted —(C₁₋₅ alkyl), unsubstituted —(C₂₋₅ alkenyl), and unsubstituted —(C₂₋₅ alkynyl), and —(C₁₋₄ alkylene)ₚheterocyclyl optionally substituted with 1-10 R⁶; wherein —(C₁₋₄ alkylene) is optionally substituted with one or more substituents as defined anywhere herein;

each R¹⁰ is independently selected from the group of unsubstituted —(C₁₋₅ alkyl), unsubstituted —(C₂₋₅ alkenyl), and unsubstituted —(C₂₋₅ alkynyl), and —(C₁₋₄ alkylene)ₚheterocyclyl optionally substituted with 1-10 R⁶; wherein —(C₁₋₄ alkylene) is optionally substituted with one or more substituents as defined anywhere herein; and

each p is independently 0 or 1.

In some embodiments of any of the methods described herein, the CLK inhibitor is a compound of Formula (IX)

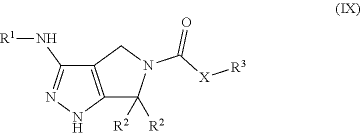

as well as prodrugs and pharmaceutically acceptable salt or solvate thereof, wherein:

R¹ is -heteroaryl optionally substituted with 1-6 R⁴;

each R² is independently selected from the group of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

R³ is —CH(R⁵)R⁶;

each R⁴ is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OR⁷, -carbocyclyl optionally substituted with 1-12 R;

R⁵ is -aryl optionally substituted with 1-5 R⁹;

R⁶ is —(C₁₋₄ alkylene)N(R¹⁰)₂; wherein —(C₁₋₄ alkylene) is optionally substituted with one or more substituents as defined anywhere herein;

each R⁷ is independently selected from the group of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

each R⁸ is independently selected from the group of halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);

each R⁹ is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, and —OR⁷;

each R¹⁰ is independently selected from the group of H, unsubstituted —(C₁₋₅ alkyl), unsubstituted —(C₂₋₅ alkenyl), and unsubstituted —(C₂₋₅ alkynyl); and

X is selected from the group of O, S, and NH.

In some embodiments of any of the methods described herein, the CLK inhibitor is a compound of Formula (X)

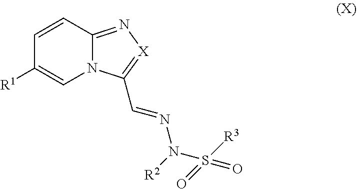

as well as prodrugs and pharmaceutically acceptable salt or solvate thereof, wherein:

R¹ is selected from the group of H, halide, unsubstituted —(C₁₋₅ alkyl), unsubstituted —(C₁₋₅ haloalkyl), and —CN;

R² is selected from the group of H, unsubstituted —(C₁₋₅ alkyl), unsubstituted —(C₂₋₅ alkenyl), and unsubstituted —(C₂₋₅ alkynyl);

R³ is -aryl optionally substituted with 1-5 R⁴;

each R⁴ is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —NO₂, —CN, and —OMe;

R⁵ is selected from the group of H, unsubstituted —(C₁₋₅alkyl), unsubstituted —(C₂₋₅ alkenyl), unsubstituted —(C₂₋₅ alkynyl), and unsubstituted —(C₁₋₅ haloalkyl); and

X is selected from the group of N and CR⁵.

In some embodiments of any of the methods described herein, the CLK inhibitor is a compound of Formula (XI)

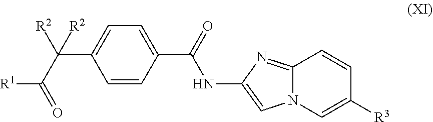

as well as prodrugs and pharmaceutically acceptable salt or solvate thereof, wherein:

R¹ is —N(R⁴)₂;

R² is selected from the group of H, unsubstituted —(C₁₋₅alkyl), unsubstituted —(C₂₋₅ alkenyl), unsubstituted —(C₂₋₅ alkynyl), and unsubstituted —(C₁₋₅ haloalkyl);

R³ is -heteroaryl optionally substituted with 1-6 R⁵;

each R⁴ is independently selected from the group of H, unsubstituted —(C₁₋₅ alkyl), unsubstituted —(C₂₋₅ alkenyl), unsubstituted —(C₂₋₅ alkynyl), and -heterocyclyl optionally substituted with 1-10 R⁶;

alternatively, two adjacent R⁴ are taken together to form a ring which is selected from the group of -heterocyclyl optionally substituted with 1-10 R⁶;

each R⁵ is independently selected from the group of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, and —OMe; and

each R⁶ is independently selected from the group of halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl).

In some embodiments of any of the methods described herein, the CLK inhibitor is a compound of Formula (XII)

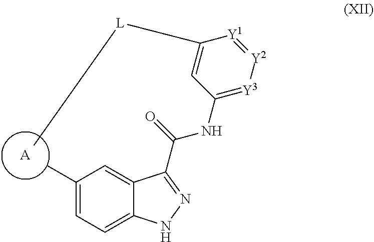

as well as prodrugs and pharmaceutically acceptable salt or solvate thereof, wherein:

Ring A is a 5-6-membered heteroaryl optionally substituted with 1-3 R¹;

L is -L¹-L²-L³-L⁴-

L¹ is selected from the group consisting of unsubstituted —(C₁₋₃ alkylene)-, —NR²—, —NR³(C═O)—, —(C═O)NR³—, and —O—;

L² is selected from the group consisting of unsubstituted —(C₁₋₆ alkylene)-, —NR²—, —NR³(C═O)—, and —(C═O)NR³—;

L³ is selected from the group consisting of unsubstituted —(C₁₋₆ alkylene)-, —O—, and carbocyclylene optionally substituted with one or more halides;

L⁴ is selected from the group consisting of unsubstituted —(C₁₋₆ alkylene)-, —O—, —NR²—, —NR³(C═O)—, —(C═O)NR³—, -arylene substituted with 1-5 R⁴, and -heteroarylene optionally substituted with 1-4 R⁵;

with the proviso that —NR²— and —O— are not adjacent to each other;

with the proviso that two —NR³(C═O)— and/or —(C═O)NR³—, are not adjacent to each other;

each R¹ is selected from the group consisting of halide, unsubstituted —(C₁₋₃ alkyl), unsubstituted —(C₁₋₃ haloalkyl), and —CN;

each R² is selected from the group consisting of H and unsubstituted —(C₁₋₆ alkyl);

each R³ is selected from the group consisting of H and unsubstituted —(C₁₋₆ alkyl);

each R⁴ is selected from the group consisting of halide, unsubstituted —(C₁₋₆ alkyl), unsubstituted —(C₁₋₆ haloalkyl), and —CN;

each R⁵ is selected from the group consisting of halide, unsubstituted —(C₁₋₆ alkyl), unsubstituted —(C₁₋₆ haloalkyl), and —CN;

Y¹, Y², and Y³ are independently selected from the group consisting of carbon and nitrogen; wherein

if Y¹ is nitrogen then Y² and Y³ are CH;

if Y² is nitrogen then Y¹ and Y³ are CH; and

if Y³ is nitrogen then Y¹ and Y² are CH.

It is to be understood that both the foregoing general description and the following detailed description are exemplary and explanatory only and are not restrictive of the disclosure, as claimed.

As used herein, “Wnt pathway activity” is an art-known term and generally refers to one or more direct Wnt/p-catenin activities in a mammalian cell and/or one or more indirect activities of Wnt/β-catenin (downstream activities resulting from Wnt/p-catenin activity) in a mammalian cell. Non-limiting examples of Wnt pathway activities include the level of expression of one or more Wnt-upregulated genes (e.g., one or more of any of the exemplary Wnt-upregulated genes described herein) in a mammalian cell, the level of β-catenin present in a nucleus of a mammalian cell, the level of expression of one or more of CLK1, CLK2, CLK3, CLK4, and β-catenin in a mammalian cell, detection of a gain-of-function mutation in a β-catenin gene, and detection of one or more of a loss-of-function mutation in one or more of a AXIN gene, a AXIN2 gene, a APC gene, a CTNNB1 gene, a Tsc1 gene, a Tsc2 gene, and a GSK3p gene. Methods for detecting a level of each of these exemplary types of Wnt pathway activity are described herein. Additional examples of Wnt pathway activities are known in the art, as well as methods for detecting a level of the same.

As used herein, “gain-of-function mutation” means one or more nucleotide substitutions, deletions, and/or insertions in a gene that results in: an increase in the level of expression of the encoded protein as compared to the level of the expression by the corresponding wildtype gene, and/or the expression of a protein encoded by the gene that has one or more increased activities in a mammalian cell as compared to the version of the protein encoded by the corresponding wildtype gene.

As used herein, “loss-of-function mutation” means one or more nucleotide substitutions, deletions, and/or insertions in a gene that results in: a decrease in the level of expression of the encoded protein as compared to the level of the expression by the corresponding wildtype gene, and/or the expression of a protein encoded by the gene that has one or more decreased activities in a mammalian cell as compared to the version of the protein encoded by the corresponding wildtype gene.

As used herein, “Wnt-upregulated gene” means a gene that exhibits an increased level of transcription when the Wnt/β-catenin signaling pathway is active in a mammalian cell. Non-limiting examples of Wnt-upregulated genes are described herein. Additional examples of Wnt-upregulated genes are known in the art. Exemplary methods of detecting the level of expression of Wnt-upregulated genes are described herein. Additional methods of detecting the level of expression of Wnt-upregulated genes are known in the art.

As used herein, “CLK inhibitor” refers to an agent (e.g., compound) that decreases the catalytic activity of one or more of CLK1, CLK2, CLK3, and CLK4 with an IC₅₀ of about 1 nM to about 10 μM (or any of the subranges of this range described herein) (e.g., determined using the exemplary in vitro assays for determining CLK1, CLK2, CLK3, and CLK4 activities described in the Examples).

As used herein, “a multi-isoform CLK inhibitor” refers to an agent (e.g., a compound that decreases the catalytic activity of two or more of CLK1, CLK2, CLK3, and CLK4 with an IC₅₀ of about 1 nM to about 10 μM (or any of the subranges of this range described herein) (e.g., determined using the exemplary in vitro assays for determining CLK1, CLK2, CLK3, and CLK4 activities described in the Examples).

As used herein, “altering mRNA splicing” means (i) changing the relative expression levels of two or more different isoforms of a protein in a mammalian cell that are encoded by the same gene, wherein the different isoforms of the protein result from mRNA splicing in the mammalian cell; and/or (ii) changing the level of activity, phosphorylation, and/or expression of one or more splicing factors in a mammalian cell.

As used herein, “aberrant mRNA splicing” means a mammalian cell that has been identified as having (i) a different relative expression levels of two or more different isoforms of a protein in a mammalian cell that are encoded by the same gene, wherein the different isoforms of the protein result from mRNA splicing in the mammalian cell; and/or (ii) a different level of activity, phosphorylation, and/or expression of one or more splicing factors, e.g., as compared to a reference level (e.g., the level in a healthy, non-cancerous cell or a corresponding non-cancerous cell).

As used herein, “alkyl” means a branched or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl, and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen, and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen, and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched, or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene, and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched, or straight chain chemical group containing only carbon and hydrogen, and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched, or straight chain chemical group containing only carbon and hydrogen, and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “alkoxy” means an alkyl-O— group in which the alkyl group is as described herein. Exemplary alkoxy groups include methoxy, ethoxy, n-propoxy, i-propoxy, n-butoxy, s-butoxy, t-butoxy, pentoxy, hexoxy, and heptoxy, and also the linear or branched positional isomers thereof.

As used herein, “haloalkoxy” means a haloalkyl-O— group in which the haloalkyl group is as described herein. Exemplary haloalkoxy groups include fluoromethoxy, difluoromethoxy, and trifluoromethoxy, and also the linear or branched positional isomers thereof.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; where at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “arylalkylene” means an aryl-alkylene- group in which the aryl and alkylene moieties are as previously described. In some embodiments, arylalkylene groups contain a C₁₋₄alkylene moiety. Exemplary arylalkylene groups include benzyl and 2-phenethyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide,” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched, alkyl, alkenyl, or alkynyl substituted with one or more chloro, bromo, fluoro, and/or iodo atom(s).

In some embodiments, a haloalkyl is a fluoroalkyls, where one or more of the hydrogen atoms have been substituted by fluoro. In some embodiments, haloalkyls are of 1 to about 3 carbons in length (e.g., 1 to about 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused rings. Heterocyclyls may be substituted or unsubstituted with one or more substituents. In some embodiments, heterocycles have 3-11 members. In six-membered monocyclic heterocycles, the heteroatom(s) are selected from one to three of O, N, or S, and where, when the heterocycle is five-membered, it can have one or two heteroatoms selected from O, N, or S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents. In some embodiments, heterocycles have 3-7 members. In six-membered monocyclic heterocycles, the heteroatom(s) are selected from one to three of O, N, or S, and where, when the heterocycle is five-membered, it can have one or two heteroatoms selected from O, N, or S. Examples of heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “bicyclic heterocyclyl” means a nonaromatic bicyclic ring system comprising at least one heteroatom in the ring system backbone. Bicyclic heterocyclyls may be substituted or unsubstituted with one or more substituents. In some embodiments, bicyclic heterocycles have 4-11 members with the heteroatom(s) being selected from one to five of 0, N, or S. Examples of bicyclic heterocyclyls include 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, and the like.

As used herein, “spirocyclic heterocyclyl” means a nonaromatic bicyclic ring system comprising at least one heteroatom in the ring system backbone and with the rings connected through just one atom. Spirocyclic heterocyclyls may be substituted or unsubstituted with one or more substituents. In some embodiments, spirocyclic heterocycles have 5-11 members with the heteroatom(s) being selected from one to five of O, N, or S. Examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 2,5-diazaspiro[3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R″; —NRR′; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′, and R″ are independently selected from H; —(C₁₋₉ alkyl); C₆₋₁₀ aryl optionally substituted with from 1-3R′″; 5-10 membered heteroaryl having from 1-4 heteroatoms independently selected from N, O, and S and optionally substituted with from 1-3 R′″; C₃_₇ carbocyclyl optionally substituted with from 1-3 R′″; and 3-8 membered heterocyclyl having from 1-4 heteroatoms independently selected from N, O, and S, and optionally substituted with from 1-3 R′″; where each R′″ is independently selected from —(C₁₋₆ alkyl), —(C₁₋₆ haloalkyl), a halide (e.g., F), a hydroxyl, —C(O)OR, —C(O)R, —(C₁₋₆alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring,” it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formulas (I)-(XII) wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), carbon, such as ¹¹C, ¹³C and ¹⁴C, chlorine, such as ³⁶Cl, fluorine, such as 18F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

The term “administration” or “administering” refers to a method of providing a dosage of a compound or pharmaceutical composition to a vertebrate or invertebrate, including a mammal, a bird, a fish, or an amphibian, where the method is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” or “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated.

Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Brunton et al. (Eds.) (2017); Goodman and Gilman's: The Pharmacological Basis of Therapeutics, 13th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally-occurring substituted amines, cyclic amines, basic ion exchange resins, and the like, specifically such as isopropylamine, trimethylamine, diethylamine, triethylamine, tripropylamine, and ethanolamine.

The term “subject” is defined herein to include animals such as mammals, including but not limited to, mice, rats, rabbits, dogs, cats, horses, goats, sheep, pigs, goats, cows, primates (e.g., humans), and the like. In preferred embodiments, the subject is a human. In some embodiments of any of the methods described herein, a subject may be referred to as a patient. In some embodiments of any of the methods described herein, the subject is 1 year old or older, 5 years old or older, 10 years old or older, 15 years old or older, 18 years old or older, 20 years old or older, 25 years old or older, 30 years old or older, 35 years old or older, 40 years old or older, 45 years old or older, 50 years old or older, 55 years old or older, 60 years old or older, 65 years old or older, 70 years old or older, 75 years old or older, 80 years old or older, 85 years old or older, 90 years old or older, 95 years old or older, 100 years old or older, or 105 years old or older.

In some embodiments of any of the methods described herein, the subject has been previously diagnosed or identified as having a cancer (e.g., any of the types of cancer described herein or known in the art). In some embodiments of any of the methods described herein, the subject is suspected of having a cancer (e.g., any of the types of cancer described herein or known in the art). In some embodiments of any of the methods described herein, the subject is presenting with one or more (e.g., two, three, four, five, or six) symptoms of a cancer (e.g., any of the types of cancer described herein or known in the art). In some embodiments, the cancer can be selected from the group of: a small cell lung cancer, a colorectal cancer, a head and neck cancer, an ovarian cancer, a melanoma, a renal cell carcinoma, a pancreatic cancer, or a non-small cell lung cancer.

In some embodiments of any of the methods described herein, the subject is a participant in a clinical trial. In some embodiments, the subject has been previously administered a different pharmaceutical composition and the different pharmaceutical composition was determined not to be therapeutically effective.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formulas (I)-(XII) in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age and medical history.

A therapeutic effect relieves, to some extent, one or more of the symptoms of the disease.

“Treat,” “treatment,” or “treating,” as used herein refers administering a compound (e.g., any of the compounds described herein) or treatment to a patient already suffering from a disease thus causing a therapeutically beneficial effect, such as ameliorating one or more existing symptoms, ameliorating the underlying metabolic causes of symptoms, postponing the further development of a disorder, and/or reducing the severity of one or more symptoms that will or are expected to develop.

The phrase “an elevated” or “an increased level” as used herein can be an increase of at least 1% (e.g., at least 2%, at least 4%, at least 6%, at least 8%, at least 10%, at least 12%, at least 14%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 120%, at least 130%, at least 140%, at least 150%, at least 160%, at least 170%, at least 180%, at least 190%, at least 200%, at least 250%, at least 300%, at least 350%, at least 400%, at least 450%, at least 500%, between 1% and 500%, between 1% and 450%, between 1% and 400%, between 1% and 350%, between 1% and 300%, between 1% and 250%, between 1% and 200%, between 1% and 180%, between 1% and 160%, between 1% and 140%, between 1% and 120%, between 1% and 100%, between 1% and 95%, between 1% and 90%, between 1% and 85%, between 1% and 80%, between 1% and 75%, between 1% and 70%, between 1% and 65%, between 1% and 60%, between 1% and 55%, between 1% and 50%, between 1% and 45%, between 1% and 40%, between 1% and 35%, between 1% and 30%, between 1% and 25%, between 1% and 20%, between 1% and 15%, between 1% and 10%, between 1% and 5%, between 5% and 500%, between 5% and 450%, between 5% and 400%, between 5% and 350%, between 5% and 300%, between 5% and 250%, between 5% and 200%, between 5% and 180%, between 5% and 160%, between 5% and 140%, between 5% and 120%, between 5% and 100%, between 5% and 95%, between 5% and 90%, between 5% and 85%, between 5% and 80%, between 5% and 75%, between 5% and 70%, between 5% and 65%, between 5% and 60%, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%, between 10% and 500%, between 10% and 450%, between 10% and 400%, between 10% and 350%, between 10% and 300%, between 10% and 250%, between 10% and 200%, between 10% and 180%, between 10% and 160%, between 10% and 140%, between 10% and 120%, between 10% and 100%, between 10% and 95%, between 10% and 90%, between 10% and 85%, between 10% and 80%, between 10% and 75%, between 10% and 70%, between 10% and 65%, between 10% and 60%, between 10% and 55%, between 10% and 50%, between 10% and 45%, between 10% and 40%, between 10% and 35%, between 10% and 30%, between 10% and 25%, between 10% and 20%, between 10% and 15%, between 20% and 500%, between 20% and 450%, between 20% and 400%, between 20% and 350%, between 20% and 300%, between 20% and 250%, between 20% and 200%, between 20% and 180%, between 20% and 160%, between 20% and 140%, between 20% and 120%, between 20% and 100%, between 20% and 95%, between 20% and 90%, between 20% and 85%, between 20% and 80%, between 20% and 75%, between 20% and 70%, between 20% and 65%, between 20% and 60%, between 20% and 55%, between 20% and 50%, between 20% and 45%, between 20% and 40%, between 20% and 35%, between 20% and 30%, between 20% and 25%, between 30% and 500%, between 30% and 450%, between 30% and 400%, between 30% and 350%, between 30% and 300%, between 30% and 250%, between 30% and 200%, between 30% and 180%, between 30% and 160%, between 30% and 140%, between 30% and 120%, between 30% and 100%, between 30% and 95%, between 30% and 90%, between 30% and 85%, between 30% and 80%, between 30% and 75%, between 30% and 70%, between 30% and 65%, between 30% and 60%, between 30% and 55%, between 30% and 50%, between 30% and 45%, between 30% and 40%, between 30% and 35%, between 40% and 500%, between 40% and 450%, between 40% and 400%, between 40% and 350%, between 40% and 300%, between 40% and 250%, between 40% and 200%, between 40% and 180%, between 40% and 160%, between 40% and 140%, between 40% and 120%, between 40% and 100%, between 40% and 95%, between 40% and 90%, between 40% and 85%, between 40% and 80%, between 40% and 75%, between 40% and 70%, between 40% and 65%, between 40% and 60%, between 40% and 55%, between 40% and 50%, between 40% and 45%, between 50% and 500%, between 50% and 450%, between 50% and 400%, between 50% and 350%, between 50% and 300%, between 50% and 250%, between 50% and 200%, between 50% and 180%, between 50% and 160%, between 50% and 140%, between 50% and 120%, between 50% and 100%, between 50% and 95%, between 50% and 90%, between 50% and 85%, between 50% and 80%, between 50% and 75%, between 50% and 70%, between 50% and 65%, between 50% and 60%, between 50% and 55%, between 60% and 500%, between 60% and 450%, between 60% and 400%, between 60% and 350%, between 60% and 300%, between 60% and 250%, between 60% and 200%, between 60% and 180%, between 60% and 160%, between 60% and 140%, between 60% and 120%, between 60% and 100%, between 60% and 95%, between 60% and 90%, between 60% and 85%, between 60% and 80%, between 60% and 75%, between 60% and 70%, between 60% and 65%, between 70% and 500%, between 70% and 450%, between 70% and 400%, between 70% and 350%, between 70% and 300%, between 70% and 250%, between 70% and 200%, between 70% and 180%, between 70% and 160%, between 70% and 140%, between 70% and 120%, between 70% and 100%, between 70% and 95%, between 70% and 90%, between 70% and 85%, between 70% and 80%, between 70% and 75%, between 80% and 500%, between 80% and 450%, between 80% and 400%, between 80% and 350%, between 80% and 300%, between 80% and 250%, between 80% and 200%, between 80% and 180%, between 80% and 160%, between 80% and 140%, between 80% and 120%, between 80% and 100%, between 80% and 95%, between 80% and 90%, between 80% and 85%, between 90% and 500%, between 90% and 450%, between 90% and 400%, between 90% and 350%, between 90% and 300%, between 90% and 250%, between 90% and 200%, between 90% and 180%, between 90% and 160%, between 90% and 140%, between 90% and 120%, between 90% and 100%, between 90% and 95%, between 100% and 500%, between 100% and 450%, between 100% and 400%, between 100% and 350%, between 100% and 300%, between 100% and 250%, between 100% and 200%, between 100% and 180%, between 100% and 160%, between 100% and 140%, between 100% and 120%, between 120% and 500%, between 120% and 450%, between 120% and 400%, between 120% and 350%, between 120% and 300%, between 120% and 250%, between 120% and 200%, between 120% and 180%, between 120% and 160%, between 120% and 140%, between 140% and 500%, between 140% and 450%, between 140% and 400%, between 140% and 350%, between 140% and 300%, between 140% and 250%, between 140% and 200%, between 140% and 180%, between 140% and 160%, between 160% and 500%, between 160% and 450%, between 160% and 400%, between 160% and 350%, between 160% and 300%, between 160% and 250%, between 160% and 200%, between 160% and 180%, between 180% and 500%, between 180% and 450%, between 180% and 400%, between 180% and 350%, between 180% and 300%, between 180% and 250%, between 180% and 200%, between 200% and 500%, between 200% and 450%, between 200% and 400%, between 200% and 350%, between 200% and 300%, between 200% and 250%, between 250% and 500%, between 250% and 450%, between 250% and 400%, between 250% and 350%, between 250% and 300%, between 300% and 500%, between 300% and 450%, between 300% and 400%, between 300% and 350%, between 350% and 500%, between 350% and 450%, between 350% and 400%, between 400% and 500%, between 400% and 450%, or about 450% to about 500%), e.g., as compared to a reference level (e.g., any of the exemplary reference levels described herein).

As used herein, a “first time point” can, e.g., refer to a designated time point, which can, e.g., be used to refer to chronologically later time points (e.g., a second time point). In some examples, a subject may not have yet received a treatment at a first time point (e.g., may not have yet received a dose of a CLK inhibitor (e.g., any of the CLK inhibitors described herein) at a first time point). In some examples, a subject may have already received a treatment that does not include a CLK inhibitor at the first time point. In some examples, the previous treatment that does not include a CLK inhibitor was identified as being ineffective prior to the first time point. In some examples, a subject has previously been identified or diagnosed as having a cancer (e.g., any of the types of cancer described herein or known in the art) at the first time point. In some examples, a subject has previously been suspected of having a cancer (e.g., any of the types of cancer described herein or known in the art) at the first time point. In other examples, a first time point can be a time point when a subject has developed at least one (e.g., 1, 2, 3, 4, 5, 6, 7, 8, 9, or 10) symptom(s) associated with a cancer and has not yet received any treatment for cancer.

As used herein, a “second time point” refers to a time point that occurs chronologically after a first designated time point. In some examples, a subject (e.g., any of the subjects described herein) can receive or has received at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) doses of a treatment (e.g., a CLK inhibitor (e.g., any of the CLK inhibitors described herein)) between the first and the second time points. In some examples, the time difference between a first and a second time point can be, e.g., 1 day to about 12 months, 1 day to about 11 months, 1 day to about 10 months, 1 day to about 9 months, 1 day to about 8 months, 1 day to about 7 months, 1 day to about 6 months, 1 day to about 22 weeks, 1 day to about 20 weeks, 1 day to about 18 weeks, 1 day to about 16 weeks, 1 day to about 14 weeks, 1 day to about 12 weeks, 1 day to about 10 weeks, 1 day to about 8 weeks, 1 day to about 6 weeks, 1 day to about 4 weeks, 1 day to about 3 weeks, 1 day to about 2 weeks, 1 day to about 1 week, about 2 days to about 12 months, about 2 days to about 11 months, about 2 days to about 10 months, about 2 days to about 9 months, about 2 days to about 8 months, about 2 days to about 7 months, about 2 days to about 6 months, about 2 days to about 22 weeks, about 2 days to about 20 weeks, about 2 days to about 18 weeks, about 2 days to about 16 weeks, about 2 days to about 14 weeks, about 2 days to about 12 weeks, about 2 days to about 10 weeks, about 2 days to about 8 weeks, about 2 days to about 6 weeks, about 2 days to about 4 weeks, about 2 days to about 3 weeks, about 2 days to about 2 weeks, about 2 days to about 1 week, about 4 days to about 12 months, about 4 days to about 11 months, about 4 days to about 10 months, about 4 days to about 9 months, about 4 days to about 8 months, about 4 days to about 7 months, about 4 days to about 6 months, about 4 days to about 22 weeks, about 4 days to about 20 weeks, about 4 days to about 18 weeks, about 4 days to about 16 weeks, about 4 days to about 14 weeks, about 4 days to about 12 weeks, about 4 days to about 10 weeks, about 4 days to about 8 weeks, about 4 days to about 6 weeks, about 4 days to about 4 weeks, about 4 days to about 3 weeks, about 4 days to about 2 weeks, about 4 days to about 1 week, about 1 week to about 12 months, about 1 week to about 11 months, about 1 week to about 10 months, about 1 week to about 9 months, about 1 week to about 8 months, about 1 week to about 7 months, about 1 week to about 6 months, about 1 week to about 22 weeks, about 1 week to about 20 weeks, about 1 week to about 18 weeks, about 1 week to about 16 weeks, about 1 week to about 14 weeks, about 1 week to about 12 weeks, about 1 week to about 10 weeks, about 1 week to about 8 weeks, about 1 week to about 6 weeks, about 1 week to about 4 weeks, about 1 week to about 3 weeks, about 1 week to about 2 weeks, about 2 weeks to about 12 months, about 2 weeks to about 11 months, about 2 weeks to about 10 months, about 2 weeks to about 9 months, about 2 weeks to about 8 months, about 2 weeks to about 7 months, about 2 weeks to about 6 months, about 2 weeks to about 22 weeks, about 2 weeks to about 20 weeks, about 2 weeks to about 18 weeks, about 2 weeks to about 16 weeks, about 2 weeks to about 14 weeks, about 2 weeks to about 12 weeks, about 2 weeks to about 10 weeks, about 2 weeks to about 8 weeks, about 2 weeks to about 6 weeks, about 2 weeks to about 4 weeks, about 2 weeks to about 3 weeks, about 3 weeks to about 12 months, about 3 weeks to about 11 months, about 3 weeks to about 10 months, about 3 weeks to about 9 months, about 3 weeks to about 8 months, about 3 weeks to about 7 months, about 3 weeks to about 6 months, about 3 weeks to about 22 weeks, about 3 weeks to about 20 weeks, about 3 weeks to about 18 weeks, about 3 weeks to about 16 weeks, about 3 weeks to about 14 weeks, about 3 weeks to about 12 weeks, about 3 weeks to about 10 weeks, about 3 weeks to about 8 weeks, about 3 weeks to about 6 weeks, about 3 weeks to about 4 weeks, about 4 weeks to about 12 months, about 4 weeks to about 11 months, about 4 weeks to about 10 months, about 4 weeks to about 9 months, about 4 weeks to about 8 months, about 4 weeks to about 7 months, about 4 weeks to about 6 months, about 4 weeks to about 22 weeks, about 4 weeks to about 20 weeks, about 4 weeks to about 18 weeks, about 4 weeks to about 16 weeks, about 4 weeks to about 14 weeks, about 4 weeks to about 12 weeks, about 4 weeks to about 10 weeks, about 4 weeks to about 8 weeks, about 4 weeks to about 6 weeks, about 6 weeks to about 12 months, about 6 weeks to about 11 months, about 6 weeks to about 10 months, about 6 weeks to about 9 months, about 6 weeks to about 8 months, about 6 weeks to about 7 months, about 6 weeks to about 6 months, about 6 weeks to about 22 weeks, about 6 weeks to about 20 weeks, about 6 weeks to about 18 weeks, about 6 weeks to about 16 weeks, about 6 weeks to about 14 weeks, about 6 weeks to about 12 weeks, about 6 weeks to about 10 weeks, about 6 weeks to about 8 weeks, about 8 weeks to about 12 months, about 8 weeks to about 11 months, about 8 weeks to about 10 months, about 8 weeks to about 9 months, about 8 weeks to about 8 months, about 8 weeks to about 7 months, about 8 weeks to about 6 months, about 8 weeks to about 22 weeks, about 8 weeks to about 20 weeks, about 8 weeks to about 18 weeks, about 8 weeks to about 16 weeks, about 8 weeks to about 14 weeks, about 8 weeks to about 12 weeks, about 8 weeks to about 10 weeks, about 10 weeks to about 12 months, about 10 weeks to about 11 months, about 10 weeks to about 10 months, about 10 weeks to about 9 months, about 10 weeks to about 8 months, about 10 weeks to about 7 months, about 10 weeks to about 6 months, about 10 weeks to about 22 weeks, about 10 weeks to about 20 weeks, about 10 weeks to about 18 weeks, about 10 weeks to about 16 weeks, about 10 weeks to about 14 weeks, about 10 weeks to about 12 weeks, about 12 weeks to about 12 months, about 12 weeks to about 11 months, about 12 weeks to about 10 months, about 12 weeks to about 9 months, about 12 weeks to about 8 months, about 12 weeks to about 7 months, about 12 weeks to about 6 months, about 12 weeks to about 22 weeks, about 12 weeks to about 20 weeks, about 12 weeks to about 18 weeks, about 12 weeks to about 16 weeks, about 12 weeks to about 14 weeks, about 14 weeks to about 12 months, about 14 weeks to about 11 months, about 14 weeks to about 10 months, about 14 weeks to about 9 months, about 14 weeks to about 8 months, about 14 weeks to about 7 months, about 14 weeks to about 6 months, about 14 weeks to about 22 weeks, about 14 weeks to about 20 weeks, about 14 weeks to about 18 weeks, about 14 weeks to about 16 weeks, about 16 weeks to about 12 months, about 16 weeks to about 11 months, about 16 weeks to about 10 months, about 16 weeks to about 9 months, about 16 weeks to about 8 months, about 16 weeks to about 7 months, about 16 weeks to about 6 months, about 16 weeks to about 22 weeks, about 16 weeks to about 20 weeks, about 16 weeks to about 18 weeks, about 18 weeks to about 12 months, about 18 weeks to about 11 months, about 18 weeks to about 10 months, about 18 weeks to about 9 months, about 18 weeks to about 8 months, about 18 weeks to about 7 months, about 18 weeks to about 6 months, about 18 weeks to about 22 weeks, about 18 weeks to about 20 weeks, about 20 weeks to about 12 months, about 20 weeks to about 11 months, about 20 weeks to about 10 months, about 20 weeks to about 9 months, about 20 weeks to about 8 months, about 20 weeks to about 7 months, about 20 weeks to about 6 months, about 20 weeks to about 22 weeks, about 22 weeks to about 12 months, about 22 weeks to about 11 months, about 22 weeks to about 10 months, about 22 weeks to about 9 months, about 22 weeks to about 8 months, about 22 weeks to about 7 months, about 22 weeks to about 6 months, about 24 weeks to about 12 months, about 24 weeks to about 11 months, about 24 weeks to about 10 months, about 24 weeks to about 9 months, about 24 weeks to about 8 months, about 24 weeks to about 7 months, about 7 months to about 12 months, about 7 months to about 11 months, about 7 months to about 10 months, about 7 months to about 9 months, about 7 months to about 8 months, about 8 months to about 12 months, about 8 months to about 11 months, about 8 months to about 10 months, about 8 months to about 9 months, about 9 months to about 12 months, about 9 months to about 11 months, about 9 months to about 10 months, about 10 months to about 12 months, about 10 months to about 11 months, or about 11 months to about 12 months.

“Drug-eluting” and/or controlled release as used herein refers to any and all mechanisms, e.g., diffusion, migration, permeation, and/or desorption by which the drug(s) incorporated in the drug-eluting material pass therefrom over time into the surrounding body tissue.

“Drug-eluting material” and/or controlled release material as used herein refers to any natural, synthetic or semi-synthetic material capable of acquiring and retaining a desired shape or configuration and into which one or more drugs can be incorporated and from which incorporated drug(s) are capable of eluting overtime.

“Elutable drug” as used herein refers to any drug or combination of drugs having the ability to pass over time from the drug-eluting material in which it is incorporated into the surrounding areas of the body.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

Other features and advantages of the disclosure will be apparent from the following detailed description and figures, and from the claims.

### BRIEF DESCRIPTION OF DRAWINGS

FIG. 1A is a graph showing the percent inhibition using CLK2, CLK3, and CDK1 kinase IC₅₀s as determined by the Thermo Fisher Scientific Z-LYTE™ platform. Inhibitory concentration (IC₅₀) values were determined from dose response curves from n=4 experiments.

FIG. 1B is a kinase dendrogram of Compound 12. Kinases with IC₅₀ values 0.01-0.05 μM are marked by small circles, whereas larger circles represent more potent IC₅₀s of 0.001-0.01 μM.

FIG. 1C is a graph showing normalized luciferase activity in SW480 colon cancer cells stably expressing the Wnt-responsive TOPflash or the control luciferase reporter under the EFla promoter and treated with Compound 12 following an 8-point dose response. Luciferase activities were measured using Bright-Glo™. Data represent the mean of two or three replicates±standard error of mean (SEM).

FIG. 1D are graphs showing Wnt pathway gene expression (AXIN2 and LEF1) in HEK-293T cells treated with Compound 12 or PRI-724 at the indicated doses for 1 hour before stimulation with Wnt3a (200 ng/mL). Fold-change in gene expression relative to unstimulated DMSO (n=3 biological replicates, Mean±SD, **P<0.01, ***P<0.001, ****P<0.0001, unpaired student's t-test vs. stimulated DMSO).

FIG. 1E are graphs showing Wnt pathway gene expression (AXIN2 and LEF1) in HEK-293T cells treated with Compound 12 or PRI-724 at the indicated doses for 1 hour before stimulation with CHIR99021 (4 μM) for 20 hours. Fold-change in gene expression relative to unstimulated DMSO (n=3 biological replicates, Mean±SD, **P<0.01, ***P<0.001, ****P<0.0001, unpaired student's t-test vs. stimulated DMSO).

FIG. 2A is a graph showing the percent activity in SW480 cells treated with a 3-fold, 10-point titration of Compound 12 or PRI-724 (0.0005-10 μM) for ˜48 hrs. Data is representative from three independent assays performed in quadruplicate.

FIG. 2B is a graph showing LGR5 gene expression in IEC-6 rat small intestinal cells treated with Compound 12 or PRI-724 at various doses and stimulated with Wnt3a for 16 h. The fold-change relative to unstimulated DMSO is shown (n=3, Mean±SD, *P<0.05, **P<0.01, ***P<0.001, ****P<0.0001, unpaired student's t-test vs. ligand). Data is representative from two independent assays.

FIG. 2C is a graph showing LGR5 gene expression in IEC-6 cells treated with Compound 12 or PRI-724 at various doses and stimulated with CHIR99021 for 16 h. Fold-change relative to unstimulated DMSO is shown (n=3, Mean±SD, *P<0.05, **P<0.01, ***P<0.001, ****P<0.0001, unpaired student's t-test vs. ligand). Data is representative from two independent assays.

FIG. 3A is a set of immunofluorescent images of SW480 cells treated with Compound 12 at test concentration 3, 1, 0.3, 0.1, and 0.03 μM with Compound 12, or with Staurosporine at 0.1 M, and stained with the CellEvent™ Caspase 3/7 Green Detection Reagent to detect activated caspase 3/7 (green) and with Hoechst 33342 to stain nuclei (blue). Images are representative of two independent assays.

FIG. 3B is a bar graph showing the percent of the total number of cells containing active caspase 3/7 following exposure to 3, 1, or 0.3 μM Compound 12 for 48 hours. Data is representative of two independent assays (n=3 biological replicates, Mean±standard deviation (SD), *P<0.05, **P<0.01, ***P<0.001, ****P<0.0001, unpaired student's t-test).

FIG. 3C is an immunoblot showing survivin, MCL-1, and cleaved PARP protein expression in SW480 cells following treatment with Compound 12 at test concentrations of 10, 3, 1, 0.3, 0.1, or 0.03 μM for 48 hours. R-actin was used as the loading control. Data is representative of two independent assays.

FIG. 4 is an image of SW480 cells treated with Compound 12 at test concentrations of 1, 0.3, 0.1, or 0.03 μM for 72 hours on a 2% agarose gel with a GelRed nucleic acid stain visualized on a UV transilluminator. Cells were also treated with Staurosporine at 1 μM for 24 hours as a positive control. Image shown is from one experiment and is representative of data from two independent assays.

FIG. 5 is an immunoblot showing cytoplasmic and nuclear localization of CLK1 (˜57 kDa), CLK2 (˜60 kDa), CLK3 (˜59 kDa), and CLK4 (˜62 kDa) in SW480 CRC cells. Protein lysates from untreated SW480 cells were separated into nuclear and cytoplasmic fractions. The Western blots were performed with antibodies for CLK1, CLK2, CLK3, and CLK4. β-actin was used as a loading control.

FIG. 6A is an immunoblot showing phosphorylated SRSF6 and SRSF5 in SW480 cells treated as indicated for 1 hour. Total SRSF5 and R-actin blots were used as loading controls. The blots are representative of two experiments.

FIG. 6B is a set of representative immunofluorescence images (×100 magnification) from SW480 cells treated with Compound 12 as indicated for 6 hours. The cells were stained with a phospho-SC35 antibody (green) and a Hoechst 33342 nuclear stain (blue). Scale bar, 10 μm.

FIG. 6C is a set of bar graphs showing qRT-PCR analysis of Wnt pathway genes AXIN2, CTNNB1, LEF1, MYC, TCF7, and TCF7L2 in SW480 cells treated with Compound 12 at indicated concentrations for 24 hours. Fold-change relative to DMSO is shown (n=3 biological replicates per group, Mean±SD, **P<0.01; ***P<0.001, student's two-tailed t-test).

FIG. 6D is an immunoblot showing Wnt pathway-related protein expression in SW480 cells treated as indicated for 24 hours. The proteins were separated into nuclear and cytoplasmic fractions. GAPDH and Lamin B1 represent cytoplasmic and nuclear protein loading controls, respectively. The blots are representative of two experiments.

FIG. 6E is an immunoblot showing Wnt pathway-related protein expression in SW480 cells treated as indicated for 48 hours. The proteins were separated into nuclear and cytoplasmic fractions. GAPDH and Lamin B1 represent cytoplasmic and nuclear protein loading controls, respectively. The blots are representative of two experiments.

FIG. 7A is a graph showing the effects of Compound 12 on Nanostring nCounter® Wnt pathway gene array. Seventeen different CRC cell lines (COLO 320 HSR, C₂BBel, HuTu 80, COLO 205, SQ1417, HT29, RKO, HCT 15, SW620, DLD-1, LoVo, LS123, T84, SW480, LS513, and HCT 116) were treated with 1 μM of Compound 12for 20-24 hrs. Diagonal lines indicating 2-fold changes are shown for both upregulated (blue) and downregulated (red) genes. The genes with absolute fold-changes greater than 2 and significant (FDR adjusted p<0.05) have labels highlighted in green.

FIG. 7B are bar graphs showing qRT-PCR analysis of the top gene hits from FIG. 7A in SW480 cells treated with Compound 12 for 24 hours. The fold-change relative to DMSO is shown (n=3 biological replicates per group, Mean±SD, *P<0.05; **P<0.01; ***P<0.001, student's two-tailed t-test).

FIG. 7C is an immunoblot showing protein expression of hits identified in FIG. 7A. SW480 cells were treated as indicated for 24 hours and proteins were separated into nuclear and cytoplasmic fractions. GAPDH and Lamin B1 represent the cytoplasmic and nuclear protein loading controls, respectively. The blots are representative of two experiments.

FIG. 8A is an immunoblot showing SRSF6 protein expression in SW480 cells treated with Nontarget, SRSF5, or SRSF6 siRNA for 5 days. R-actin is a loading control. Blots are representative of two experiments.

FIG. 8B is an immunoblot showing SRSF5 protein expression in SW480 cells treated with Nontarget, SRSF5 or SRSF6 siRNA for 5 days. R-actin was used as a loading control. The blots are representative of two experiments.

FIG. 8C is an immunoblot showing phospho-SRSF protein expression in SW480 cells treated with Nontarget, SRSF5, or SRSF6 siRNA for 5 days. -actin was used as a loading control. The blots are representative of two experiments.

FIG. 8D is an immunoblot showing phospho-SR protein expression in SW480 cells treated with Nontarget, SRSF6 siRNA for 5 days. -actin was used as a loading control. The blots are representative of two experiments.

FIG. 9A is a set of representative immunofluorescence images (×100 magnification) of SW480 cells treated with indicated concentrations for 6 hours. The cells were stained with a phospho-SC35 antibody (green) and a Hoechst 33342 nuclear stain (blue). Scale bar, 10 μm.

FIG. 9B is two graphs showing percent activity (left) and cell viability (right) of SW480 cells treated with a 3-fold 10-point titration of doses of Compound 12, CC-671, or Harmine (0.0005-10 μM) for 48 hrs (Wnt reporter assay) or 4 days (cell viability assay). The data is representative from three independent assays performed in quadruplicate.

FIG. 9C is a set of bar graphs showing qRT-PCR analysis of Wnt pathway genes in SW480 cells treated with Compound 12 for 24 hours. The data are presented as Mean±SD (n=3 biological replicates per group. *P<0.05; **P<0.01; ***P<0.001, student's two-tailed t test).

FIG. 10A is set of bar graphs showing qRT-PCR analysis of top gene hits from the Nanostring assay in SW480 cells treated with Compound 12 for 24 hours. The data are presented as Mean±SD (n=3 biological replicates per group, ***P<0.001, student's two-tailed t test).

FIG. 10B is an immunoblot of the indicated proteins in cytoplasmic and nuclear fractions from SW480 cells. GAPDH blot is a cytoplasmic loading control and Lamin B1 blot is a nuclear loading control. The blots are representative of two experiments.

FIG. 11A is a bar graph showing CTNNB1 gene expression in SW480 cells treated with Nontarget, or CTN/pBI siRNA for 5 days. The fold-change relative to Nontarget control is shown (n=3 biological replicates per group, Mean±SD, **P<0.01; ***P<0.001, student's two-tailed t-test).

FIG. 11B is a bar graph showing CLK2 gene expression in SW480 cells treated with Nontarget or CLK2 siRNA for 5 days. The fold-change relative to Nontarget control is shown (n=3 biological replicates per group, Mean±SD, **P<0.01; ***P<0.001, student's two-tailed t-test).

FIG. 11C is a bar graph showing CLK3 gene expression in SW480 cells treated with Nontarget or CLK3 siRNA for 5 days. The fold-change relative to Nontarget control is shown (n=3 biological replicates per group, Mean±SD, **P<0.01; ***P<0.001, student's two-tailed t-test).

FIG. 11D is an immunoblot showing CLK2, CLK3, and β-catenin protein expression in siRNA-treated cells. R-actin was used as a loading control.

FIG. 11E is an immunoblot showing protein expression of phosphorylated and total SRSF6 in siRNA-treated cells. R-actin was used as a loading control.

FIG. 11F is a bar graph showing analysis of the TOPflash reporter activity of SW480 cells treated for 5 days as indicated.

FIG. 11G is a bar graph showing cell viability of SW480 cells treated for 5 days as indicated.

FIG. 11H is a set of bar graphs showing qRT-PCR analysis of Wnt pathway-related genes (AXIN2, BTRC, DVL2, LEF1, LRP5, MYC, TCF7, and TCFL2) in siRNA-treated SW480 cells. The fold-change relative to Nontarget control is shown (n=3 biological replicates per group, Mean SD, *P<0.05; **P<0.01; ***P<0.001, student's two-tailed t-test).

FIG. 11I is an immunoblot of nuclear and cytoplasmic-fractionated protein of genes identified in FIG. 11H in siRNA-treated SW480 cells. GAPDH, Lamin B1, and R-actin were used as loading controls. Each panel is representative of three independent experiments.

FIG. 12 is an immunoblot of cytoplasmic and nuclear protein from SW480 cells for CLK1. GAPDH blot was used as a cytoplasmic loading control and Lamin B1 blot was used as a nuclear loading control. The blots are representative of two experiments (n=3 biological replicates per group).

FIG. 13 is a set of bar graphs showing qRT-PCR analysis for LRP6, MAPK8, BTRC, and FRZB in SW480-TOPflash cells treated with Nontarget, CTNNB1, CLK2, or CLK3 siRNA for 5 days. The fold-change relative to DMSO is shown (n=3 biological replicates per group, Mean SD, *P<0.05; **P<0.01; ***P<0.001, student's two-tailed t-test).

FIG. 14A is an immunoblot showing nuclear protein expression of CLK3, CLK2, and CLK1 in CLK3-CRISPR clonal cell lines. Lamin B1 was used as a loading control. The blots are representative of two experiments.

FIG. 14B is an immunoblot showing phosphorylated and total SRSF6 in WT and CLK3 KO SW480 clonal cells. The blots are representative of two experiments.

FIG. 14C is a bar graph showing MYC gene expression levels in CLK3 CRISPR clonal cell lines as determined by qRT-PCR. The fold-change relative to Cas9 WT (n=5 replicates per each group, Mean±SEM, **P<0.01; ****P<0.0001, student's two-tailed t-test).

FIG. 14D is an immunoblot for nuclear protein MYC in CLK3 CRISPR clonal cell lines. Lamin B1 was used as a loading control. The blots are representative of two experiments. The relative band intensity of MYC was determined after normalization with each Lamin B1 band and averaging WT and CLK3 KO clones (Mean±SEM, *P<0.05, student's two-tailed t-test).

FIG. 14E is a graph showing tumor growth curves of SW480 xenografts injected with WT, CLK3 KO clone 3, or CLK3 KO clone 5 cells. Tumor volumes were measured twice per week. Data presented as Mean±SEM (n=8-10 mice per group, ****P<0.0001, student's two-tailed t-test).

FIG. 14F are representative images of tumor pictures of WT and CLK3 KO clonal SW480 tumors at the end of study (day 28).

FIG. 14G is a bar graph showing CLK3 gene expression levels in WT and CLK3 KO clonal SW480 tumors at day 28 as determined by qRT-PCR. The data are presented as Mean±SEM (n=7-10 mice per each group. ****P<0.0001, student's two-tailed t-test).

FIG. 14H is an immunoblot for MYC in WT and CLK3 KO SW480 tumors collected at day 28. β-actin was used as a loading control. The relative band intensity of MYC was determined after normalization with each β-actin band and averaging WT and each CLK3 KO clonal tumors. The data are presented as Mean±SEM (*P<0.05, student's two-tailed t-test).

FIG. 15A is a graph showing cell growth of WT SW480 cells and CLK3 KO cells cultured in 10% FBS. BrdU cell proliferation ELISA was performed at day 4 and day 6 or 7 after plating the cells. The data are presented as Mean±SEM (n=3-10 biological replicates per group, ***P<0.001; ****P<0.0001, student's two-tailed t-test vs. WT).

FIG. 15B is a graph showing cell viability of WT SW480 cells and CLK3 KO cells cultured in 10% FBS. The data are presented as Mean±SEM (n=3-10 biological replicates per group, ***P <0.001; ****P<0.0001, student's two-tailed t-test vs. WT).

FIG. 15C is a graph showing cell growth of WT SW480 cells and CLK3 KO cells cultured in 1% FBS. BrdU cell proliferation ELISA was performed at day 4 and day 6 or 7 after plating the cells. The cells were adjusted to the low serum condition for two weeks before assays. CellTiter-Glo® luminescent cell viability assays were performed at day 4 and day 6 or 7 after plating the cells. The data are presented as Mean±SEM (n=3-10 biological replicates per group, ***P<0.001; ****P<0.0001, student's two-tailed t-test vs. WT).

FIG. 15D is a graph showing cell viability of WT SW480 cells and CLK3 KO cells cultured in 1% FBS. The cells were adjusted to the low-serum condition for two weeks before assays. CellTiter-Glo® luminescent cell viability assays were performed at day 4 and day 6 or 7 after plating the cells. The data are presented as Mean±SEM (n=3-10 biological replicates per group, ***P<0.001; ****P<0.0001, student's two-tailed t-test vs. WT).

FIG. 15E is a set of representative images of WT or CLK3 KO cells cultured for 5 days in 10% FBS media. The images are representative of data from two independent assays.

FIG. 15F are representative images of WT or CLK3 KO cells cultured for 5 days in 1% FBS media. The images are representative of data from two independent assays.

FIG. 16 is a graph showing mean plasma concentration versus time profiles of Compound 12. Following a single Intravenous (IV) Bolus or Oral (PO) Dose to Male Balb/c Mice, approximately 0.1 mL whole blood was collected via the cheek vein (submandibular) according to an alternate bleeding schedule (n=3/time point/route) at 0.083 (IV), 0.25, 0.5, 1, 2, 4, 6, 8, and 24 hours post-dose into tubes containing K₂EDTA anticoagulant and plasma was harvested by centrifugation.

FIG. 17A is a graph showing tumor volume in SW480 tumor-bearing mice that were orally administered doses of Compound 12, 6.25-25 mg/kg, at the indicated frequencies (QD=daily; QOD=every other day). Dosing was initiated when tumors were 100-200 mm³ in size and measurements performed twice per week. The data are presented as Mean±SEM (n=6-7 mice per each group. *P<0.05, student's t-test).

FIG. 17B is a graph showing tumor volume in HCT-116 tumor-bearing mice that were orally administered doses of Compound 12, 6.25-25 mg/kg, at the indicated frequencies (QD=daily; QOD=every other day). Dosing was initiated when tumors were 100-200 mm3 in size and measurements performed twice per week. The data are presented as Mean±SEM (n=6-7 mice per each group. *P<0.05, student's t-test).

FIG. 17C is a graph showing tumor volume in PDX-CR2545 (Crown Biosciences) tumor-bearing mice that were orally administered doses of Compound 12, 6.25-25 mg/kg, at the indicated frequencies (QD=daily; QOD=every other day). Dosing was initiated when tumors were 100-200 mm3 in size and measurements performed twice per week. The data are presented as Mean±SEM (n=6-7 mice per each group. *P<0.05, student's t-test).

FIG. 17D is an immunoblot showing tumor pharmacodynamics in athymic nude mice bearing SW480 tumors. After a single dose of Compound 12, tumors were harvested at 4, 8, and 24 hours and the effect on SR phosphorylation was evaluated, with total SRSF6, total SRSF5, and R-actin used as loading controls.

FIG. 17E is a set of graphs showing qRT-PCR analysis of Wnt pathways genes on RNA extracted from the SW480 tumors. The fold-change relative to vehicle is shown (n=3 biological replicates per group, Mean±SD, *P<0.05; **P<0.01; ***P<0.001, student's two-tailed t-test).

FIG. 18A is a graph showing the effect of Compound 12 on body weight in CRC-SW480 tumor-bearing athymic nude mice. CRC tumor xenograft-bearing mice were administered Compound 12 or vehicle by oral administration at the indicated doses and frequencies starting on day 0. Body weights in grams (g) were determined every 3-4 days. The data are presented as Mean±SEM (n=6-7 mice per each group.

*P<0.05, student's t-test). The percent body weight change represents the total change in body weight relative to the baseline body weight on day 0 prior to the first dose.

FIG. 18B is a graph showing the effect of Compound 12 on body weight in CRC-HCT116 tumor-bearing athymic nude mice. CRC tumor xenograft-bearing mice were administered Compound 12 or vehicle by oral administration at the indicated doses and frequencies starting on day 0. The body weights in grams (g) were determined every 3-4 days. The data are presented as Mean±SEM (n=6-7 mice per each group. *P<0.05, student's t-test). The percent body weight change represents the total change in body weight relative to the baseline body weight on day 0 prior to the first dose.

FIG. 18C is a graph showing the effect of Compound 12 on body weight in CRC-PDX CR2545 (Crown Biosciences) tumor-bearing Balb/c nude female mice. CRC tumor xenograft-bearing mice were administered Compound 12 or vehicle by oral administration at the indicated doses and frequencies starting on day 0. The body weights in grams (g) were determined every 3-4 days. The data are presented as Mean±SEM (n=6-7 mice per each group. *P<0.05, student's t-test). The percent body weight change represents the total change in body weight relative to the baseline body weight on day 0 prior to the first dose.

FIG. 19 is a set of bar graphs showing qRT-PCR analysis of central Wnt pathway genes on RNA extracted from SW480 tumors isolated 4, 8, and 24 hours after SW480 tumor-bearing athymic nude mice were given a single dose of Compound 12, 25 mg/kg. The data are presented as Mean SD (n=3 biological replicates per group, *P<0.05; ***P<0.001, student's two-tailed t-test).

FIG. 20 is a graph showing tumor volume in NCI-N87 GC tumor xenograft-bearing mice that were administered Compound 12 or vehicle by oral administration at the indicated doses and frequencies starting on day 0 to day 21 (22-day dosing period). QD=daily; QOD=every other day.

The tumor volumes were measured twice a week. Each data point represents Mean±SEM (n=7 mice per group, *P<0.05, student's two-tailed t-test).

FIGS. 21A-O are boxplots representing the distribution of log 2FC values for each compound across multiple cell lines. Compounds on the x-axis are sorted by average viability EC₅₀ across 50 cell lines (See Table 18), and each graph represents a single gene biomarker. A significant regression model (p<0.05) suggests gene expression differences are correlated with compound efficacy. Gene biomarkers represented are FIG. 21A, APC; FIG. 21B, TIAM1; FIG. 21C, CSNK2A1; FIG. 21D, CTGF; FIG. 21E, DVL2; FIG. 21F, FRZB; FIG. 21G, FZD6; FIG. 21H, GSK3B; FIG. 21I, HDAC3; FIG. 21J, LRP5; FIG. 21K, MYC; FIG. 21L, PLCB4; FIG. 21M, RUVBL1; FIG. 21N, SRSF5; and FIG. 21O, TCF7.

### DETAILED DESCRIPTION

The present disclosure is based on the discovery that Compound 12, a CDC-like kinase (CLK) inhibitor, modulates mRNA splicing in mammalian cells and downregulates Wnt signaling activity in cancer cells. In view of these discoveries, provided herein are methods of treating a cancer in a subject, methods of selecting a treatment for a subject, methods of selecting a subject for treatment, and methods of selecting a subject for participation in a clinical trial, that each include identifying a subject having a cancer cell (e.g., any of the types of cancer cell described herein) that has an elevated level of Wnt pathway activity as compared to a reference level. Also provided herein are methods of determining the efficacy of a CLK inhibitor in a subject that include detecting a level of Wnt/β-catenin signaling activity in a cancer cell obtained from the subject. Also provided are methods of decreasing the activity of one or more of CLK1, CLK2, CLK3, and CLK4 (e.g., in vitro or in a mammalian cell) that include the use of any of the CLK inhibitors or pharmaceutically acceptable salts or solvates thereof described herein. Also provided herein are methods of alternative mRNA splicing in a mammalian cell having aberrant mRNA splicing activity that include the use of any of the CLK inhibitors or pharmaceutically acceptable salts or solvates thereof described herein. Also provided herein are methods of treating a cancer using a CLK inhibitor, methods of selecting a treatment including a CLK inhibitor for a subject, methods of selecting a subject for treatment with a CLK inhibitor, and methods of selecting a subject for participation in a clinical trial, that each include the use of a CLK inhibitor, that include a step of identifying a subject having aberrant mRNA splicing activity.

Non-limiting aspects of these methods are described below and can be used in any combination without limitation. Additional aspects of these methods are known in the art.

### Methods of Treating—Type A

Provided herein are methods of treating a cancer (e.g., any of the exemplary cancers described herein or known in the art) in a subject that include: identifying a subject having a cancer cell that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein); and administering to the identified subject a therapeutically effective amount of a CLK inhibitor as well as prodrugs and pharmaceutically acceptable salt or solvate thereof (e.g., any of the exemplary CLK inhibitors described herein or known in the art).

Also provided herein are methods of treating a cancer in a subject that include: administering a therapeutically effective amount of a CLK inhibitor as well as prodrugs and pharmaceutically acceptable salt or solvate thereof (e.g., any of the exemplary CLK inhibitors described herein or known in the art) to a subject (e.g., any of the subjects described herein) identified as having a cancer cell that has an elevated level (e.g., an increase of 1% to about 500%, or any of the subranges of this range described herein) of Wnt pathway activity (e.g., any of the exemplary types of Wnt pathway activity described herein or known in the art) as compared to a reference level (e.g., any of the exemplary reference levels described herein).

Also provided herein are methods of treating a subject (e.g., any of the subjects described herein) having a cancer (e.g., any of the exemplary cancers described herein or known in the art) that include: (a) administering to the subject a therapeutic agent (e.g., any therapeutic agent that is not a CLK inhibitor or any therapeutic regimen that does not include a CLK inhibitor as a monotherapy); (b) after (a), identifying the subject as having a cancer cell that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the reference levels described herein); and (c) administering to the identified subject a treatment comprising a therapeutically effective amount of a CLK inhibitor as well as prodrugs and pharmaceutically acceptable salt or solvate thereof (e.g., any of the exemplary CLK inhibitors described herein or known in the art). In some embodiments, the subject is also administered the previously administered therapeutic agent (e.g., any cancer therapeutic agent known in the art, e.g., chemotherapy, radiation therapy, other kinase inhibitors, or a biologic), in addition to the CLK inhibitor or the pharmaceutically acceptable salt of solvate thereof.

Also provided herein are methods of treating a subject (e.g., any of the subjects described herein) having a cancer (e.g., any of the types of cancer described herein or known in the art) that include: identifying a subject previously administered a therapeutic agent (e.g., any therapeutic agent that is not a CLK inhibitor or any therapeutic regimen that does not include a CLK inhibitor as a monotherapy), as having a cancer cell that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein); and administering to the identified subject a treatment comprising a therapeutically effective amount of a CLK inhibitor as well as prodrugs and pharmaceutically acceptable salt or solvate thereof (e.g., any of the CLK inhibitors described herein or known in the art). In some embodiments, the subject is also administered the previously administered therapeutic agent (e.g., any cancer therapeutic agent known in the art, e.g., chemotherapy, radiation therapy, other kinase inhibitors, or a biologic), in addition to the CLK inhibitor or the pharmaceutically acceptable salt of solvate thereof.

Also provided herein are methods of treating a subject (e.g., any of the subjects described herein) having a cancer (e.g., any of the exemplary cancers described herein or known in the art) that include: administering to a subject previously administered a therapeutic agent (e.g., any therapeutic agent that is not a CLK inhibitor or any therapeutic regimen that does not include a CLK inhibitor as a monotherapy) and later identified as having an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein), a therapeutically effective amount of a CLK inhibitor as well as prodrugs and pharmaceutically acceptable salt or solvate thereof (e.g., any of the exemplary CLK inhibitors described herein or known in the art). In some embodiments, the subject is also administered the previously administered therapeutic agent (e.g., any cancer therapeutic agent known in the art, e.g., chemotherapy, surgery, radiation therapy, other kinase inhibitors, or a biologic), in addition to the CLK inhibitor or the pharmaceutically acceptable salt of solvate thereof.

Non-limiting types of Wnt pathway activity are described below and can be used in any of the methods of treatment described herein. For example, in some embodiments of any of the methods of treatment described herein, the Wnt pathway activity can be the level of CLK1, CLK2, CLK3, CLK4, or β-catenin expression, where an increased level (e.g., an increase of 1% to 500%, or any of the subranges of this range described herein) of expression of CLK1, CLK2, CLK3, CLK4, or β-catenin protein or mRNA expression, as compared to a reference level (e.g., any of the reference levels described herein) indicates an increased level of Wnt pathway activity.

For example, in some embodiments of any of the methods of treatment described herein, the Wnt pathway activity can be the level of β-catenin in the nucleus of a mammalian cell, where an increased level (e.g., an increase of 1% to 500%, or any of the subranges of this range described herein) of p-catenin in the nucleus of a mammalian cell as compared to a reference level (e.g., any of the reference levels described herein) indicates an increased level of Wnt pathway activity.

For example, in some embodiments of any of the methods of treatment described herein, the Wnt pathway activity can be detection of a mutation in a Wnt pathway gene selected from the group consisting of: gain-of-function mutation in a β-catenin gene (e.g., a gene encoding a β-catenin protein including a 41A, 45F, or 45P amino acid substitution, a mutation in exon 3, or deletion in exon 3) (Le Guellac et al., Modern Pathology 25: 1551, 2012), a loss-of-function mutation in an AXIN gene (e.g., c.178_1597del, c.266_1585del, c.355_1712del, c.1938_2704del, c.2168_3098del, c.2426_3101del, or c.2325_3106del, or a gene encoding an AXIN protein including a P218S, S226C, P263T, A360V, R382C, G433E, V517F, P661L, A740T, F824K, S828G, E842K, K397X, T58M, L101P, R103M, L106R, T122A, K203M, S215L, P263T, N370K, P345L, R349H, R353H, H394N, R395C, E41 iD, M4181, G425S, D495E, G583S, G650S, R841Q, P848L, E852G, W247X, Y305X, or E406X amino acid substitution (Mazzoni and Fearon, Cancer Lett 355(1): 1-8, 2014)), a loss-of-function mutation in an AXIN2 gene (e.g., c.1209insAT (V506X), c.1994delG (L688X), c.2013_2024del, or c.1926insA (E706X), or a gene encoding an AXIN2 protein including a S658C, R659W, Q696R, S738F, S762N, S738F, R656X, or W663X amino acid substitution (Mazzoni and Fearon, Cancer Lett 355(1): 1-8, 2014)), a loss-of-function mutation in a APC gene (e.g., 2-bp deletion in exon 7, 904C-T transition in exon 8, or 1-bp deletion in exon 10, or a gene encoding an APC protein including a R414C, R302X, S280X, Q1338X, Q541X, G1120E, R554X, or Y935X amino acid substitution), a loss-of-function mutation in a CTNNB1 gene (e.g., a gene encoding a CTNN11 protein including a Q558X or R710C amino acid substitution), a loss-of-function mutation in a Tsc1 gene (e.g., 4-bp deletion in exon 15, or a gene encoding a Tsc1 protein including a H732Y, K587R, M224R, L180P, R22W, or R204C amino acid substitution), a loss-of-function mutation in a Tsc2 gene (e.g., del5151OA or del4590C, or a gene encoding a Tsc2 protein including a K12X, R505X, R611Q, L717R, P1675L, Q2503P, R905Q, R905W, R905G, or V1547I amino acid substitution), and a loss-of-function mutation GSK3D gene.

For example, in some embodiments of any of the methods of treatment described herein, the Wnt pathway activity can be detection of an elevated level of expression of one or more Wnt-regulated genes as compared to a reference level (e.g., any of the reference levels described herein). Non-limiting examples of Wnt-upregulated genes include CCND1, CSNK2A1, CXCL12, LRP5, MMP7, MMP9, LEF1, AXIN2, MYC, TCF7L2, TCF7, LRP6, DVL2, BIRC, ERRB2, MAPK8, PKN1, AXIN2, ABCB1, ADAM10, ALEX1, ASCL2, BAMBI, BCL2L2, BIRC5, BMI1, BMP4, CCND1, CD44, CDKN2A, CDX1, CEBPD, CLDN1, COX2, DNMT1, EDN1, EFNB1, ENC1, EPHB2, EPHB3, FGF18, FGFBP, FRA1, FSCN1, FZD6, FZD7, FZD8, GAST, HDAC3, HEF1, HES1, ID2, ITF2, JAG1, JUN, LICAM, LAMC2, LGR5, MENA, MET, MMP14, MYB, MYCBP, NOS2, NOTCH2, NRCAM, PLAU, PLAUR, PLCB4, PPARD, RUVBL1, S100A4, S100A6, SGK1, SMC3, SOX9, SP5, SRSF3, SUZ12, TCF1, TIAM1, TIMP-1. TN-C, VEGF, WNT-5a, WNT-5b, WNT11, and YAP.

For example, in some embodiments of any of the methods of treatment described herein, the Wnt pathway activity can be detection of a decrease level of expression of one or more of APC, FRZB, CTGF, and GSK3B.

Non-limiting examples of Wnt-downregulated genes include secreted frizzled related protein 1 (FRP), disheveled associated activator of morphogenesis 1 (DAAM1) human ortholog of atonal 1 (HATH1), and cadherin 1 (CDH1). See, e.g., Slattery et al., Oncotarget 9(5): 6075-6085, 2018; Herbst et al., BMC Genomics 15:74, 2014. An elevated level of Wnt pathway activity can be detection of a decreased level of expression of one or more of these Wnt-downregulated genes (e.g., any of the Wnt-downregulated genes described herein or known in the art) as compared to a reference level (e.g., any of the reference levels described herein).

In some embodiments of any of the methods of treatment described herein, the cancer is a small cell lung cancer, a colorectal cancer, ahead and neck cancer, an ovarian cancer, a melanoma, a renal cell carcinoma, a pancreatic cancer, or a non-small cell lung cancer.

In some embodiments of any of the methods described herein, the method can result in an increased life span of the subject (e.g., as compared to a similar subject having a similar cancer but receiving a different treatment).

In some embodiments of any of the methods described herein, the cancer can be:

1) Breast cancers, including, for example ER⁺ breast cancer, ER⁻ breast cancer, her2⁻ breast cancer, her2⁺ breast cancer, stromal tumors, such as fibroadenomas, phyllodes tumors, and sarcomas, and epithelial tumors, such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, luminal B, basal A, basal B, and triple negative breast cancer, which is estrogen receptor negative (ER⁻), progesterone receptor negative, and Her2 negative (Her2⁻). In some embodiments, the breast cancer may have a high risk Oncotype score.

2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.

3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma.

4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, for example, hepatoma, e.g., hepatocellular carcinoma; cholangiocarcinoma; hepatoblastoma; angiosarcoma; hepatocellular adenoma; and hemangioma.

7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, oligodendrocytoma, schwannoma, retinoblastoma, and congenital tumors; and cancers of the spinal cord, e.g., neurofibroma, meningioma, glioma, and sarcoma.

9) Gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma.

10) Hematologic cancers, including, for example, cancers of the blood, e.g., acute myeloid leukemia, chronic myeloid leukemia, acute lymphoblastic leukemia, chronic lymphocytic leukemia, myeloproliferative diseases, chronic myeloid leukemia, multiple myeloma, and myelodysplastic syndrome, Hodgkin's lymphoma, non-Hodgkin's lymphoma (malignant lymphoma) and Waldenstrom's macroglobulinemia.

11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.

12) Adrenal gland cancers, including, for example, neuroblastoma.

More particularly, cancer in any of the methods described herein can be:

1) Astrocytic tumors, e.g., diffuse astrocytoma (fibrillary, protoplasmic, gemistocytic, mixed), anaplastic (malignant) astrocytoma, glioblastoma multiforme (giant cell glioblastoma and gliosarcoma), pilocytic astrocytoma (pilomyxoid astrocytoma), pleomorphic xanthoastrocytoma, subependymal giant cell astrocytoma, and gliomatosis cerebri.

2) Oligodendroglial tumors, e.g., oligodendroglioma and anaplastic oligodendroglioma.

3) Oligoastrocytic tumors, e.g., oligoastrocytoma and anaplastic oligoastrocytoma.

4) Ependymal tumors, e.g., subependymoma, myxopapillary ependymoma, ependymoma, (cellular, papillary, clear cell, tanycytic), and anaplastic (malignant) ependymoma.

5) Choroid plexus tumors, e.g., choroid plexus papilloma, atypical choroid plexus papilloma, and choroid plexus carcinoma.

6) Neuronal and mixed neuronal-glial tumors, e.g., gangliocytoma, ganglioglioma, dysembryoplastic neuroepithelial tumor (DNET), dysplastic gangliocytoma of the cerebellum (Lhermitte-Duclos), desmoplastic infantile astrocytoma/ganglioglioma, central neurocytoma, anaplastic ganglioglioma, extraventricular neurocytoma, cerebellar liponeurocytoma, Papillary glioneuronal tumor, Rosette-forming glioneuronal tumor of the fourth ventricle, and paraganglioma of the filum terminale.

7) Pineal tumors, e.g., pineocytoma, pineoblastoma, papillary tumors of the pineal region, and pineal parenchymal tumor of intermediate differentiation.

8) Embryonal tumors, e.g., medulloblastoma (medulloblastoma with extensive nodularity, anaplastic medulloblastoma, desmoplastic, large cell, melanotic, medullomyoblastoma), medulloepithelioma, supratentorial primitive neuroectodermal tumors, and primitive neuroectodermal tumors (PNETs) such as neuroblastoma, ganglioneuroblastoma, ependymoblastoma, and atypical teratoid/rhabdoid tumor.

9) Neuroblastic tumors, e.g., olfactory (esthesioneuroblastoma), olfactory neuroepithelioma, and neuroblastomas of the adrenal gland and sympathetic nervous system.

10) Glial tumors, e.g., astroblastoma, chordoid glioma of the third ventricle, and angiocentric glioma.

11) Tumors of cranial and paraspinal nerves, e.g., schwannoma, neurofibroma Perineurioma, and malignant peripheral nerve sheath tumor.

12) Tumors of the meninges such as tumors of meningothelial cells, e.g., meningioma (atypical meningioma and anaplastic meningioma); mesenchymal tumors, e.g., lipoma, angiolipoma, hibernoma, liposarcoma, solitary fibrous tumor, fibrosarcoma, malignant fibrous histiocytoma, leiomyoma, leiomyosarcoma, rhabdomyoma, rhabdomyosarcoma, chondroma, chondrosarcoma, osteoma, osteosarcoma, osteochondroma, haemangioma, epithelioid hemangioendothelioma, haemangiopericytoma, anaplastic haemangiopericytoma, angiosarcoma, Kaposi Sarcoma, and Ewing Sarcoma; primary melanocytic lesions, e.g., diffuse melanocytosis, melanocytoma, malignant melanoma, meningeal melanomatosis; and hemangioblastomas.

13) Tumors of the hematopoietic system, e.g., malignant Lymphomas, plasmocytoma, and granulocytic sarcoma.

14) Germ cell tumors, e.g., germinoma, embryonal carcinoma, yolk sac tumor, choriocarcinoma, teratoma, and mixed germ cell tumors.

15) Tumors of the sellar region, e.g., craniopharyngioma, granular cell tumor, pituicytoma, and spindle cell oncocytoma of the adenohypophysis.

Cancers may be solid tumors that may or may not be metastatic. Cancers may also occur, as in leukemia, as a diffuse tissue. Thus, the term “cancer cell,” as provided herein, includes a cell afflicted by any one of the above identified disorders or cancers.

In some embodiments of any of the methods described herein, the cancer is chosen from: hepatocellular carcinoma, colon cancer, breast cancer, pancreatic cancer, chronic myeloid leukemia (CML), chronic myelomonocytic leukemia, chronic lymphocytic leukemia (CLL), acute myeloid leukemia, acute lymphocytic leukemia, Hodgkin lymphoma, lymphoma, sarcoma, and ovarian cancer.

In some embodiments of any of the methods described herein, the cancer is chosen from: lung cancer—non-small cell, lung cancer—small cell, multiple myeloma, nasopharyngeal cancer, neuroblastoma, osteosarcoma, penile cancer, pituitary tumors, prostate cancer, retinoblastoma, rhabdomyosarcoma, salivary gland cancer, skin cancer—basal and squamous cell, skin cancer -melanoma, small intestine cancer, stomach (gastric) cancers, testicular cancer, thymus cancer, thyroid cancer, uterine sarcoma, vaginal cancer, vulvar cancer, laryngeal or hypopharyngeal cancer, kidney cancer, Kaposi sarcoma, gestational trophoblastic disease, gastrointestinal stromal tumor, gastrointestinal carcinoid tumor, gallbladder cancer, eye cancer (melanoma and lymphoma), Ewing tumor, esophagus cancer, endometrial cancer, colorectal cancer, cervical cancer, brain or spinal cord tumor, bone metastasis, bone cancer, bladder cancer, bile duct cancer, anal cancer and adrenal cortical cancer.

In some embodiments, the cancer is hepatocellular carcinoma.

In some embodiments, the cancer is colon cancer.

In some embodiments, the cancer is colorectal cancer.

In some embodiments, the cancer is breast cancer.

In some embodiments, the cancer is pancreatic cancer.

In some embodiments, the cancer is chronic myeloid leukemia (CML).

In some embodiments, the cancer is chronic myelomonocytic leukemia.

In some embodiments, the cancer is chronic lymphocytic leukemia (CLL).

In some embodiments, the cancer is acute myeloid leukemia.

In some embodiments, the cancer is acute lymphocytic leukemia.

In some embodiments, the cancer is Hodgkin lymphoma.

In some embodiments, the cancer is lymphoma.

In some embodiments, the cancer is tumors of the hematopoietic and lymphoid tissues.

In some embodiments, the cancer is hematological malignancies.

In some embodiments, the cancer is sarcoma.

In some embodiments, the cancer is ovarian cancer.

In some embodiments, the cancer is lung cancer—non-small cell.

In some embodiments, the cancer is lung cancer—small cell.

In some embodiments, the cancer is multiple myeloma.

In some embodiments, the cancer is nasopharyngeal cancer.

In some embodiments, the cancer is neuroblastoma.

In some embodiments, the cancer is osteosarcoma.

In some embodiments, the cancer is penile cancer.

In some embodiments, the cancer is pituitary tumors.

In some embodiments, the cancer is prostate cancer.

In some embodiments, the cancer is retinoblastoma.

In some embodiments, the cancer is rhabdomyosarcoma.

In some embodiments, the cancer is salivary gland cancer.

In some embodiments, the cancer is skin cancer—basal and squamous cell.

In some embodiments, the cancer is skin cancer—melanoma.

In some embodiments, the cancer is small intestine cancer.

In some embodiments, the cancer is stomach (gastric) cancers.

In some embodiments, the cancer is testicular cancer.

In some embodiments, the cancer is thymus cancer.

In some embodiments, the cancer is thyroid cancer.

In some embodiments, the cancer is uterine sarcoma.

In some embodiments, the cancer is vaginal cancer.

In some embodiments, the cancer is vulvar cancer.

In some embodiments, the cancer is Wilms tumor.

In some embodiments, the cancer is laryngeal or hypopharyngeal cancer.

In some embodiments, the cancer is kidney cancer.

In some embodiments, the cancer is Kaposi sarcoma.

In some embodiments, the cancer is gestational trophoblastic disease.

In some embodiments, the cancer is gastrointestinal stromal tumor.

In some embodiments, the cancer is gastrointestinal carcinoid tumor.

In some embodiments, the cancer is gallbladder cancer.

In some embodiments, the cancer is eye cancer (melanoma and lymphoma).

In some embodiments, the cancer is Ewing tumor.

In some embodiments, the cancer is esophagus cancer.

In some embodiments, the cancer is endometrial cancer.

In some embodiments, the cancer is colorectal cancer.

In some embodiments, the cancer is cervical cancer.

In some embodiments, the cancer is brain or spinal cord tumor.

In some embodiments, the cancer is bone metastasis.

In some embodiments, the cancer is bone cancer.

In some embodiments, the cancer is bladder cancer.

In some embodiments, the cancer is bile duct cancer.

In some embodiments, the cancer is anal cancer.

In some embodiments, the cancer is adrenal cortical cancer.

### Methods of Selecting a Treatment—Type A

Provided herein are methods of selecting a treatment for a subject (e.g., any of the subjects described herein) that include: identifying a subject having a cancer cell that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein); and selecting for the identified subject a treatment comprising a therapeutically effective amount of a CLK inhibitor (e.g., any of the CLK inhibitors described herein) as well as prodrugs and pharmaceutically acceptable salt or solvate thereof. In some embodiments, the selected treatment can further include another treatment or therapeutic agent (e.g., any cancer therapeutic agent known in the art, e.g., chemotherapy, surgery, radiation therapy, other kinase inhibitors, or a biologic), in addition to the CLK inhibitor or the pharmaceutically acceptable salt of solvate thereof.

Provided herein are methods of selecting a treatment for a subject (e.g., any of the subjects described herein) that include selecting a treatment comprising a therapeutically effective amount of a CLK inhibitor (e.g., any of the CLK inhibitors described herein or known in the art)) as well as prodrugs and pharmaceutically acceptable salt or solvate thereof for a subject identified as having a cancer cell that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein). In some embodiments, the selected treatment can further include another treatment or therapeutic agent (e.g., any cancer therapeutic agent known in the art, e.g., chemotherapy, surgery, radiation therapy, other kinase inhibitors, or a biologic), in addition to the CLK inhibitor or the pharmaceutically acceptable salt of solvate thereof.

In some embodiments of any of the methods of selecting a treatment described herein, the cancer cell is a small cell lung cancer cell, a colorectal cancer cell, a head and neck cancer cell, an ovarian cancer cell, a melanoma cell, a renal cell carcinoma cell, a pancreatic cancer cell, or a non-small cell lung cancer cell. In some embodiments of any of the methods of selecting a treatment described herein, the cancer can be any of the cancers described herein or known in the art.

Non-limiting types of Wnt pathway activity are described below and can be used in any of the methods of selecting a treatment described herein. For example, in some embodiments of any of the methods of selecting a treatment described herein, the Wnt pathway activity can be the level of CLK1, CLK2, CLK3, CLK4, or β-catenin expression, where an increased level (e.g., an increase of 1% to 500%, or any of the subranges of this range described herein) of expression of CLK1, CLK2, CLK3, CLK4, or β-catenin protein or mRNA expression, as compared to a reference level (e.g., any of the reference levels described herein) indicates an increased level of Wnt pathway activity.

For example, in some embodiments of any of the methods of selecting a treatment described herein, the Wnt pathway activity can be the level of β-catenin in the nucleus of a mammalian cell, where an increased level (e.g., an increase of 1% to 500%, or any of the subranges of this range described herein) of β-catenin in the nucleus of a mammalian cell as compared to a reference level (e.g., any of the reference levels described herein) indicates an increased level of Wnt pathway activity.

For example, in some embodiments of any of the methods of selecting a treatment described herein, the Wnt pathway activity can be detection of a mutation in a Wnt pathway gene selected from the group consisting of: gain-of-function mutation in a β-catenin gene, a loss-of-function mutation in an AXIN gene, a loss-of-function mutation in an AXIN2 gene, a loss-of-function mutation in a APC gene, a loss-of-function mutation in a CTNNB1 gene, a loss-of-function mutation in a Tsc1 gene, a loss-of-function mutation in a Tsc2 gene, and a loss-of-function mutation GSK3D gene.

For example, in some embodiments of any of the methods of selecting a treatment described herein, the Wnt pathway activity can be detection of an elevated level of expression of one or more Wnt-regulated genes as compared to a reference level (e.g., any of the reference levels described herein). Non-limiting examples of Wnt-upregulated genes include CCND1, CSNK2A1, CXCL12, LRP5, MMP7, MMP9, LEF1, AXIN2, MYC, TCF7L2, TCF7, LRP6, DVL2, BIRC, ERRB2, MAPK8, PKN1, AXIN2, ABCB1, ADAM10, ALEX1, ASCL2, BAMBI, BCL2L2, BIRC5, BMI1, BMP4, CCND1, CD44, CDKN2A, CDX1, CEBPD, CLDN1, COX2, DNMT1, EDN1, EFNB1, ENC1, EPHB2, EPHB3, FGF18, FGFBP, FRA1, FSCN1, FZD6, FZD7, FZD8, GAST, HDAC3, HEF1, HES1, ID2, ITF2, JAG1, JUN, LlCAM, LAMC2, LGR5, MENA, MET, MMP14, MYB, MYCBP, NOS2, NOTCH2, NRCAM, PLAU, PLAUR, PLCB4, PPARD, RUVBL1, S100A4, S100A6, SGK1, SMC3, SOX9, SP5, SRSF3, SUZ12, TCF1, TIAM1, TIMP-1. TN-C, VEGF, WNT-5a, WNT-5b, WNT11, and YAP.

For example, in some embodiments of any of the methods of selecting a treatment described herein, the Wnt pathway activity can be detection of a decreased level of expression of one or more of APC, FRZB, CTGF, and GSK3B.

In some embodiments of any of the methods described herein, the cancer is a small cell lung cancer, a colorectal cancer, a head and neck cancer, an ovarian cancer, a melanoma, a renal cell carcinoma, a pancreatic cancer, or a non-small cell lung cancer.

### Methods of Selecting a Subject for Treatment—Type A

Provided herein are methods of selecting a subject for participation in a clinical trial that include: identifying a subject (e.g., any of the subjects described herein) having a cancer cell (e.g., any of the types of cancer cells described herein or known in the art) that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein); and selecting the identified subject for participation in a clinical trial that comprises administration of a therapeutically effective amount of a CLK inhibitor (e.g., any of the exemplary CLK inhibitors described herein or known in the art) as well as prodrugs and pharmaceutically acceptable salt or solvate thereof. In some embodiments, the subject can be selected for a treatment that further includes another treatment or therapeutic agent (e.g., any cancer therapeutic agent known in the art, e.g., chemotherapy, surgery, radiation therapy, other kinase inhibitors, or a biologic), in addition to the CLK inhibitor or the pharmaceutically acceptable salt of solvate thereof.

Provided herein are methods of selecting a subject (e.g., any of the subjects described herein) for participation in a clinical trial that include selecting a subject identified as having a cancer cell (e.g., any of the types of cancer cells described herein or known in the art) that has an elevated level (e.g., an increase of 1% to 500%, or any of the subranges of this range described herein) of Wnt pathway activity (e.g., any of the exemplary types of Wnt pathway activity described herein or known in the art) as compared to a reference level for participation in a clinical trial that comprises administration of a therapeutically effective amount of a CLK inhibitor (e.g., any of the CLK inhibitors described herein or known in the art) as well as prodrugs and pharmaceutically acceptable salt or solvate thereof. In some embodiments, the subject can be selected for a treatment that further includes another treatment or therapeutic agent (e.g., any cancer therapeutic agent known in the art, e.g., chemotherapy, surgery, radiation therapy, other kinase inhibitors, or a biologic), in addition to the CLK inhibitor or the pharmaceutically acceptable salt of solvate thereof.

In some embodiments of any of the methods of selecting a subject for treatment described herein, the cancer cell is a small cell lung cancer cell, a colorectal cancer cell, a head and neck cancer cell, an ovarian cancer cell, a melanoma cell, a renal cell carcinoma cell, a pancreatic cancer cell, or a non-small cell lung cancer cell. In some embodiments of any of the methods of selecting a subject for treatment described herein, the cancer can be any of the cancers described herein or known in the art.

Non-limiting types of Wnt pathway activity are described below and can be used in any of the methods of selecting a subject for treatment described herein. For example, in some embodiments of any of the methods of selecting a subject for treatment described herein, the Wnt pathway activity can be the level of CLK1, CLK2, CLK3, CLK4, or β-catenin expression, where an increased level (e.g., an increase of 1% to 500%, or any of the subranges of this range described herein) of expression of CLK1, CLK2, CLK3, CLK4, or β-catenin protein or mRNA expression, as compared to a reference level (e.g., any of the reference levels described herein) indicates an increased level of Wnt pathway activity.

For example, in some embodiments of any of the methods of selecting a subject for treatment described herein, the Wnt pathway activity can be the level of β-catenin in the nucleus of a mammalian cell, where an increased level (e.g., an increase of 1% to 500%, or any of the subranges of this range described herein) of β-catenin in the nucleus of a mammalian cell as compared to a reference level (e.g., any of the reference levels described herein) indicates an increased level of Wnt pathway activity.

For example, in some embodiments of any of the methods of selecting a subject for treatment described herein, the Wnt pathway activity can be detection of a mutation in a Wnt pathway gene selected from the group consisting of: gain-of-function mutation in a β-catenin gene, a loss-of-function mutation in an AXIN gene, a loss-of-function mutation in an AXIN2 gene, a loss-of-function mutation in a APC gene, a loss-of-function mutation in a CTNNB1 gene, a loss-of-function mutation in a Tsc1 gene, a loss-of-function mutation in a Tsc2 gene, and a loss-of-function mutation GSK3D gene.

For example, in some embodiments of any of the methods of selecting a subject for treatment described herein, the Wnt pathway activity can be detection of an elevated level of expression of one or more Wnt-regulated genes as compared to a reference level (e.g., any of the reference levels described herein). Non-limiting examples of Wnt-upregulated genes include CCND1, CSNK2A1, CXCL12, LRP5, MMP7, MMP9, LEF1, AXIN2, MYC, TCF7L2, TCF7, LRP6, DVL2, BIRC, ERRB2, MAPK8, PKN1, AXIN2, ABCB1, ADAM10, ALEX1, ASCL2, BAMBI, BCL2L2, BIRC5, BMI1, BMP4, CCND1, CD44, CDKN2A, CDX1, CEBPD, CLDN1, COX2, DNMT1, EDN1, EFNB1, ENC1, EPHB2, EPHB3, FGF18, FGFBP, FRA1, FSCN1, FZD6, FZD7, FZD8, GAST, HDAC3, HEF1, HES1, ID2, ITF2, JAG1, JUN, LlCAM, LAMC2, LGR5, MENA, MET, MMP14, MYB, MYCBP, NOS2, NOTCH2, NRCAM, PLAU, PLAUR, PLCB4, PPARD, RUVBL1, S100A4, S100A6, SGK1, SMC3, SOX9, SP5, SRSF3, SUZ12, TCF1, TIAM1, TIMP-1. TN-C, VEGF, WNT-5a, WNT-5b, WNT11, and YAP.

For example, in some embodiments of any of the methods of selecting a subject for treatment described herein, the Wnt pathway activity can be detection of a decreased level of expression of one or more of APC, FRZB, CTGF, and GSK3B.

In some embodiments of any of the methods of selecting a subject for treatment described herein, the cancer is a small cell lung cancer, a colorectal cancer, a head and neck cancer, an ovarian cancer, a melanoma, a renal cell carcinoma, a pancreatic cancer, or a non-small cell lung cancer.

### Methods of Selecting a Subject for Participation in a Clinical Study

Also provided herein are methods of selecting a subject (e.g., any of the subjects described herein) for participation in a clinical trial that include: identifying a subject having a cancer cell (e.g., any of the types of cancer cells described herein or known in the art) that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein); and selecting the identified subject for participation in a clinical trial that includes administration of a therapeutically effective amount of a CLK inhibitor (e.g., any of the exemplary CLK inhibitors described herein or known in the art) or a pharmaceutically acceptable salt or solvate thereof. In some embodiments of any of the methods of selecting a subject for participation in a clinical study, the clinical trial further includes administration of another treatment or therapeutic agent (e.g., any cancer therapeutic agent known in the art, e.g., chemotherapy, surgery, radiation therapy, other kinase inhibitors, or a biologic), in addition to the CLK inhibitor or the pharmaceutically acceptable salt of solvate thereof.

Also provided herein are methods of selecting a subject (e.g., any of the subjects described herein or known in the art) for participation in a clinical trial that include selecting a subject identified as having a cancer cell (e.g., any of the types of cancer cells described herein or known in the art) that has an elevated level (e.g., an increase of 1% to 500%, or any of the subranges of this range described herein) of Wnt pathway activity (e.g., any of the exemplary types of Wnt pathway activity described herein or known in the art) as compared to a reference level (e.g., any of the exemplary reference levels described herein) for participation in a clinical trial that comprises administration of a therapeutically effective amount of a CLK inhibitor (e.g., any of the exemplary CLK inhibitors described herein or known in the art) or a pharmaceutically acceptable salt or solvate thereof. In some embodiments of any of the methods of selecting a subject for participation in a clinical study, the clinical trial further includes administration of another treatment or therapeutic agent (e.g., any cancer therapeutic agent known in the art, e.g., chemotherapy, surgery, radiation therapy, other kinase inhibitors, or a biologic), in addition to the CLK inhibitor or the pharmaceutically acceptable salt of solvate thereof.

In some embodiments of any of the methods of selecting a subject for participation in a clinical study described herein, the cancer cell is a small cell lung cancer cell, a colorectal cancer cell, a head and neck cancer cell, an ovarian cancer cell, a melanoma cell, a renal cell carcinoma cell, a pancreatic cancer cell, or a non-small cell lung cancer cell. In some embodiments of any of the methods of selecting a subject for participation in a clinical study described herein, the cancer can be any of the cancers described herein or known in the art.

Non-limiting types of Wnt pathway activity are described below and can be used in any of the methods of selecting a subject for participation in a clinical study described herein. For example, in some embodiments of any of the methods of selecting a subject for participation in a clinical study described herein, the Wnt pathway activity can be the level of CLK1, CLK2, CLK3, CLK4, or β-catenin expression, where an increased level (e.g., an increase of 1% to 500%, or any of the subranges of this range described herein) of expression of CLK1, CLK2, CLK3, CLK4, or β-catenin protein or mRNA expression, as compared to a reference level (e.g., any of the reference levels described herein) indicates an increased level of Wnt pathway activity.

For example, in some embodiments of any of the methods of selecting a subject for participation in a clinical study described herein, the Wnt pathway activity can be the level of β-catenin in the nucleus of a mammalian cell, where an increased level (e.g., an increase of 1% to 500%, or any of the subranges of this range described herein) of β-catenin in the nucleus of a mammalian cell as compared to a reference level (e.g., any of the reference levels described herein) indicates an increased level of Wnt pathway activity.

For example, in some embodiments of any of the methods of selecting a subject for participation in a clinical study described herein, the Wnt pathway activity can be detection of a mutation in a Wnt pathway gene selected from the group consisting of: gain-of-function mutation in a β-catenin gene, a loss-of-function mutation in an AXIN gene, a loss-of-function mutation in an AXIN2 gene, a loss-of-function mutation in a APC gene, a loss-of-function mutation in a CTNNB1 gene, a loss-of-function mutation in a Tsc1 gene, a loss-of-function mutation in a Tsc2 gene, and a loss-of-function mutation GSK3D gene.

For example, in some embodiments of any of the methods of selecting a subject for participation in a clinical study described herein, the Wnt pathway activity can be detection of an elevated level of expression of one or more Wnt-regulated genes as compared to a reference level (e.g., any of the reference levels described herein). Non-limiting examples of Wnt-upregulated genes include CCND1, CSNK2A1, CXCL12, LRP5, MMP7, MMP9, LEF1, AXIN2, MYC, TCF7L2, TCF7, LRP6, DVL2, BIRC, ERRB2, MAPK8, PKN1, AXIN2, ABCB1, ADAM10, ALEX1, ASCL2, BAMBI, BCL2L2, BIRC5, BMI1, BMP4, CCND1, CD44, CDKN2A, CDX1, CEBPD, CLDN1, COX2, DNMT1, EDN1, EFNB1, ENC1, EPHB2, EPHB3, FGF18, FGFBP, FRA1, FSCN1, FZD6, FZD7, FZD8, GAST, HDAC3, HEF1, HES1, ID2, ITF2, JAG1, JUN, L1CAM, LAMC2, LGR5, MENA, MET, MMP14, MYB, MYCBP, NOS2, NOTCH2, NRCAM, PLAU, PLAUR, PLCB4, PPARD, RUVBL1, S100A4, S100A6, SGK1, SMC3, SOX9, SP5, SRSF3, SUZ12, TCF1, TIAM1, TIMP-1. TN-C, VEGF, WNT-5a, WNT-5b, WNT11, and YAP.

For example, in some embodiments of any of the methods of selecting a subject for participation in a clinical study described herein, the Wnt pathway activity can be detection of a decreased level of expression of one or both of APC and FZD6.

In some embodiments of any of the methods of selecting a subject for participation in a clinical study described herein, the cancer is a small cell lung cancer, a colorectal cancer, a head and neck cancer, an ovarian cancer, a melanoma, a renal cell carcinoma, a pancreatic cancer, or a non-small cell lung cancer.

### Methods of Determining Efficacy of a CLK Inhibitor—Type A

Also provided herein are methods of determining the efficacy of a CLK inhibitor (e.g., any of the exemplary CLK inhibitors described herein or known in the art) or a pharmaceutically acceptable salt or solvate thereof in a subject (e.g., any of the subjects described herein) that include: (a) determining a first level of Wnt pathway activity (e.g., any of the exemplary types of Wnt pathway activity described herein or known in the art) in a cancer cell (e.g., any of the exemplary cancer cells described herein or known in the art) obtained from a subject at a first time point; (b) administering to the subject after the first time a CLK inhibitor or a pharmaceutically acceptable salt or solvate thereof, (c) determining a second level of the Wnt pathway activity in a cancer cell obtained from the subject at a second time point; and (d) determining that the CLK inhibitor is effective in a subject having a second level of Wnt pathway activity that is decreased (e.g., 10% to about 99% decreased, 1% to about 95% decreased, 10% to about 90% decreased, 1% to about 85% decreased, 1% to about 80% decreased, 1% to about 75% decreased, 1% to about 70% decreased, 1% to about 650% decreased, 1% to about 60% decreased, 1% to about 550% decreased, 1% to about 50% decreased, 1% to about 45% decreased, 1% to about 40% decreased, 1% to about 35% decreased, 1% to about 30% decreased, 1% to about 25% decreased, 1% to about 20% decreased, 1% to about 15% decreased, 1% to about 10% decreased, 1% to about 5% decreased, about 5% to about 99% decreased, about 5% to about 95% decreased, about 5% to about 90% decreased, about 5% to about 85% decreased, about 5% to about 80% decreased, about 5% to about 75% decreased, about 5% to about 70% decreased, about 5% to about 65% decreased, about 5% to about 60% decreased, about 5% to about 55% decreased, about 5% to about 50% decreased, about 5% to about 45% decreased, about 5% to about 40% decreased, about 5% to about 35% decreased, about 5% to about 30% decreased, about 5% to about 25% decreased, about 5% to about 20% decreased, about 5% to about 15% decreased, about 5% to about 10% decreased, about 10% to about 99% decreased, about 10% to about 95% decreased, about 10% to about 90% decreased, about 10% to about 85% decreased, about 10% to about 80% decreased, about 10% to about 75% decreased, about 10% to about 70% decreased, about 10% to about 65% decreased, about 10% to about 60% decreased, about 10% to about 55% decreased, about 10% to about 50% decreased, about 10% to about 45% decreased, about 10% to about 40% decreased, about 10% to about 35% decreased, about 10% to about 30% decreased, about 10% to about 25% decreased, about 10% to about 20% decreased, about 10% to about 15% decreased, about 15% to about 99% decreased, about 15% to about 95% decreased, about 15% to about 90% decreased, about 15% to about 85% decreased, about 15% to about 80% decreased, about 15% to about 75% decreased, about 15% to about 70% decreased, about 15% to about 65% decreased, about 15% to about 60% decreased, about 15% to about 55% decreased, about 15% to about 50% decreased, about 15% to about 45% decreased, about 15% to about 40% decreased, about 15% to about 35% decreased, about 15% to about 30% decreased, about 15% to about 25% decreased, about 15% to about 20% decreased, about 20% to about 99% decreased, about 20% to about 95% decreased, about 20% to about 90% decreased, about 20% to about 85% decreased, about 20% to about 80% decreased, about 20% to about 75% decreased, about 20% to about 70% decreased, about 20% to about 65% decreased, about 20% to about 60% decreased, about 20% to about 55% decreased, about 20% to about 50% decreased, about 20% to about 45% decreased, about 20% to about 40% decreased, about 20% to about 35% decreased, about 20% to about 30% decreased, about 20% to about 25% decreased, about 25% to about 99% decreased, about 25% to about 95% decreased, about 25% to about 90% decreased, about 25% to about 85% decreased, about 25% to about 80% decreased, about 25% to about 75% decreased, about 25% to about 70% decreased, about 25% to about 65% decreased, about 25% to about 60% decreased, about 25% to about 55% decreased, about 25% to about 50% decreased, about 25% to about 45% decreased, about 25% to about 40% decreased, about 25% to about 35% decreased, about 25% to about 30% decreased, about 30% to about 99% decreased, about 30% to about 95% decreased, about 30% to about 90% decreased, about 30% to about 85% decreased, about 30% to about 80% decreased, about 30% to about 75% decreased, about 30% to about 70% decreased, about 30% to about 65% decreased, about 30% to about 60% decreased, about 30% to about 55% decreased, about 30% to about 50% decreased, about 30% to about 45% decreased, about 30% to about 40% decreased, about 30% to about 35% decreased, about 35% to about 99% decreased, about 35% to about 95% decreased, about 35% to about 90% decreased, about 35% to about 85% decreased, about 35% to about 80% decreased, about 35% to about 75% decreased, about 35% to about 70% decreased, about 35% to about 65% decreased, about 35% to about 60% decreased, about 35% to about 55% decreased, about 35% to about 50% decreased, about 35% to about 45% decreased, about 35% to about 40% decreased, about 40% to about 99% decreased, about 40% to about 95% decreased, about 40% to about 90% decreased, about 40% to about 85% decreased, about 40% to about 80% decreased, about 40% to about 75% decreased, about 40% to about 70% decreased, about 40% to about 65% decreased, about 40% to about 60% decreased, about 40% to about 55% decreased, about 40% to about 50% decreased, about 40% to about 45% decreased, about 45% to about 99% decreased, about 45% to about 95% decreased, about 45% to about 90% decreased, about 45% to about 85% decreased, about 45% to about 80% decreased, about 45% to about 75% decreased, about 45% to about 70% decreased, about 45% to about 65% decreased, about 45% to about 60% decreased, about 45% to about 55% decreased, about 45% to about 50% decreased, about 50% to about 99% decreased, about 50% to about 95% decreased, about 50% to about 90% decreased, about 50% to about 85% decreased, about 50% to about 80% decreased, about 50% to about 75% decreased, about 50% to about 70% decreased, about 50% to about 65% decreased, about 50% to about 60% decreased, about 50% to about 55% decreased, about 55% to about 99% decreased, about 55% to about 95% decreased, about 55% to about 90% decreased, about 55% to about 85% decreased, about 55% to about 80% decreased, about 55% to about 75% decreased, about 55% to about 70% decreased, about 55% to about 65% decreased, about 55% to about 60% decreased, about 60% to about 99% decreased, about 60% to about 95% decreased, about 60% to about 90% decreased, about 60% to about 85% decreased, about 60% to about 80% decreased, about 60% to about 75% decreased, about 60% to about 70% decreased, about 60% to about 65% decreased, about 65% to about 99% decreased, about 65% to about 95% decreased, about 65% to about 90% decreased, about 65% to about 85% decreased, about 65% to about 80% decreased, about 65% to about 75% decreased, about 65% to about 70% decreased, about 70% to about 99% decreased, about 70% to about 95% decreased, about 70% to about 90% decreased, about 70% to about 85% decreased, about 70% to about 80% decreased, about 70% to about 75% decreased, about 75% to about 99% decreased, about 75% to about 95% decreased, about 75% to about 90% decreased, about 75% to about 85% decreased, about 75% to about 80% decreased, about 80% to about 99% decreased, about 80% to about 95% decreased, about 80% to about 90% decreased, about 80% to about 85% decreased, about 85% to about 99% decreased, about 85% to about 95% decreased, about 85% to about 90% decreased, about 90% to about 99% decreased, about 90% to about 95% decreased, or about 95% to about 99% decreased) as compared to the first level of Wnt pathway activity.

In some embodiments of any of the methods described herein, the method further includes: (e) after (d), administering one or more (e.g., 1, 2, 3, 4, 5, 6, 7, 8, 9, 10, 11, 12, 13, 14, 15, 16, 17, 18, 19, 20, 22, 24, 26, 28, 30, 40, 50, 60, 70, 80, 90, or 100) additional doses of the CLK inhibitor to the subject.

In some embodiments of any of the methods further include a step of selecting a subject having cancer or diagnosing a subject as having cancer. For example, a subject having cancer can have previously been administered a treatment for cancer, and the previous treatment was unsuccessful. Some embodiments of any of the methods described herein can further include obtaining a cancer cell from the subject at the first and second time points.

In some embodiments of any of the methods described herein, the method further includes recording the identified efficacy of the CLK inhibitor in the subject's medical record (e.g., a computer readable medium).

In some embodiments of any of the methods described herein, the method further includes informing the subject, the subject's family, and/or the subject's primary care physician or attending physician of the determined efficacy of the CLK inhibitor.

In some embodiments of any of the methods described herein, the method further includes monitoring the subject. For example, the method can include authorizing a refill of the CLK inhibitor administered to the subject between the first and second time points and determined to be effective.

In some embodiments of any of the methods of determining the efficacy of treatment described herein, the cancer cell is a small cell lung cancer cell, a colorectal cancer cell, a head and neck cancer cell, an ovarian cancer cell, a melanoma cell, a renal cell carcinoma cell, a pancreatic cancer cell, or a non-small cell lung cancer cell. In some embodiments of any of the methods of determining the efficacy of treatment described herein, the cancer can be any of the cancers described herein or known in the art.

Non-limiting types of Wnt pathway activity are described below and can be used in any of the methods of determining the efficacy of treatment described herein. For example, in some embodiments of any of the methods of determining the efficacy of treatment described herein, the Wnt pathway activity can be the level of CLK1, CLK2, CLK3, CLK4, or β-catenin expression, where an increase in the second level (e.g., an increase of 1% to 500%, or any of the subranges of this range described herein) of expression of CLK1, CLK2, CLK3, CLK4, or β-catenin protein or mRNA expression, as compared to the first level of CLK1, CLK2, CLK3, CLK4, or β-catenin protein or mRNA expression indicates that the CLK inhibitor was effective in the subject.

For example, in some embodiments of any of the methods of determining the efficacy of treatment described herein, the Wnt pathway activity can be the level of β-catenin in the nucleus of a mammalian cell, where an increase in the second level (e.g., an increase of 1% to 500%, or any of the subranges of this range described herein) of p-catenin in the nucleus of a mammalian cell as compared to the first level of β-catenin in the nucleus of a mammalian cell indicates that the CLK inhibitor was effective in the subject.

For example, in some embodiments of any of the methods of determining the efficacy of treatment described herein, the Wnt pathway activity can be detection of first and second levels of expression of one or more Wnt-regulated genes, where an decreased second level (e.g., a 1% to a 99% decrease, or any of the subranges of this range described herein) of expression of the one or more Wnt-regulated genes as compared to the first level of expression of the one or more Wnt-regulated genes indicates that the CLK inhibitor was effective in the subject. Non-limiting examples of Wnt-upregulated genes include CCND1, CSNK2A1 CXCL12, LRP5, MMP7, MMP9, LEF1, AXIN2, MYC, TCF7L2, TCF7, LRP6, DVL2, BIRC, ERRB2, MAPK8, PKN1, AXIN2, ABCB1, ADAM10, ALEX1, ASCL2, BAMBI, BCL2L2, BIRC5, BMI1, BMP4, CCND1, CD44, CDKN2A, CDX1, CEBPD, CLDN1, COX2, DNMT1, EDN1, EFNB1, ENC1, EPHB2, EPHB3, FGF18, FGFBP, FRA1, FSCN1, FZD6, FZD7, FZD8, GAST, HDAC3, HEF1, HES1, ID2, ITF2, JAG1, JUN, LICAM, LAMC2, LGR5, MENA, MET, MMP14, MYB, MYCBP, NOS2, NOTCH2, NRCAM, PLAU, PLAUR, PLCB4, PPARD, RUVBL1, S100A4, S100A6, SGK1, SMC3, SOX9, SP5, SRSF3, SUZ12, TCF1, TIAM1, TIMP-1. TN-C, VEGF, WNT-5a, WNT-5b, WNT11, and YAP.

In some embodiments of any of the methods of determining the efficacy of treatment described herein, the Wnt pathway activity can be detection of first and second levels of expression of one or more of APC, FRZB, CTGF, and GSK3B, where an increased (e.g., a 1% to a 500% increase or any of the subranges of this range described herein) second level of expression of the one or more of APC, FRZB, CTGF, and GSK3B, as compared to the first level of expression of one or more of APC, FRZB, CTGF, and GSK3B indicates that the CLK inhibitor was effective in the subject In some embodiments of any of the methods of determining the efficacy of treatment described herein, the cancer is a small cell lung cancer, a colorectal cancer, a head and neck cancer, an ovarian cancer, a melanoma, a renal cell carcinoma, a pancreatic cancer, or a non-small cell lung cancer.

### Methods of Determining the Level of Wnt Pathway Activity

In some embodiments of any of the methods described herein, the level of Wnt pathway activity is the level of CLK1, CLK2, CLK3, CLK4, or β-catenin expression. In some embodiments, the level of CLK1, CLK2, CLK3, CLK4, or β-catenin expression is the level of CLK1, CLK2, CLK3, CLK4, or β-catenin protein in any of the cells described herein. In some embodiments, the level of CLK1, CLK2, CLK3, CLK4, or β-catenin expression is the level of CLK1, CLK2, CLK3, CLK4, or β-catenin mRNA in any of the cells described herein.

In some embodiments of any of the methods described herein, the level of Wnt pathway activity is the level of β-catenin in the nucleus of any of the cells described herein.

In some embodiments of any of the methods described herein, the Wnt pathway activity is detection of a mutation in a Wnt pathway gene selected from the group consisting of: gain-of-function mutation in a β-catenin gene (e.g., any of the exemplary gain-of-function mutations in a β-catenin gene described herein), a loss-of-function mutation in an AXIN gene, a loss-of-function mutation in an AXIN2 gene, a loss-of-function mutation in a APC gene, a loss-of-function mutation in a CTNNB1 gene, a loss-of-function mutation in a Tsc1 gene, a loss-of-function mutation in a Tsc2 gene, and a loss-of-function mutation GSK3D gene.

In some embodiments of any of the methods described herein, the Wnt pathway activity is an increased level of expression of one or more (e.g., 1, 2, 3, 4, 5, 6, 7, or 8) Wnt-upregulated genes.

In some embodiments, the one or more Wnt-upregulated genes are selected from the group consisting of: cyclin D1 (CCND1), casein kinase 2 alpha 1 (CSNK2A1), C—X—C motif chemokine ligand 12 (CXCL12), low density lipoprotein receptor-related protein 5 (LRP5), matrix metallopeptidase 7 (MMP7), matrix metallopeptidase 9 (MMP9), lymphoid enhancer binding factor 1 (LEF1), axin 2 (AXIN2), MYC proto-oncogene (MYC), transcription factor 7 like 2 (TCF7L2), transcription factor 7 (TCF7), low density lipoprotein receptor-related protein 6 (LRP6), disheveled segment polarity protein 2 (DVL2), NLR family apoptosis inhibitory protein pseudogene (BIRC), estrogen-related receptor beta type 2 (ERRB2), mitogen-activated protein kinase 8 (MAPK8), protein kinase N1 (PKN1), axin 2 (AXIN2), ATP binding cassette subfamily B member 1 (ABCB1), a disintegrin and metallopeptidase domain 10 (ADAM1O), armadillo repeat containing X-linked 1 (ALEX1), achaete-scute family bHLH transcription factor 2 (ASCL2), BMP and activin membrane bound inhibitor (BAMBI), BLCL2-like 2 (BCL2L2), baculoviral IAP repeat containing 5 (BIRC5), BMI1 proto-oncogene (BMI1), bone morphogenetic protein 4 (BMP4), CCND1, CD44 molecule (CD44), cyclin dependent kinase inhibitor 2A (CDKN2A), caudal type homeobox 1 (CDX1), CCAAT enhancer binding protein delta (CEBPD), claudin 1 (CLDN1), cytochrome c oxidase subunit II (COX2), DNA methyltransferase I (DNMT1), endothelin 1 (EDN1), ephrin B1 (EFNB1), ectodermal-neural cortex 1 (ENC1), Eph receptor B2 (EPHB2), Eph receptor B3 (EPHB3), fibroblast growth factor 18 (FGF18), fibroblast growth factor binding protein 1 (FGFBP), FOS-like 1 (FRA1), facin actin-bundling protein 1 (FSCN1), frizzled class receptor 6 (FZD6), frizzled class receptor 7 (FZD7), frizzled class receptor 8 (FZD8), gastrin (GAST), histone deacetylase 3 (HDAC3), neural precursor cell expressed developmentally down-regulated 9 (HEF1), hes family bHLH transcription factor 1 (HES1), inhibitor of DNA binding 2 (ID2), transcription factor 4 (ITF2), jagged 1 (JAG1), Jun proto-oncogene (JUN), L1 cell adhesion molecule (LiCAM), laminin subunit gamma 2 (LAMC2), leucine rich containing G protein coupled receptor 5 (LGR5), ENAH (MENA), MET proto-oncogene (MET), matrix metallopeptidase 14 (MMP14), MYB proto-oncogene (MYB), MYC binding protein (MYCBP), nitric oxide synthase 2 (NOS2), notch 2 (NOTCH2), neuronal cell adhesion molecule (NRCAM), plasminogen activator urokinase (PLAU), plasminogen activator urokinase receptor (PLAUR), phospholipase C beta 4 (PLCB4), peroxisome proliferator activated receptor delta (PPARD), RuvB Like AAA ATPase 1 (RUVBL1), S100 calcium binding protein A4 (S100A4), S100 calcium binding protein A6 (S100A6), serum/glucocorticoid regulated kinase 1 (SGK1), structural maintenance of chromosomes 3 (SMC3), sex determining region Y-box 9 (SOX9), trans-acting transcription factor 5 (SP5), serine and arginine rich splicing factor 3 (SRSF3), SUZ12 polycomb repressive complex 2 subunit (SUZ12), HNF1 homeobox A (TCF1), T cell lymphoma invasion and metastasis 1 (TIAM1), tissue inhibitor of metalloproteinase 1 (TIMP-1), tenascin C (TN-C), vascular endothelial growth factor (VEGF), wingless-type family member 5A (WNT-5a), wingless-type family member 5B (WNT-5b), wingless-type family member 11 (WNT11), and Yes associated protein 1 (YAP).

In some embodiments of any of the methods described herein, the Wnt pathway activity is a decreased level of expression of one or more of APC Regulator of Wnt Signaling Pathway (APC), Frizzled Related Protein (FRZB), Connective Tissue Growth Factor (CTGF), and Glycogen Synthase Kinase 3 Beta (GSK3B).

In some embodiments, the Wnt pathway activity is the activity determined by assessing the expression level (e.g., protein or mRNA) of one or more (e.g., 1, 2, 3, 4, 5, 6, 7, 8, 9, 10, 11, 12, 13, 14, 15, 16, 17, 18, 19, 20 or more) of: AXIN (NCBI Accession NG_012267.1), AXIN2 (NCBI Accession NG_012142.1), APC (NCBI Accession NG_008481.4), CSNK2A1 (NCBI Accession No. BC011668.2), CTGF (NCBI Accession AY395801.1), CTNNB1 (NCBI Accession NG_013302.2), Tsc1 (NCBI Accession NG_012386.1), Tsc2 (NCBI Accession NG_005895.1), GSK3β (NCBI Accession NG_012922.1), CCND1 (NCBI Accession NG_-7375.1), CXCL12 (NCBI Accession NG_016861.1), LRP5 (NCBI Accession NG_015835.1), MMP7 (NCBI Accession NM_002423.4), MMP9 (NCBI Accession NG_004994.2), LEF1 (NCBI Accession NG_015798.1), AXIN2 (NCBI Accession NG_012142.1), MYC (NCBI Accession NG_007161.2), TCF7L2 (NCBI Accession NG_012631.1), TCF7 (NCBI Accession NG_030367.1), LRP6 (NCBI Accession NG_01618.1), DVL2 (NCBI Accession NG_033038.1), BIRC (NCBI Accession NG_008752.1), ERRB2 (NCBI Accession NG_007503.1), MAPK8 (NCBI Accession NG_029053.2), PKN1 (NCBI Accession NG_), AXIN2 (NCBI Accession NG_00019.10), ABCB1 (NCBI Accession NG_011513.1), ADAM10 (NCBI Accession NG_033876.1), ALEX1 (NCBI Accession NG_015846.1), ASCL2 (NCBI Accession NM_005170.2), BAMBI (NCBI Accession NM_012342.2), BCL2L2 (NCBI Accession NM_001199839.1), BIRC5 (NCBI Accession NG_029069.1), BMI1 (NCBI Accession NM_005180.8), BMP4 (NCBI Accession NG_009215.1), CD44 (NCBI Accession NG_008937.1), CDKN2A (NCBI Accession NG_007485.1), CDX1 (NCBI Accession NG_046970.1), CEBPD (NCBI Accession NM_005195.3), CLDN1 (NCBI Accession NG_021418.1), COX2 (NCBI Accession NG_028206.2), DNMT1 (NCBI Accession NG_028016.3), EDN1 (NCBI Accession NG_016196.1), EFNB1 (NCBI Accession NG_008887.1), ENC1 (NCBI Accession NM_001256575.1), EPHB2 (NCBI Accession NG_011804.2), EPHB3 (NCBI Accession NM_004443.3), FGF18 (NCBI Accession NG_029158.1), FGFBP (NCBI Accession NM_005130.4), FRA1 (NCBI Accession NM_005438.4), FRZB (NCBI Accession NM_001463.4), FSCN (NCBI Accession NG_030004.1), FZD6 (NCBI Accession NM_003506.4), FZD7 (NCBI Accession NM_003507.1), FZD8 (NCBI Accession NG_029968.1), GAST (NCBI Accession NM_00805.4), GSK3B (NCBI Accession NM_002093.4), HDAC3 (NCBI Accession NM_001355039.2), HEF1 (NCBI Accession NM_006403.3), HES1 (NCBI Accession NM_005524.3), ID2 (NCBI Accession NM_002166.4), ITF2 (NCBI Accession NG_011716.2), JAG1 (NCBI Accession NG_007496.1), JUN (NCBI Accession NG_047027.1), LICAM (NCBI Accession NG_009645.3), LAMC2 (NCBI Accession NG_007079.2), LGR5 (NCBI Accession NM_003667.3), MENA (NCBI Accession NG_051578.1), MET (NCBI Accession NG_008996.1), MMP14 (NCBI Accession NG_046989.1), MYB (NCBI Accession NG_012330.1), MYCBP (NCBI Accession NM_012333.4), NOS2 (NCBI Accession NG_011470.1), NOTCH2 (NCBI Accession NG_008163.1), NRCAM (NCBI Accession NG_029898.1), PLAU (NCBI Accession NG_011904.1), PLAUR (NCBI Accession NG_032898.1), PLCB4 (NCBI Accession NM_000933.3), PPARD (NCBI Accession NG_012345.1), RUVBL1 (NCBI Accession NM_003707.3), S100A4 (NCBI Accession NG_027993.1), S100A6 (NCBI Accession NM_014624.3), SGK1 (NCBI Accession NM_005627.3), SMC3, (NCBI Accession NG_012217.1), SOX9 (NCBI Accession NG_012490.1), SP5 (NCBI Accession NM_001003845.2), SRSF3 (NCBI Accession NM_003017.4), SUZ12 (NCBI Accession NG_009237.1), TCF1 (NCBI Accession NG_011731.2), TIAM1 (NCBI Accession NM_001353693.1), TIMP-1 (NCBI Accession NG_012533.1), TN-C(NCBI Accession NG_029637.1), VEGF (NCBI Accession NG_008732.1), WNT-5a (NCBI Accession NG_031992.1), WNT-5b (NCBI Accession NM_032642.2), WNT11 (NCBI Accession NG_046931.1), YAP (NCBI Accession NG_029530.1), SRSF1 (NCBI Accession NM_006924.4), SRSF2 (NCBI Accession NG_032905.1), SRSF3 (NCBI Accession NM_003017.4), SF3B1 (NCBI Accession NG_032903.2), SRSF4 (NCBI Accession NM_005626.4), SRSF5 (NCBI Accession NM_001039465.1), SRSF6 (NCBI Accession NM_006275.5), SRSF10 (NCBI Accession NM_006625.5), U2AF1 (NCBI Accession NG_029455.1), and ZRSR2 (NCBI Accession NG_012746.1).

In any of the methods described herein, the level of at least one (e.g., 2, 3, 4, 5, 6, 7, 8, 9, 10, 11, 12, 13, 14, 15, 16, 17, 18, 19, 20, 25) Wnt pathway activity can be determined (e.g., in any combination).

The biological activity of the compounds described herein can be tested using any suitable assay known to those of skill in the art, see, e.g., WO 2001/053268 and WO 2005/009997. For example, the activity of a compound may be tested using one or more of the test methods outlined below.

In one example, tumor cells may be screened for Wnt independent growth. In such a method, tumor cells of interest are contacted with a compound (i.e. inhibitor) of interest, and the proliferation of the cells, e.g. by uptake of tritiated thymidine, is monitored. Non-limiting examples of assays that can be used to determine cell proliferation include: BrdU incorporation assay, EdU incorporation assay, MTT assay, XTT cell proliferation assay, proliferating cell nuclear antigen (PCNA) immunohistochemistry assay, Ki67 immunohistochemistry, minichromosome maintenance complex component 2 (MCM2) immunohistochemistry. In some embodiments, cell proliferation is determined by conducting a cell growth curve. In some embodiments, a proliferation assay is carried out using flow cytometry.

In some embodiments, tumor cells may be isolated from a candidate patient who has been screened for the presence of a cancer that is associated with a mutation in the Wnt signaling pathway. Candidate cancers include, without limitation, those described herein.

In another example, one may utilize in vitro assays for Wnt biological activity, e.g., stabilization of β-catenin and promoting growth of stem cells. Assays for biological activity of Wnt include stabilization of p-catenin, which can be measured, for example, by serial dilutions of a candidate inhibitor composition. An exemplary assay for Wnt biological activity contacts a candidate inhibitor with cells containing constitutively active Wnt/β-catenin signaling. The cells are cultured for a period of time sufficient to stabilize p-catenin, usually at least about 1 hour, and lysed. The cell lysate is resolved by SDS PAGE, then transferred to nitrocellulose and probed with antibodies specific for p-catenin.

In a further example, the activity of a candidate compound can be measured in a Xenopus secondary axis bioassay (Leyns, L. et al. Cell (1997), 88(6), 747-756).

In some embodiments, Wnt pathway activity is determined using a Wnt reporter assay. Briefly, cells are transfected with a reporter vector (e.g., a luciferase reporter) in which a reporter gene is operatively-linked to a gene regulatory element (e.g., a promoter, a responsive element) of a Wnt pathway target gene (e.g., TCF/LEF). Untransfected cells can serve as a negative control, while transfected cells cultured in the presence of a known Wnt pathway agonist (e.g., a Wnt pathway ligand) can serve as a positive control.

Determination of expression levels and/or detection of any of the mutations described herein may be performed by any suitable method including, but are not limited to, methods based on analyses of polynucleotide expression, sequencing of polynucleotides, and/or analyses of protein expression. For example, determination of expression levels may be performed by detecting the expression of mRNA expressed from the genes of interest, and/or by detecting the expression of a polypeptide encoded by the genes.

Commonly used methods for the analysis of polynucleotides (e.g., detection of any of the mutations described herein and/or detection of the expression level of any of the mRNAs described herein), include Southern blot analysis, Northern blot analysis, in situ hybridization, RNAse protection assays, and polymerase chain reaction (PCR)-based methods, such as reverse transcription polymerase chain reaction (RT-PCR), quantitative PCR (qPCR), real-time PCR, TaqMan™, TaqMan™ low density array (TLDA), anchored PCR, competitive PCR, rapid amplification of cDNA ends (RACE), and microarray analyses. RT-PCR is a quantitative method that can be used to compare mRNA levels in different samples to examine gene expression profiles. A variation of RT-PCR is real time quantitative PCR, which measures PCR product accumulation through a dual-labeled fluorigenic probe (e.g., TaqMan™ probe). There are many other PCR-based techniques known to one of skill in the art, including but not limited to, differential display, amplified fragment length polymorphism, BeadArray™ technology, high coverage expression profiling (HiCEP) and digital PCR. Representative methods for sequencing-based gene expression analyses include Serial Analysis of Gene Expression (SAGE), Massively Parallel Signature Sequencing (MPSS), and NexGen sequencing analysis, including mRNA sequencing.

In certain embodiments, the biomarker expression is determined using a qPCR assay. For example, total RNA is extracted from a fresh frozen (FF) tissue sample or total RNA is extracted from a macro-dissected formalin-fixed paraffin embedded (FFPE) tissue sample. The quantity and quality of the total RNA is assessed by standard spectrophotometry and/or any other appropriate method (e.g., an Agilent Bioanalyzer). Following RNA extraction, the RNA sample is reverse transcribed using standard methods and/or a commercially available cDNA synthesis kit (e.g., Roche Transcriptor First Strand cDNA synthesis kit). The resultant cDNA is pre-amplified using, for example, an ABI pre-amplification kit. Expression of the biomarker(s) are assessed on, for example, a Roche Lightcycler 480 system (Roche Diagnostics) using an ABI TaqMan Gene Expression Mastermix. qPCR reactions are performed in triplicate. For each assay a subset of the samples is run without reverse transcription (the RT-neg control), as well as, control samples run without template. A universal human reference RNA sample is included on each plate to act as a positive control. Suitable reference genes are identified from a standard panel of reference genes. Candidate reference genes are selected with different cellular functions to eliminate risk of co-regulation. The most suitable reference genes are evaluated and selected using specific software and algorithms (e.g., Genex software; GeNorm and Normfinder algorithms). The expression level of each biomarker is normalized using the selected optimum reference genes. In some embodiments, these normalized (or standardized) expression values for each biomarker are used to calculate the decision value of the sample. In some embodiments, these normalized (or standardized) expression values for each biomarker are used to calculate an expression level.

In some embodiments, the detection of any of the mutations described herein and/or detection of the level of any of the mRNAs described herein can be performed using a PCR-based assay comprising specific primers and/or probes. As used herein, the term “probe” refers to any molecule that is capable of selectively binding a specifically intended target biomolecule. Probes can be synthesized by one of skill in the art using known techniques or derived from biological preparations. Probes may include but are not limited to, RNA, DNA, proteins, peptides, aptamers, antibodies, and organic molecules. The term “primer” or “probe” encompasses oligonucleotides that have a sequence of a specific SEQ ID NO or oligonucleotides that have a sequence complementary to a specific SEQ ID NO. In some embodiments, the probe is modified. In some embodiments, the probe is modified with a quencher. In some embodiments, the probe is labeled. Labels can include, but are not limited to, colorimetric, fluorescent, chemiluminescent, or bioluminescent labels.

In some embodiments, the expression level of any of the proteins described herein can be determined by immunohistochemistry (IHC) of formalin fixed paraffin embedded tissue samples or overexpressed gene expression.

In some embodiments, the expression level of any of the mRNAs described herein can be determined by qPCR methods.

In some embodiments, the expression level of any of the proteins described herein or any of the phosphorylated proteins described herein can be determined from tumor biopsy samples by immunohistochemistry (IHC) of formalin fixed paraffin embedded tissue samples.

In some embodiments, the expression level of any of the mRNAs described herein can be determined from tumor biopsy samples by qPCR methods.

Commonly used methods for determining the level of any of the proteins described herein (or the level of any of the phosphorylated proteins described herein), include but are not limited to, immunohistochemistry (IHC)-based, antibody-based, and mass spectrometry-based methods. Antibodies, generally monoclonal antibodies, may be used to detect expression of a gene product (e.g., protein). In some embodiments, the antibodies can be detected by direct labeling of the antibodies themselves. In other embodiments, an unlabeled primary antibody is used in conjunction with a labeled secondary antibody Immunohistochemistry methods and/or kits are well known in the art and are commercially available.

In some embodiments, the level or expression level of any of the proteins described herein (or any of the phosphorylated proteins described herein) can be determined using methods known in the art, including but not limited to, multi-analyte profile test, enzyme-linked immunosorbent assay (ELISA), radioimmunoassay, Western blot assay, immunofluorescent assay, enzyme immunoassay, immunoprecipitation assay, chemiluminescent assay, immunohistochemical assay, dot blot assay, slot blot assay, and SDS-PAGE. In some embodiments, wherein an antibody is used in the assay the antibody is detectably labeled. The antibody labels may include, but are not limited to, immunofluorescent label, chemiluminescent label, phosphorescent label, enzyme label, radiolabel, avidin/biotin, colloidal gold particles, colored particles and magnetic particles.

Other suitable methods for determining the level of any of the proteins described herein (or any of the phosphorylated proteins described herein) include proteomics-based methods. Proteomics includes, among other things, study of the global changes of protein expression in a sample. In some embodiments, a proteomic method comprises the following steps: (1) separation of individual proteins in a sample by 2-D electrophoresis (2-D PAGE), (2) identification of individual proteins recovered from the gel (e.g., by mass spectrometry or N-terminal sequencing), and (3) analysis of the data using bioinformatics. In some embodiments, a proteomic method comprises using a tissue microarray (TMA). Tissue arrays may be constructed according to a variety of techniques known to one of skill in the art. In certain embodiments, a manual tissue arrayer is used to remove a “core” from a paraffin block prepared from a tissue sample. The core is then inserted into a separate paraffin block in a designated location on a grid. Cores from as many as about 400 samples can be inserted into a single recipient block. The resulting tissue array may be processed into thin sections for analysis. In some embodiments, a proteomic method comprises an antibody microarray. In some embodiments, a proteomic method comprises using mass spectrometry, including but not limited to, SELDI, MALDI, electro spray, and surface plasmon resonance methods. In some embodiments, a proteomic method comprises bead-based technology, including but not limited to, antibodies on beads in an array format. In some embodiments, the proteomic method comprises a reverse phase protein microarray (RPPM). In some embodiments, the proteomic method comprises multiplexed protein profiling, including but not limited to, the Global Proteome Survey (GPS) method.

In some embodiments, the level of expression of any of the mRNAs described herein in a mammalian cell (e.g., a cancer cell) obtained from the subject can be compared to a reference level of expression in a control cell (e.g., a non-cancerous cell or a healthy cell from the same subject or from a similar non-cancerous tissue from a similar subject) using gene microarray (e.g., Affimetrix chips). The comparison of the expression level of any of the mRNAs described herein in a cell obtained from a subject as compared to a reference level of expression in a control cell (e.g., a non-cancerous cell) can be determined from gene microarray using statistical methods. The statistical methods may include, but are not limited to, cluster analysis, supported vector machines (SVM) analysis, supported vector machines-recursive feature elimination (SVM-RFE) analysis, Platt scaling, neural networks, and other algorithms, t-test analysis, and paired-sample empirical Baysian analysis.

In some embodiments, the Wnt pathway activity is determined by Western blotting, immunohistochemistry, or immunofluorescence. For example, a readout for increased Wnt pathway activity can be an increase in the level of β-catenin (e.g., an increase in non-phosphorylated β-catenin), an increase in the phosphorylation of Dishevelled, or an increase in the phosphorylation of LRP.

Some embodiments of any of the methods described herein can include a step of performing an assay to determine a level or levels (e.g., a first and a second level) of a Wnt pathway gene in a cancer cell obtained from the subject at a first and a second time point. Non-limiting assays that may be used to detect a level or levels of a Wnt pathway gene are described herein. Additional assays that may be used to detect a level or levels of a Wnt pathway gene are known in the art.

Additional non-limiting assays that can be used to detect a level of a Wnt pathway protein include: immunohistochemistry, immunofluorescence, Western blotting, mass spectrometry, flow cytometry, immunoassays (e.g., sandwich enzyme-linked immunosorbent assays, enzyme-linked immunosorbent assays, and immunoprecipitation).

Additional non-limiting assays that can be used to detect a level of a Wnt pathway gene expression include: reverse transcription polymerase chain reaction (rt-PCR), real time quantitative reverse transcription polymerase chain reaction (qRT-PCR), microarray, next generation sequencing,

### Reference Levels

In some embodiments of any of the methods described herein, the reference can be a corresponding level detected in a non-cancerous cell obtained from a subject (e.g., a non-cancerous cell from a similar non-cancerous tissue in a heathy subject who does not have a cancer and does not have a family history of cancer). For example, the reference level can be a corresponding level detected in a non-cancer cell of the same cell type as the cancerous cell. In some embodiments, the reference level can be a corresponding level detected in a non-cancerous skin cell (e.g., a melanocyte), and the cancer cell is a melanoma cell. In some embodiments, a reference level can be a corresponding level detected in a non-cancerous cell obtained from the breast, and the cancer cell is a breast cancer cell. In some embodiments, a reference level can be a corresponding level detected in a non-cancerous cell obtained from the prostate, and the cancer cell is a prostate cancer cell.

In some embodiments, a reference level can be a corresponding level detected in a non-cancerous cell obtained from the subject prior to the subject having been identified and/or diagnosed with a cancer (e.g., any of the cancers described herein). In some embodiments, a reference level can be a corresponding level in an intestinal stem cell (e.g., an intestinal stem cell obtained from the subject).

In some embodiments, a reference level can be a corresponding threshold level.

In some embodiments, a reference level can be a percentile value (e.g., mean value, 99% percentile, 95% percentile, 90% percentile, 85% percentile, 80% percentile, 75% percentile, 70% percentile, 65% percentile, 60% percentile, 55% percentile, or 50% percentile) of the corresponding levels detected in similar samples in a population of healthy subjects (e.g., subjects that are not diagnosed or identified as having a cancer (e.g., any of the cancers described herein), do not present with a symptom of cancer, and are not considered to have an elevated risk of developing cancer). In some embodiments, a reference level can be a threshold numerical value.

In some embodiments, a reference level can be a corresponding level detected in a similar sample obtained from the subject at an earlier time point.

### Methods of Decreasing Activity of One or More of CLK1, CLK2, CLK3, and CLK4

Also provided herein are methods of decreasing (e.g., a 1% to 99% decrease, or any of the subranges of this range described herein) the activity of one or more of CLK1, CLK2, CLK3, and CLK4 that include: contacting one or more (e.g., one, two, three, or four) of CLK1, CLK2, CLK3, and CLK4 with an effective amount of a CLK inhibitor or a pharmaceutically acceptable salt or solvate thereof. In some embodiments, the method includes contacting one or both of CLK2 and CLK3 with an effective amount of a CLK inhibitor or a pharmaceutically acceptable salt or solvate thereof.

Provided herein are methods of decreasing (e.g., a 1% to 99% decrease, or any of the subranges of this range described herein) the activity of one or more (e.g., one, two, three, or four) of CLK1, CLK2, CLK3 and CLK4 in a mammalian cell (e.g., any of the types of cells described herein, e.g., any of the types of cancer cells described herein) that include: contacting the mammalian cell with an effective amount of a CLK inhibitor or a pharmaceutically acceptable salt or solvate thereof. In some embodiments, the contacting results in a decrease in the activity of one or both of CLK2 and CLK3 in the mammalian cell. In some embodiments of any of the methods described herein, the mammalian cell is a cancer cell (e.g., any of the types of cancer cells described herein or known in the art). For example, the mammalian cell can be a cancer cell (e.g., any of the types of cancer cells described herein or known in the art) that has been identified as having an elevated level of Wnt pathway activity as compared to a reference level.

Various methods are known in the art to determine the activity of one or more (e.g., one, two, three, or four) of CLK1, CLK2, CLK3 and CLK4, including the methods described in the Examples).

### CLKs

The CLK family of kinases contains four characterized isoforms (CLK1, CLK2, CLK3 and CLK4). CLKs are proposed to exert their function by directly phosphorylating serine and arginine rich splicing factor (SRSF) proteins. SRSFs are reported to play an important role in spliceosome assembly and regulation of alternative splicing and gene expression.

Exemplary human CLK1, CLK2, CLK3, and CLK4 protein sequences are SEQ ID NO: 1, 3, 5, 7, 9, 11, 13, 15, and 17. Exemplary cDNA sequences that encode CLK1, CLK2, CLK3, and CLK4 are SEQ ID NO: 2, 4, 6, 8, 10, 12, 14, 16, and 18.

Human CLK1 protein isoform 1
(SEQ ID NO: 1)
MRHSKRTYCPDWDDKDWDYGKWRSSSSHKRRKRSHSSAQENKRCKYNHSKMCDSHYLESRSINEK
DYHSRRYIDEYRNDYTQGCEPGHRQRDHESRYQNHSSKSSGRSGRSSYKSKHRIHHSTSHRRSHG
KSHRRKRTRSVEDDEEGHLICQSGDVLSARYEIVDTLGEGAFGKVVECIDHKAGGRHVAVKIVKN
VDRYCEAARSEIQVLEHLNTTDPNSTFRCVQMLEWFEHHGHICIVFELLGLSTYDFIKENGFLPF
RLDHIRKMAYQICKSVNFLHSNKLTHTDLKPENILFVQSDYTEAYNPKIKRDERTLINPDIKVVD
FGSATYDDEHHSTLVSTRHYRAPEVILALGWSQPCDVWSIGCILIEYYLGFTVFPTHDSKEHLAM
MERILGPLPKHMIQKTRKRKYFHHDRLDWDEHSSAGRYVSRRCKPLKEFMLSQDVEHERL
FDLIQKMLEYDPAKRITLREALKHPFFDLLKKSI
Human CLK1 cDNA isoform 1
(SEQ ID NO: 2)
atgagacac tcaaagagaa cttactgtcc tgattgggat gacaaggatt gggattatgg
aaaatggagg agcagcagca gtcataaaag aaggaagaga tcacatagca gtgcccagga
gaacaagcgc tgcaaataca atcactctaa aatgtgtgat agccattatt tggaaagcag
gtctataaat gagaaagatt atcatagtcg acgctacatt gatgagtaca gaaatgacta
cactcaagga tgtgaacctg gacatcgcca aagagaccat gaaagccggt atcagaacca
tagtagcaag tcttctggta gaagtggaag aagtagttat aaaagcaaac acaggattca
ccacagtact tcacatcgtc gttcacatgg gaagagtcac cgaaggaaaa gaaccaggag
tgtagaggat gatgaggagg gtcacctgat ctgtcagagt ggagacgtac taagtgcaag
atatgaaatt gttgatactt taggtgaagg agcttttgga aaagttgtgg agtgcatcga
tcataaagcg ggaggtagac atgtagcagt aaaaatagtt aaaaatgtgg atagatactg
tgaagctgct cgctcagaaa tacaagttct ggaacatctg aatacaacag accccaacag
tactttccgc tgtgtccaga tgttggaatg gtttgagcat catggtcaca tttgcattgt
ttttgaacta ttgggactta gtacttacga cttcattaaa gaaaatggtt ttctaccatt
tcgactggat catatcagaa agatggcata tcagatatgc aagtctgtga attttttgca
cagtaataag ttgactcaca cagacttaaa gcctgaaaac atcttatttg tgcagtctga
ctacacagag gcgtataatc ccaaaataaa acgtgatgaa cgcaccttaa taaatccaga
tattaaagtt gtagactttg gtagtgcaac atatgatgac gaacatcaca gtacattggt
atctacaaga cattatagag cacctgaagt tattttagcc ctagggtggt cccaaccatg
tgatgtctgg agcataggat gcattcttat tgaatactat cttgggttta ccgtatttcc
aacacacgat agtaaggagc atttagcaat gatggaaagg attcttggac ctctaccaaa
acatatgata cagaaaacca ggaaacgtaa atattttcac cacgatcgat tagactggga
tgaacacagt tctgccggca gatatgtttc aagacgctgt aaacctctga aggaatttat
gctttctcaa gatgttgaac atgagcgtct ctttgacctc attcagaaaa tgttggagta
tgatccagcc aaaagaatta ctctcagaga agccttaaag catcctttct ttgaccttct
gaagaaaagt atatag
Human CLK1 protein isoform 2
(SEQ ID NO: 3)
MAAGRRPASALWPERRGSPLRGDLLGFQNVREPSSCGETLSGMRHSKRTYCPDWDDKDWDYGKWR
SSSSHKRRKRSHSSAQENKRCKYNHSKMCDSHYLESRSINEKDYHSRRYIDEYRNDYTQGCEPGH
RQRDHESRYQNHSSKSSGRSGRSSYKSKHRIHHSTSHRRSHGKSHRRKRIRSVEDDEEGHLICQS
GDVLSARYEIVDTLGEGAFGKVVECIDHKAGGRHVAVKIVKNVDRYCEAARSEIQVLEHLNTTDP
NSTFRCVQMLEWFEHHGHICIVFELLGLSTYDFIKENGFLPFRLDHIRKMAYQICKSVNFLHSNK
LTHTDLKPENILFVQSDYTEAYNPKIKRDERTLINPDIKVVDFGSATYDDEHHSTLVSTRHYRAP
EVILALGWSQPCDVWSIGCILIEYYLGFTVFPTHDSKEHLAMMERILGPLPKHMIQKTRKRKYFH
HDRLDWDEHSSAGRYVSRRCKPLKEFMLSQDVEHERLFDLIQKMLEYDPAKRI
TLREALKHPFFDLLKKSI
Human CLK1 cDNA isoform 2
(SEQ ID NO: 4)
atggc ggctgggcgg aggccggctt cggccctgtg gccggaaagg cgaggctccc
cgttgagggg ggatttgctg gggttccaga atgtgcgtga gccaagcagc tgtggggaaa
cgttgtctgg aatgagacac tcaaagagaa cttactgtcc tgattgggat gacaaggatt
gggattatgg aaaatggagg agcagcagca gtcataaaag aaggaagaga tcacatagca
gtgcccagga gaacaagcgc tgcaaataca atcactctaa aatgtgtgat agccattatt
tggaaagcag gtctataaat gagaaagatt atcatagtcg acgctacatt gatgagtaca
gaaatgacta cactcaagga tgtgaacctg gacatcgcca aagagaccat gaaagccggt
atcagaacca tagtagcaag tcttctggta gaagtggaag aagtagttat aaaagcaaac
acaggattca ccacagtact tcacatcgtc gttcacatgg gaagagtcac cgaaggaaaa
gaaccaggag tgtagaggat gatgaggagg gtcacctgat ctgtcagagt ggagacgtac
taagtgcaag atatgaaatt gttgatactt taggtgaagg agcttttgga aaagttgtgg
agtgcatcga tcataaagcg ggaggtagac atgtagcagt aaaaatagtt aaaaatgtgg
atagatactg tgaagctgct cgctcagaaa tacaagttct ggaacatctg aatacaacag
accccaacag tactttccgc tgtgtccaga tgttggaatg gtttgagcat catggtcaca
tttgcattgt ttttgaacta ttgggactta gtacttacga cttcattaaa gaaaatggtt
ttctaccatt tcgactggat catatcagaa agatggcata tcagatatgc aagtctgtga
attttttgca cagtaataag ttgactcaca cagacttaaa gcctgaaaac atcttatttg
tgcagtctga ctacacagag gcgtataatc ccaaaataaa acgtgatgaa cgcaccttaa
taaatccaga tattaaagtt gtagactttg gtagtgcaac atatgatgac gaacatcaca
gtacattggt atctacaaga cattatagag cacctgaagt tattttagcc ctagggtggt
cccaaccatg tgatgtctgg agcataggat gcattcttat tgaatactat cttgggttta
ccgtatttcc aacacacgat agtaaggagc atttagcaat gatggaaagg attcttggac
ctctaccaaa acatatgata cagaaaacca ggaaacgtaa atattttcac cacgatcgat
tagactggga tgaacacagt tctgccggca gatatgtttc aagacgctgt aaacctctga
aggaatttat gctttctcaa gatgttgaac atgagcgtct ctttgacctc attcagaaaa
tgttggagta tgatccagcc aaaagaatta ctctcagaga agccttaaag catcctttct
ttgaccttct gaagaaaagt atatag
Human CLK2 protein isoform 1
(SEQ ID NO: 5)
MPHPRRYHSSERGSRGSYREHYRSRKHKRRRSRSWSSSSDRTRRRRREDSYHVRSRSSYDDRSSD
RRVYDRRYCGSYRRNDYSRDRGDAYYDTDYRHSYEYQRENSSYRSQRSSRRKHRRRRRRSRTFSR
SSSQHSSRRAKSVEDDAEGHLIYHVGDWLQERYEIVSTLGEGTFGRVVQCVDHRRGGARVALKII
KNVEKYKEAARLEINVLEKINEKDPDNKNLCVQMFDWFDYHGHMCISFELLGLSTFDFLKDNNYL
PYPIHQVRHMAFQLCQAVKFLHDNKLTHTDLKPENILFVNSDYELTYNLEKKRDERSVKSTAVRV
VDFGSATFDHEHHSTIVSTRHYRAPEVILELGWSQPCDVWSIGCIIFEYYVGFTLFQTHDNREHL
AMMERILGPIPSRMIRKTRKQKYFYRGRLDWDENTSAGRYVRENCKPLRRYLTSEAEEHHQLFDL
IESMLEYEPAKRLTLGEALQHPFFARLRAEPPNKLWDSSRDISR
Human CLK2 cDNA isoform 1
(SEQ ID NO: 6)
a tgccgcatcc tcgaaggtac cactcctcag agcgaggcag ccgggggagt
taccgtgaac actatcggag ccgaaagcat aagcgacgaa gaagtcgctc ctggtcaagt
agtagtgacc ggacacgacg gcgtcggcga gaggacagct accatgtccg ttctcgaagc
agttatgatg atcgttcgtc cgaccggagg gtgtatgacc ggcgatactg tggcagctac
agacgcaacg attatagccg ggatcgggga gatgcctact atgacacaga ctatcggcat
tcctatgaat atcagcggga gaacagcagt taccgcagcc agcgcagcag ccggaggaag
cacagacggc ggaggaggcg cagccggaca tttagccgct catcttcgca gcacagcagc
cggagagcca agagtgtaga ggacgacgct gagggccacc tcatctacca cgtcggggac
tggctacaag agcgatatga aatcgttagc accttaggag aggggacctt cggccgagtt
gtacaatgtg ttgaccatcg caggggtggg gctcgagttg ccctgaagat cattaagaat
gtggagaagt acaaggaagc agctcgactt gagatcaacg tgctagagaa aatcaatgag
aaagaccctg acaacaagaa cctctgtgtc cagatgtttg actggtttga ctaccatggc
cacatgtgta tctcctttga gcttctgggc cttagcacct tcgatttcct caaagacaac
aactacctgc cctaccccat ccaccaagtg cgccacatgg ccttccagct gtgccaggct
gtcaagttcc tccatgataa caagctgaca catacagacc tcaagcctga aaatattctg
tttgtgaatt cagactatga gctcacctac aacctagaga agaagcgaga tgagcgcagt
gtgaagagca cagctgtgcg ggtggtagac tttggcagtg ccacctttga ccatgagcac
catagcacca ttgtctccac tcgccattac cgagcaccag aagtcatcct tgagttgggc
tggtcacagc cttgtgatgt gtggagtata ggctgcatca tctttgaata ctatgtggga
ttcaccctct tccagaccca tgacaacaga gagcatctag ccatgatgga aaggatcttg
ggtcctatcc cttcccggat gatccgaaag acaagaaagc agaaatattt ttaccggggt
cgcctggatt gggatgagaa cacatcagct gggcgctatg ttcgtgagaa ctgcaaaccg
ctgcggcggt atctgacctc agaggcagag gaacaccacc agctcttcga tctgattgaa
agcatgctag agtatgaacc agctaagcgg ctgaccttgg gtgaagccct tcagcatcct
ttcttcgccc gccttcgggc tgagccgccc aacaagttgt gggactccag tcgggatatc
agtcggtga
Human CLK2 protein isoform 2
(SEQ ID NO: 7)
MPHPRRYHSSERGSRGSYREHYRSRKHKRRRSRSWSSSSDRTRRRRREDSYHVRSRSSYDDRSSD
RRVYDRRYCGSYRRNDYSRDRGDAYYDTDYRHSYEYQRENSSYRSQRSSRRKHRRRRRRSRTFSR
SSSHSSRRAKSVEDDAEGHLIYHVGDWLQERYEIVSTLGEGTFGRVVQCVDHRRGGARVALKIIK
NVEKYKEAARLEINVLEKINEKDPDNKNLCVQMFDWFDYHGHMCISFELLGLSTFDFLKDNNYLP
YPIHQVRHMAFQLCQAVKFLHDNKLTHTDLKPENILFVNSDYELTYNLEKKRDERSVKSTAVRVV
DFGSATFDHEHHSTIVSTRHYRAPEVILELGWSQPCDVWSIGCIIFEYYVGFTLFQTHDNREHLA
MMERILGPIPSRMIRKTRKQKYFYRGRLDWDENTSAGRYVRENCKPLRRYLTSEAEEHHQLFDLI
ESMLEYEPAKRLTLGEALQHPFFARLRAEPPNKLWDSSRDISR
Human CLK2 cDNA isoform 2
(SEQ ID NO: 8)
a tgccgcatcc tcgaaggtac cactcctcag agcgaggcag ccgggggagt
taccgtgaac actatcggag ccgaaagcat aagcgacgaa gaagtcgctc ctggtcaagt
agtagtgacc ggacacgacg gcgtcggcga gaggacagct accatgtccg ttctcgaagc
agttatgatg atcgttcgtc cgaccggagg gtgtatgacc ggcgatactg tggcagctac
agacgcaacg attatagccg ggatcgggga gatgcctact atgacacaga ctatcggcat
tcctatgaat atcagcggga gaacagcagt taccgcagcc agcgcagcag ccggaggaag
cacagacggc ggaggaggcg cagccggaca tttagccgct catcttcgca cagcagccgg
agagccaaga gtgtagagga cgacgctgag ggccacctca tctaccacgt cggggactgg
ctacaagagc gatatgaaat cgttagcacc ttaggagagg ggaccttcgg ccgagttgta
caatgtgttg accatcgcag gggtggggct cgagttgccc tgaagatcat taagaatgtg
gagaagtaca aggaagcagc tcgacttgag atcaacgtgc tagagaaaat caatgagaaa
gaccctgaca acaagaacct ctgtgtccag atgtttgact ggtttgacta ccatggccac
atgtgtatct cctttgagct tctgggcctt agcaccttcg atttcctcaa agacaacaac
tacctgccct accccatcca ccaagtgcgc cacatggcct tccagctgtg ccaggctgtc
aagttcctcc atgataacaa gctgacacat acagacctca agcctgaaaa tattctgttt
gtgaattcag actatgagct cacctacaac ctagagaaga agcgagatga gcgcagtgtg
aagagcacag ctgtgcgggt ggtagacttt ggcagtgcca cctttgacca tgagcaccat
agcaccattg tctccactcg ccattaccga gcaccagaag tcatccttga gttgggctgg
tcacagcctt gtgatgtgtg gagtataggc tgcatcatct ttgaatacta tgtgggattc
accctcttcc agacccatga caacagagag catctagcca tgatggaaag gatcttgggt
cctatccctt cccggatgat ccgaaagaca agaaagcaga aatattttta ccggggtcgc
ctggattggg atgagaacac atcagctggg cgctatgttc gtgagaactg caaaccgctg
cggcggtatc tgacctcaga ggcagaggaa caccaccagc tcttcgatct gattgaaagc
atgctagagt atgaaccagc taagcggctg accttgggtg aagcccttca gcatcctttc
ttcgcccgcc ttcgggctga gccgcccaac aagttgtggg actccagtcg ggatatcagt
cggtga
Human CLK2 protein isoform 3
(SEQ ID NO: 9)
MFDWFDYHGHMCISFELLGLSTFDFLKDNNYLPYPIHQVRHMAFQLCQAVKFLHDNKLTHTDLKP
ENILFVNSDYELTYNLEKKRDERSVKSTAVRVVDEGSATFDHEHHSTIVSTRHYRAPEVILELGW
SQPCDVWSIGCIIFEYYVGFTLFQTHDNREHLAMMERILGPIPSRMIRKTRKQKYFYRGRLDWDE
NTSAGRYVRENCKPLRRYLTSEAEEHHQLFDLIESMLEYEPAKRLTLGEALQHPFFARLRAEPPN
KLWDSSRDISR
Human CLK2 cDNA isoform 3
(SEQ ID NO: 10)
atgtt tgactggttt gactaccatg gccacatgtg tatctccttt gagcttctgg
gccttagcac cttcgatttc ctcaaagaca acaactacct gccctacccc atccaccaag
tgcgccacat ggccttccag ctgtgccagg ctgtcaagtt cctccatgat aacaagctga
cacatacaga cctcaagcct gaaaatattc tgtttgtgaa ttcagactat gagctcacct
acaacctaga gaagaagcga gatgagcgca gtgtgaagag cacagctgtg cgggtggtag
actttggcag tgccaccttt gaccatgagc accatagcac cattgtctcc actcgccatt
accgagcacc agaagtcatc cttgagttgg gctggtcaca gccttgtgat gtgtggagta
taggctgcat catctttgaa tactatgtgg gattcaccct cttccagacc catgacaaca
gagagcatct agccatgatg gaaaggatct tgggtcctat cccttcccgg atgatccgaa
agacaagaaa gcagaaatat ttttaccggg gtcgcctgga ttgggatgag aacacatcag
ctgggcgcta tgttcgtgag aactgcaaac cgctgcggcg gtatctgacc tcagaggcag
aggaacacca ccagctcttc gatctgattg aaagcatgct agagtatgaa ccagctaagc
ggctgacctt gggtgaagcc cttcagcatc ctttcttcgc ccgccttcgg gctgagccgc
ccaacaagtt gtgggactcc agtcgggata tcagtcggtg a
Human CLK2 protein isoform 4
(SEQ ID NO: 11)
MPHPRRYHSSERGSRGSYREHYRSRKHKRRRSRSWSSSSDRTRRRRREDSYHVRSRSYDDRSSDR
RVYDRRYCGSYRRNDYSRDRGDAYYDTDYRHSYEYQRENSSYRSQRSSRRKHRRRRRRSRTFSRS
SSHSSRRAKSVEDDAEGHLIYHVGDWLQERYEIVSTLGEGTFGRVVQCVDHRRGGARVALKIIKN
VEKYKEAARLEINVLEKINEKDPDNKNLCVQMFDWFDYHGHMCISFELLGLSTFDFLKDNNYLPY
PIHQVRHMAFQLCQAVKFLHDNKLTHTDLKPENILFVNSDYELTYNLEKKRDERSVKSTAVRVVD
FGSATFDHEHHSTIVSTRHYRAPEVILELGWSQPCDVWSIGCIIFEYYVGFTLFQTHDNREHLAM
MERILGPIPSRMIRKTRKQKYFYRGRLDWDENTSAGRYVRENCKPLRRYLTSEAEEHHQLFDLIE
SMLEYEPAKRLTLGEALQHPFFARLRAEPPNKLWDSSRDISR
Human CLK2 cDNA isoform 4
(SEQ ID NO: 12)
a tgccgcatcc tcgaaggtac cactcctcag agcgaggcag ccgggggagt
taccgtgaac actatcggag ccgaaagcat aagcgacgaa gaagtcgctc ctggtcaagt
agtagtgacc ggacacgacg gcgtcggcga gaggacagct accatgtccg ttctcgaagt
tatgatgatc gttcgtccga ccggagggtg tatgaccggc gatactgtgg cagctacaga
cgcaacgatt atagccggga tcggggagat gcctactatg acacagacta tcggcattcc
tatgaatatc agcgggagaa cagcagttac cgcagccagc gcagcagccg gaggaagcac
agacggcgga ggaggcgcag ccggacattt agccgctcat cttcgcacag cagccggaga
gccaagagtg tagaggacga cgctgagggc cacctcatct accacgtcgg ggactggcta
caagagcgat atgaaatcgt tagcacctta ggagagggga ccttcggccg agttgtacaa
tgtgttgacc atcgcagggg tggggctcga gttgccctga agatcattaa gaatgtggag
aagtacaagg aagcagctcg acttgagatc aacgtgctag agaaaatcaa tgagaaagac
cctgacaaca agaacctctg tgtccagatg tttgactggt ttgactacca tggccacatg
tgtatctcct ttgagcttct gggccttagc accttcgatt tcctcaaaga caacaactac
ctgccctacc ccatccacca agtgcgccac atggccttcc agctgtgcca ggctgtcaag
ttcctccatg ataacaagct gacacataca gacctcaagc ctgaaaatat tctgtttgtg
aattcagact atgagctcac ctacaaccta gagaagaagc gagatgagcg cagtgtgaag
agcacagctg tgcgggtggt agactttggc agtgccacct ttgaccatga gcaccatagc
accattgtct ccactcgcca ttaccgagca ccagaagtca tccttgagtt gggctggtca
cagccttgtg atgtgtggag tataggctgc atcatctttg aatactatgt gggattcacc
ctcttccaga cccatgacaa cagagagcat ctagccatga tggaaaggat cttgggtcct
atcccttccc ggatgatccg aaagacaaga aagcagaaat atttttaccg gggtcgcctg
gattgggatg agaacacatc agctgggcgc tatgttcgtg agaactgcaa accgctgcgg
cggtatctga cctcagaggc agaggaacac caccagctct tcgatctgat tgaaagcatg
ctagagtatg aaccagctaa gcggctgacc ttgggtgaag cccttcagca tcctttcttc
gcccgccttc gggctgagcc gcccaacaag ttgtgggact ccagtcggga tatcagtcgg
tga
Human CLK3 protein isoform 1
(SEQ ID NO: 13)
MPVLSARRRELADHAGSGRRSGPSPTARSGPHLSALRAQPARAAHLSGRGTYVRRDTAGGGPGQA
RPLGPPGTSLLGRGARRSGEGWCPGAFESGARAARPPSRVEPRLATAASREGAGLPRAEVAAGSG
RGARSGEWGLAAAGAWETMHHCKRYRSPEPDPYLSYRWKRRRSYSREHEGRLRYPSRREPPPRRS
RSRSHDRLPYQRRYRERRDSDTYRCEERSPSFGEDYYGPSRSRHRRRSRERGPYRTRKHAHHCHK
RRTRSCSSASSRSQQSSKRSSRSVEDDKEGHLVCRIGDWLQERYEIVGNLGEGTFGKVVECLDHA
RGKSQVALKIIRNVGKYREAARLEINVLKKIKEKDKENKFLCVLMSDWFNFHGHMCIAFELLGKN
TFEFLKENNFQPYPLPHVRHMAYQLCHALRFLHENQLTHTDLKPENILFVNSEFETLYNEHKSCE
EKSVKNTSIRVADFGSATFDHEHHTTIVATRHYRPPEVILELGWAQPCDVWSIGCILFEYYRGFT
LFQTHENREHLVMMEKILGPIPSHMIHRTRKQKYFYKGGLVWDENSSDGRYVKENCKPLKSYMLQ
DSLEHVQLFDLMRRMLEFDPAQRITLAEALLHPFFAGLT PEERSFHTSRNPSR
Human CLK3 cDNA isoform 1
(SEQ ID NO: 14)
a atgcccgtc ctctccgcgc gcaggaggga gttggcggac cacgcggggt cggggcgacg
gagcgggccc agccccacgg ccaggtcggg gccccacctc tcggctctga gagcccagcc
ggcccgggcc gcgcacctgt caggtcgggg gacctacgtg cgccgcgaca cggcgggagg
cgggccgggc caggctcgtc ccctcggccc tcccggaact agtctcctag gccgcggcgc
ccgccggagc ggagagggct ggtgccccgg agccttcgag tcgggggcta gagcggccag
gcctccgagc cgggtcgagc cgaggctggc gacggctgcg tcacgcgagg gggcggggct
gccacgggcg gaggtcgcag ccggaagcgg aagaggcgct cggagcgggg agtggggcct
agctgcagcc ggagcctggg agacgatgca tcactgtaag cgataccgct cccctgaacc
agacccgtac ctgagctacc gatggaagag gaggaggtcc tacagtcggg aacatgaagg
gagactgcga tacccgtccc gaagggagcc tcccccacga agatctcggt ccagaagcca
tgaccgcctg ccctaccaga ggaggtaccg ggagcgccgt gacagcgata cataccggtg
tgaagagcgg agcccatcct ttggagagga ctactatgga ccttcacgtt ctcgtcatcg
tcggcgatcg cgggagaggg ggccataccg gacccgcaag catgcccacc actgccacaa
acgccgcacc aggtcttgta gcagcgcctc ctcgagaagc caacagagca gtaagcgcag
cagccggagt gtggaagatg acaaggaggg tcacctggtg tgccggatcg gcgattggct
ccaagagcga tatgagattg tggggaacct gggtgaaggc acctttggca aggtggtgga
gtgcttggac catgccagag ggaagtctca ggttgccctg aagatcatcc gcaacgtggg
caagtaccgg gaggctgccc ggctagaaat caacgtgctc aaaaaaatca aggagaagga
caaagaaaac aagttcctgt gtgtcttgat gtctgactgg ttcaacttcc acggtcacat
gtgcatcgcc tttgagctcc tgggcaagaa cacctttgag ttcctgaagg agaataactt
ccagccttac cccctaccac atgtccggca catggcctac cagctctgcc acgcccttag
atttctgcat gagaatcagc tgacccatac agacttgaaa ccagagaaca tcctgtttgt
gaattctgag tttgaaaccc tctacaatga gcacaagagc tgtgaggaga agtcagtgaa
gaacaccagc atccgagtgg ctgactttgg cagtgccaca tttgaccatg agcaccacac
caccattgtg gccacccgtc actatcgccc gcctgaggtg atccttgagc tgggctgggc
acagccctgt gacgtctgga gcattggctg cattctcttt gagtactacc ggggcttcac
actcttccag acccacgaaa accgagagca cctggtgatg atggagaaga tcctagggcc
catcccatca cacatgatcc accgtaccag gaagcagaaa tatttctaca aagggggcct
agtttgggat gagaacagct ctgacggccg gtatgtgaag gagaactgca aacctctgaa
gagttacatg ctccaagact ccctggagca cgtgcagctg tttgacctga tgaggaggat
gttagaattt gaccctgccc agcgcatcac actggccgag gccctgctgc accccttctt
tgctggcctg acccctgagg agcggtcctt ccacaccagc cgcaacccaa gcagatga
Human CLK3 protein isoform 2
(SEQ ID NO: 15)
MHHCKRYRSPEPDPYLSYRWKRRRSYSREHEGRLRYPSRREPPPRRSRSRSHDRLPYQRRYRERR
DSDTYRCEERSPSFGEDYYGPSRSRHRRRSRERGPYRTRKHAHHCHKRRTRSCSSASSRSQQSSK
RSSRSVEDDKEGHLVCRIGDWLQERYEIVGNLGEGTFGKVVECLDHARGKSQVALKIIRNVGKYR
EAARLEINVLKKIKEKDKENKFLCVLMSDWFNFHGHMCIAFELLGKNTFEFLKENNFQPYPLPHV
RHMAYQLCHALRFLHENQLTHTDLKPENILFVNSEFETLYNEHKSCEEKSVKNTSIRVADFGSAT
FDHEHHTTIVATRHYRPPEVILELGWAQPCDVWSIGCILFEYYRGFTLFQTHENREHLVMMEKIL
GPIPSHMIHRTRKQKYFYKGGLVWDENSSDGRYVKENCKPLKSYMLQDSLEHVQLFDLMR
RMLEFDPAQRITLAEALLHPFFAGLTPEERSFHTSRNPSR
Human CLK3 cDNA isoform 2
(SEQ ID NO: 16)
atgca tcactgtaag cgataccgct cccctgaacc agacccgtac ctgagctacc
gatggaagag gaggaggtcc tacagtcggg aacatgaagg gagactgcga tacccgtccc
gaagggagcc tcccccacga agatctcggt ccagaagcca tgaccgcctg ccctaccaga
ggaggtaccg ggagcgccgt gacagcgata cataccggtg tgaagagcgg agcccatcct
ttggagagga ctactatgga ccttcacgtt ctcgtcatcg tcggcgatcg cgggagaggg
ggccataccg gacccgcaag catgcccacc actgccacaa acgccgcacc aggtcttgta
gcagcgcctc ctcgagaagc caacagagca gtaagcgcag cagccggagt gtggaagatg
acaaggaggg tcacctggtg tgccggatcg gcgattggct ccaagagcga tatgagattg
tggggaacct gggtgaaggc acctttggca aggtggtgga gtgcttggac catgccagag
ggaagtctca ggttgccctg aagatcatcc gcaacgtggg caagtaccgg gaggctgccc
ggctagaaat caacgtgctc aaaaaaatca aggagaagga caaagaaaac aagttcctgt
gtgtcttgat gtctgactgg ttcaacttcc acggtcacat gtgcatcgcc tttgagctcc
tgggcaagaa cacctttgag ttcctgaagg agaataactt ccagccttac cccctaccac
atgtccggca catggcctac cagctctgcc acgcccttag atttctgcat gagaatcagc
tgacccatac agacttgaaa ccagagaaca tcctgtttgt gaattctgag tttgaaaccc
tctacaatga gcacaagagc tgtgaggaga agtcagtgaa gaacaccagc atccgagtgg
ctgactttgg cagtgccaca tttgaccatg agcaccacac caccattgtg gccacccgtc
actatcgccc gcctgaggtg atccttgagc tgggctgggc acagccctgt gacgtctgga
gcattggctg cattctcttt gagtactacc ggggcttcac actcttccag acccacgaaa
accgagagca cctggtgatg atggagaaga tcctagggcc catcccatca cacatgatcc
accgtaccag gaagcagaaa tatttctaca aagggggcct agtttgggat gagaacagct
ctgacggccg gtatgtgaag gagaactgca aacctctgaa gagttacatg ctccaagact
ccctggagca cgtgcagctg tttgacctga tgaggaggat gttagaattt gaccctgccc
agcgcatcac actggccgag gccctgctgc accccttctt tgctggcctg acccctgagg
agcggtcctt ccacaccagc cgcaacccaa gcagatga
Human CLK4 protein
(SEQ ID NO: 17)
MRHSKRTHCPDWDSRESWGHESYRGSHKRKRRSHSSTQENRHCKPHHQFKESDCHYLEARSLNER
DYRDRRYVDEYRNDYCEGYVPRHYHRDIESGYRIHCSKSSVRSRRSSPKRKRNRHCSSHQSRSKS
HRRKRSRSIEDDEEGHLICQSGDVLRARYEIVDTLGEGAFGKVVECIDHGMDGMHVAVKIVKNVG
RYREAARSEIQVLEHLNSTDPNSVERCVQMLEWFDHHGHVCIVFELLGLSTYDFIKENSFLPFQI
DHIRQMAYQICQSINFLHHNKLTHTDLKPENILFVKSDYVVKYNSKMKRDERTLKNIDIKVVDFG
SATYDDEHHSTLVSTRHYRAPEVILALGWSQPCDVWSIGCILIEYYLGFTVFQTHDSKEHLAMME
RILGPIPQHMIQKTRKRKYFHHNQLDWDEHSSAGRYVRRRCKPLKEFMLCHDEEHEKLFD
LVRRMLEYDPTQRITLDEALQHPFFDLLKKK
Human CLK4 cDNA
(SEQ ID NO: 18)
at gcggcattcc aaaagaactc actgtcctga ttgggatagc agagaaagct
ggggacatga aagctatcgt ggaagtcaca agcggaagag gagatctcat agtagcacac
aagagaacag gcattgtaaa ccacatcacc agtttaaaga atctgattgt cattatttag
aagcaaggtc cttgaatgag cgagattatc gggaccggag atacgttgac gaatacagga
atgactactg tgaaggatat gttcctagac attatcacag agacattgaa agcgggtatc
gaatccactg cagtaaatct tcagtccgca gcaggagaag cagtcctaaa aggaagcgca
atagacactg ttcaagtcat cagtcacgtt cgaagagcca ccgaaggaaa agatccagga
gtatagagga tgatgaggag ggtcacctga tctgtcaaag tggagacgtt ctaagagcaa
gatatgaaat cgtggacact ttgggtgaag gagcctttgg caaagttgta gagtgcattg
atcatggcat ggatggcatg catgtagcag tgaaaatcgt aaaaaatgta ggccgttacc
gtgaagcagc tcgttcagaa atccaagtat tagagcactt aaatagtact gatcccaata
gtgtcttccg atgtgtccag atgctagaat ggtttgatca tcatggtcat gtttgtattg
tgtttgaact actgggactt agtacttacg atttcattaa agaaaacagc tttctgccat
ttcaaattga ccacatcagg cagatggcgt atcagatctg ccagtcaata aattttttac
atcataataa attaacccat acagatctga agcctgaaaa tattttgttt gtgaagtctg
actatgtagt caaatataat tctaaaatga aacgtgatga acgcacactg aaaaacacag
atatcaaagt tgttgacttt ggaagtgcaa cgtatgatga tgaacatcac agtactttgg
tgtctacccg gcactacaga gctcccgagg tcattttggc tttaggttgg tctcagcctt
gtgatgtttg gagcataggt tgcattctta ttgaatatta ccttggtttc acagtctttc
agactcatga tagtaaagag cacctggcaa tgatggaacg aatattagga cccataccac
aacacatgat tcagaaaaca agaaaacgca agtattttca ccataaccag ctagattggg
atgaacacag ttctgctggt agatatgtta ggagacgctg caaaccgttg aaggaattta
tgctttgtca tgatgaagaa catgagaaac tgtttgacct ggttcgaaga atgttagaat
atgatccaac tcaaagaatt accttggatg aagcattgca gcatcctttc tttgacttat
taaaaaagaa atga

Methods of Altering mRNA Splicing

Also provided herein are methods of altering mRNA splicing in a mammalian cell (e.g., any of the exemplary mammalian cells described herein, e.g., any of the exemplary types of cancer cells described herein) having aberrant mRNA splicing activity that include: contacting the mammalian cell with an effective amount of a CLK inhibitor (e.g., any of the exemplary CLK inhibitors described herein or known in the art) or a pharmaceutically acceptable salt or solvate thereof.

In some aspects, the mammalian cell is a cancer cell (e.g., any of the exemplary types of cancer cells described herein or known in the art). For example, the mammalian cell is a cancer cell having aberrant mRNA spicing activity has one or more (e.g., two, three, four, five, or six) of: an increased level of phosphorylated serine and arginine rich splicing factor 6 (SRSF6) as compared to a reference level (e.g., any of the reference levels described herein); an increased level of phosphorylated serine and arginine rich splicing factor 5 (SRSF5) as compared to a reference level (e.g., any of the reference levels described herein); a mutation in a splicing factor 3b subunit 1 (SF3B1) gene, a serine and arginine rich splicing factor 1 (SRSF1) gene, a serine and arginine rich splicing factor 2 (SRSF2) gene, a small nuclear RNA auxiliary factor 1 (U2AF1) gene, or a zinc finger CCCH-type, RNA binding motif and serine/arginine rich 2 (ZRSR2) gene; and an increased level of SRSF1, SRSF2, serine and arginine rich splicing factor 3 (SRSF3), serine and arginine rich splicing factor 4 (SRSF4), SRSF5, SRSF6, and serine and arginine rich splicing factor 10 (SRSF10) as compared to a reference level (e.g., any of the exemplary reference levels described herein).

In some embodiments of any of the methods described herein, the level of aberrant mRNA splicing is determined by detecting: the level of SRSF6 phosphorylation in the cell; the level of SRSF5 phosphorylation in the cell; the level of a ˜55 kDa isoform of SRSF6 in the cell; or the level of ˜35 kDa isoform of SRSF1 in the cell. Exemplary methods for detecting the level of SRSF6 phosphorylation, the level of SRSF5 phosphorylation, the level of the ˜55 kDa isoform of SRSF6, and the level of the ˜35 kDa isoform of SRSF1 are described in the Examples. Additional methods for determining the level of SRSF6 phosphorylation, the level of SRSF5 phosphorylation, the level of the ˜55 kDa isoform of SRSF6, and the level of the ˜35 kDa isoform of SRSF1 are known in the art. Exemplary sequences for human SRSF1, SRSF2, SRSF3, SF3B1, SRSF4, SRSF5, SRSF6, SRSF10, U2AF1, and ZRSR2 proteins are shown below.

SRSF1 (NCBI Accession NM_006924.4)
(SEQ ID NO: 19)
MSGGGVIRGPAGNNDCRIYVGNLPPDIRTKDIEDVFYKYGAIRDIDLKNRRGGPPFAFVEFEDPR
DAEDAVYGRDGYDYDGYRLRVEFPRSGRGTGRGGGGGGGGGAPRGRYGPPSRRSENRVVVSGLPP
SGSWQDLKDHMREAGDVCYADVYRDGTGVVEFVRKEDMTYAVRKLDNTKFRSHEGETAYIRVKVD
GPRSPSYGRSRSRSRSRSRSRSR SNSRSRSYSPRRSRGSPRYSPRHSRSRSRT
SRSF2 (NCBI Accession NM_001195427.1)
(SEQ ID NO: 20)
MSYGRPPPDVEGMTSLKVDNLTYRTSPDTLRRVFEKYGRVGDVYIPRDRYTKESRGFAFVRFHDK
RDAEDAMDAMDGAVLDGRELRVQMARYGRPPDSHHSRRGPPPRRYGGGGYGRRSRSPRRRRRSRS
RSRSRSRSRSRSRYSRSKSRSRTRSRSRSTSKSRSARRSKSKSSSVSRSRSRSRSRSRSRSPPPV
SKRESKSRSRSKSPPKSPEEEGAVSS
SRSF3 (NCBI Accession NM_003017.4)
(SEQ ID NO: 21)
MHRDSCPLDCKVYVGNLGNNGNKTELERAFGYYGPLRSVWVARNPPGFAFVEFEDPRDAADAVRE
LDGRTLCGCRVRVELSNGEKRSRNRGPPPSWGRRPRDDYRRRSPPPRRRSPRRRSFSRSRSRSLS
RDRRRERSLSRERNHKPSRSFSRSRSRSRSNERK
SF3B1 (NCBI Accession NM_012433.3)
(SEQ ID NO: 22)
MAKIAKTHEDIEAQIREIQGKKAALDEAQGVGLDSTGYYDQEIYGGSDSRFAGYVTSIAATELED
DDDDYSSSTSLLGQKKPGYHAPVALLNDIPQSTEQYDPFAEHRPPKIADREDEYKKHRRTMIISP
ERLDPFADGGKTPDPKMNARTYMDVMREQHLTKEEREIRQQLAEKAKAGELKVVNGAAASQPPSK
RKRRWDQTADQTPGATPKKLSSWDQAETPGHTPSLRWDETPGRAKGSETPGATPGSKIWDPTPSH
TPAGAATPGRGDTPGHATPGHGGATSSARKNRWDETPKTERDTPGHGSGWAETPRTDRGGDSIGE
TPTPGASKRKSRWDETPASQMGGSTPVLTPGKTPIGTPAMNMATPTPGHIMSMTPEQLQAWRWER
EIDERNRPLSDEELDAMFPEGYKVLPPPAGYVPIRTPARKLTATPTPLGGMTGFHMQTEDRTMKS
VNDQPSGNLPFLKPDDIQYFDKLLVDVDESTLSPEEQKERKIMKLLLKIKNGTPPMRKAALRQIT
DKAREFGAGPLFNQILPLLMSPTLEDQERHLLVKVIDRILYKLDDLVRPYVHKILVVIEPLLIDE
DYYARVEGREIISNLAKAAGLATMISTMRPDIDNMDEYVRNTTARAFAVVASALGIPSLLPFLKA
VCKSKKSWQARHTGIKIVQQIAILMGCAILPHLRSLVEIIEHGLVDEQQKVRTISALAIAALAEA
ATPYGIESFDSVLKPLWKGIRQHRGKGLAAFLKAIGYLIPLMDAEYANYYTREVMLILIREFQSP
DEEMKKIVLKVVKQCCGTDGVEANYIKTEILPPFFKHFWQHRMALDRRNYRQLVDTTVELANKVG
AAEIISRIVDDLKDEAEQYRKMVMETIEKIMGNLGAADIDHKLEEQLIDGILYAFQEQTTEDSVM
LNGFGTVVNALGKRVKPYLPQICGTVLWRLNNKSAKVRQQAADLISRTAVVMKTCQEEKLMGHLG
VVLYEYLGEEYPEVLGSILGALKAIVNVIGMHKMTPPIKDLLPRLTPILKNRHEKVQENCIDLVG
RIADRGAEYVSAREWMRICFELLELLKAHKKAIRRATVNTFGYIAKAIGPHDVLATLLNNLKVQE
RQNRVCTTVAIAIVAETCSPFTVLPALMNEYRVPELNVQNGVLKSLSFLFEYIGEMGKDYIYAVT
PLLEDALMDRDLVHRQTASAVVQHMSLGVYGFGCEDSLNHLLNYVWPNVFETSPHVIQAVMGALE
GLRVAIGPCRMLQYCLQGLFHPARKVRDVYWKIYNSIYIGSQDALIAHYPRIYNDDKNTYIRYEL
DYIL
SRSF4 (NCBI Accession NM_005626.4)
(SEQ ID NO: 23)
MPRVYIGRLSYQARERDVERFFKGYGKILEVDLKNGYGFVEFDDLRDADDAVYELNGKDLCGERV
IVEHARGPRRDGSYGSGRSGYGYRRSGRDKYGPPTRTEYRLIVENLSSRCSWQDLKDYMRQAGEV
TYADAHKGRKNEGVIEFVSYSDMKRALEKLDGTEVNGRKIRLVEDKPGSRRRRSYSRSRSHSRSR
SRSRHSRKSRSRSGSSKSSHSKSRSRSRSGSRSRSKSRSRSQSRSRSKKEKSRSPSKEKSRSRSH
SAGKSRSKSKDQAEEKIQNNDNVGKPKSRSPSRHKSKSKSRSRSQERRVEEEKRGSVSRGRSQEK
SLRQSRSRSRSKGGSRSRSRSRSKSKDKRKGRKRSREESRSRSRSRSKSERSRKRGSKRDSKAGS
SKKKKKEDTDRSQSRSPSRSVSKEREHAKSESSQREGRGESENAGTNQETRSRSRSNSKS
KPNLPSESRSRSKSASKTRSRSKSRSRSASRSPSRSRSRSHSRS
SRSF5 (NCBI Accession NM_001039465.1)
(SEQ ID NO: 24)
MSGCRVFIGRLNPAAREKDVERFFKGYGRIRDIDLKRGFGFVEFEDPRDADDAVYELDGKELCSE
RVTIEHARARSRGGRGRGRYSDRFSSRRPRNDRRNAPPVRTENRLIVENLSSRVSWQDLKDFMRQ
AGEVTFADAHRPKLNEGVVEFASYGDLKNAIEKLSGKEINGRKIKLIEGSKRHSRSRSRSRSRTR
SSSRSRSRSRSRSRKSYSRSRSRSRSRSRSKSRSVSRSPVPEKSQKRGSSSRSKSPASVDRQRSR
SRSRSRSVDSGN
SRSF6 (NCBI Accession NM_006275.5)
(SEQ ID NO: 25)
MPRVYIGRLSYNVREKDIQRFFSGYGRLLEVDLKNGYGFVEFEDSRDADDAVYELNGKELCGERV
IVEHARGPRRDRDGYSYGSRSGGGGYSSRRTSGRDKYGPPVRTEYRLIVENLSSRCSWQDLKDFM
RQAGEVTYADAHKERTNEGVIEFRSYSDMKRALDKLDGTEINGRNIRLIEDKPRTSHRRSYSGSR
SRSRSRRRSRSRSRRSSRSRSRSISKSRSRSRSRSKGRSRSRSKGRKSRSKSKSKPKSDRGSHSH
SRSRSKDEYEKSRSRSRSRSPKENGKGDIKSKSRSRSQSRSNSPLPVPPSKARSVSPPPKRATSR
SRSRSRSKS RSRSRSSSRD
SRSF10 (NCBI Accession NM_006625.5)
(SEQ ID NO: 26)
MSRYLRPPNTSLFVRNVADDTRSEDLRREFGRYGPIVDVYVPLDFYTRRPRGFAYVQFEDVRDAE
DALHNLDRKWICGRQIEIQFAQGDRKTPNQMKAKEGRNVYSSSRYDDYDRYRRSRSRSYERRRSR
SRSFDYNYRRSYSPRNSRPTGRPRRSRSHSDNDRPNCSWNTQYSSAYYTSRKI
U2AF1 (NCBI Accession NM_001025203.1)
(SEQ ID NO: 27)
MAEYLASIFGTEKDKVNCSFYFKIGACRHGDRCSRLHNKPTFSQTILIQNIYRNPQNSAQTADGS
HCAVSDVEMQEHYDEFFEEVFTEMEEKYGEVEEMNVCDNLGDHLVGNVYVKFRREEDAEKAVIDL
NNRWFNGQPIHAELSPVTDFREACCRQYEMGECTRGGFCNFMHLKPISRELRRELYGRRRKKHRS
RSRSRERRSRSRDRGRGGGGGGG GGGGGRERDRRRSRDRERSGRF
ZRSR2 (NCBI Accession NM_005089.3)
(SEQ ID NO: 28)
MAAPEKMTFPEKPSHKKYRAALKKEKRKKRRQELARLRDSGLSQKEEEEDTFIEEQQLEEEKLLE
RERQRLHEEWLLREQKAQEEFRIKKEKEEAAKKRQEEQERKLKEQWEEQQRKEREEEEQKRQEKK
EKEEALQKMLDQAENELENGTTNQNPEPPVDFRVMEKDRANCPFYSKTGACRFGDRCSRKHNFPT
SSPTLLIKSMFTTFGMEQCRRDDYDPDASLEYSEEETYQQFLDFYEDVLPEFKNVGKVIQFKVSC
NLEPHLRGNVYVQYQSEEECQAALSLFNGRWYAGRQLQCEFCPVTRWKMAICGLFEIQQCPRGKH
CNFLHVFRNPNNEFWEANRDIYLSPDRTGSSFGKNSERRERMGHHDDYYSRLRGRRNPSPDHSYK
RNGESERKSSRHRGKKSHKRTSKSRERHNSRSRGRNRDRSRDRSRGRGSRSRSRSRSRRS
RRSRSQSSSRSRSRGRRRSGNRDRTVQSPKSK

Exemplary methods for detecting a mutation in a SF3B1 gene, a SRSF1 gene, a SRSF2 gene, a U2AF1 gene, or a ZRSR2 gene are also described herein.

Additional methods of identifying or detecting aberrant mRNA splicing in a mammalian cell are known in the art.

### Methods of Treating a Subject—Type B

Also provided herein are methods of treating a cancer (e.g., any of the exemplary types of cancer described herein or known in the art) in a subject (e.g., any of the subjects described herein) that include: identifying a subject having a cancer cell (e.g., any of the exemplary types of cancer cells described herein or known in the art) that has aberrant mRNA splicing activity as compared to a reference level (e.g., any of the exemplary reference levels described herein); and administering to the identified subject a therapeutically effective amount of a CLK inhibitor (e.g., any of the exemplary CLK inhibitors described herein or known in the art) or a pharmaceutically acceptable salt or solvate thereof.

Also provided herein are methods of treating a cancer (e.g., any of the exemplary types of cancer described herein or known in the art) in a subject (e.g., any of the subjects described herein) that include administering a therapeutically effective amount of a CLK inhibitor (e.g., any of the exemplary CLK inhibitors described herein or known in the art) or a pharmaceutically acceptable salt or solvent thereof to a subject identified as having a cancer cell (e.g., any of the exemplary types of cancer cell described herein or known in the art) that has aberrant mRNA splicing activity as compared to a reference level (e.g., any of the reference levels described herein).

Also provided herein are methods of treating a subject (e.g., any of the subjects described herein) having a cancer (e.g., any of the cancers described herein or known in the art) that include: (a) administering to the subject a therapeutic agent (e.g., any therapeutic agent that is not a CLK inhibitor or any therapeutic regimen that does not include a CLK inhibitor as a monotherapy); (b) after (a), identifying the subject as having a cancer cell (e.g., any of the types of cancer cell described herein or known in the art) that has aberrant mRNA splicing activity as compared to a reference level (e.g., any of the reference levels described herein); and (c) administering to the identified subject a treatment comprising a therapeutically effective amount of a CLK inhibitor (e.g., any of the CLK inhibitors described herein) or a pharmaceutically acceptable salt or solvent thereof.

Also provided herein are methods of treating a subject (e.g., any of the subjects described herein) having a cancer (e.g., any of the types of cancer described herein or known in the art) that include: identifying a subject previously administered a therapeutic agent (e.g., any therapeutic agent that is not a CLK inhibitor or any therapeutic regimen that does not include a CLK inhibitor as a monotherapy), as having a cancer cell (e.g., any of the types of cancer cell described herein or known in the art) that has aberrant mRNA splicing activity as compared to a reference level (e.g., any of the exemplary reference levels described herein); and administering to the identified subject a treatment comprising a therapeutically effective amount of a CLK inhibitor (e.g., any of the exemplary types of CLK inhibitors described herein or known in the art) or a pharmaceutically acceptable salt or solvent thereof.

Also provided herein are methods of treating a subject (e.g., any of the subjects described herein) having cancer (e.g., any of the examples of cancer described herein or known in the art) that include administering to a subject previously administered a therapeutic agent (e.g., any therapeutic agent that is not a CLK inhibitor or any therapeutic regimen that does not include a CLK inhibitor as a monotherapy) and later identified as having aberrant mRNA splicing activity as compared to a reference level (e.g., any of the exemplary reference levels described herein), a therapeutically effective amount of a CLK inhibitor (e.g., any of the exemplary CLK inhibitors described herein or known in the art) or a pharmaceutically acceptable salt or solvent thereof.

In some embodiments of any of the methods of treating described herein, the cancer cell having aberrant mRNA spicing activity can have one or more (e.g., two, three, four, five, or six) of: an increased level of phosphorylated serine and arginine rich splicing factor 6 (SRSF6) as compared to a reference level (e.g., any of the reference levels described herein); an increased level of phosphorylated serine and arginine rich splicing factor 5 (SRSF5) as compared to a reference level (e.g., any of the reference levels described herein); a mutation in a splicing factor 3b subunit 1 (SF3B1) gene, a serine and arginine rich splicing factor 1 (SRSF1) gene, a serine and arginine rich splicing factor 2 (SRSF2) gene, a small nuclear RNA auxiliary factor 1 (U2AF1) gene, or a zinc finger CCCH-type, RNA binding motif and serine/arginine rich 2 (ZRSR2) gene; and an increased level of SRSF1, SRSF2, serine and arginine rich splicing factor 3 (SRSF3), serine and arginine rich splicing factor 4 (SRSF4), SRSF5, SRSF6, and serine and arginine rich splicing factor 10 (SRSF10) as compared to a reference level (e.g., any of the exemplary reference levels described herein).

In some embodiments of any of the methods of treating described herein, the level of aberrant mRNA splicing is determined by detecting: the level of SRSF6 phosphorylation in the cancer cell; the level of SRSF5 phosphorylation in the cancer cell; the level of a 55 kDa isoform of SRSF6 in the cancer cell; or the level of ˜35 kDa isoform of SRSF1 in the cancer cell. Exemplary methods for detecting the level of SRSF6 phosphorylation, the level of SRSF5 phosphorylation, the level of the ˜55 kDa isoform of SRSF6, and the level of the ˜35 kDa isoform of SRSF1 are described in the Examples. Additional methods for determining the level of SRSF6 phosphorylation, the level of SRSF5 phosphorylation, the level of the ˜55 kDa isoform of SRSF6, and the level of the ˜35 kDa isoform of SRSF1 are known in the art.

### Methods of Selecting a Treatment—Type B

Also provided herein are methods of selecting a treatment for a subject (e.g., any of the subjects described herein) that include: identifying a subject having a cancer cell (e.g., any of the exemplary types of cancer cell described herein or known in the art) that has aberrant mRNA splicing activity as compared to a reference level (e.g., any of the exemplary reference levels described herein); and selecting for the identified subject a treatment including a therapeutically effective amount of a CLK inhibitor (e.g., any of the exemplary CLK inhibitors described herein or known in the art) or a pharmaceutically acceptable salt or solvent thereof.

Also provided herein are methods of selecting a treatment for a subject (e.g., any of the subjects described herein) that include selecting a treatment including a therapeutically effective amount of a CLK inhibitor (e.g., any of the exemplary CLK inhibitors described herein or known in the art) or a pharmaceutically acceptable salt or solvent thereof for a subject identified as having a cancer cell (e.g., any of the exemplary types of cancer cell described herein or known in the art) that has aberrant mRNA splicing activity as compared to a reference level (e.g., any of the exemplary reference levels described herein).

In some embodiments of any of the methods of selecting a treatment described herein, the cancer cell having aberrant mRNA spicing activity can have one or more (e.g., two, three, four, five, or six) of: an increased level of phosphorylated serine and arginine rich splicing factor 6 (SRSF6) as compared to a reference level (e.g., any of the reference levels described herein); an increased level of phosphorylated serine and arginine rich splicing factor 5 (SRSF5) as compared to a reference level (e.g., any of the reference levels described herein); a mutation in a splicing factor 3b subunit 1 (SF3B1) gene, a serine and arginine rich splicing factor 1 (SRSF1) gene, a serine and arginine rich splicing factor 2 (SRSF2) gene, a small nuclear RNA auxiliary factor 1 (U2AF1) gene, or a zinc finger CCCH-type, RNA binding motif and serine/arginine rich 2 (ZRSR2) gene; and an increased level of SRSF1, SRSF2, serine and arginine rich splicing factor 3 (SRSF3), serine and arginine rich splicing factor 4 (SRSF4), SRSF5, SRSF6, and serine and arginine rich splicing factor 10 (SRSF10) as compared to a reference level (e.g., any of the exemplary reference levels described herein).

In some embodiments of any of the methods of selecting a treatment described herein, the level of aberrant mRNA splicing is determined by detecting: the level of SRSF6 phosphorylation in the cancer cell; the level of SRSF5 phosphorylation in the cancer cell; the level of a 55 kDa isoform of SRSF6 in the cancer cell; or the level of 35 kDa isoform of SRSF1 in the cancer cell. Exemplary methods for detecting the level of SRSF6 phosphorylation, the level of SRSF5 phosphorylation, the level of the ˜55 kDa isoform of SRSF6, and the level of the ˜35 kDa isoform of SRSF1 are described in the Examples. Additional methods for determining the level of SRSF6 phosphorylation, the level of SRSF5 phosphorylation, the level of the ˜55 kDa isoform of SRSF6, and the level of the ˜35 kDa isoform of SRSF1 are known in the art.

### Methods of Selecting a Subject for Treatment—Type B

Also provided herein are methods of selecting a subject (e.g., any of the exemplary subjects described herein) for treatment that include: identifying a subject having a cancer cell (e.g., any of the exemplary types of cancer cells described herein or known in the art) that has aberrant mRNA splicing activity as compared to a reference level (e.g., any of the reference levels described herein); and selecting an identified subject for treatment with a therapeutically effective amount of a CLK inhibitor (e.g., any of the exemplary CLK inhibitors described herein or known in the art) or a pharmaceutically acceptable salt or solvent thereof.

Also provided herein are methods of selecting a subject (e.g., any of the subjects described herein or known in the art) for treatment that include selecting a subject identified as having a cancer cell (e.g., any of the exemplary types of cancer cell described herein or known in the art) that has aberrant mRNA splicing activity as compared to a reference level (e.g., any of the exemplary reference levels described herein), for treatment with a therapeutically effective amount of a CLK inhibitor (e.g., any of the exemplary CLK inhibitors described herein or known in the art) or a pharmaceutically acceptable salt or solvent thereof.

In some embodiments of any of the methods of selecting a subject for treatment described herein, the cancer cell having aberrant mRNA spicing activity can have one or more (e.g., two, three, four, five, or six) of: an increased level of phosphorylated serine and arginine rich splicing factor 6 (SRSF6) as compared to a reference level (e.g., any of the reference levels described herein); an increased level of phosphorylated serine and arginine rich splicing factor 5 (SRSF5) as compared to a reference level (e.g., any of the reference levels described herein); a mutation in a splicing factor 3b subunit 1 (SF3B1) gene, a serine and arginine rich splicing factor 1 (SRSF1) gene, a serine and arginine rich splicing factor 2 (SRSF2) gene, a small nuclear RNA auxiliary factor 1 (U2AF1) gene, or a zinc finger CCCH-type, RNA binding motif and serine/arginine rich 2 (ZRSR2) gene; and an increased level of SRSF1, SRSF2, serine and arginine rich splicing factor 3 (SRSF3), serine and arginine rich splicing factor 4 (SRSF4), SRSF5, SRSF6, and serine and arginine rich splicing factor 10 (SRSF10) as compared to a reference level (e.g., any of the exemplary reference levels described herein).

In some embodiments of any of the methods of selecting a subject for treatment described herein, the level of aberrant mRNA splicing is determined by detecting: the level of SRSF6 phosphorylation in the cancer cell; the level of SRSF5 phosphorylation in the cancer cell; the level of a ˜55 kDa isoform of SRSF6 in the cancer cell; or the level of ˜35 kDa isoform of SRSF1 in the cancer cell. Exemplary methods for detecting the level of SRSF6 phosphorylation, the level of SRSF5 phosphorylation, the level of the ˜55 kDa isoform of SRSF6, and the level of the ˜35 kDa isoform of SRSF1 are described in the Examples. Additional methods for determining the level of SRSF6 phosphorylation, the level of SRSF5 phosphorylation, the level of the ˜55 kDa isoform of SRSF6, and the level of the ˜35 kDa isoform of SRSF1 are known in the art.

### Methods of Selecting a Subject for Participation in a Clinical Study—Type B

Also provided herein are methods of selecting a subject (e.g., any of the exemplary subjects described herein) for participation in a clinical trial that include: identifying a subject having a cancer cell (e.g., any of the exemplary types of cancer cells described herein) that has aberrant mRNA splicing activity as compared to a reference level (e.g., any of the exemplary reference levels described herein); and selecting the identified subject for participation in a clinical trial that comprises administration of a therapeutically effective amount of a CLK inhibitor (e.g., any of the exemplary CLK inhibitors described herein or known in the art) or a pharmaceutically acceptable salt or solvent thereof.

Also provided herein are methods of selecting a subject (e.g., any of the exemplary subjects described herein) for participation in a clinical trial that include selecting a subject identified as having a cancer cell (e.g., any of the exemplary types of cancer cells described herein or known in the art) that has aberrant mRNA splicing activity as compared to a reference level (e.g., any of the exemplary reference levels described herein) for participation in a clinical trial that comprises administration of a therapeutically effective amount of a CLK inhibitor (e.g., any of the exemplary CLK inhibitors described herein or known in the art) or a pharmaceutically acceptable salt or solvent thereof.

In some embodiments of any of the methods of selecting a subject for participation in a clinical trial described herein, the cancer cell having aberrant mRNA spicing activity can have one or more (e.g., two, three, four, five, or six) of: an increased level of phosphorylated serine and arginine rich splicing factor 6 (SRSF6) as compared to a reference level (e.g., any of the reference levels described herein); an increased level of phosphorylated serine and arginine rich splicing factor 5 (SRSF5) as compared to a reference level (e.g., any of the reference levels described herein); a mutation in a splicing factor 3b subunit 1 (SF3B1) gene, a serine and arginine rich splicing factor 1 (SRSF1) gene, a serine and arginine rich splicing factor 2 (SRSF2) gene, a small nuclear RNA auxiliary factor 1 (U2AF1) gene, or a zinc finger CCCH-type, RNA binding motif and serine/arginine rich 2 (ZRSR2) gene; and an increased level of SRSF1, SRSF2, serine and arginine rich splicing factor 3 (SRSF3), serine and arginine rich splicing factor 4 (SRSF4), SRSF5, SRSF6, and serine and arginine rich splicing factor 10 (SRSF10) as compared to a reference level (e.g., any of the exemplary reference levels described herein).

In some embodiments of any of the methods of selecting a subject for participation in a clinical trial described herein, the level of aberrant mRNA splicing is determined by detecting: the level of SRSF6 phosphorylation in the cancer cell; the level of SRSF5 phosphorylation in the cancer cell; the level of a 55 kDa isoform of SRSF6 in the cancer cell; or the level of 35 kDa isoform of SRSF1 in the cancer cell. Exemplary methods for detecting the level of SRSF6 phosphorylation, the level of SRSF5 phosphorylation, the level of the ˜55 kDa isoform of SRSF6, and the level of the ˜35 kDa isoform of SRSF1 are described in the Examples. Additional methods for determining the level of SRSF6 phosphorylation, the level of SRSF5 phosphorylation, the level of the ˜55 kDa isoform of SRSF6, and the level of the ˜35 kDa isoform of SRSF1 are known in the art.

### Methods of Determining the Efficacy of a CLK Inhibitor—Type B

Also provided herein are methods of determining the efficacy of a CLK inhibitor (e.g., any of the exemplary CLK inhibitors described herein or known in the art) or a pharmaceutically acceptable salt or solvate thereof in a subject (e.g., any of the subjects described herein) that include: (a) determining a first level of SRSF6 phosphorylation and/or SRSF5 phosphorylation in a cancer cell (e.g., any of the exemplary types of cancer cells described herein or known in the art) obtained from a subject at a first time point; (b) administering to the subject after the first time a CLK inhibitor or a pharmaceutically acceptable salt or solvent thereof, (c) determining a second level of SRSF6 phosphorylation and/or SRSF5 phosphorylation in a cancer cell obtained from the subject at a second time point; and (d) determining that the CLK inhibitor is effective in a subject having a second level that is decreased (e.g., a 1% to about 99% decrease, or any of the subranges of this range described herein) as compared to the first level.

Also provided herein are methods of determining the efficacy of a CLK inhibitor (e.g., any of the exemplary CLK inhibitors described herein or known in the art) or a pharmaceutically acceptable salt or solvate thereof that include: (a) determining a first level of a ˜55 kDa isoform of SRSF6 in a cancer cell (e.g., any of the exemplary types of cancer cell described herein or known in the art) obtained from a subject (e.g., any of the subjects described herein) at a first time point; (b) administering to the subject after the first time a CLK inhibitor or a pharmaceutically acceptable salt or solvent thereof, (c) determining a second level of the ˜55 kDa isoform of SRSF6 in a cancer cell obtained from the subject at a second time point; and (d) determining that the CLK inhibitor is effective in a subject having a second level of the ˜55 kDa isoform of SRSF6 that is increased (e.g., a 1% to 500% increase, or any of the subranges of this range described herein) as compared to the first level of the ˜55 kDa isoform of SRSF6.

Also provided herein are method of determining the efficacy of a CLK inhibitor (e.g., any of the exemplary CLK inhibitors described herein or known in the art) (e.g., a compound of any one of Formulas (I)-(XII) or a pharmaceutically acceptable salt or solvent thereof in a subject (e.g., any of the subjects described herein) that include: (a) determining a first level of a ˜35 kDa isoform of SRSF1 in a cancer cell (e.g., any of the exemplary types of cancer cells described herein or known in the art) obtained from a subject at a first time point; (b) administering to the subject after the first time point a compound of a CLK inhibitor or a pharmaceutically acceptable salt or solvent thereof, (c) determining a second level of the ˜35 kDa isoform of SRSF1 in a cancer cell obtained from the subject at a second time point; and (d) determining that the CLK inhibitor is effective in a subject having a second level of the ˜35 kDa isoform of SRSF1 that is increased (e.g., a 1% to 500% increase, or any of the subranges of this range described herein) as compared to the first level of the ˜35 kDa isoform of SRSF1.

In some embodiments of any of the methods described herein, the method further includes: (e) after (d), administering one or more (e.g., 1, 2, 3, 4, 5, 6, 7, 8, 9, 10, 11, 12, 13, 14, 15, 16, 17, 18, 19, 20, 22, 24, 26, 28, 30, 40, 50, 60, 70, 80, 90, or 100) additional doses of the CLK inhibitor to the subject.

In some embodiments of any of the methods further include a step of selecting a subject having cancer or diagnosing a subject as having cancer. For example, a subject having cancer can have previously been administered a treatment for cancer, and the previous treatment was unsuccessful. Some embodiments of any of the methods described herein can further include obtaining a cancer cell from the subject at the first and second time points.

In some embodiments of any of the methods described herein, the method further includes recording the identified efficacy of the CLK inhibitor in the subject's medical record (e.g., a computer readable medium).

In some embodiments of any of the methods described herein, the method further includes informing the subject, the subject's family, and/or the subject's primary care physician or attending physician of the determined efficacy of the CLK inhibitor.

In some embodiments of any of the methods described herein, the method further includes monitoring the subject. For example, the method can include authorizing a refill of the CLK inhibitor administered to the subject between the first and second time points and determined to be effective.

In some embodiments of any of the methods of determining the efficacy of treatment described herein, the cancer cell is a small cell lung cancer cell, a colorectal cancer cell, a head and neck cancer cell, an ovarian cancer cell, a melanoma cell, a renal cell carcinoma cell, a pancreatic cancer cell, or a non-small cell lung cancer cell. In some embodiments of any of the methods of determining the efficacy of treatment described herein, the cancer can be any of the cancers described herein or known in the art.

Exemplary methods for detecting the level of SRSF6 phosphorylation, the level of SRSF5 phosphorylation, the level of the ˜55 kDa isoform of SRSF6, and the level of the ˜35 kDa isoform of SRSF1 are described in the Examples. Additional methods for determining the level of SRSF6 phosphorylation, the level of SRSF5 phosphorylation, the level of the ˜55 kDa isoform of SRSF6, and the level of the ˜35 kDa isoform of SRSF1 are known in the art.

### Samples

Some embodiments of any of the methods described herein can further including obtaining a cell (e.g., a cancer cell or any of the other types of cells) from the subject. For example, the cell (e.g., cancer cell) can be obtained from the subject in the form of a biological sample, e.g., any clinically relevant tissue sample, such as a tumor biopsy, a core biopsy tissue sample, a fine needle aspirate, a hair follicle, or a sample of bodily fluid, such as blood, plasma, serum, lymph, ascitic fluid, cystic fluid, or urine.

In some embodiments, the biological sample is taken from a patient having a tumor or cancer. In some embodiments, the biological sample is a primary tumor. In some embodiments, the biological sample is a metastasis. The biological sample may be taken from a human, or from non-human mammals such as, mice, rats, non-human primates, canines, felines, ruminants, swine, or sheep. In some embodiments, biological samples are taken from a subject at multiple time points, for example, before treatment, during treatment, and/or after treatment. In some embodiments, biological samples are taken from different locations in the subject, for example, a sample from a primary tumor and a sample from a metastasis in a distant location.

In some embodiments, the biological sample is a paraffin-embedded fixed tissue sample. In some embodiments, the sample is a formalin-fixed paraffin embedded (FFPE) tissue sample. In some embodiments, the sample is a fresh tissue (e.g., tumor) sample. In some embodiments, the sample is a frozen tissue sample. In some embodiments, the sample is a fresh frozen (FF) tissue (e.g., tumor) sample. In some embodiments, the sample is a cell isolated from a fluid. In some embodiments, the sample comprises circulating tumor cells (CTCs). In some embodiments, the sample is an archival tissue sample. In some embodiments, the sample is an archival tissue sample with known diagnosis, treatment, and/or outcome history. In some embodiments, the sample is a block of tissue. In some embodiments, the sample is dispersed cells. In some embodiments, the sample size is from about 1 cell to about 1×10⁶ cells or more. In some embodiments, the sample size is about 10 cells to about 1×10⁵ cells. In some embodiments, the sample size is about 10 cells to about 10,000 cells. In some embodiments, the sample size is about 10 cells to about 1,000 cells. In some embodiments, the sample size is about 10 cells to about 100 cells. In some embodiments, the sample size is about 1 cell to about 10 cells. In some embodiments, the sample size is a single cell.

In some embodiments, the sample is processed to isolate DNA or RNA.

In some embodiments, RNA is isolated from the sample. In some embodiments, mRNA is isolated from the sample. In some embodiments, RNA is isolated from cells by procedures that involve cell lysis and denaturation of the proteins contained therein. In some embodiments, DNase is added to remove DNA. In some embodiments, RNase inhibitors are added to the lysis buffer. In some embodiments, a protein denaturation/digestion step is added to the protocol.

Methods for preparing total and mRNA are well known in the art and RNA isolation kits are commercially available (e.g., RNeasy mini kit, Qiagen, USA). In some embodiments, the RNA is amplified by PCR-based techniques.

### Exemplary CLK Inhibitors

Compound 12 is small molecule CLK inhibitor which acts a Wnt signaling inhibitor by downregulating Wnt pathway gene expression in cancer cells. Compound 12 was phenotypically screened and discovered on its ability to inhibit Wnt reporter activity driven by constitutively active Wnt signaling in SW480 CRC cells. Compound 12's ability to block Wnt signaling was further confirmed by inhibition of Wnt-3a and GSK-3D-inhibitor stimulated Wnt signaling in non-cancerous cell types such as 293T and IEC-6 rat intestinal cells.

In some embodiments, the CLK inhibitor is a multi-isoform CLK inhibitor.

In some embodiments, the CLK inhibitor has an IC₅₀ of between about 1 nM and about 10 μM (e.g., between about 1 nM and about 9 μM, between about 1 nM and about 8 μM, between about 1 nM and about 7 μM, between about 1 nM and about 6 μM, between about 1 nM and about 5 μM, between about 1 nM and about 4 μM, between about 1 nM and about 3 μM, between about 1 nM and about 2 μM, between about 1 nM and about 1 μM, between about 1 nM and about 950 nM, between about 1 nM and about 900 nM, between about 1 nM and about 850 nM, between about 1 nM and about 800 nM, between about 1 nM and about 750 nM, between about 1 nM and about 700 nM, between about 1 nM and about 650 nM, between about 1 nM and about 600 nM, between about 1 nM and about 550 nM, between about 1 nM and about 500 nM, between about 1 nM and about 450 nM, between about 1 nM and about 400 nM, between about 1 nM and about 350 nM, between about 1 nM and about 300 nM, between about 1 nM and about 250 nM, between about 1 nM and about 200 nM, between about 1 nM and about 150 nM, between about 1 nM and about 100 nM, between about 1 nM and about 95 nM, between about 1 nM and about 90 nM, between about 1 nM and about 85 nM, between about 1 nM and about 80 nM, between about 1 nM and about 75 nM, between about 1 nM and about 70 nM, between about 1 nM and about 65 nM, between about 1 nM and about 60 nM, between about 1 nM and about 55 nM, between about 1 nM and about 50 nM, between about 1 nM and about 45 nM, between about 1 nM and about 40 nM, between about 1 nM and about 35 nM, between about 1 nM and about 30 nM, between about 1 nM and about 25 nM, between about 1 nM and about 20 nM, between about 1 nM and about 15 nM, between about 1 nM and about 10 nM, between about 1 nM and about 5 nM, between about 1 nM and about 4 nM, between about 1 nM and about 3 nM, between about 1 nM and about 2 nM, between about 2 nM and about 10 μM, between about 2 nM and about 9 μM, between about 2 nM and about 8 μM, between about 2 nM and about 7 μM, between about 2 nM and about 6 μM, between about 2 nM and about 5 μM, between about 2 nM and about 4 μM, between about 2 nM and about 3 μM, between about 2 nM and about 2 μM, between about 2 nM and about 1 μM, between about 2 nM and about 950 nM, between about 2 nM and about 900 nM, between about 2 nM and about 850 nM, between about 2 nM and about 800 nM, between about 2 nM and about 750 nM, between about 2 nM and about 700 nM, between about 2 nM and about 650 nM, between about 2 nM and about 600 nM, between about 2 nM and about 550 nM, between about 2 nM and about 500 nM, between about 2 nM and about 450 nM, between about 2 nM and about 400 nM, between about 2 nM and about 350 nM, between about 2 nM and about 300 nM, between about 2 nM and about 250 nM, between about 2 nM and about 200 nM, between about 2 nM and about 150 nM, between about 2 nM and about 100 nM, between about 2 nM and about 95 nM, between about 2 nM and about 90 nM, between about 2 nM and about 85 nM, between about 2 nM and about 80 nM, between about 2 nM and about 75 nM, between about 2 nM and about 70 nM, between about 2 nM and about 65 nM, between about 2 nM and about 60 nM, between about 2 nM and about 55 nM, between about 2 nM and about 50 nM, between about 2 nM and about 45 nM, between about 2 nM and about 40 nM, between about 2 nM and about 35 nM, between about 2 nM and about 30 nM, between about 2 nM and about 25 nM, between about 2 nM and about 20 nM, between about 2 nM and about 15 nM, between about 2 nM and about 10 nM, between about 2 nM and about 5 nM, between about 2 nM and about 4 nM, between about 2 nM and about 3 nM, between about 5 nM and about M, between about 5 nM and about 9 μM, between about 5 nM and about 8 μM, between about 5 nM and about 7 μM, between about 5 nM and about 6 μM, between about 5 nM and about 5 μM, between about 5 nM and about 4 μM, between about 5 nM and about 3 μM, between about 5 nM and about 2 μM, between about 5 nM and about 1 μM, between about 5 nM and about 950 nM, between about 5 nM and about 900 nM, between about 5 nM and about 850 nM, between about 5 nM and about 800 nM, between about 5 nM and about 750 nM, between about 5 nM and about 700 nM, between about 5 nM and about 650 nM, between about 5 nM and about 600 nM, between about 5 nM and about 550 nM, between about 5 nM and about 500 nM, between about 5 nM and about 450 nM, between about 5 nM and about 400 nM, between about 5 nM and about 350 nM, between about 5 nM and about 300 nM, between about 5 nM and about 250 nM, between about 5 nM and about 200 nM, between about 5 nM and about 150 nM, between about 5 nM and about 100 nM, between about 5 nM and about 95 nM, between about 5 nM and about 90 nM, between about 5 nM and about 85 nM, between about 5 nM and about 80 nM, between about 5 nM and about 75 nM, between about 5 nM and about 70 nM, between about 5 nM and about 65 nM, between about 5 nM and about 60 nM, between about 5 nM and about 55 nM, between about 5 nM and about 50 nM, between about 5 nM and about 45 nM, between about 5 nM and about 40 nM, between about 5 nM and about 35 nM, between about 5 nM and about 30 nM, between about 5 nM and about 25 nM, between about 5 nM and about 20 nM, between about 5 nM and about 15 nM, between about 5 nM and about 10 nM, between about 10 nM and about 10 μM, between about 10 nM and about 9 μM, between about 10 nM and about 8 μM, between about 10 nM and about 7 μM, between about 10 nM and about 6 μM, between about 10 nM and about 5 μM, between about 10 nM and about 4 μM, between about 10 nM and about 3 μM, between about 10 nM and about 2 μM, between about 10 nM and about 1 μM, between about 10 nM and about 950 nM, between about 10 nM and about 900 nM, between about 10 nM and about 850 nM, between about 10 nM and about 800 nM, between about 10 nM and about 750 nM, between about 10 nM and about 700 nM, between about 10 nM and about 650 nM, between about 10 nM and about 600 nM, between about 10 nM and about 550 nM, between about 10 nM and about 500 nM, between about 10 nM and about 450 nM, between about 10 nM and about 400 nM, between about 10 nM and about 350 nM, between about 10 nM and about 300 nM, between about 10 nM and about 250 nM, between about 10 nM and about 200 nM, between about 10 nM and about 150 nM, between about 10 nM and about 100 nM, between about 10 nM and about 95 nM, between about 10 nM and about 90 nM, between about 10 nM and about 85 nM, between about 10 nM and about 80 nM, between about 10 nM and about 75 nM, between about 10 nM and about 70 nM, between about 10 nM and about 65 nM, between about 10 nM and about 60 nM, between about 10 nM and about 55 nM, between about 10 nM and about 50 nM, between about 10 nM and about 45 nM, between about 10 nM and about 40 nM, between about 10 nM and about 35 nM, between about 10 nM and about 30 nM, between about 10 nM and about 25 nM, between about 10 nM and about 20 nM, between about 10 nM and about 15 nM, between about 50 nM and about 10 μM, between about 50 nM and about 9 μM, between about 50 nM and about 8 μM, between about 50 nM and about 7 μM, between about 50 nM and about 6 μM, between about 50 nM and about 5 μM, between about 50 nM and about 4 μM, between about 50 nM and about 3 μM, between about 50 nM and about 2 μM, between about 50 nM and about 6 μM, between about 50 nM and about 950 nM, between about 50 nM and about 900 nM, between about 50 nM and about 850 nM, between about 50 nM and about 800 nM, between about 50 nM and about 750 nM, between about 50 nM and about 700 nM, between about 50 nM and about 650 nM, between about 50 nM and about 600 nM, between about 50 nM and about 550 nM, between about 50 nM and about 500 nM, between about 50 nM and about 450 nM, between about 50 nM and about 400 nM, between about 50 nM and about 350 nM, between about 50 nM and about 300 nM, between about 50 nM and about 250 nM, between about 50 nM and about 200 nM, between about 50 nM and about 150 nM, between about 50 nM and about 100 nM, between about 50 nM and about 95 nM, between about 50 nM and about 90 nM, between about 50 nM and about 85 nM, between about 50 nM and about 80 nM, between about 50 nM and about 75 nM, between about 50 nM and about 70 nM, between about 50 nM and about 65 nM, between about 50 nM and about 60 nM, between about 50 nM and about 55 nM, between about 100 nM and about 10 μM, between about 100 nM and about 9 μM, between about 100 nM and about 8 μM, between about 100 nM and about 7 μM, between about 100 nM and about 6 μM, between about 100 nM and about 5 μM, between about 100 nM and about 4 μM, between about 100 nM and about 3 μM, between about 100 nM and about 2 μM, between about 100 nM and about 1 μM, between about 100 nM and about 950 nM, between about 100 nM and about 900 nM, between about 100 nM and about 850 nM, between about 100 nM and about 800 nM, between about 100 nM and about 750 nM, between about 100 nM and about 700 nM, between about 100 nM and about 650 nM, between about 100 nM and about 600 nM, between about 100 nM and about 550 nM, between about 100 nM and about 500 nM, between about 100 nM and about 450 nM, between about 100 nM and about 400 nM, between about 100 nM and about 350 nM, between about 100 nM and about 300 nM, between about 100 nM and about 250 nM, between about 100 nM and about 200 nM, between about 100 nM and about 150 nM, between about 200 nM and about 10 μM, between about 200 nM and about 9 μM, between about 200 nM and about 8 μM, between about 200 nM and about 7 μM, between about 200 nM and about 6 μM, between about 200 nM and about 5 μM, between about 200 nM and about 4 μM, between about 200 nM and about 3 μM, between about 200 nM and about 2 μM, between about 200 nM and about 1 μM, between about 200 nM and about 950 nM, between about 200 nM and about 900 nM, between about 200 nM and about 850 nM, between about 200 nM and about 800 nM, between about 200 nM and about 750 nM, between about 200 nM and about 700 nM, between about 200 nM and about 650 nM, between about 200 nM and about 600 nM, between about 200 nM and about 550 nM, between about 200 nM and about 500 nM, between about 200 nM and about 450 nM, between about 200 nM and about 400 nM, between about 200 nM and about 350 nM, between about 200 nM and about 300 nM, between about 200 nM and about 250 nM, between about 250 nM and about 10 μM, between about 250 nM and about 9 μM, between about 250 nM and about 8 μM, between about 250 nM and about 7 μM, between about 250 nM and about 6 μM, between about 250 nM and about 5 μM, between about 250 nM and about 4 μM, between about 250 nM and about 3 μM, between about 250 nM and about 2 μM, between about 250 nM and about 1 μM, between about 250 nM and about 950 nM, between about 250 nM and about 900 nM, between about 250 nM and about 850 nM, between about 250 nM and about 800 nM, between about 250 nM and about 750 nM, between about 250 nM and about 700 nM, between about 250 nM and about 650 nM, between about 250 nM and about 600 nM, between about 250 nM and about 550 nM, between about 250 nM and about 500 nM, between about 250 nM and about 450 nM, between about 250 nM and about 400 nM, between about 250 nM and about 350 nM, between about 250 nM and about 300 nM, between about 500 nM and about 10 μM, between about 500 nM and about 9 μM, between about 500 nM and about 8 μM, between about 500 nM and about 7 μM, between about 500 nM and about 6 μM, between about 500 nM and about 5 μM, between about 500 nM and about 4 μM, between about 500 nM and about 3 μM, between about 500 nM and about 2 μM, between about 500 nM and about 1 μM, between about 500 nM and about 950 nM, between about 500 nM and about 900 nM, between about 500 nM and about 850 nM, between about 500 nM and about 800 nM, between about 500 nM and about 750 nM, between about 500 nM and about 700 nM, between about 500 nM and about 650 nM, between about 500 nM and about 600 nM, between about 500 nM and about 550 nM, between about 750 nM and about 10 μM, between about 750 nM and about 9 μM, between about 750 nM and about 8 μM, between about 750 nM and about 7 μM, between about 750 nM and about 6 μM, between about 750 nM and about 5 μM, between about 750 nM and about 4 μM, between about 750 nM and about 3 μM, between about 750 nM and about 2 μM, between about 750 nM and about 1 μM, between about 750 nM and about 950 nM, between about 750 nM and about 900 nM, between about 750 nM and about 850 nM, between about 750 nM and about 800 nM, between about 950 nM and about 10 μM, between about 950 nM and about 9 μM, between about 950 nM and about 8 μM, between about 950 nM and about 7 μM, between about 950 nM and about 6 μM, between about 950 nM and about 5 μM, between about 950 nM and about 4 μM, between about 950 nM and about 3 μM, between about 950 nM and about 2 μM, between about 950 nM and about 1 μM, between about 1 μM and about 10 μM, between about 1 μM and about 9 μM, between about 1 μM and about 8 μM, between about 1 μM and about 7 μM, between about 1 μM and about 6 μM, between about 1 μM and about 5 μM, between about 1 μM and about 4 μM, between about 1 μM and about 3 μM, between about 1 μM and about 2 μM, between about 2 μM and about 10 μM, between about 2 μM and about 9 μM, between bout 2 μM and about 8 μM, between about 2 μM and about 7 μM, between about 2 μM and about 6 μM, between about 2 μM and about 5 μM, between about 2 μM and about 4 μM, between about 2 μM and about 3 μM, between about 4 μM and about 10 μM, between about 4 μM and about 9 μM, between about 4 μM and about 8 μM, between about 4 μM and about 7 μM, between about 4 μM and about 6 μM, between about 4 μM and about 5 μM, between about 5 μM and about 10 μM, between about 5 μM and about 9 μM, between about 5 μM and about 8 μM, between about 5 μM and about 7 μM, between about 5 μM and about 6 μM, between about 6 μM and about 10 μM, between about 6 μM and about 9 μM, between about 6 μM and about 8 μM, between about 6 μM and about 7 μM; between about 7 μM and about 10 μM, between about 7 μM and about 9 μM, between about 7 μM and about 8 μM, between about 8 μM and about 10 μM, between about 8 μM and about 9 μM, or between about 9 μM and about 10 μM) for one or both of CLK2 and CLK3.

In some embodiments, the CLK inhibitor has an IC₅₀ of between about 1 nM and about 1 μM (or any of the subranges of this range described herein) for each of CLK3 and CLK4. In some embodiments, the CLK inhibitor has an IC₅₀ of between about 1 nM and about 10 μM (or any of the subranges of this range) for each of CLK1 and CLK3. In some embodiments, the CLK inhibitor has an IC₅₀ of between about 1 nM and about 10 μM (or any of the subranges of this range described herein) for each of CLK1 and CLK2. In some embodiments, the CLK inhibitor has an IC₅₀ of between about 1 nM and about 10 μM (or any of the subranges of this range described herein) for each of CLK1 and CLK4. In some embodiments, the CLK inhibitor has an IC₅₀ of between about 1 nM and about 10 μM (or any of the subranges of this range described herein) for each of CLK2 and CLK4. In some embodiments, the CLK inhibitor has an IC₅₀ of between about 1 nM and about M (or any of the subranges of this range described herein) for each of CLK1, CLK2, and/or CLK3. In some embodiments, the m CLK inhibitor has an IC₅₀ of between about 1 nM and about M (or any of the subranges of this range described herein) for each of CLK1, CLK2 and CLK4. In some embodiments, the CLK inhibitor has an IC₅₀ of between about 1 nM and about 10 μM (or any of the subranges of this range described herein) for each of CLK2, CLK3 and CLK4. In some embodiments, the CLK inhibitor has an IC₅₀ of between about 1 nM and about 10 μM (or any of the subranges of this range described herein) for each of CLK1, CLK2, CLK3 and CLK4.

In some embodiments, the CLK inhibitor is a compound of Formula I or a pharmaceutically acceptable salt or solvate thereof.

In some embodiments, the CLK inhibitor is a compound of Formula II or a pharmaceutically acceptable salt or solvate thereof.

In some embodiments, the CLK inhibitor is a compound of Formula III or a pharmaceutically acceptable salt or solvate thereof.

In some embodiments, the CLK inhibitor is a compound of Formula IV or a pharmaceutically acceptable salt or solvate thereof.

In some embodiments, the CLK inhibitor is a compound of Formula V or a pharmaceutically acceptable salt or solvate thereof.

In some embodiments, the CLK inhibitor is a compound of Formula VI or a pharmaceutically acceptable salt or solvate thereof.

In some embodiments, the CLK inhibitor is a compound of Formula VII or a pharmaceutically acceptable salt or solvate thereof.

In some embodiments, the CLK inhibitor is a compound of Formula VIII or a pharmaceutically acceptable salt or solvate thereof.

In some embodiments, the CLK inhibitor is a compound of Formula IX or a pharmaceutically acceptable salt or solvate thereof.

In some embodiments, the CLK inhibitor is a compound of Formula X or a pharmaceutically acceptable salt or solvate thereof.

In some embodiments, the CLK inhibitor is a compound of Formula XI or a pharmaceutically acceptable salt or solvate thereof.

In some embodiments, the CLK inhibitor is a compound of Formula XII or a pharmaceutically acceptable salt or solvate thereof.

In some embodiments, compounds for use as CLK2 or CLK2/CLK3 inhibitors include the compounds set forth below as described in the following journal articles, U.S. patents and U.S. patent applications.

U.S. provisional applications 62/793,428 and 62/831,478 describe compounds having Formula I and are hereby incorporated by reference in their entirety.

One embodiment disclosed herein includes a compound having the structure of Formula I:

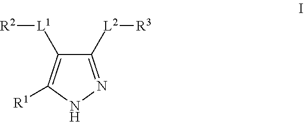

as well as prodrugs and pharmaceutically acceptable salt or solvate thereof.

In some embodiments of Formula (I):

R¹ is selected from the group consisting of H, halide (e.g., F, Cl, Br, I), and unsubstituted —(C₁₋₃ alkyl);

R² is selected from the group consisting of unsubstituted —(C₁₋₃ alkyl), unsubstituted —(C₂₋₉ alkenyl), unsubstituted —(C₁₋₉ haloalkyl), —(C₁₋₂ alkylene)ₚ(C₃₋₆ carbocyclyl) optionally substituted with 1-12 R⁴, -monocyclic heterocyclyl optionally substituted with 1-10 R, -phenyl substituted with 1-5 R⁶, -heteroaryl optionally substituted with 1-4 R⁷, —CO₂R, —OR⁹, and —(C═O)R″; wherein heteroaryl selected from the group consisting of pyridinyl, pyrimidinyl, pyrazinyl, oxazolyl, oxadiazolyl, thiazolyl, 2,3-dihydrobenzo[b]dioxinyl, 5,6,7,8-tetrahydroimidazo[1,2-a]pyrazinyl, 4,5,6,7-tetrahydro-1H-imidazo[4,5-c]pyridinyl, isoquinolinyl, and quinolinyl; wherein —(C₁₋₄ alkylene) is optionally substituted with one or more substituents as defined anywhere herein;

with the proviso that when L¹ is a bond, R² is selected from the group consisting of -phenyl substituted with 1-5 R⁶ and -heteroaryl optionally substituted with 1-4 R⁷; wherein heteroaryl selected from the group consisting of pyridinyl, oxazolyl, oxadiazolyl, thiazolyl, 2,3-dihydrobenzo[b]dioxinyl, 5,6,7,8-tetrahydroimidazo[1,2-a]pyrazinyl, 4,5,6,7-tetrahydro-1H-imidazo[4,5-c]pyridinyl, isoquinolinyl, and quinolinyl;

R³ is selected from the group consisting of -heterocyclyl substituted with 1-10 R″, —(C₁₋₄ alkylene)ₚphenyl substituted with 1-5 R¹², -heteroaryl optionally substituted with 1-4 R¹³, and —(C₁₋₄ alkylene)OR¹⁴; wherein heteroaryl selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein

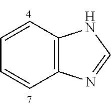

is only substituted at positions 4 and 7; wherein each —(C₁₋₄ alkylene) is, independently, optionally substituted with one or more substituents as defined anywhere herein;

with the proviso that when L² is a bond, R³ is selected from -heteroaryl optionally substituted with 1-4 R¹³; wherein heteroaryl selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein

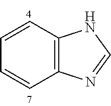

is only substituted at positions 4 and 7;

each R⁴ is halide;

each R⁵ is independently selected from the group consisting of halide (e.g., F, Cl, Br, I), Me, and Et;

each R⁶ is independently selected from the group consisting of methyl, —CH₂F, —CHF₂, —CF₃, —OR^{15a}, and —(C₁₋₄ alkylene)ₚN(R^{16a})(R^{16b}); wherein —(C₁₋₄ alkylene) is optionally substituted with one or more substituents as defined anywhere herein;

each R⁷ is independently selected from the group consisting of F, methyl, —CH₂F, —CHF₂, —CF₃, —CF₂CH₃, —OR^{15a}, —CO₂R¹⁷, —NR¹⁸(C═O)R¹⁹, —(C₁₋₄ alkylene)ₚheterocyclyl optionally substituted with 1-10 R^{20b}, and —(C₁₋₄ alkylene)ₚN(R^{16a})(R^{16b}); wherein each —(C₁₋₄ alkylene) is, independently, optionally substituted with one or more substituents as defined anywhere herein;

R⁸ is unsubstituted —(C₁₋₉ alkyl);

R⁹ is unsubstituted —(C₁₋₉ alkyl);

R¹⁰ is -aryl optionally substituted with 1-5 R²¹;

each R¹¹ is independently selected from the group consisting of halide (e.g., F, Cl, Br, I), methyl, and ethyl;

each R¹² is independently selected from the group consisting of —(C₁₋₄ alkylene)ₚheterocyclyl optionally substituted with 1-10 R^{20a}, -aryl optionally substituted with 1-5 R²², —(C₁₋₄ alkylene)N(R^{16a})(R^{16b}), and —OR^{23a}; wherein heterocyclyl selected from the group consisting of azetidinyl, pyrrolidinyl, piperidinyl, and piperazinyl; wherein each —(C₁₋₄ alkylene) is, independently, optionally substituted with one or more substituents as defined anywhere herein;

each R¹³ is independently selected from the group consisting of F, methyl, —CH₂F, —CHF₂, —CF₃, —(C₁₋₄ alkylene)ₚN(R^{16a})₂, —OR^{23b}, —(C₁₋₄ alkylene)ₚheterocyclyl optionally substituted with 1-10 R^{20b}, -aryl optionally substituted with 1-5 R²², and -heteroaryl substituted with 1-4 R²⁴; wherein —(C₁₋₄ alkylene) is optionally substituted with one or more substituents as defined anywhere herein;

R¹⁴ is selected from the group consisting of unsubstituted —(C₁₋₄ alkyl) and -aryl optionally substituted with 1-5 R²²;

each R^{15a} is independently selected from the group consisting of unsubstituted —(C₂₋₃ alkyl), and -heterocyclyl optionally substituted with 1-10 R^{20b};

each R^{15b} is independently selected from the group consisting of H, unsubstituted —(C₂₋₉ alkyl), and -heterocyclyl optionally substituted with 1-10 R^{20b};

each R^{16a} is independently selected from the group consisting of H and unsubstituted —(C₁₋₂ alkyl);

each R^{16b} is unsubstituted —(C₁₋₂ alkyl);

each R¹⁷ is unsubstituted —(C₁₋₉ alkyl);

each R¹⁸ is independently selected from the group consisting of H and Me;

each R¹⁹ is unsubstituted —(C₁₋₉ alkyl);

each R^{20a} is independently selected from the group consisting of halide and unsubstituted —(C₂₋₉ alkyl);

each R^{20b} is independently selected from the group consisting of halide and unsubstituted —(C₁₋₉ alkyl);

each R²¹ is independently selected from the group consisting of halide and unsubstituted —(C₁₋₉ alkyl);

each R²² is independently selected from the group consisting of halide and unsubstituted —(C₁₋₉ alkyl);

each R^{23a} is independently selected from the group consisting of unsubstituted —(C₂₋₉ alkyl), —(C₁₋₄ alkylene)OR²⁵, and —(C₁₋₄ alkylene)ₚheterocyclyl optionally substituted with 1-10 R^{20b}; wherein each —(C₁₋₄ alkylene) is, independently, optionally substituted with one or more substituents as defined anywhere herein;

each R^{23b} is independently selected from the group consisting of unsubstituted —(C₁₋₉ alkyl), —(C₁₋₄ alkylene)OR²⁵, and —(C₁₋₄ alkylene)ₚheterocyclyl optionally substituted with 1-10 R^{20b}; wherein each —(C₁₋₄ alkylene) is, independently, optionally substituted with one or more substituents as defined anywhere herein;

each R²⁴ is independently selected from the group consisting of halide and unsubstituted —(C₁₋₉ alkyl);

each R²⁵ is independently selected from the group consisting of H and unsubstituted —(C₁₋₉ alkyl);

L¹ is selected from the group consisting of a bond, —CH═CH—,

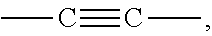

(CH₂)ₚNR¹⁸ (C═O)—, —(C═O)NR¹⁸(CH₂)ₚ—, —NR¹⁸(C═O)NR¹⁸—, —NH(CH₂)ₚ—, and —(CH₂)ₚNH—;

L² is selected from the group consisting of a bond, —(C═O)NR¹⁸—, —NR¹⁸(C═O)—, —NHCH₂—, and —CH₂NH—; and

each p is independently an integer of 0 or 1.

In some embodiments of Formula I, R¹ is selected from the group consisting of H, halide (e.g., F, Cl, Br, I), and unsubstituted —(C₁₋₃ alkyl).

In some embodiments of Formula I, R¹ is H.

In some embodiments of Formula I, R¹ is F.

In some embodiments of Formula I, R¹ is Me.

In some embodiments of Formula I, R² is a -monocyclic heterocyclyl optionally substituted with 1-2 R.

In some embodiments of Formula I, R² is a -monocyclic heterocyclyl optionally substituted with 1 Me.

In some embodiments of Formula I, R³ is -heterocyclyl substituted with 1-2 R¹¹.

In some embodiments of Formula I, R³ is -heterocyclyl substituted with 1 Me

In some embodiments of Formula I, R is —(C₁₋₂ alkylene)phenyl substituted with 1-2 R¹².

In some embodiments of Formula I, R³ is -phenyl substituted with 1-2 R¹².

In some embodiments of Formula I, R³ is -heteroaryl optionally substituted with 1-2 R¹³.

In some embodiments of Formula I, R³ is -pyridinyl optionally substituted with 1-2 R¹³.

In some embodiments of Formula I, R³ is R

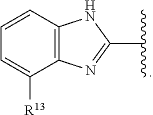

In some embodiments of Formula I, R³ is R

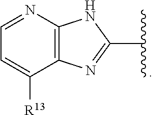

In some embodiments of Formula I, R³ is R

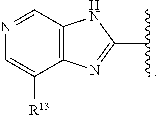

In some embodiments of Formula I, R³ is R

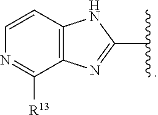

In some embodiments of Formula I, L¹ is selected from the group consisting of a bond, —C(═O)NH—, —CH═CH—, and

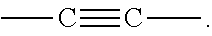

In some embodiments of Formula I, L¹ is a bond; in some embodiments of Formula I, L¹ is —C(═O)NH—; in some embodiments of Formula I, L¹ is —CH═CH—; and in some embodiments of Formula I, L¹ is

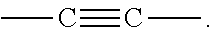

In some embodiments of Formula I, L² is selected from the group consisting of a bond and —C(═O)NH—.

In some embodiments of Formula I, L² is a bond.

In some embodiments of Formula I, L² is —C(═O)NH—.

U.S. provisional application 62/685,764 describes compounds having Formula II and is hereby incorporated by reference in their entirety.

One embodiment disclosed herein includes a compound having the structure of Formula II:

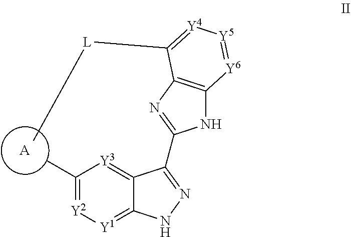

as well as prodrugs and pharmaceutically acceptable salt or solvate thereof.

In some embodiments of Formula (II):

Ring A is a 5-6-membered heteroaryl optionally substituted with 1-4 R¹;

L is -L¹-L²-L³-L⁴-;

L¹ is selected from the group consisting of unsubstituted —(C₁₋₃ alkylene)-, —NR²—, —NR³(C═O)—, —(C═O)NR³—, and —O—;

L² is selected from the group consisting of unsubstituted —(C₁₋₆ alkylene)- and —NR²—;

L³ is selected from the group consisting of unsubstituted —(C₁₋₆ alkylene)-, —O—, and -carbocyclylene- optionally substituted with one or more halides;

L⁴ is selected from the group consisting of unsubstituted —(C₁₋₆ alkylene)-, —O—, —NR²—, —NR³(C═O)—, —(C═O)NR³—, -arylene- optionally substituted with 1-5 R⁴, and -heteroarylene-optionally substituted with 1-4 R⁵;

with the proviso that —NR²— and —O— are not adjacent to each other;

with the proviso that two —NR³(C═O)— and/or —(C═O)NR³—, are not adjacent to each other;

each R¹ is selected from the group consisting of halide (e.g., F, Cl, Br, I), unsubstituted —(C₁₋₃ alkyl), unsubstituted —(C₁₋₃ haloalkyl), and —CN;

each R² is selected from the group consisting of H and unsubstituted —(C₁₋₆ alkyl);

each R³ is selected from the group consisting of H and unsubstituted —(C₁₋₆ alkyl);

each R⁴ is selected from the group consisting of halide (e.g., F, Cl, Br, I), unsubstituted —(C₁₋₆ alkyl), unsubstituted —(C₁₋₆ haloalkyl), and —CN;

each R is selected from the group consisting of halide (e.g., F, Cl, Br, I), unsubstituted —(C₁₋₆ alkyl), unsubstituted —(C₁₋₆ haloalkyl), and —CN;

Y¹, Y², Y³, Y⁴, Y⁵, and Y⁶ are independently selected from the group consisting of carbon and nitrogen; wherein

if Y¹ is nitrogen then Y² and Y³ are CH;

if Y² is nitrogen then Yi and Y³ are CH;

if Y³ is nitrogen then Yi and Y² are CH;

if Y⁴ is nitrogen then Y⁵ and Y⁶ are CH;

if Y⁵ is nitrogen then Y⁴ and Y⁶ are CH; and

if Y⁶ is nitrogen then Y⁴ and Y⁵ are CH.

In some embodiments of Formula II, Ring A is a 5-membered heteroaryl and is selected from the group consisting of

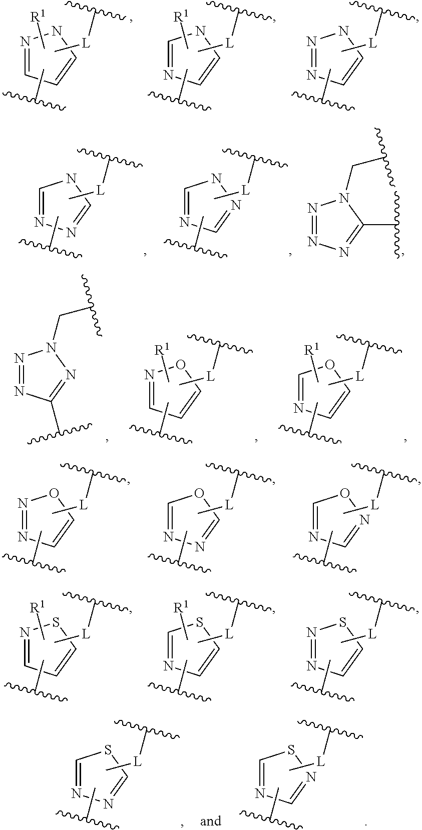

In some embodiments of Formula II, Ring A is a 6-membered heteroaryl and is selected from the group consisting of

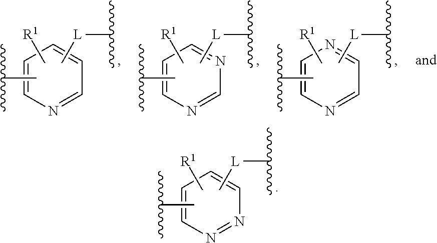

In some embodiments of Formula II, Ring A is a 5-6-membered heteroaryl and is selected from the group consisting of

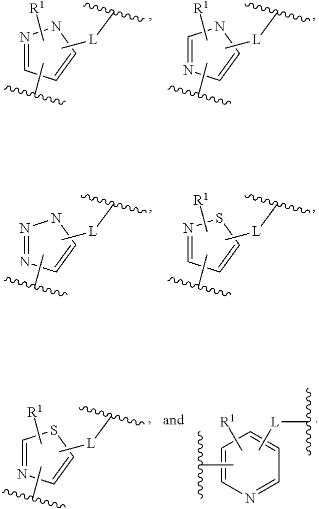

In some embodiments of Formula II, Ring A is a 5-6-membered heteroaryl and is selected from the group consisting of

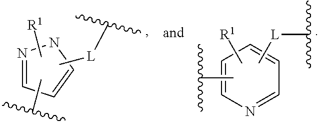

In some embodiments of Formula II, L¹ is selected from the group consisting of —(CH₂)—, —NH—, —NMe-, —NH(C═O)—, —(C═O)NH—, and —O—; In some embodiments of Formula II, L¹ is —(CH₂)—; In some embodiments of Formula II, L¹ is —NH—; In some embodiments of Formula II, L¹ is —NMe-; In some embodiments of Formula II, L¹ is —NH(C═O)—; In some embodiments of Formula II, L¹ is —(C═O)NH—; In some embodiments of Formula II, L¹ is —O—.

In some embodiments of Formula II, L² is selected from the group consisting of —(CH₂)—, —(CH₂CH₂)—, —(CH₂CH₂CH₂)—, —NH—, and —NMe-; In some embodiments of Formulas II, L² is —(CH₂)—; In some embodiments of Formulas II, L² is —(CH₂CH₂)—; In some embodiments of Formulas II, L² is —(CH₂CH₂CH₂)—; In some embodiments of Formulas II, L² is —NH—; In some embodiments of Formulas II, L² is —NMe-.

In some embodiments of Formula II, L³ is selected from the group consisting of —(CH₂)—, —(CH₂CH₂)—, —(CH₂CH₂CH₂)—, —(CH₂CH₂CH₂CH₂)—, —O—, and

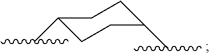 [SMILES: *C1CCC(*)CC1]

In some embodiments of Formula II, L³ is —(CH₂)—; In some embodiments of Formula II, L³ is —(CH₂CH₂)—; In some embodiments of Formula II, L³ is —(CH₂CH₂CH₂)—; In some embodiments of Formula II, L³ is —(CH₂CH₂CH₂CH₂)—; In some embodiments of Formula II, L³ is —O—; In some embodiments of Formula II, L³ is

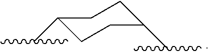 [SMILES: *C1CCC(*)CC1]

In some embodiments of Formula II, L⁴ is selected from the group consisting of —(CH₂)—, —(CH₂CH₂)—, —(CH₂CH₂CH₂)—, —(CH₂CH₂CH₂CH₂)—, —O—, —NH—, —NMe-, —NH(C═O)—, and —(C═O)NH—,

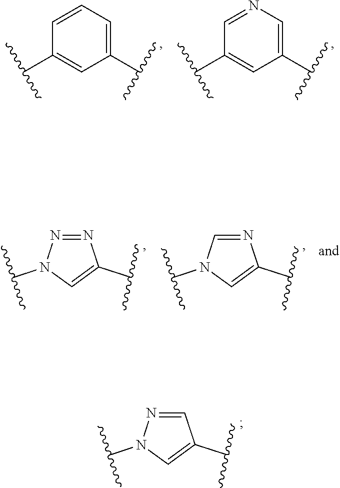

In some embodiments of Formula II, L⁴ is —(CH₂)—; In some embodiments of Formula II, L⁴ is —(CH₂CH₂)—; In some embodiments of Formula II, L⁴ is —(CH₂CH₂CH₂)—; In some embodiments of Formula II, L⁴ is —(CH₂CH₂CH₂CH₂)—; In some embodiments of Formula II, L⁴ is —O—; In some embodiments of Formula II, L⁴ is —NH—; In some embodiments of Formula II, L⁴ is —NMe-; In some embodiments of Formula II, L⁴ is —NH(C═O)—; In some embodiments of Formula II, L⁴ is —(C═O)NH—; In some embodiments of Formula II, L⁴ is

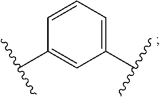 [SMILES: *c1cccc(*)c1]

In some embodiments of Formula II, L⁴ is

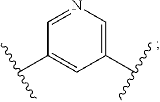 [SMILES: *c1cncc(*)c1]

In some embodiments of Formula II, L⁴ is

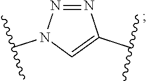 [SMILES: *c1cn(*)nn1]

In some embodiments of Formula II, L⁴ is

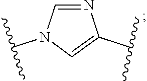 [SMILES: *c1cn(*)cn1]

In some embodiments of Formula II, L⁴ is

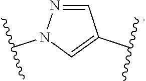 [SMILES: *c1cnn(*)c1]

Bioorganic & Medicinal Chemistry Letters (2006), 16(14), 3740-3744 and U.S. application Ser. Nos. 10/295,833 and 10/317,914 describe compounds having Formula III and are hereby incorporated by reference in their entirety.

One embodiment disclosed herein includes a compound having the structure of Formula III:

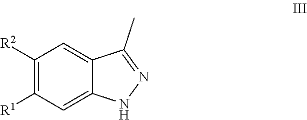

as well as prodrugs and pharmaceutically acceptable salt or solvate thereof.

In some embodiments of Formula (III):

R¹ is selected from the group consisting of H and halide (e.g., F, Cl, Br, I);

R² is a 6-membered -heteroaryl substituted with 1-4 (e.g., 1-3, 1-2, 1) R³;

each R³ is selected from the group consisting of —OR⁴, —NHR⁵, and —(C₁₋₄ alkylene)ₚheterocyclyl optionally substituted with 1-10 (e.g., 1-9, 1-8, 1-7, 1-6, 1-5, 1-4, 1-3, 1-2, 1) R⁶; wherein —(C₁₋₄ alkylene) is optionally substituted with one or more substituents as defined anywhere herein;

each R⁴ is independently selected from the group consisting of -heterocyclyl optionally substituted with 1-10 (e.g., 1-9, 1-8, 1-7, 1-6, 1-5, 1-4, 1-3, 1-2, 1) R⁷ and —CH₂CH(R⁸)NH₂;

each R is independently selected from the group consisting of —(C₁₋₄ alkylene)ₚheterocyclyl optionally substituted with 1-10 (e.g., 1-9, 1-8, 1-7, 1-6, 1-5, 1-4, 1-3, 1-2, 1) R⁹ and -carbocyclyl optionally substituted with 1-12 (e.g., 1-11, 1-10, 1-9, 1-8, 1-7, 1-6, 1-5, 1-4, 1-3, 1-2, 1) R¹⁰; wherein —(C₁₋₄ alkylene) is optionally substituted with one or more substituents as defined anywhere herein;

each R⁶ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R⁷ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R⁸ is independently selected from the group consisting of —(C₁₋₄ alkylene)aryl optionally substituted with 1-5 (e.g., 1-4, 1-3, 1-2, 1) R¹¹ and —(C¹⁻⁴ alkylene)heteroaryl optionally substituted with 1-4 (e.g., 1-3, 1-2, 1) R¹²; wherein each —(C₁₋₄ alkylene) is, independently, optionally substituted with one or more substituents as defined anywhere herein;

each R⁹ is independently selected from the group consisting of halide (e.g., F, Cl, Br, I), —OH, —NH₂, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆haloalkyl) (e.g., C₁₋₅, C₁₋₄, C₁₋₃, C₁₋₂, C₁);

each R¹⁰ is independently selected from the group consisting of halide (e.g., F, Cl, Br, I), —OH, —NH₂, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆haloalkyl) (e.g., C₁₋₅, C₁₋₄, C₁₋₃, C₁₋₂, C₁);

each R¹¹ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R¹² is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂-s, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁); and

each p is independently 0 or 1.

In some embodiments of Formula III, R¹ is halide.

In some embodiments of Formula III, R¹ is F.

In some embodiments of Formula III, R¹ is H.

In some embodiments of Formula III, R² is pyridinyl substituted with one R³;

In some embodiments of Formula III, R² is pyrazinyl substituted with one R³;

In some embodiments of Formula III, R³ is selected from the group consisting of —OR⁴, —NHR⁵, and —(CH₂)heterocyclyl optionally substituted with one R⁶.

In some embodiments of Formula III, R³ is —OR⁴; in some embodiments of Formula III, R³ is —NHR⁵; and in some embodiments of Formula III, R is —(CH₂)heterocyclyl optionally substituted with one R.

U.S. provisional application 62/634,656 and U.S. Pat. Nos. 9,221,793 and 9,745,271 describe compounds having Formula IV and are hereby incorporated by reference in their entirety.

One embodiment disclosed herein includes a compound having the structure of Formula IV:

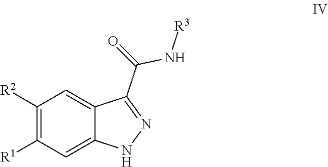

as well as prodrugs and pharmaceutically acceptable salt or solvate thereof.

In some embodiments of Formula (IV):

R¹ is selected from the group consisting of H and halide (e.g., F, Cl, Br, I);

R² is a -heteroaryl optionally substituted with 1-4 (e.g., 1-3, 1-2, 1) R⁴;

R³ is selected from the group consisting of -aryl optionally substituted with 1-5 (e.g., 1-4, 1-3, 1-2, 1) R⁵ and -heteroaryl optionally substituted with 1-4 (e.g., 1-3, 1-2, 1) R⁶;

each R⁴ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —(C₁₋₄ alkylene)ₚN(R⁷)(R⁸), —NHC(═O)R⁹, —(C₁₋₄ alkylene)ₚOR¹, unsubstituted -carbocyclyl, —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁴, —(C₁₋₄ alkylene)ₚaryl optionally substituted with 1-5 (e.g., 1-4, 1-3, 1-2, 1) R¹, and —(C₁₋₄ alkylene)ₚheteroaryl optionally substituted with 1-4 (e.g., 1-3, 1-2, 1) R¹²; wherein each —(C₁₋₄ alkylene) is, independently, optionally substituted with one or more substituents as defined anywhere herein;

each R is independently selected from the group consisting of halide (e.g., F, Cl, Br, I), —CN, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —(C₁₋₄ alkylene)ₚaryl optionally substituted with 1-5 (e.g., 1-4, 1-3, 1-2, 1) R¹³, —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁴, —C(═O)N(R⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;

each R⁶ is independently selected from the group consisting of halide (e.g., F, Cl, Br, I), —CN, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —(C₁₋₄ alkylene)ₚaryl optionally substituted with 1-5 (e.g., 1-4, 1-3, 1-2, 1) R¹³, —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁴, —C(═O)N(R⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;

each R⁷ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R⁸ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and -heterocyclyl optionally substituted with 1-10 (e.g., 1-9, 1-8, 1-7, 1-6, 1-5, 1-4, 1-3, 1-2, 1) R²¹;

alternatively, R⁷ and R⁸ are taken together to form a -heterocyclyl ring optionally substituted with 1-10 (e.g., 1-9, 1-8, 1-7, 1-6, 1-5, 1-4, 1-3, 1-2, 1) R²¹;

each R⁹ is independently selected from the group consisting of —N(R²²)₂, -carbocyclyl optionally substituted with 1-12 (e.g., 1-11, 1-10, 1-9, 1-8, 1-7, 1-6, 1-5, 1-4, 1-3, 1-2, 1) R²³, -heterocyclyl optionally substituted with 1-10 (e.g., 1-9, 1-8, 1-7, 1-6, 1-5, 1-4, 1-3, 1-2, 1) R²¹, and -aryl optionally substituted with 1-5 (e.g., 1-4, 1-3, 1-2, 1) R²⁴;

each R¹⁰ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆alkynyl) (e.g., C₂₋₅, C₂₋₄, C₂₋₃, C₂), unsubstituted —(C₁₋₆haloalkyl) (e.g., C₁-s, C₁₋₄, C₁₋₃, C₁₋₂, C₁), and -heterocyclyl optionally substituted with 1-10 (e.g., 1-9, 1-8, 1-7, 1-6, 1-5, 1-4, 1-3, 1-2, 1) R²¹;

each R¹¹ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂-s, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R¹² is independently selected from the group consisting of halide (e.g., F, Cl, Br, I), —(C₁₋₄ alkylene)pOH, unsubstituted —(C₁₋₆ alkyl) (e.g., C₁₋₅, C₁₋₄, C_{1_3}, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁); wherein —(C₁₋₄ alkylene) is optionally substituted with one or more substituents as defined anywhere herein;

each R¹³ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R¹⁴ is independently selected from the group consisting of halide (e.g., F, Cl, Br, I), —(C₁₋₄ alkylene)pOH, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁); wherein —(C₁₋₄ alkylene) is optionally substituted with one or more substituents as defined anywhere herein;

each R¹⁵ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and -carbocyclyl optionally substituted with 1-12 (e.g., 1-11, 1-10, 1-9, 1-8, 1-7, 1-6, 1-5, 1-4, 1-3, 1-2, 1) R²³;

alternatively, two adjacent R¹⁵ are taken together to form a -heterocyclyl ring optionally substituted with 1-10 (e.g., 1-9, 1-8, 1-7, 1-6, 1-5, 1-4, 1-3, 1-2, 1) R²¹;

each R¹⁶ is independently selected from the group consisting of unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and -carbocyclyl optionally substituted with 1-12 (e.g., 1-11, 1-10, 1-9, 1-8, 1-7, 1-6, 1-5, 1-4, 1-3, 1-2, 1) R²³;

each R¹⁷ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R¹⁸ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), —(C₁₋₄ alkylene)NMe₂, and -heterocyclyl ring optionally substituted with 1-10 (e.g., 1-9, 1-8, 1-7, 1-6, 1-5, 1-4, 1-3, 1-2, 1) R²¹; wherein —(C₁₋₄ alkylene) is optionally substituted with one or more substituents as defined anywhere herein;

each R¹⁹ is independently selected from the group consisting of unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R²⁰ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆haloalkyl) (e.g., C₁₋₅, C₁₋₄, C₁₋₃, C₁₋₂, C₁), —CH(CH₂OH)₂, —(C₁₋₄ alkylene)ₚheterocyclyl ring optionally substituted with 1-10 (e.g., 1-9, 1-8, 1-7, 1-6, 1-5, 1-4, 1-3, 1-2, 1) R²¹, and -aryl optionally substituted with 1-5 (e.g., 1-4, 1-3, 1-2, 1) R²⁴; wherein —(C₁₋₄ alkylene) is optionally substituted with one or more substituents as defined anywhere herein;

each R²¹ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R²² is independently selected from the group consisting of unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R²³ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R²⁴ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₃, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁); and

each p is independently 0 or 1.

In some embodiments of Formula IV, R¹ is halide.

In some embodiments of Formula IV, R¹ is F.

In some embodiments of Formula IV, R¹ is H.

In some embodiments of Formula IV, R² is a 5-membered -heteroaryl optionally substituted with 1-2 R⁴;

In some embodiments of Formula IV, R² is selected from the group consisting of pyrazolyl, imidazolyl, 1,2,3-triazolyl, isoxazolyl, oxazolyl, isothiazolyl, and thiazolyl; wherein each are optionally substituted with 1-2 R⁴.

In some embodiments of Formula IV, R² is pyrazolyl optionally substituted with 1-2 R⁴; in some embodiments of Formula IV, R² is imidazolyl optionally substituted with 1-2 R⁴; in some embodiments of Formula IV, R² is 1,2,3-triazolyl optionally substituted with 1-2 R⁴; in some embodiments of Formula IV, R² is isoxazolyl optionally substituted with 1-2 R⁴; in some embodiments of Formula IV, R² is oxazolyl optionally substituted with 1-2 R⁴; in some embodiments of Formula IV, R² is isothiazolyl optionally substituted with 1-2 R⁴; and in some embodiments of Formula IV, R² is thiazolyl optionally substituted with 1-2 R⁴.

In some embodiments of Formula IV, R⁴ is selected from the group consisting of unsubstituted —(C₁₋₃ alkyl) and -heterocyclyl optionally substituted with one R¹⁴.

In some embodiments of Formula IV, R⁴ is unsubstituted —(C₁₋₃ alkyl) and in some embodiments of Formula IV, R⁴ is -heterocyclyl optionally substituted with one R¹⁴.

In some embodiments of Formula IV, R² is a 6-membered -heteroaryl optionally substituted with 1-2 R⁴;

In some embodiments of Formula IV, R² is pyridinyl optionally substituted with one R⁴.

In some embodiments of Formula IV, R³ is selected from the group consisting of -phenyl optionally substituted with 1-2 R⁵, -pyridinyl optionally substituted with 1-2 R⁶, -pyrimidinyl optionally substituted with 1-2 R⁶, -pyrazinyl optionally substituted with 1-2 R⁶, -pyrazolyl optionally substituted with 1-2 R⁶, -isothiazolyl optionally substituted with 1-2 R⁶, and -thiazolyl optionally substituted with 1-2 R⁶.

In some embodiments of Formula IV, R³ is -phenyl optionally substituted with 1-2 R⁵; in some embodiments of Formula IV, R³ is -pyridinyl optionally substituted with 1-2 R⁶; in some embodiments of Formula IV, R³ is -pyrimidinyl optionally substituted with 1-2 R⁶; in some embodiments of Formula IV, R³ is -pyrazinyl optionally substituted with 1-2 R⁶; in some embodiments of Formula IV, R³ is -pyrazolyl optionally substituted with 1-2 R⁶; in some embodiments of Formula IV, R³ is -isothiazolyl optionally substituted with 1-2 R⁶; and in some embodiments of Formula IV, R³ is -thiazolyl optionally substituted with 1-2 R⁶.

In some embodiments of Formula IV, R⁵ is selected from the group consisting of F, —(CH₂)N(C₁₋₃ alkyl)(C₁₋₃ alkyl), —(CH₂)ₚheterocyclyl optionally substituted with 1-2 R¹⁴, and —O(heterocyclyl optionally substituted with 1-2 R²).

In some embodiments of Formula IV, R⁵ is F; in some embodiments of Formula IV, R is —(CH₂)N(C₁₋₃ alkyl)(C₁₋₃ alkyl); in some embodiments of Formula IV, R⁵ is —(CH₂)ₚheterocyclyl optionally substituted with 1-2 R¹⁴; and in some embodiments of Formula IV, R⁵ is —O(heterocyclyl optionally substituted with 1-2 R²¹).

In some embodiments of Formula IV, R⁶ is selected from the group consisting of F, Me, —(CH₂)N(C₁₋₃ alkyl)(C₁₋₃ alkyl), —(CH₂)ₚheterocyclyl optionally substituted with 1-2 R¹⁴, —OMe, —OCHF₂, —OCF₃, —O(heterocyclyl optionally substituted with 1-2 R²), and —C(═O)N(R⁵)₂.

In some embodiments of Formula IV, R⁶ is F; in some embodiments of Formula IV, R⁶ is Me; in some embodiments of Formula IV, R⁶ is —(CH₂)N(C₁₋₃ alkyl)(C₁₋₃ alkyl); in some embodiments of Formula IV, R⁶ is —(CH₂)ₚheterocyclyl optionally substituted with 1-2 R¹⁴; in some embodiments of Formula IV, R⁶ is —OMe; in some embodiments of Formula IV, R⁶ is —OCHF₂; in some embodiments of Formula IV, R⁶ is —OCF₃; in some embodiments of Formula IV, R⁶ is —O(heterocyclyl optionally substituted with 1-2 R²¹); and in some embodiments of Formula IV, R⁶ is —C(═O)N(R⁵)₂.

U.S. application Ser. Nos. 15/749,910, 15/749,922, 15/749,923, and 15/749,929, and U.S. Pat. Nos. 8,252,812, 8,450,340, 8,673,936, 8,883,822, 9,908,867, 9,475,807, 9,475,825, 9,493,487, 9,540,398, 9,546,185, 9,657,016, 9,738,638, and 9,758,531 describe compounds having Formula V and are hereby incorporated by reference in their entirety.

One embodiment disclosed herein includes a compound having the structure of Formula V:

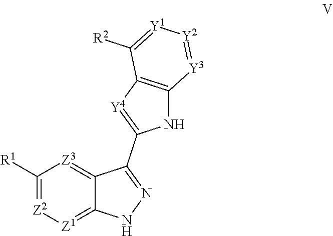

as well as prodrugs and pharmaceutically acceptable salt or solvate thereof.

In some embodiments of Formula (V):

R¹ is a -heteroaryl optionally substituted with 1-2 R³;

R² is selected from the group consisting of H, halide (e.g., F, Cl, Br, I), -aryl optionally substituted with 1-5 (e.g., 1-4, 1-3, 1-2, 1) R⁴-heteroaryl optionally substituted with 1-4 (e.g., 1-3, 1-2, 1) R⁵, and -heterocyclyl ring optionally substituted with 1-10 (e.g., 1-9, 1-8, 1-7, 1-6, 1-5, 1-4, 1-3, 1-2, 1) R⁶;

each R³ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, Cl-3, C₁₋₂, C₁), —(C₁₋₄ alkylene)ₚheterocyclyl optionally substituted with 1-10 (e.g., 1-9, 1-8, 1-7, 1-6, 1-5, 1-4, 1-3, 1-2, 1) R⁷, —C(═O)N(RW)₂, —NHC(═O)R⁹, —(C₁₋₄ alkylene)ₚN(R¹⁰)(R¹¹), —(C₁₋₄ alkylene)ₚOR¹², and -carbocyclyl optionally substituted with 1-12 (e.g., 1-11, 1-10, 1-9, 1-8, 1-7, 1-6, 1-5, 1-4, 1-3, 1-2, 1) R¹³; wherein each —(C₁₋₄ alkylene) is, independently, optionally substituted with one or more substituents as defined anywhere herein;

each R⁴ is independently selected from the group consisting of halide (e.g., F, Cl, Br, I), —CN, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —(C₁₋₄ alkylene)ₚNHSO₂R¹⁴, —NR⁵(C₁₋₄ alkylene)NR¹⁵R¹⁶, —(C₁₋₄ alkylene)ₚNR¹⁵R¹⁶, —OR¹⁷, and -heterocyclyl optionally substituted with 1-10 (e.g., 1-9, 1-8, 1-7, 1-6, 1-5, 1-4, 1-3, 1-2, 1) R¹⁹; wherein each —(C₁₋₄ alkylene) is, independently, optionally substituted with one or more substituents as defined anywhere herein;

each R⁵ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), and —C(═O)R¹⁸;

each R⁶ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R⁷ is independently selected from the group consisting of halide (e.g., F, Cl, Br, I), —NH₂, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R⁹ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂), -heterocyclyl optionally substituted with 1-10 (e.g., 1-9 , 1-8, 1-7, 1-6, 1-5, 1-4, 1-3, 1-2, 1) R¹⁹, —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; wherein —(C₁₋₄ alkylene) is optionally substituted with one or more substituents as defined anywhere herein;

each R⁹ is independently selected from the group consisting of unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁹, —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; —(C₁₋₄ alkylene)ₚaryl optionally substituted with 1-5 (e.g., 1-4, 1-3, 1-2, 1) R²¹, —(C₁₋₄ alkylene)ₚN(R²²)₂; wherein each —(C₁₋₄ alkylene) is, independently, optionally substituted with one or more substituents as defined anywhere herein;

each R¹⁰ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R¹¹ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; and —(C₁₋₄ alkylene)ₚaryl optionally substituted with 1-5 (e.g., 1-4, 1-3, 1-2, 1) R²¹; wherein each —(C₁₋₄ alkylene) is, independently, optionally substituted with one or more substituents as defined anywhere herein;

each R¹² is independently selected from the group consisting of H unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆alkynyl) (e.g., C₂₋₅, C₂₋₄, C₂₋₃, C₂), unsubstituted —(C₁₋₆haloalkyl) (e.g., C₁₋₅, C₁₋₄, C₁₋₃, C₁₋₂, C₁), —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁹, —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R²⁰; —(C₁₋₄ alkylene)ₚaryl optionally substituted with 1-5 (e.g., 1-4, 1-3, 1-2, 1) R²¹, —(C₁₋₄ alkylene)ₚN(R²²)₂; wherein each —(C₁₋₄ alkylene) is, independently, optionally substituted with one or more substituents as defined anywhere herein;

each R¹³ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R¹⁴ is independently selected from the group consisting of unsubstituted —(C₁₋₆ alkyl) (e.g., C₁₋₅, C₁₋₄, C₁₋₃, C₁₋₂, C₁), and unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂);

each R¹⁵ is independently selected from the group consisting of H, unsubstituted —(C₁₋₆ alkyl) (e.g., C₁₋₅, C₁₋₄, C₁₋₃, C₁₋₂, C₁), and unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂);

each R¹⁶ is independently selected from the group consisting of H, unsubstituted —(C₁₋₆ alkyl) (e.g., C₁₋₅, C₁₋₄, C₁₋₃, C₁₋₂, C₁), and unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂);

each R¹⁷ is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆alkynyl) (e.g., C₂₋₅, C₂₋₄, C₂₋₃, C₂), unsubstituted —(C₁₋₆haloalkyl) (e.g., C₁₋₅, C₁₋₄, C₁₋₃, C₁₋₂, C₁), —(C₁₋₄ alkylene)ₚheterocyclyl optionally substituted with 1-10 (e.g., 1-9, 1-8, 1-7, 1-6, 1-5, 1-4, 1-3, 1-2, 1) R¹⁹, and, —(C₁₋₄ alkylene)ₚN(R²²)₂; wherein each —(C₁₋₄ alkylene) is, independently, optionally substituted with one or more substituents as defined anywhere herein;

each R¹⁸ is independently selected from the group consisting of unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R¹⁹ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R²⁰ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R²¹ is independently selected from the group consisting of halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆alkenyl) (e.g., C₂-s, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R²² is independently selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R²³ is independently selected from the group consisting of H and halide (e.g., F, Cl, Br, I);

Y¹, Y², and Y³ are independently selected from the group consisting of —CR²³═ and —N═;

Y⁴ is selected from the group of —CH═ and —N═;

Z¹, Z², and Z³ are independently selected from the group consisting of —CR²³═ and —N═; and

each p is independently 0 or 1.

In some embodiments of Formula V, R¹ is selected from the group consisting of -pyridinyl optionally substituted with 1-2 R³, -pyrimidinyl optionally substituted with 1-2 R³, -pyrazinyl optionally substituted with 1-2 R³, -pyrazolyl optionally substituted with 1-2 R³, -isothiazolyl optionally substituted with 1-2 R³, and -thiazolyl optionally substituted with 1-2 R³.

In some embodiments of Formula V, R¹ is -pyridinyl optionally substituted with 1-2 R³; in some embodiments of Formula V, R¹ is -pyrimidinyl optionally substituted with 1-2 R³; in some embodiments of Formula V, R¹ is -pyrazinyl optionally substituted with 1-2 R³; in some embodiments of Formula V, R¹ is -pyrazolyl optionally substituted with 1-2 R³; in some embodiments of Formula V, R¹ is -isothiazolyl optionally substituted with 1-2 R³; and in some embodiments of Formula V, R¹ is -thiazolyl optionally substituted with 1-2 R³.

In some embodiments of Formula V, R² is selected from the group consisting of -phenyl optionally substituted with 1-2 R⁴-pyridinyl optionally substituted with one R⁵, -thiophenyl optionally substituted with one R⁵, -furanyl optionally substituted with one R⁵, -piperidinyl ring optionally substituted with one R⁶, and -piperazinyl ring optionally substituted with one R⁶.

In some embodiments of Formula V, R² is -phenyl optionally substituted with 1-2 R⁴; in some embodiments of Formula V, R² is -pyridinyl optionally substituted with one R⁵; in some embodiments of Formula V, R² is -thiophenyl optionally substituted with one R⁵; in some embodiments of Formula V, R² is -furanyl optionally substituted with one R⁵; in some embodiments of Formula V, R² is -piperidinyl ring optionally substituted with one R⁶; and in some embodiments of Formula V, R² is -piperazinyl ring optionally substituted with one R⁶.

In some embodiments of Formula V, R³ is selected from the group consisting of unsubstituted —(C₁₋₃ alkyl), —(CH₂)ₚheterocyclyl optionally substituted with 1-2 R⁷, —OH, —O((CH₂CH₂)heterocyclyl), —O(heterocyclyl), —O((CH₂)N(C₁₋₃ alkyl)(C₁₋₃ alkyl)), —NH₂, —(CH₂)N(C₁₋₃ alkyl)(C₁₋₃ alkyl), —(CH₂)NH(C₁₋₃ alkyl), —N(C₁₋₃ alkyl)(C₁₋₃ alkyl), —NHC(═O)(C₁₋₅ alkyl), and —NHC(═O)(—(CH₂)ₚheterocyclyl).

In some embodiments of Formula V, R³ is unsubstituted —(C₁₋₃ alkyl); in some embodiments of Formula V, R³ is —(CH₂)ₚheterocyclyl optionally substituted with 1-2 R⁷; in some embodiments of Formula V, R³ is —OH; in some embodiments of Formula V, R³ is —O((CH₂CH₂)heterocyclyl); in some embodiments of Formula V, R³ is —O(heterocyclyl); in some embodiments of Formula V, R³ is —O((CH₂)N(C₁₋₃ alkyl)(C₁₋₃ alkyl)); in some embodiments of Formula V, R³ is —NH₂; in some embodiments of Formula V, R³ is —(CH₂)N(C₁₋₃ alkyl)(C₁₋₃ alkyl); in some embodiments of Formula V, R³ is —(CH₂)NH(C₁₋₃ alkyl); in some embodiments of Formula V, R³ is —N(C₁₋₃ alkyl)(C₁₋₃ alkyl); in some embodiments of Formula V, R³ is —NHC(═O)(C₁₋₅ alkyl); and in some embodiments of Formula V, R³ is —NHC(═O)(—(CH₂)ₚheterocyclyl).

In some embodiments of Formula V, Y¹, Y², Y³, and Y⁴ are all —CH═; in some embodiments of Formula V, Y¹ is —N═ and Y², Y³, and Y⁴ are all —CH═; in some embodiments of Formula V, Y² is —N═ and Y¹, Y³, and Y⁴ are all —CH═; in some embodiments of Formula V, Y³ is —N═ and Y¹, Y², and Y⁴ are all —CH═; in some embodiments of Formula V, Y⁴ is —N═ and Y¹, Y², and Y³ are all —CH═.

In some embodiments of Formula V, Z¹, Z², and Z³ are all —CH═; in some embodiments of Formula V, Z² is —CF═ and Z¹ and Z³ are both —CH═; in some embodiments of Formula V, Z¹ is —N═ and Z² and Z³ are both —CH═; in some embodiments of Formula V, Z² is —N═ and Z¹ and Z³ are both —CH═; in some embodiments of Formula V, Z³ is —N═ and Z¹ and Z² are both —CH═.

In some embodiments of Formula V, Y¹, Y², Y³, Y⁴, Z¹, Z², and Z³ are all —CH═; in some embodiments of Formula V, Z² is —CF═ and Y¹, Y², Y³, Y⁴, Z¹ and Z³ are all —CH═; in some embodiments of Formula V, Y⁴ is —N═ and Y¹, Y², Y³, Z¹, Z² and Z³ are all —CH═; in some embodiments of Formula V, Z² is —CF═, Y⁴ is —N═ and Y¹, Y², Y³, Z¹ and Z³ are all —CH═.

In some embodiments of Formula V, Y¹ is —N═ and Y², Y³, Y⁴, Z¹, Z², and Z³ are all —CH═; in some embodiments of Formula V, Y² is —N═ and Y¹, Y², Y³, Y⁴, Z, Z², and Z³ are all —CH═; in some embodiments of Formula V, Y³ is —N═ and Y¹, Y², Y⁴, Z¹, Z², and Z³ are all —CH═; in some embodiments of Formula V, Y¹ is —N═, Z² is —CF═ and Y², Y³, Y⁴, Z¹, and Z³ are all —CH═; in some embodiments of Formula V, Y² is —N═, Z² is —CF═ and Y¹, Y³, Y⁴, Z¹, and Z³ are all —CH═; and in some embodiments of Formula V, Y³ is —N═, Z² is —CF═ and Y¹, Y², Y⁴, Z¹, and Z³ are all —CH═; in some embodiments of Formula V, Y¹ and Y⁴ are —N═ and Y², Y³, Z¹, Z², and Z³ are all —CH═; in some embodiments of Formula V, Y² and Y⁴ are —N═ and Y¹, Y³, Z¹, Z², and Z³ are all —CH═; in some embodiments of Formula V, Y³ and Y⁴ are —N═ and Y¹, Y², Z¹, Z², and Z³ are all —CH═; in some embodiments of Formula V, Y¹ and Y⁴ are —N═, Z² is —CF═ and Y², Y³, Z¹, and Z³ are all —CH═; in some embodiments of Formula V, Y² and Y⁴ are —N═, Z² is —CF═ and Y¹, Y³, Z¹, and Z³ are all —CH═; and in some embodiments of Formula V, Y³ and Y⁴ are —N═, Z² is —CF═ and Y¹, Y², Z¹, and Z³ are all —CH═.

In some embodiments of Formula V, Z¹ is —N═ and Y¹, Y², Y³, Y⁴, Z² and Z³ are all —CH═; in some embodiments of Formula V, Z² is —N═ and Y¹, Y², Y³, Y⁴, Z¹ and Z³ are all —CH═; and in some embodiments of Formula V, Z³ is —N═ and Y¹, Y², Y³, Y⁴, Z¹ and Z² are all —CH═; in some embodiments of Formula V, Z¹ and Y⁴ are —N═ and Y¹, Y², Y³, Z² and Z³ are all —CH═; in some embodiments of Formula V, Z² and Y⁴ are —N═ and Y¹, Y², Y³, Z¹ and Z³ are all —CH═; and in some embodiments of Formula V, Z³ and Y⁴ are —N═ and Y¹, Y², Y³, Z¹ and Z² are all —CH═.

In some embodiments of Formula V, Y¹ and Z¹ are —N═ and Y², Y³, Y⁴, Z², and Z³ are all —CH═; in some embodiments of Formula V, Y¹ and Z² are —N═ and Y², Y³, Y⁴, Z¹, and Z³ are all —CH═; Y¹ and Z³ are —N═ and Y², Y³, Y⁴, Z¹, and Z² are all —CH═; in some embodiments of Formula V, Y² and Z¹ are —N═ and Y¹, Y³, Y⁴, Z², and Z³ are all —CH═; in some embodiments of Formula V, Y² and Z² are —N═ and Y¹, Y³, Y⁴, Z, and Z³ are all —CH═; in some embodiments of Formula V, Y² and Z³ are —N═ and Y¹, Y³, Y⁴, Z, and Z² are all —CH═; in some embodiments of Formula V, Y³ and Z¹ are —N═ and Y¹, Y², Y⁴, Z², and Z³ are all —CH═; in some embodiments of Formula V, Y³ and Z² are —N═ and Y¹, Y², Y⁴, Z¹, and Z³ are all —CH═; and in some embodiments of Formula V, Y³ and Z³ are —N═ and Y¹, Y², Y⁴, Z¹, and Z² are all —CH═;

in some embodiments of Formula V, Y¹, Z¹, and Y⁴ are —N═ and Y², Y³, Z², and Z³ are all —CH═; in some embodiments of Formula V, Y¹, Z², and Y⁴ are —N═ and Y², Y³, Z¹, and Z³ are all —CH═; Y¹, Z³, and Y⁴ are —N═ and Y², Y³, Z¹, and Z² are all —CH═; in some embodiments of Formula V, Y², Z¹, and Y⁴ are —N═ and Y¹, Y³, Z², and Z³ are all —CH═; in some embodiments of Formula V, Y², Z², and Y⁴ are —N═ and Y¹, Y³, Z¹, and Z³ are all —CH═; in some embodiments of Formula V, Y², Z³, and Y⁴ are —N═ and Y¹, Y³, Z¹, and Z² are all —CH═; in some embodiments of Formula V, Y³, Z¹, and Y⁴ are —N═ and Y¹, Y², Z², and Z³ are all —CH═; in some embodiments of Formula V, Y³, Z², and Y⁴ are —N═ and Y¹, Y², Z¹, and Z³ are all —CH═; and in some embodiments of Formula V, Y³, Z³, and Y⁴ are —N═ and Y¹, Y², Z¹, and Z² are all —CH═.

Kazuho Nishimura, Masahiro Yaguchi, Yukiko Yamamoto, Shunsuke Ebara, Kawakita Yoichi, Ryo Mizojiri, Yusuke Nakayama, Kozo Hayashi, Shuichi Miyakawa, Kenichi Iwai, Toshiyuki Nomura. Takeda Pharmaceutical Company Limited, Cambridge, Mass., Small Molecule Inhibitor of Pre-mRNA Splicing Evokes Antitumor Activity via MDM4-p53. Poster presented at: Molecular Targets and Cancer Therapeutics: Discovery, Biology, and Clinical Applications. AACR-NCI-EORTC International Conference. 2017 Oct. 27-30, Philadelphia, Pa., Journal of Medicinal Chemistry (2017), 60(21), 8989-9002, and U.S. Pat. Nos. 9,346,812 and 9,428,509 describe compounds having Formula VI and are hereby incorporated by reference in their entirety.

One embodiment disclosed herein includes a compound having the structure of Formula VI:

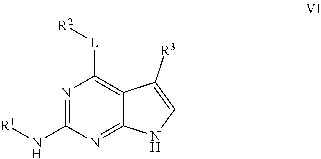

as well as prodrugs and pharmaceutically acceptable salt or solvate thereof.

In some embodiments of Formula (VI):

R¹ is selected from the group consisting of H, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂), and -heteroaryl optionally substituted with 1-4 (e.g., 1-3, 1-2, 1) R⁴, -aryl optionally substituted with 1-5 (e.g., 1-4, 1-3, 1-2, 1) R⁵;

R² is selected from the group consisting of H, —(C₁₋₄ alkylene)ₚheteroaryl optionally substituted with 1-4 (e.g., 1-3, 1-2, 1) R⁶, —(C₁₋₄ alkylene)ₚheterocyclyl optionally substituted with 1-10 (e.g., 1-9, 1-8, 1-7, 1-6, 1-5, 1-4, 1-3, 1-2, 1) R⁷, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R; wherein each —(C₁₋₄ alkylene) is, independently, optionally substituted with one or more substituents as defined anywhere herein;

R³ is selected from the group consisting of -heteroaryl optionally substituted with 1-4 (e.g., 1-3, 1-2, 1) R⁹ and -aryl optionally substituted with 1-5 (e.g., 1-4, 1-3, 1-2, 1) R¹⁰;

each R⁴ is independently selected from the group consisting of halide (e.g., F, Cl, Br, I), —CN, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —OR¹¹, —C(═O)N(R¹²)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R¹³, —SO₂R¹⁴, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R⁵; wherein each —(C₁₋₄ alkylene) is, independently, optionally substituted with one or more substituents as defined anywhere herein;

each R⁵ is independently selected from the group consisting of halide (e.g., F, Cl, Br, I), —CN, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —OR¹¹, —C(═O)N(R¹²)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R¹³, —SO₂R¹⁴, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R⁵; wherein each —(C₁₋₄ alkylene) is, independently, optionally substituted with one or more substituents as defined anywhere herein;

each R⁶ is independently selected from the group consisting of halide (e.g., F, Cl, Br, I), —CN, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —OR¹¹, —C(═O)N(R¹²)₂, and —SO₂R¹⁴;

each R⁷ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R⁸ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R⁹ is independently selected from the group consisting of halide (e.g., F, Cl, Br, I), —CN, unsubstituted —(C₁₋₆ alkyl) (e.g., C₁₋₅, C₁₋₄, C_{1_3}, C₁₋₂, C₁), unsubstituted —(C₂₋₆ alkenyl) (e.g., C₂-s, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —OR¹¹, —C(═O)N(R¹²)₂, and —SO₂R¹⁴;

each R¹⁰ is independently selected from the group consisting of halide (e.g., F, Cl, Br, I), —CN, unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), unsubstituted —(C₁₋₆ haloalkyl) (e.g., C₁₋₅, C₁₋₄, C₁₋₃, C₁₋₂, C₁), —OR¹¹, —C(═O)N(R¹²)₂, and —SO₂R¹⁴;

each R¹¹ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

each R¹² is independently selected from the group consisting of H, halide (e.g., F, Cl, Br, I), unsubstituted —(C₁₋₆ alkyl) (e.g., C₁₋₅, C₁₋₄, C₁₋₃, C₁₋₂, C₁), unsubstituted —(C₂₋₆ alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R¹³ is independently selected from the group consisting of halide (e.g., F, Cl, Br, I), unsubstituted —(C₁₋₆alkyl) (e.g., C₁₋₃, C₁₋₄, C₁₋₃, C₁₋₂, C₁₎, unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₃, C₁₋₄, C₁₋₃, C₁₋₂, C₁);

each R¹⁴ is independently selected from the group consisting of halide (e.g., F, Cl, Br, I), unsubstituted —(C₁₋₆alkyl) (e.g., C₁₋₃, C₁₋₄, C₁₋₃, C₁₋₂, C₁), unsubstituted —(C₂₋₆alkenyl) (e.g., C₂₋₅, C₂₋₄, C₂₋₃, C₂), and unsubstituted —(C₂₋₆ alkynyl) (e.g., C₂₋₅, C₂₋₄, C₂₋₃, C₂);

each R¹⁵ is independently selected from the group consisting of halide (e.g., F, Cl, Br, I), unsubstituted —(C₁₋₆alkyl) (e.g., C₁₋₅, C₁₋₄, C₁₋₃, C₁₋₂, C₁), unsubstituted —(C₂₋₆alkenyl) (e.g., C₂₋₅, C₂₋₄, C₂₋₃, C₂), unsubstituted —(C₂₋₆ alkynyl) (e.g., C₂₋₅, C₂₋₄, C₂₋₃, C₂), and unsubstituted —(C₁₋₆ haloalkyl) (e.g., C₁₋₅, C₁₋₄, C₁₋₃, C₁₋₂, C₁);

L is selected from the group consisting of a bond, —O—, and —NH—; and

each p is independently 0 or 1.

In some embodiments of Formula VI, R¹ is selected from the group consisting of unsubstituted —(C₁₋₃ alkyl) and -phenyl substituted with 1-2 R.

In some embodiments of Formula VI, R¹ is unsubstituted —(C₁₋₃ alkyl); in some embodiments of Formula VI, R¹ is Me; and in some embodiments of Formula VI, R¹ is -phenyl substituted with 1-2 R¹.

In some embodiments of Formula VI, R² is selected from the group consisting of —(CH₂)ₚheteroaryl optionally substituted with 1-2 R⁶ and -carbocyclyl optionally substituted with 1-2 R⁸.

In some embodiments of Formula VI, R² is —(CH₂)ₚheteroaryl optionally substituted with 1-2 R⁶; in some embodiments of Formula VI, R² is —(CH₂)ₚpyridinyl optionally substituted with 1-2 R⁶; in some embodiments of Formula VI, R² is —(CH₂)ₚpyrimidinyl optionally substituted with 1-2 R⁶; in some embodiments of Formula VI, R² is —(CH₂)ₚpyrazinyl optionally substituted with 1-2 R⁶; in some embodiments of Formula VI, R² is —(CH₂)ₚpyrazolyl optionally substituted with 1-2 R⁶; in some embodiments of Formula VI, R² is —(CH₂)ₚisothiazolyl optionally substituted with 1-2 R⁶; in some embodiments of Formula VI, R² is —(CH₂)ₚthiazolyl optionally substituted with 1-2 R⁶ in some embodiments of Formula VI, R² is -carbocyclyl optionally substituted with 1-2 R; in some embodiments of Formula VI, R² is -cyclopropyl optionally substituted with 1-2 R⁸; in some embodiments of Formula VI, R² is -cyclobutyl optionally substituted with 1-2 R⁸; in some embodiments of Formula VI, R² is -cyclopentyl optionally substituted with 1-2 R⁸; and in some embodiments of Formula VI, R² is -cyclohexyl optionally substituted with 1-2 R⁸.

In some embodiments of Formula VI, R³ is selected from the group consisting of -heteroaryl optionally substituted with 1-2 R⁹ and -phenyl optionally substituted with 1-2 R¹⁰.

In some embodiments of Formula VI, R³ is -heteroaryl optionally substituted with 1-2 R⁹; in some embodiments of Formula VI, R³ is -pyridinyl optionally substituted with 1-2 R⁹; in some embodiments of Formula VI, R³ is -quinolinyl optionally substituted with 1-2 R⁹; in some embodiments of Formula VI, R³ is -isoquinolinyl optionally substituted with 1-2 R⁹; in some embodiments of Formula VI, R³ is -benzoxazolyl optionally substituted with 1-2 R⁹; in some embodiments of Formula VI, R³ is -benzothiazolyl optionally substituted with 1-2 R⁹; in some embodiments of Formula VI, R³ is -benzoimidiazolyl optionally substituted with 1-2 R⁹; and in some embodiments of Formula VI, R³ is -phenyl optionally substituted with 1-2 R¹⁰.

In some embodiments of Formula VI, L is a bond; in some embodiments of Formula VI, L is —O—, and in some embodiments of Formula VI, L is —NH—.

U.S. provisional applications 62/577,818, 62/578,370, 62/578,691, and 62/579,883, U.S. application Ser. Nos. 15/498,990 and 15/499,013, and U.S. Pat. No. 9,951,048 describe compounds having Formula VII and are hereby incorporated by reference in their entirety.

One embodiment disclosed herein includes a compound having the structure of Formula VII:

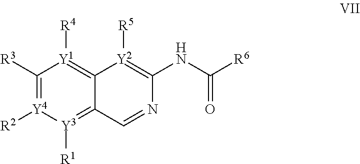

as well as prodrugs and pharmaceutically acceptable salt or solvate thereof.

In some embodiments of Formula (VII):

R¹, R², R⁴, and R⁵ are independently absent or selected from the group consisting of H and halide (e.g., F, Cl, Br, I);

R³ is selected from the group of -heteroaryl optionally substituted with 1-4 (e.g., 1-3, 1-2, 1) R⁸ and -Xheterocyclyl optionally substituted with one or more halides (e.g., F, Cl, Br, I) or one or more unsubstituted —(C₁₋₅ alkyl) (e.g., C₁₋₄, C₁₋₃, C₁₋₂, C₁);

R⁶ is selected from the group consisting of -aryl substituted with 1-5 (e.g., 1-4, 1-3, 1-2, 1) R⁹, —(C₂₋₄ alkenylene)aryl substituted with 1-5 (e.g., 1-4, 1-3, 1-2, 1) R⁹, —(C₁₋₄ alkylene)ₚheteroaryl optionally substituted with 1-6 (e.g., 1-5, 1-4, 1-3, 1-2, 1) R¹⁰; -heterocyclyl optionally substituted with 1-10 (e.g., 1-9, 1-8, 1-7, 1-6, 1-5, 1-4, 1-3, 1-2, 1) R¹¹, -carbocyclyl optionally substituted with 1-12 (e.g., 1-11, 1-10, 1-9, 1-8, 1-7, 1-6, 1-5, 1-4, 1-3, 1-2, 1) R¹², and —(C₂₋₉ alkynyl) optionally substituted with one or more halides (e.g., F, Cl, Br, I)s; wherein each —(C₁₋₄ alkylene) is, independently, optionally substituted with one or more substituents as defined anywhere herein; wherein —(C₁₋₄ alkenylene) is, optionally substituted with one or more substituents as defined anywhere herein;

with the proviso that R⁶ is heterocyclyl only when R³ is a 6-membered heteroaryl;

each R⁸ is independently selected from the group consisting of halide (e.g., F, Cl, Br, I), unsubstituted —(C₁₋₉ alkyl) (e.g., C₁₋₆, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C_{2_8}, C_{2_7}, C_{2_6}, C₂₋₅, C₂₋₄, C₂₋₃, C₂), unsubstituted —(C₁₋₉ haloalkyl) (e.g., C₁₋₈, C_{1_7}, C₁₋₆, C₁₋₅, C₁₋₄, C₁₋₃, C₁₋₂, C₁), —CN, —N(R¹⁵)(R¹⁸), —(C₁₋₄ alkylene)ₚXR¹⁹, —C(═O)N(R⁵)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R²⁰, and -carbocyclyl optionally substituted with 1-12 (e.g., 1-11, 1-10, 1-9, 1-8, 1-7, 1-6, 1-5, 1-4, 1-3, 1-2, 1) R²¹; wherein each —(C₁₋₄ alkylene) is, independently, optionally substituted with one or more substituents as defined anywhere herein;

alternatively, two adjacent R⁸ are taken together to form a ring which is selected from the group consisting of -heterocyclyl optionally substituted with 1-10 (e.g., 1-9, 1-8, 1-7, 1-6, 1-5, 1-4, 1-3, 1-2, 1) R²² and -carbocyclyl optionally substituted with 1-12 (e.g., 1-11, 1-10, 1-9, 1-8, 1-7, 1-6, 1-5, 1-4, 1-3, 1-2, 1) R²¹;

each R⁹ is independently selected from the group consisting of D, halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), —XR²³, —(C₁₋₄ alkylene)ₚN(R²⁴)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R²²; wherein each —(C₁₋₄ alkylene) is, independently, optionally substituted with one or more substituents as defined anywhere herein;

each R¹⁰ is independently selected from the group consisting of halide (e.g., F, Cl, Br, I), unsubstituted —(C₁₋₉ alkyl) (e.g., C₁₋₈, C₁₋₇, C₁₋₆, C₁₋₅, C₁₋₄, C₁₋₃, C₁₋₂, C₁), unsubstituted —(C₂₋₉ alkenyl) (e.g., C₂₋₈, C₂₋₇, C₂₋₆, C₂₋₅, C₂₋₄, C₂₋₃, C₂), unsubstituted —(C₂₋₉ alkynyl) (e.g., C₂₋₈, C₂₋₇, C₂₋₆, C₂₋₅, C₂₋₄, C₂₋₃, C₂), unsubstituted —(C₁₋₉ haloalkyl) (e.g., C₁₋₈, C₁₋₇, C₁₋₆, C₁₋₅, C₁₋₄, C₁₋₃, C₁₋₂, C₁), —CN, —XR²³, —C(═O)N(R⁵)₂, —(C₁₋₄ alkylene)ₚN(R²⁴)₂, -heterocyclyl optionally substituted with 1-10 (e.g., 1-9, 1-8, 1-7, 1-6, 1-5, 1-4, 1-3, 1-2, 1) R²², and -carbocyclyl optionally substituted with 1-12 (e.g., 1-11, 1-10, 1-9, 1-8, 1-7, 1-6, 1-5, 1-4, 1-3, 1-2, 1) R²¹; wherein —(C₁₋₄ alkylene) is optionally substituted with one or more substituents as defined anywhere herein;

each R¹¹ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), and unsubstituted —(C₁₋₉ haloalkyl);

each R¹² is independently selected from the group consisting of halide (e.g., F, Cl, Br, I), —(C₁₋₄ alkylene)ₚOR¹⁹; wherein —(C₁₋₄ alkylene) is optionally substituted with one or more substituents as defined anywhere herein;

each R¹⁵ is selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁);

R¹⁸ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), and —(C₁₋₄ alkylene)ₚheterocyclyl optionally substituted with one or more halides (e.g., F, Cl, Br, I) or one or more unsubstituted —(C₁₋₃ alkyl) (e.g., C₁₋₄, C₁₋₃, C₁₋₂, C₁); wherein —(C₁₋₄ alkylene) is, independently,

optionally substituted with one or more substituents as defined anywhere herein; each R¹⁹ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₄ haloalkyl) (e.g., C₁₋₄, C₁₋₃, C₁₋₂, C₁), —(C₁₋₄ alkylene)ₚheterocyclyl optionally substituted with one or more halides (e.g., F, Cl, Br, I)s or one or more unsubstituted —(C₁₋₃ alkyl) (e.g., C₁₋₄, C₁₋₃, C₁₋₂, C₁); wherein —(C₁₋₄ alkylene) is optionally substituted with one or more substituents as defined anywhere herein;

each R²⁰ independently is selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂_s alkynyl) (e.g., C₂₋₄, C₂₋₃, C₂), unsubstituted —(C₁₋₅ haloalkyl) (e.g., C₁₋₄, C₁₋₃, C₁₋₂, C₁), and —OH;

each R²¹ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), and —CN;

each R²² is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), —OH, —N(R⁵)₂, —C(═O)R³⁴, and -carbocyclyl optionally substituted with 1-12 (e.g., 1-11, 1-10, 1-9, 1-8, 1-7, 1-6, 1-5, 1-4, 1-3, 1-2, 1) R²¹;

each R²³ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), —(C₁₋₄ alkylene)N(R¹⁵)₂, -heterocyclyl optionally substituted with 1-10 (e.g., 1-9, 1-8, 1-7, 1-6, 1-5, 1-4, 1-3, 1-2, 1) R³¹, and -carbocyclyl optionally substituted with 1-12 (e.g., 1-11, 1-10, 1-9, 1-8, 1-7, 1-6, 1-5, 1-4, 1-3, 1-2, 1) R²¹; wherein —(C₁₋₄ alkylene) is optionally substituted with one or more substituents as defined anywhere herein;

each R²⁴ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), —(C₁₋₄ alkylene)ₚheterocyclyl optionally substituted with one or more halides (e.g., F, Cl, Br, I) or one or more unsubstituted —(C₁₋₅ alkyl), and —(C₁₋₄ alkylene)N(R⁵)₂; wherein each —(C₁₋₄ alkylene) is, independently, optionally substituted with one or more substituents as defined anywhere herein;

each R³¹ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁);

each R³⁴ is independently selected from the group consisting of —O(C₁₋₅ alkyl) and a heteroaryl optionally substituted with 1-6 (e.g., 1-5, 1-4, 1-3, 1-2, 1) R³⁵;

each R³⁵ is a -heterocyclyl optionally substituted with one or more halides (e.g., F, Cl, Br, I) or one or more unsubstituted —(C₁₋₅ alkyl) (e.g., C₁₋₄, C₁₋₃, C₁₋₂, C₁);

each X is selected from the group consisting of O and S;

Y¹, Y², Y³, and Y⁴ are independently selected from the group consisting of carbon and nitrogen; wherein

if Y¹ is nitrogen then Y², Y³, and Y⁴ are carbon, and R⁴ is absent;

if Y² is nitrogen then Y¹, Y³, and Y⁴ are carbon, and R⁵ is absent;

if Y³ is nitrogen then Y¹, Y², and Y⁴ are carbon, and R¹ is absent;

if Y⁴ is nitrogen then Y¹, Y², and Y³ are carbon, and R² is absent; and

each p is independently 0 or 1.

In some embodiments of Formula VII, R¹, R², R⁴, and R⁵ are all H or absent; in some embodiments of Formula VII, R¹ is F and R², R⁴, and R⁵ are all H or absent; in some embodiments of Formula VII, R² is F and R¹, R⁴, and R⁵ are all H or absent; in some embodiments of Formula VII, R⁴ is F and R¹, R², and R⁵ are all H or absent; and in some embodiments of Formula VII, R⁵ is F and R¹, R², and R⁴ are all H or absent.

In some embodiments of Formula VII, R³ is selected from the group consisting of -pyridinyl optionally substituted with 1-2 R, -pyrimidinyl optionally substituted with 1-2 R⁸, -pyrazinyl optionally substituted with 1-2 R, -pyrazolyl optionally substituted with 1-2 R⁸, -isothiazolyl optionally substituted with 1-2 R, -thiazolyl optionally substituted with 1-2 R⁸, -pyrazolyl optionally substituted with 1-2 R⁸, -imidazolyl optionally substituted with 1-2 R⁸, -1,2,3-triazolyl optionally substituted with 1-2 R⁸, -isoxazolyl optionally substituted with 1-2 R⁸, and -oxazolyl optionally substituted with 1-2 R⁸;

In some embodiments of Formula VII, R³ is -pyridinyl optionally substituted with 1-2 R⁸; in some embodiments of Formula VII, R³ is -pyrimidinyl optionally substituted with 1-2 R⁸; in some embodiments of Formula VII, R³ is -pyrazinyl optionally substituted with 1-2 R⁸; in some embodiments of Formula VII, R³ is -pyrazolyl optionally substituted with 1-2 R⁸; in some embodiments of Formula VII, R³ is -isothiazolyl optionally substituted with 1-2 R⁸; in some embodiments of Formula VII, R is -thiazolyl optionally substituted with 1-2 R⁸; in some embodiments of Formula VII, R³ is -pyrazolyl optionally substituted with 1-2 R⁸; in some embodiments of Formula VII, R is -imidazolyl optionally substituted with 1-2 R⁸; in some embodiments of Formula VII, R³ is -1,2,3-triazolyl optionally substituted with 1-2 R⁸; in some embodiments of Formula VII, R³ is -isoxazolyl optionally substituted with 1-2 R⁸; and in some embodiments of Formula VII, R³ is -oxazolyl optionally substituted with 1-2 R⁸.

In some embodiments of Formula VII, R⁶ is selected from the group consisting of -phenyl substituted with 1-2 R⁹, -heteroaryl optionally substituted with 1-2 R¹⁰; -heterocyclyl optionally substituted with 1-2 R¹¹, and -carbocyclyl optionally substituted with 1-2 R¹².

In some embodiments of Formula VII, R⁶ is -phenyl substituted with 1-2 R⁹; in some embodiments of Formula VII, R⁶ is -pyridinyl optionally substituted with 1-2 R¹⁰; in some embodiments of Formula VII, R⁶ is -pyrazolyl optionally substituted with 1-2 R¹⁰; in some embodiments of Formula VII, R⁶ is -thiazolyl optionally substituted with 1-2 R¹⁰; in some embodiments of Formula VII, R⁶ is -imidazolyl optionally substituted with 1-2 R¹⁰; in some embodiments of Formula VII, R⁶ is -isoindolinyl optionally substituted with 1-2 R¹⁰; in some embodiments of Formula VII, R⁶ is -tetrahydroisoquinolinyl optionally substituted with 1-2 R¹⁰; in some embodiments of Formula VII, R⁶ is -1,2,3-triazolyl optionally substituted with 1-2 R¹⁰; in some embodiments of Formula VII, R⁶ is -benzimidazolyl optionally substituted with 1-2 R¹⁰; in some embodiments of Formula VII, R⁶ is -indazolyl optionally substituted with 1-2 R¹⁰; in some embodiments of Formula VII, R⁶ is -cyclopropyl optionally substituted with 1-2 R¹²; in some embodiments of Formula VII, R⁶ is -cyclobutyl optionally substituted with 1-2 R¹²; in some embodiments of Formula VII, R⁶ is -cyclopentyl optionally substituted with 1-2 R¹²; and in some embodiments of Formula VII, R⁶ is -cyclohexyl optionally substituted with 1-2 R¹².

In some embodiments of Formula VII, R⁸ is selected from the group consisting of F, unsubstituted —(C₁₋₃ alkyl), —CN, —NH₂, —NH(C₁₋₄ alkyl), —N(C₁₋₄ alkyl)₂, —NH(heterocyclyl), —NH(CH₂heterocyclyl), —OMe, —CH₂OH, and —(CH₂)ₚheterocyclyl optionally substituted with 1-2 R²⁰.

In some embodiments of Formula VII, R⁸ is F; in some embodiments of Formula VII, R⁸ is unsubstituted —(C₁₋₃ alkyl); in some embodiments of Formula VII, R⁸ is —NH₂; in some embodiments of Formula VII, R⁸ is —NH(C₁₋₄ alkyl); in some embodiments of Formula VII, R⁸ is —N(C₁₋₄ alkyl)₂; in some embodiments of Formula VII, R⁸ is —NH(heterocyclyl); in some embodiments of Formula VII, R⁸ is —NH(CH₂heterocyclyl); in some embodiments of Formula VII, R⁸ is —OMe, in some embodiments of Formula VII, R⁸ is —CH₂OH; and in some embodiments of Formula VII, R⁸ is —(CH₂)ₚheterocyclyl optionally substituted with 1-2 R²⁰.

In some embodiments of Formula VII, R¹⁰ is selected from the group consisting of F, unsubstituted —(C₁₋₅ alkyl), —CN, —OR²³, —NH(heterocyclyl), —N(C₁₋₃ alkyl)(heterocyclyl), —O(C₁₋₃ alkyl), —O(heterocyclyl), —S(heterocyclyl), —C(═O)NH(C₁₋₃ alkyl), —N(R²⁴)₂, -heterocyclyl optionally substituted with 1-2 R²², and -carbocyclyl optionally substituted with 1-2 R²¹.

In some embodiments of Formula VII, R¹⁰ is F; in some embodiments of Formula VII, R¹⁰ is unsubstituted —(C₁₋₅ alkyl); in some embodiments of Formula VII, R¹⁰ is —CN; in some embodiments of Formula VII, R¹⁰ is —OR²³; in some embodiments of Formula VII, R¹⁰ is —NH(heterocyclyl); in some embodiments of Formula VII, R¹⁰ is —N(C₁₋₃ alkyl)(heterocyclyl); in some embodiments of Formula VII, R¹⁰ is —O(C₁₋₃ alkyl); in some embodiments of Formula VII, R¹⁰ is —O(heterocyclyl); in some embodiments of Formula VII, R¹⁰ is —S(heterocyclyl); in some embodiments of Formula VII, R¹⁰ is —C(═O)NH(C₁₋₃ alkyl); in some embodiments of Formula VII, R¹⁰ is —N(R²⁴)₂; in some embodiments of Formula VII, R¹⁰ is -heterocyclyl optionally substituted with 1-2 R²²; and in some embodiments of Formula VII, R¹⁰ is -carbocyclyl optionally substituted with 1-2 R²¹.

In some embodiments of Formula VII, Y¹, Y², Y³, and Y⁴ are all carbon; in some embodiments of Formula VII, Y¹ is nitrogen and Y², Y³, and Y⁴ are all carbon; in some embodiments of Formula VII, Y² is nitrogen and Y¹, Y³, and Y⁴ are all carbon; in some embodiments of Formula VII, Y³ is nitrogen and Y¹, Y², and Y⁴ are all carbon; in some embodiments of Formula VII, Y⁴ is nitrogen and Y¹, Y², and Y³ are all carbon.

U.S. Pat. No. 8,119,655 describes compounds having Formula VIII and is hereby incorporated by reference in its entirety.

One embodiment disclosed herein includes a compound having the structure of Formula VIII:

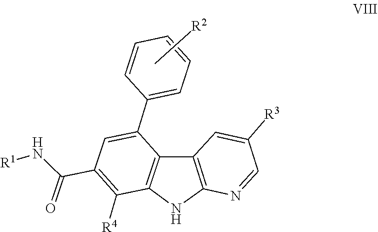

as well as prodrugs and pharmaceutically acceptable salt or solvate thereof.

In some embodiments of Formula (VIII):

R¹ is selected from the group consisting of —(C₁₋₄ alkylene)N(R⁵)₂, —(C₁₋₄ alkylene)ₚheterocyclyl optionally substituted with 1-10 (e.g., 1-9, 1-8, 1-7, 1-6, 1-5, 1-4, 1-3, 1-2, 1) R⁶, and —(C₁₋₄ alkylene)ₚcarbocyclyl optionally substituted with 1-12 (e.g., 1-11, 1-10, 1-9, 1-8, 1-7, 1-6, 1-5, 1-4, 1-3, 1-2, 1) R⁷; wherein each —(C₁₋₄ alkylene) is, independently, optionally substituted with one or more substituents as defined anywhere herein;

R² is selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), —CN, —OR, —C(═O)NHR⁹, —NHC(═O)(R¹⁰), —SO₂R¹⁰, —NHSO₂R¹⁰, and —SO₂NHR⁹;

R³ is selected from the group consisting of H, halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁);

R⁴ is selected from the group consisting of H, halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁);

each R⁵ is independently selected from the group consisting of H, unsubstituted —(C₁₋₅ alkyl) (e.g., C₁₋₄, Cl-3, C₁₋₂, C₁), unsubstituted —(C₂₋₅ alkenyl) (e.g., C₂₋₄, C₂₋₃, C₂), and unsubstituted —(C₂₋₅ alkynyl) (e.g., C₂₋₄, C₂₋₃, C₂);

each R⁶ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂_s alkynyl) (e.g., C₂₋₄, C₂₋₃, C₂), unsubstituted —(C₁₋₅ haloalkyl) (e.g., C₁₋₄, C₁₋₃, C₁₋₂, C₁), —OH, and —CN;

each R⁷ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂_s alkynyl) (e.g., C₂₋₄, C₂₋₃, C₂), unsubstituted —(C₁₋₅ haloalkyl) (e.g., C₁₋₄, C₁₋₃, C₁₋₂, C₁), —OH, and —CN;

R⁸ is selected from the group consisting of H, unsubstituted —(C₁₋₃ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and —(C₁₋₄ alkylene)ₚheterocyclyl optionally substituted with 1-10 (e.g., 1-9, 1-8, 1-7, 1-6, 1-5, 1-4, 1-3, 1-2, 1) R⁶; wherein —(C₁₋₄ alkylene) is optionally substituted with one or more substituents as defined anywhere herein;

each R⁹ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and —(C₁₋₄ alkylene)ₚheterocyclyl optionally substituted with 1-10 (e.g., 1-9, 1-8, 1-7, 1-6, 1-5, 1-4, 1-3, 1-2, 1) R⁶; wherein —(C₁₋₄ alkylene) is optionally substituted with one or more substituents as defined anywhere herein;

each R¹⁰ is independently selected from the group consisting of unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and —(C₁₋₄ alkylene)ₚheterocyclyl optionally substituted with 1-10 (e.g., 1-9, 1-8, 1-7, 1-6, 1-5, 1-4, 1-3, 1-2, 1) R⁶; wherein —(C₁₋₄ alkylene) is optionally substituted with one or more substituents as defined anywhere herein; and

each p is independently 0 or 1.

In some embodiments of Formula VIII, R¹ is selected from the group consisting of —(C₁₋₂ alkylene)N(C₁₋₃ alkyl)₂, —(CH₂)ₚheterocyclyl optionally substituted with 1-2 R⁶, and —(CH₂)ₚcarbocyclyl optionally substituted with 1-2 R⁷.

In some embodiments of Formula VIII, R¹ is —(C₁₋₂ alkylene)N(C₁₋₃ alkyl)₂; in some embodiments of Formula VIII, R¹ is —(CH₂)ₚheterocyclyl optionally substituted with 1-2 R⁶; and in some embodiments of Formula VIII, R¹ is —(CH₂)ₚcarbocyclyl optionally substituted with 1-2 R⁷.

In some embodiments of Formula VIII, R² is selected from the group consisting of halide (e.g., F, Cl, Br, I), unsubstituted —(C₁₋₄ alkyl), unsubstituted —(C₁₋₄ haloalkyl), —CN, —O(C₁₋₄ alkyl), —O(heterocyclyl), —C(═O)NH(C₁₋₅ alkyl), —NHC(═O)(C₁₋₄ alkyl), —SO₂(C₁₋₄ alkyl), —NHSO₂(C₁₋₄ alkyl), and —SO₂NH(C₁₋₄ alkyl).

In some embodiments of Formula VIII, R² is F; in some embodiments of Formula VIII, R² is unsubstituted —(C₁₋₄ alkyl); in some embodiments of Formula VIII, R² is unsubstituted —(C₁₋₄ haloalkyl); in some embodiments of Formula VIII, R² is —CN; in some embodiments of Formula VIII, R² is —O(C₁₋₄ alkyl); in some embodiments of Formula VIII, R² is —O(heterocyclyl); in some embodiments of Formula VIII, R² is —C(═O)NH(C₁₋₅ alkyl); in some embodiments of Formula VIII, R² is —NHC(═O)(C₁₋₄ alkyl); in some embodiments of Formula VIII, R² is —SO₂(C₁₋₄ alkyl); in some embodiments of Formula VIII, R² is —NHSO₂(C₁₋₄ alkyl); and in some embodiments of Formula VIII, R² is —SO₂NH(C₁₋₄ alkyl).

In some embodiments of Formula VIII, R³ is selected from the group consisting of H, halide (e.g., F, Cl, Br, I), unsubstituted —(C₁₋₃ alkyl), and unsubstituted —(C₁₋₃ haloalkyl).

In some embodiments of Formula VIII, R³ is H; in some embodiments of Formula VIII, R³ is F; in some embodiments of Formula VIII, R³ is unsubstituted —(C₁₋₃ alkyl); and in some embodiments of Formula VIII, R³ is unsubstituted —(C₁₋₃ haloalkyl).

In some embodiments of Formula VIII, R⁴ is selected from the group consisting of H, halide (e.g., F, Cl, Br, I), unsubstituted —(C₁₋₃ alkyl), and unsubstituted —(C₁₋₃ haloalkyl).

In some embodiments of Formula VIII, R⁴ is H; in some embodiments of Formula VIII, R⁴ is F; in some embodiments of Formula VIII, R⁴ is unsubstituted —(C₁₋₃ alkyl); and in some embodiments of Formula VIII, R⁴ is unsubstituted —(C₁₋₃ haloalkyl).

U.S. Pat. No. 8,067,591 describes compounds having Formula IX and is hereby incorporated by reference in its entirety.

One embodiment disclosed herein includes a compound having the structure of Formula IX:

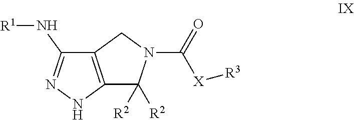

as well as prodrugs and pharmaceutically acceptable salt or solvate thereof.

In some embodiments of Formula (IX):

R¹ is -heteroaryl optionally substituted with 1-6 (e.g., 1-5, 1-4, 1-3, 1-2, 1) R⁴; each R² is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁); R³ is —CH(R⁵)R⁶;

each R⁴ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), —CN, —OR⁷, -carbocyclyl optionally substituted with 1-12 (e.g., 1-11, 1-10, 1-9, 1-8, 1-7, 1-6, 1-5, 1-4, 1-3, 1-2, 1) R;

R⁵ is -aryl optionally substituted with 1-5 (e.g., 1-4, 1-3, 1-2, 1) R⁹;

R⁶ is —(C₁₋₄ alkylene)N(R¹⁰)₂; wherein —(C₁₋₄ alkylene) is optionally substituted with one or more substituents as defined anywhere herein;

each R⁷ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁);

each R⁸ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁);

each R⁹ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), unsubstituted —(C₁₋₅ haloalkyl) (e.g., C₁₋₄, C₁₋₃, C₁₋₂, C₁), —CN, and —OR⁷;

each R¹⁰ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), and unsubstituted —(C₂₋₅ alkynyl) (e.g., C₂₋₄, C₂₋₃, C₂); and

X is selected from the group consisting of O, S, and NH.

In some embodiments of Formula IX, R¹ is -heteroaryl optionally substituted with 1-2 R⁴.

In some embodiments of Formula IX, R¹ is a 6-10 membered -heteroaryl optionally substituted with 1-2 R⁴.

In some embodiments of Formula IX, R¹ is -pyridinyl optionally substituted with 1-2 R⁴; in some embodiments of Formula IX, R¹ is -pyrimidinyl optionally substituted with 1-2 R⁴; in some embodiments of Formula IX, R¹ is -pyrazinyl optionally substituted with 1-2 R⁴; in some embodiments of Formula IX, in some embodiments of Formula IX, R¹ is -benzimidazolyl optionally substituted with 1-2 R⁴; in some embodiments of Formula IX, R¹ is -indazolyl optionally substituted with 1-2 R⁴; in some embodiments of Formula IX, R¹ is -thieno[3,2-d]pyrimidinyl optionally substituted with 1-2 R⁴; in some embodiments of Formula IX, R¹ is -thiazolo[4,5-d]pyrimidinyl optionally substituted with 1-2 R⁴; in some embodiments of Formula IX, R¹ is -benzo[b]thiophenyl optionally substituted with 1-2 R⁴; in some embodiments of Formula IX, R¹ is -benzo[d]thiazolyl optionally substituted with 1-2 R⁴; in some embodiments of Formula IX, R¹ is -thieno[2,3-c]pyridinyl optionally substituted with 1-2 R⁴; and in some embodiments of Formula IX, R¹ is -thieno[3,2-b]pyridinyl optionally substituted with 1-2 R⁴.

In some embodiments of Formula IX, R² is selected from the group consisting of H, unsubstituted —(C₁₋₃ alkyl) and unsubstituted —(C₁₋₃ haloalkyl).

In some embodiments of Formula IX, R² is H; in some embodiments of Formula IX, R² is unsubstituted —(C₁₋₃ alkyl); and in some embodiments of Formula IX, R² is unsubstituted —(C₁₋₃ haloalkyl).

In some embodiments of Formula IX, R³ is —CH(phenyl)(C₁₋₂ alkylene)N(C₁₋₃ alkyl)₂.

In some embodiments of Formula IX, X is O; in some embodiments of Formula IX, X is S; and in some embodiments of Formula IX, X is NH.

Bioorganic & Medicinal Chemistry (2007), 15(17), 5837-5844, World Intellectual Property Organization, WO2001083481, and Spanish patent application 2,244,613 describe compounds having Formula X and are hereby incorporated by reference in their entirety.

One embodiment disclosed herein includes a compound having the structure of Formula X:

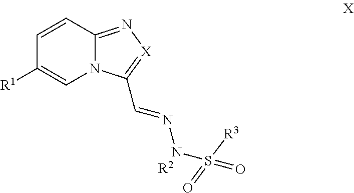

as well as prodrugs and pharmaceutically acceptable salt or solvate thereof.

In some embodiments of Formula (X):

R¹ is selected from the group consisting of H, halide (e.g., F, Cl, Br, I), unsubstituted —(C₁₋₅ alkyl) (e.g., C₁₋₄, C₁₋₃, C₁₋₂, C₁), unsubstituted —(C₁₋₅ haloalkyl) (e.g., C₁₋₄, C₁₋₃, C₁₋₂, C₁), and —CN;

R² is selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), and unsubstituted —(C₂₋₅ alkynyl) (e.g., C₂₋₄, C₂₋₃, C₂);

R³ is -aryl optionally substituted with 1-5 (e.g., 1-4, 1-3, 1-2, 1) R⁴;

each R⁴ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂_s alkynyl) (e.g., C₂₋₄, C₂₋₃, C₂), unsubstituted —(C₁₋₅ haloalkyl) (e.g., C₁₋₄, C₁₋₃, C₁₋₂, C₁), —NO₂, —CN, and —OMe;

R⁵ is selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁); and

X is selected from the group consisting of N and CR⁵.

In some embodiments of Formula X, R¹ is selected from the group consisting of H, halide (e.g., F, Cl, Br, I), unsubstituted —(C₁₋₃ alkyl), unsubstituted —(C₁₋₃ haloalkyl), and —CN.

In some embodiments of Formula X, R¹ is H; in some embodiments of Formula X, R¹ is F; in some embodiments of Formula X, R¹ is unsubstituted —(C₁₋₃ alkyl); in some embodiments of Formula X, R¹ is unsubstituted —(C₁₋₃ haloalkyl); and in some embodiments of Formula X, R¹ is —CN.

In some embodiments of Formula X, R² is selected from the group consisting of H and unsubstituted —(C₁₋₃ alkyl).

In some embodiments of Formula X, R² is H; and in some embodiments of Formula X, R² is unsubstituted —(C₁₋₃ alkyl).

In some embodiments of Formula X, R³ is -phenyl optionally substituted with 1-2 R⁴;

In some embodiments of Formula X, X is selected from the group consisting of N and CH.

In some embodiments of Formula X, X is N; and in some embodiments of Formula X, X is CH.

Bioorganic & Medicinal Chemistry (2012), 22(24), 7326-7329, Bioorganic & Medicinal Chemistry Letters (2014), 24(18), 4418-4423 and Nature Communications (2017), 8(7), 1-15 describe compounds having Formula XI and are hereby incorporated by reference in their entirety.

One embodiment disclosed herein includes a compound having the structure of Formula XI:

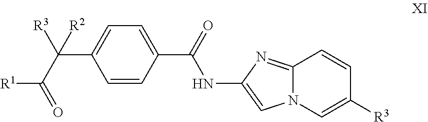

as well as prodrugs and pharmaceutically acceptable salt or solvate thereof.

In some embodiments of Formula (XI):

R¹ is —N(R⁴)₂;

R² is selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁);

R³ is -heteroaryl optionally substituted with 1-6 (e.g., 1-5, 1-4, 1-3, 1-2, 1) R⁵;

each R⁴ is independently selected from the group consisting of H, unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and -heterocyclyl optionally substituted with 1-10 (e.g., 1-9, 1-8, 1-7, 1-6, 1-5, 1-4, 1-3, 1-2, 1) R⁶;

alternatively, two adjacent R⁴ are taken together to form a ring which is selected from the group consisting of -heterocyclyl optionally substituted with 1-10 (e.g., 1-9, 1-8, 1-7, 1-6, 1-5, 1-4, 1-3, 1-2, 1) R⁶;

each R⁵ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂_s alkynyl) (e.g., C₂₋₄, C₂₋₃, C₂), unsubstituted —(C₁₋₅ haloalkyl) (e.g., C₁₋₄, C₁₋₃, C₁₋₂, C₁), —CN, —OH, and —OMe; and

each R⁶ is independently selected from the group consisting of halide (e.g., F, Cl, Br, I), unsubstituted —(C₁₋₅ alkyl) (e.g., C₁₋₄, C₁₋₃, C₁₋₂, C₁), unsubstituted —(C₂₋₅ alkenyl) (e.g., C₂₋₄, C₂₋₃, C₂), unsubstituted —(C₂₋₅ alkynyl) (e.g., C₂₋₄, C₂₋₃, C₂), and unsubstituted —(C₁₋₅ haloalkyl) (e.g., C₁₋₄, C₁₋₃, C₁₋₂, C₁).

In some embodiments of Formula XI, R¹ is selected from the group consisting of —N(C₁₋₃ alkyl)₂, —NH(C₁₋₃ alkyl), —NH(heterocyclyl), and -heterocyclyl optionally substituted with 1-2 R⁶.

In some embodiments of Formula XI, R¹ is —N(C₁₋₃ alkyl)₂; in some embodiments of Formula XI, R¹ is —NH(C₁₋₃ alkyl); in some embodiments of Formula XI, R¹ is —NH(heterocyclyl); and in some embodiments of Formula XI, R¹ is -heterocyclyl optionally substituted with 1-2 R⁶.

In some embodiments of Formula XI, R² is selected from the group consisting of H, unsubstituted —(C₁₋₃ alkyl), and unsubstituted —(C₁₋₃ haloalkyl).

In some embodiments of Formula XI, R² is H; in some embodiments of Formula XI, R² is unsubstituted —(C₁₋₃ alkyl); and in some embodiments of Formula XI, R² is unsubstituted —(C₁₋₃ haloalkyl).

In some embodiments of Formula XI, R³ is selected from the group consisting of -pyridinyl optionally substituted with 1-2 R⁵, -pyrazolyl optionally substituted with 1-2 R⁵, -thiazolyl optionally substituted with 1-2 R⁵, -imidazolyl optionally substituted with 1-2 R⁵, and -1,2,3-triazolyl optionally substituted with 1-2 R⁵.

In some embodiments of Formula XI, R³ is -pyridinyl optionally substituted with 1-2 R⁵; in some embodiments of Formula XI, R³ is -pyrazolyl optionally substituted with 1-2 R⁵; in some embodiments of Formula XI, R³ is -thiazolyl optionally substituted with 1-2 R⁵; in some embodiments of Formula XI, R³ is -imidazolyl optionally substituted with 1-2 R⁵; and in some embodiments of Formula XI, R³ is -1,2,3-triazolyl optionally substituted with 1-2 R⁵.

U.S. provisional application 62/685,764 describes compounds having Formula XII and is hereby incorporated by reference in their entirety.

One embodiment disclosed herein includes a compound having the structure of Formula XII:

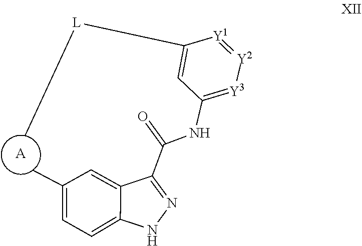

as well as prodrugs and pharmaceutically acceptable salt or solvate thereof.

In some embodiments of Formula (XII):

Ring A is a 5-6-membered heteroaryl optionally substituted with 1-3 R¹;

L is -L¹-L²-L³-L⁴-

L¹ is selected from the group consisting of unsubstituted —(C₁₋₃ alkylene)-, —NR²—, —NR³(C═O)—, —(C═O)NR³—, and —O—;

L² is selected from the group consisting of unsubstituted —(C₁₋₆ alkylene)-, —NR²—, —NR³(C═O)—, and —(C═O)NR³—;

L³ is selected from the group consisting of unsubstituted —(C₁₋₆ alkylene)-, —O—, and carbocyclylene optionally substituted with one or more halides;

L⁴ is selected from the group consisting of unsubstituted —(C₁₋₆ alkylene)-, —O—, —NR²—, —NR³(C═O)—, —(C═O)NR³—, -arylene substituted with 1-5 R⁴, and -heteroarylene optionally substituted with 1-4 R⁵;

with the proviso that —NR²— and —O— are not adjacent to each other;

with the proviso that two —NR³(C═O)— and/or —(C═O)NR³—, are not adjacent to each other;

each R¹ is selected from the group consisting of halide, unsubstituted —(C₁₋₃ alkyl), unsubstituted —(C₁₋₃ haloalkyl), and —CN;

each R² is selected from the group consisting of H and unsubstituted —(C₁₋₆ alkyl);

each R³ is selected from the group consisting of H and unsubstituted —(C₁₋₆ alkyl);

each R⁴ is selected from the group consisting of halide, unsubstituted —(C₁₋₆ alkyl), unsubstituted —(C₁₋₆ haloalkyl), and —CN;

each R⁵ is selected from the group consisting of halide, unsubstituted —(C₁₋₆ alkyl), unsubstituted —(C₁₋₆ haloalkyl), and —CN;

Y¹, Y², and Y³ are independently selected from the group consisting of carbon and nitrogen; wherein

if Y¹ is nitrogen then Y² and Y³ are CH;

if Y² is nitrogen then Y¹ and Y³ are CH; and

if Y³ is nitrogen then Y¹ and Y² are CH.

In some embodiments of Formula XII, Ring A is a 5-membered heteroaryl and is selected from the group consisting of

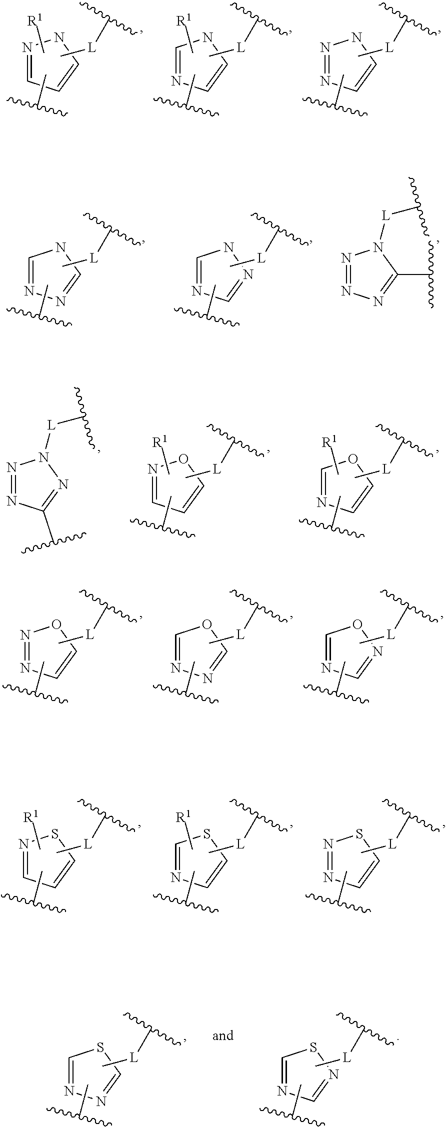

In some embodiments of Formula XII, Ring A is a 6-membered heteroaryl and is selected from the group consisting of

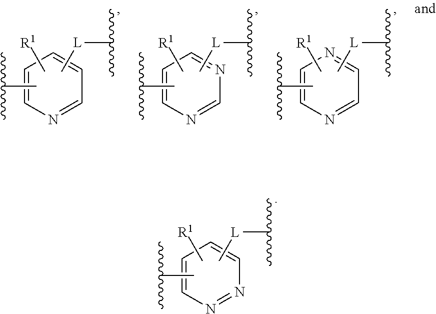

In some embodiments of Formula XII, Ring A is a 5-6-membered heteroaryl and is selected from the group consisting of

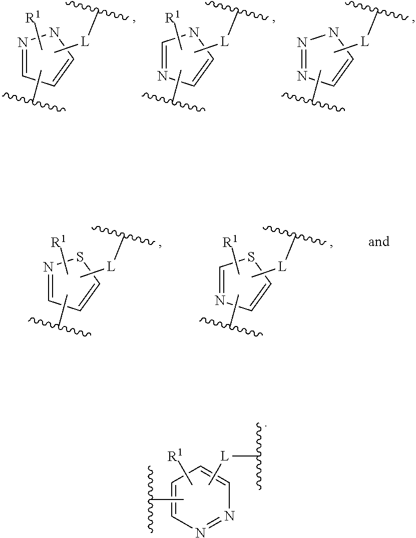

In some embodiments of Formula XII, Ring A is a 5-6-membered heteroaryl and is selected from the group consisting of

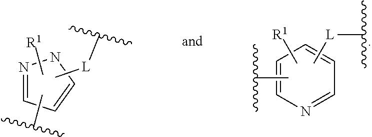

In some embodiments of Formula XII, L¹ is selected from the group consisting of —(CH₂)—, —NH—, —NMe-, —NH(C═O)—, —(C═O)NH—, and —O—; In some embodiments of Formula XII, L¹ is —(CH₂)—; In some embodiments of Formula XII, L¹ is —NH—; In some embodiments of Formula XII, L¹ is —NMe-; In some embodiments of Formula XII, L¹ is —NH(C═O)—; In some embodiments of Formula XII, L¹ is —(C═O)NH—; In some embodiments of Formula XII, L¹ is —O—.

In some embodiments of Formula XII, L² is selected from the group consisting of —(CH₂)—, —(CH₂CH₂)—, —(CH₂CH₂CH₂)—, —NH—, —NMe-, —NH(C═O)—, and —(C═O)NH—; In some embodiments of Formula XII, L² is —(CH₂)—; In some embodiments of Formula XII, L² is —(CH₂CH₂)—; In some embodiments of Formula XII, L² is —(CH₂CH₂CH₂)—; In some embodiments of Formula XII, L² is —NH—; In some embodiments of Formula XII, L² is —NMe-; In some embodiments of Formula XII, L² is —NH(C═O)—; In some embodiments of Formula XII, L² is —(C═O)NH—.

In some embodiments of Formula XII, L³ is selected from the group consisting of —(CH₂)—, —(CH₂CH₂)—, —(CH₂CH₂CH₂)—, —(CH₂CH₂CH₂CH₂)—, —O—, and

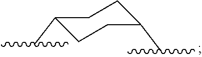 [SMILES: *C1CCC(*)CC1]

In some embodiments of Formula XII, L³ is —(CH₂)—; In some embodiments of Formula XII, L³ is —(CH₂CH₂)—; In some embodiments of Formula XII, L³ is —(CH₂CH₂CH₂)—; In some embodiments of Formula XII, L³ is —(CH₂CH₂CH₂CH₂)—; In some embodiments of Formula XII, L³ is —O—; In some embodiments of Formula XII, L³ is

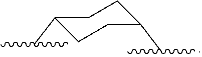 [SMILES: *C1CCC(*)CC1]

In some embodiments of Formula XII, L⁴ is selected from the group consisting of —(CH₂)—, —(CH₂CH₂)—, —(CH₂CH₂CH₂)—, —(CH₂CH₂CH₂CH₂)—, —O—, —NH—, —NMe-, —NH(C═O)—, and —(C═O)NH—,

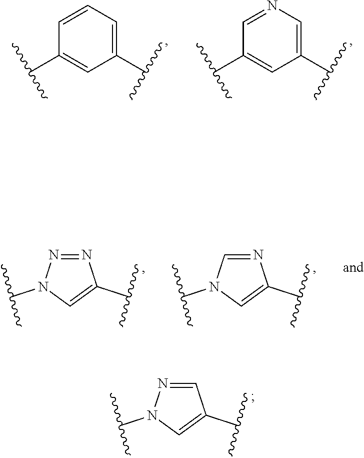 [SMILES: *c1cccc(*)c1 ; *c1cncc(*)c1 ; *c1cn(*)nn1 ; *c1cn(*)cn1 ; *c1cnn(*)c1]

In some embodiments of Formula XII, L⁴ is —(CH₂)—; In some embodiments of Formula XII, L⁴ is —(CH₂CH₂)—; In some embodiments of Formula XII, L⁴ is —(CH₂CH₂CH₂)—; In some embodiments of Formula XII, L⁴ is —(CH₂CH₂CH₂CH₂)—; In some embodiments of Formula XII, L⁴ is —O—; In some embodiments of Formula XII, L⁴ is —NH—; In some embodiments of Formula XII, L⁴ is —NMe-; In some embodiments of Formula XII, L⁴ is —NH(C═O)—; In some embodiments of Formula XII, L⁴ is —(C═O)NH—; In some embodiments of Formula XII, L⁴ is

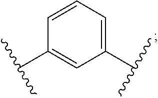 [SMILES: *c1cccc(*)c1]

In some embodiments of Formula XII, L⁴ is

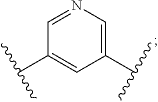 [SMILES: *c1cncc(*)c1]

In some embodiments of Formula XII, L⁴ is

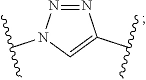 [SMILES: *c1cn(*)nn1]

In some embodiments of Formula XII, L⁴ is

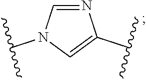 [SMILES: *c1cn(*)cn1]

In some embodiments of Formula XII, L⁴ is

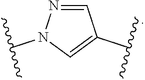 [SMILES: *c1cnn(*)c1]

In some embodiments of Formulas (I) and (III)-(VIII), each p is 0 or 1; in some embodiments of Formulas (I)-(VIII), p is 0; in some embodiments of Formulas (I)-(VIII), p is 1.

In some embodiments of Formulas (I) and (III)-(VIII), each —(C₁₋₄alkylene) is —(C₁₋₃ alkylene).

In some embodiments of Formulas (I) and (III)-(VIII), each —(C₁₋₄alkylene) is —(C₁₋₂ alkylene).

In some embodiments of Formulas (I) and (III)-(VIII), each —(C₁₋₄alkylene) is —(C₁ alkylene).

In some embodiments of Formulas (I) and (III)-(VIII), each —(C₁₋₄ alkylene) is —CH₂—.

In some embodiments of Formulas (I) and (III)-(VIII), each —(C₁₋₄ alkylene) is optionally substituted with halide (e.g., F, Cl, Br, I).

In some embodiments of Formulas (I) and (III)-(VIII), each —(C₁₋₄ alkylene) is optionally substituted with F.

Illustrative compounds of Formulas (I)-(XII) are shown in Table 1.

TABLE 1

| | |
|---|---|
| 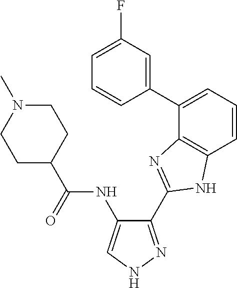 [SMILES: CN1CCC(C(=O)Nc2c[nH]nc2-c2nc3c(-c4cccc(F)c4)cccc3[nH]2)CC1] | 1 |
| 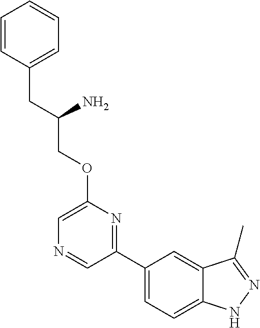 [SMILES: Cc1n[nH]c2ccc(-c3cncc(OC[C@H](N)Cc4ccccc4)n3)cc12] | 2 |
| 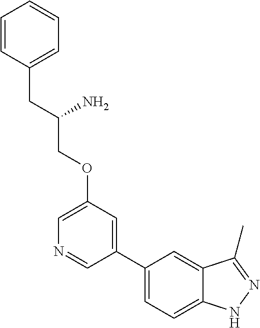 [SMILES: Cc1n[nH]c2ccc(-c3cncc(OC[C@@H](N)Cc4ccccc4)c3)cc12] | 3 |
| 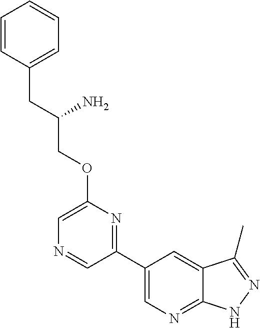 [SMILES: Cc1n[nH]c2ncc(-c3cncc(OC[C@@H](N)Cc4ccccc4)n3)cc12] | 4 |
| 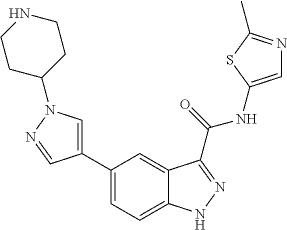 [SMILES: Cc1ncc(NC(=O)c2n[nH]c3ccc(-c4cnn(C5CCNCC5)c4)cc23)s1] | 5 |
| 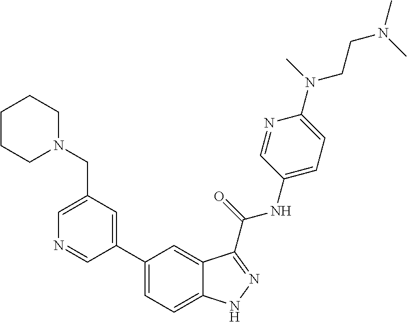 [SMILES: CN(C)CCN(C)c1ccc(NC(=O)c2n[nH]c3ccc(-c4cncc(CN5CCCCC5)c4)cc23)cn1] | 6 |
| 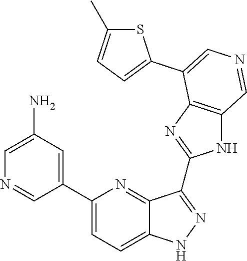 [SMILES: Cc1ccc(-c2cncc3[nH]c(-c4n[nH]c5ccc(-c6cncc(N)c6)nc45)nc23)s1] | 7 |
| 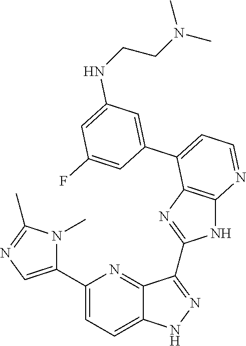 [SMILES: Cc1ncc(-c2ccc3[nH]nc(-c4nc5c(-c6cc(F)cc(NCCN(C)C)c6)ccnc5[nH]4)c3n2)n1C] | 8 |
| 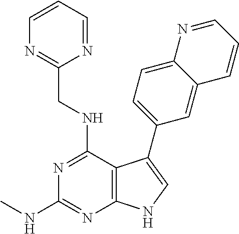 [SMILES: CNc1nc(NCc2ncccn2)c2c(-c3ccc4ncccc4c3)c[nH]c2n1] | 9 |
| 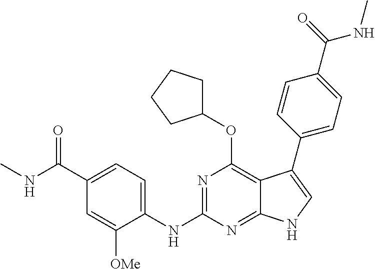 | 10 |
| 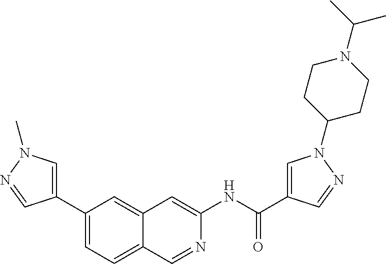 [SMILES: CC(C)N1CCC(n2cc(C(=O)Nc3cc4cc(-c5cnn(C)c5)ccc4cn3)cn2)CC1] | 11 |
| 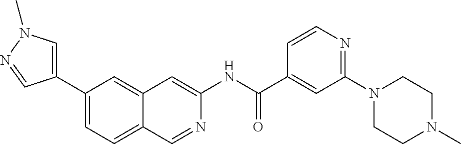 [SMILES: CN1CCN(c2cc(C(=O)Nc3cc4cc(-c5cnn(C)c5)ccc4cn3)ccn2)CC1] | 12 |
| 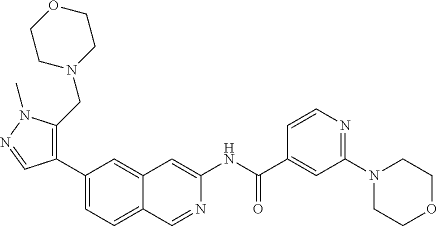 [SMILES: Cn1ncc(-c2ccc3cnc(NC(=O)c4ccnc(N5CCOCC5)c4)cc3c2)c1CN1CCOCC1] | 13 |
| 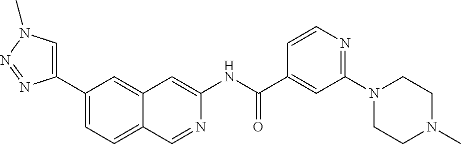 [SMILES: CN1CCN(c2cc(C(=O)Nc3cc4cc(-c5cn(C)nn5)ccc4cn3)ccn2)CC1] | 14 |
| 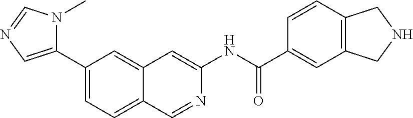 [SMILES: Cn1cncc1-c1ccc2cnc(NC(=O)c3ccc4c(c3)CNC4)cc2c1] | 15 |
| 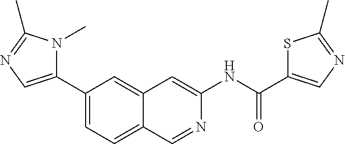 [SMILES: Cc1ncc(C(=O)Nc2cc3cc(-c4cnc(C)n4C)ccc3cn2)s1] | 16 |
| 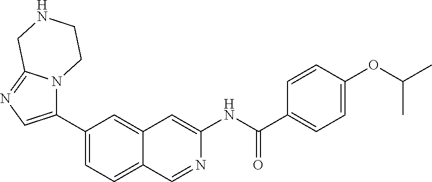 [SMILES: CC(C)Oc1ccc(C(=O)Nc2cc3cc(-c4cnc5n4CCNC5)ccc3cn2)cc1] | 17 |
| 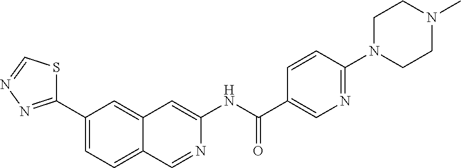 [SMILES: CN1CCN(c2ccc(C(=O)Nc3cc4cc(-c5nncs5)ccc4cn3)cn2)CC1] | 18 |
| 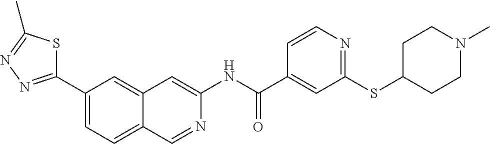 [SMILES: Cc1nnc(-c2ccc3cnc(NC(=O)c4ccnc(SC5CCN(C)CC5)c4)cc3c2)s1] | 19 |
| 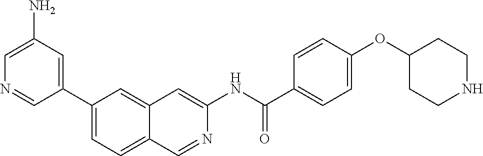 [SMILES: Nc1cncc(-c2ccc3cnc(NC(=O)c4ccc(OC5CCNCC5)cc4)cc3c2)c1] | 20 |
| 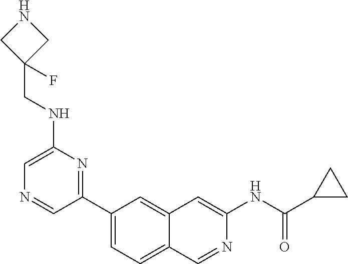 [SMILES: O=C(Nc1cc2cc(-c3cncc(NCC4(F)CNC4)n3)ccc2cn1)C1CC1] | 21 |
| 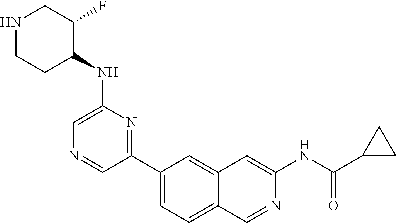 | 22 |
| 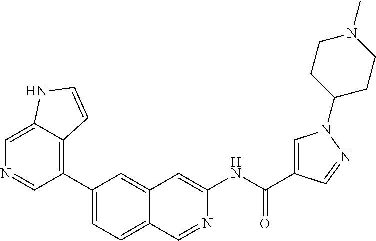 [SMILES: CN1CCC(n2cc(C(=O)Nc3cc4cc(-c5cncc6[nH]ccc56)ccc4cn3)cn2)CC1] | 23 |
| 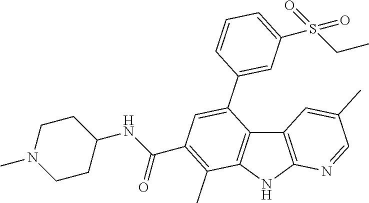 [SMILES: CCS(=O)(=O)c1cccc(-c2cc(C(=O)NC3CCN(C)CC3)c(C)c3[nH]c4ncc(C)cc4c23)c1] | 24 |
| 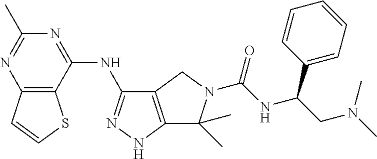 | 25 |
| 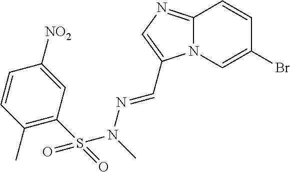 [SMILES: Cc1ccc([N+](=O)[O-])cc1S(=O)(=O)N(C)/N=C/c1cnc2ccc(Br)cn12] | 26 |
| 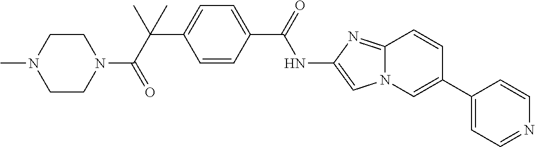 [SMILES: CN1CCN(C(=O)C(C)(C)c2ccc(C(=O)Nc3cn4cc(-c5ccncc5)ccc4n3)cc2)CC1] | 27 |
| 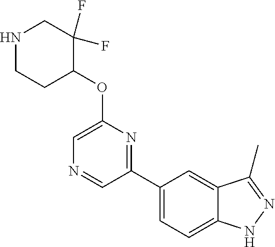 [SMILES: Cc1n[nH]c2ccc(-c3cncc(OC4CCNCC4(F)F)n3)cc12] | 28 |
| 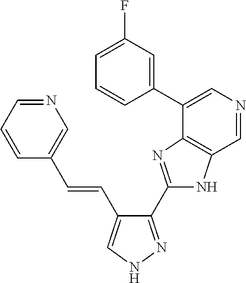 [SMILES: Fc1cccc(-c2cncc3[nH]c(-c4n[nH]cc4/C=C/c4cccnc4)nc23)c1] | 29 |
| 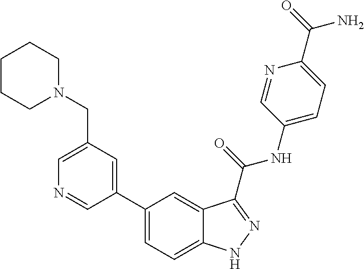 [SMILES: NC(=O)c1ccc(NC(=O)c2n[nH]c3ccc(-c4cncc(CN5CCCCC5)c4)cc23)cn1] | 30 |
| 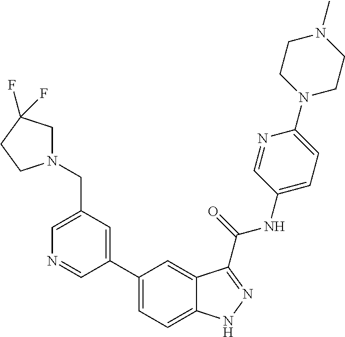 [SMILES: CN1CCN(c2ccc(NC(=O)c3n[nH]c4ccc(-c5cncc(CN6CCC(F)(F)C6)c5)cc34)cn2)CC1] | 31 |
| 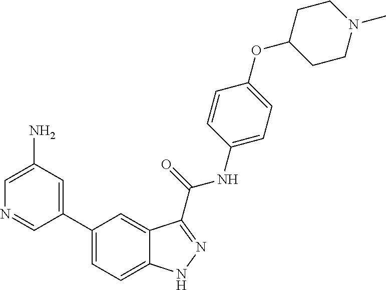 [SMILES: CN1CCC(Oc2ccc(NC(=O)c3n[nH]c4ccc(-c5cncc(N)c5)cc34)cc2)CC1] | 32 |
| 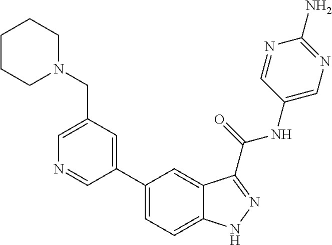 [SMILES: Nc1ncc(NC(=O)c2n[nH]c3ccc(-c4cncc(CN5CCCCC5)c4)cc23)cn1] | 33 |
| 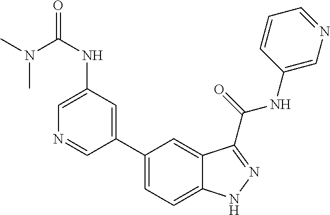 [SMILES: CN(C)C(=O)Nc1cncc(-c2ccc3[nH]nc(C(=O)Nc4cccnc4)c3c2)c1] | 34 |
| 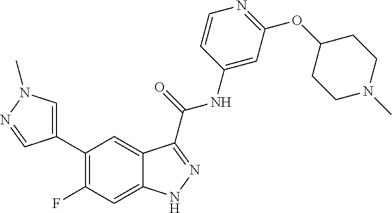 [SMILES: CN1CCC(Oc2cc(NC(=O)c3n[nH]c4cc(F)c(-c5cnn(C)c5)cc34)ccn2)CC1] | 35 |
| 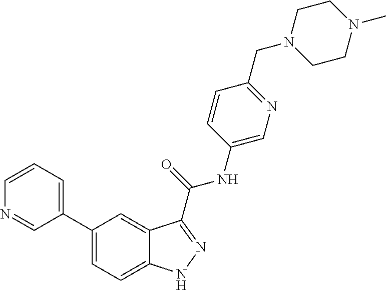 [SMILES: CN1CCN(Cc2ccc(NC(=O)c3n[nH]c4ccc(-c5cccnc5)cc34)cn2)CC1] | 36 |
| 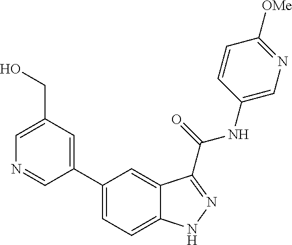 [SMILES: COc1ccc(NC(=O)c2n[nH]c3ccc(-c4cncc(CO)c4)cc23)cn1] | 37 |
| 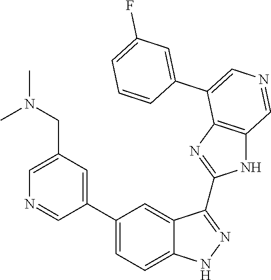 [SMILES: CN(C)Cc1cncc(-c2ccc3[nH]nc(-c4nc5c(-c6cccc(F)c6)cncc5[nH]4)c3c2)c1] | 38 |
| 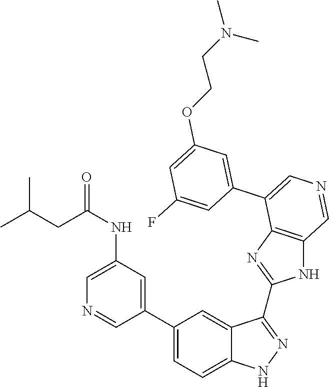 [SMILES: CC(C)CC(=O)Nc1cncc(-c2ccc3[nH]nc(-c4nc5c(-c6cc(F)cc(OCCN(C)C)c6)cncc5[nH]4)c3c2)c1] | 39 |
| 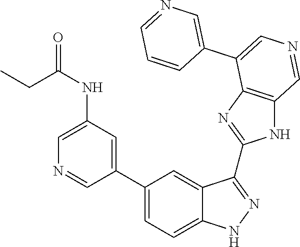 [SMILES: CCC(=O)Nc1cncc(-c2ccc3[nH]nc(-c4nc5c(-c6cccnc6)cncc5[nH]4)c3c2)c1] | 40 |
| 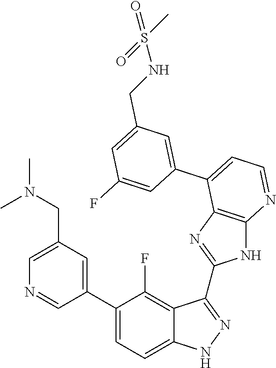 [SMILES: CN(C)Cc1cncc(-c2ccc3[nH]nc(-c4nc5c(-c6cc(F)cc(CNS(C)(=O)=O)c6)ccnc5[nH]4)c3c2F)c1] | 41 |
| 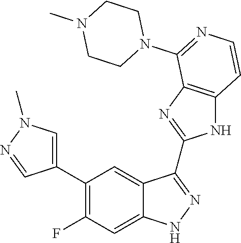 [SMILES: CN1CCN(c2nccc3[nH]c(-c4n[nH]c5cc(F)c(-c6cnn(C)c6)cc45)nc23)CC1] | 42 |
| 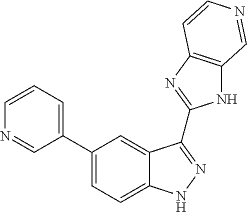 [SMILES: c1cncc(-c2ccc3[nH]nc(-c4nc5ccncc5[nH]4)c3c2)c1] | 43 |
| 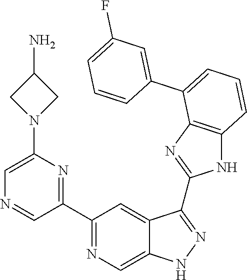 [SMILES: NC1CN(c2cncc(-c3cc4c(-c5nc6c(-c7cccc(F)c7)cccc6[nH]5)n[nH]c4cn3)n2)C1] | 44 |
| 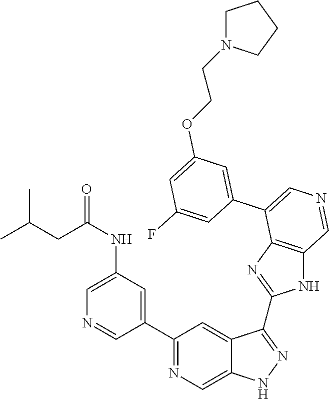 [SMILES: CC(C)CC(=O)Nc1cncc(-c2cc3c(-c4nc5c(-c6cc(F)cc(OCCN7CCCC7)c6)cncc5[nH]4)n[nH]c3cn2)c1] | 45 |
| 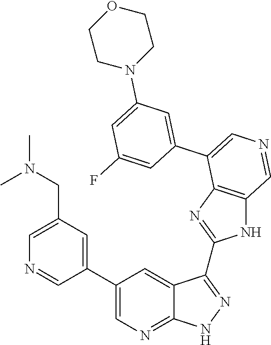 [SMILES: CN(C)Cc1cncc(-c2cnc3[nH]nc(-c4nc5c(-c6cc(F)cc(N7CCOCC7)c6)cncc5[nH]4)c3c2)c1] | 46 |
| 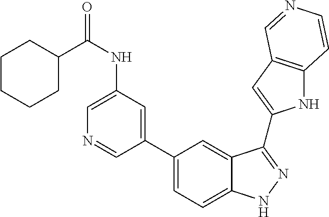 [SMILES: O=C(Nc1cncc(-c2ccc3[nH]nc(-c4cc5cnccc5[nH]4)c3c2)c1)C1CCCCC1] | 47 |
| 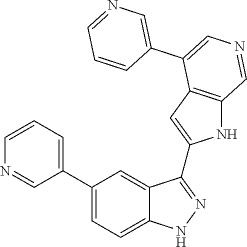 [SMILES: c1cncc(-c2ccc3[nH]nc(-c4cc5c(-c6cccnc6)cncc5[nH]4)c3c2)c1] | 48 |
| 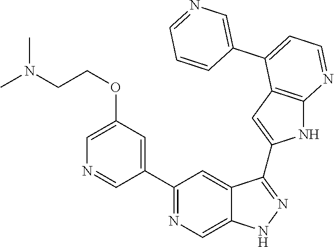 [SMILES: CN(C)CCOc1cncc(-c2cc3c(-c4cc5c(-c6cccnc6)ccnc5[nH]4)n[nH]c3cn2)c1] | 49 |
| 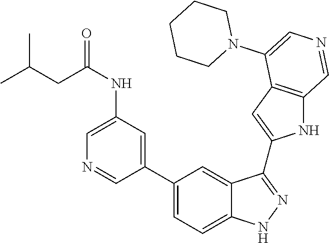 [SMILES: CC(C)CC(=O)Nc1cncc(-c2ccc3[nH]nc(-c4cc5c(N6CCCCC6)cncc5[nH]4)c3c2)c1] | 50 |
| 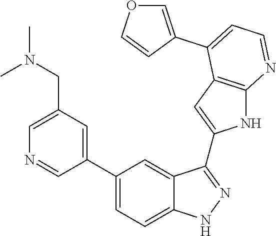 [SMILES: CN(C)Cc1cncc(-c2ccc3[nH]nc(-c4cc5c(-c6ccoc6)ccnc5[nH]4)c3c2)c1] | 51 |
| 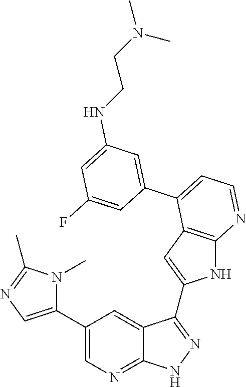 [SMILES: Cc1ncc(-c2cnc3[nH]nc(-c4cc5c(-c6cc(F)cc(NCCN(C)C)c6)ccnc5[nH]4)c3c2)n1C] | 52 |
| 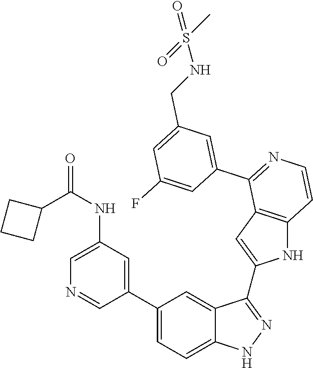 [SMILES: CS(=O)(=O)NCc1cc(F)cc(-c2nccc3[nH]c(-c4n[nH]c5ccc(-c6cncc(NC(=O)C7CCC7)c6)cc45)cc23)c1] | 53 |
| 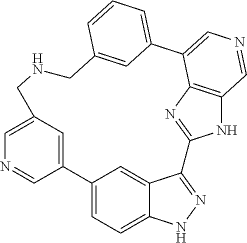 [SMILES: c1cc2cc(c1)-c1cncc3[nH]c(nc13)-c1n[nH]c3ccc(cc13)-c1cncc(c1)CNC2] | 54 |
| 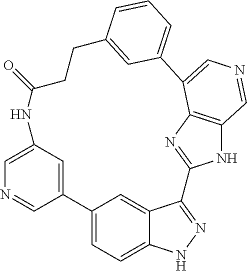 [SMILES: O=C1CCc2cccc(c2)-c2cncc3[nH]c(nc23)-c2n[nH]c3ccc(cc23)-c2cncc(c2)N1] | 55 |
| 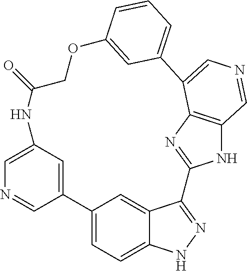 [SMILES: O=C1COc2cccc(c2)-c2cncc3[nH]c(nc23)-c2n[nH]c3ccc(cc23)-c2cncc(c2)N1] | 56 |
| 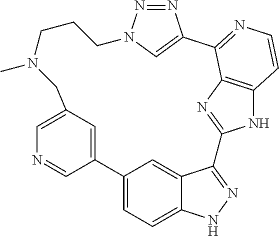 [SMILES: CN1CCCn2cc(nn2)-c2nccc3[nH]c(nc23)-c2n[nH]c3ccc(cc23)-c2cncc(c2)C1] | 57 |
| 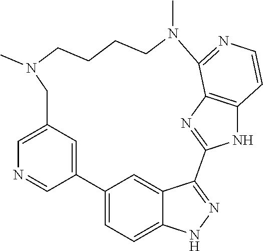 [SMILES: CN1CCCCN(C)c2nccc3[nH]c(nc23)-c2n[nH]c3ccc(cc23)-c2cncc(c2)C1] | 58 |
| 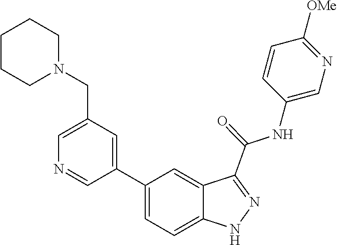 [SMILES: COc1ccc(NC(=O)c2n[nH]c3ccc(-c4cncc(CN5CCCCC5)c4)cc23)cn1] | 59 |
| 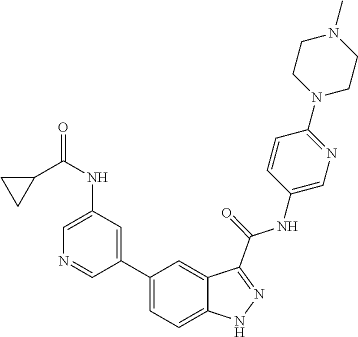 [SMILES: CN1CCN(c2ccc(NC(=O)c3n[nH]c4ccc(-c5cncc(NC(=O)C6CC6)c5)cc34)cn2)CC1] | 60 |
| 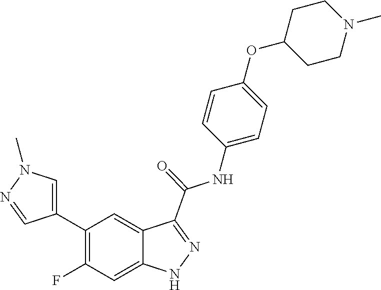 [SMILES: CN1CCC(Oc2ccc(NC(=O)c3n[nH]c4cc(F)c(-c5cnn(C)c5)cc34)cc2)CC1] | 61 |
| 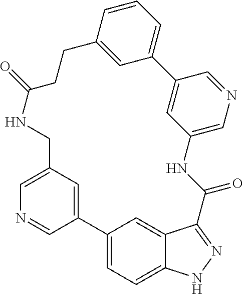 [SMILES: O=C1CCc2cccc(c2)-c2cncc(c2)NC(=O)c2n[nH]c3ccc(cc23)-c2cncc(c2)CN1] | 62 |
| 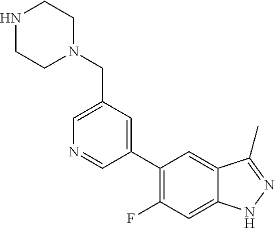 [SMILES: Cc1n[nH]c2cc(F)c(-c3cncc(CN4CCNCC4)c3)cc12] | 63 |
| 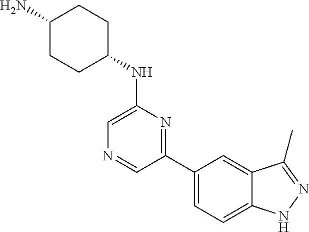 [SMILES: Cc1n[nH]c2ccc(-c3cncc(N[C@H]4CC[C@@H](N)CC4)n3)cc12] | 64 |
| 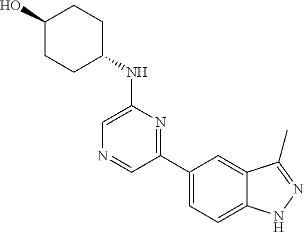 [SMILES: Cc1n[nH]c2ccc(-c3cncc(N[C@H]4CC[C@H](O)CC4)n3)cc12] | 65 |
| 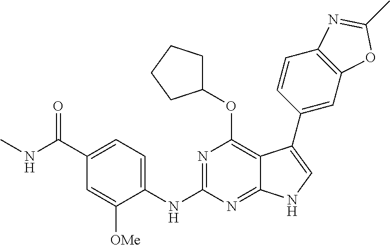 | 66 |
| 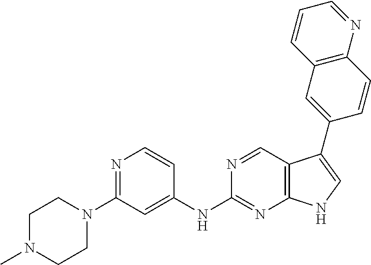 [SMILES: CN1CCN(c2cc(Nc3ncc4c(-c5ccc6ncccc6c5)c[nH]c4n3)ccn2)CC1] | 67 |
| 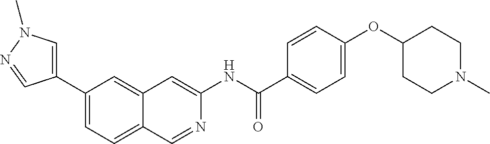 [SMILES: CN1CCC(Oc2ccc(C(=O)Nc3cc4cc(-c5cnn(C)c5)ccc4cn3)cc2)CC1] | 68 |
| 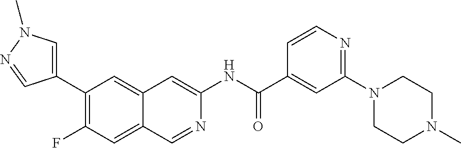 [SMILES: CN1CCN(c2cc(C(=O)Nc3cc4cc(-c5cnn(C)c5)c(F)cc4cn3)ccn2)CC1] | 69 |
| 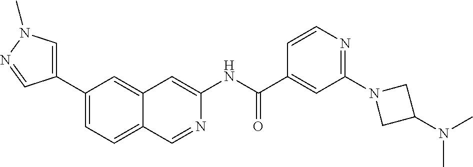 [SMILES: CN(C)C1CN(c2cc(C(=O)Nc3cc4cc(-c5cnn(C)c5)ccc4cn3)ccn2)C1] | 70 |
| 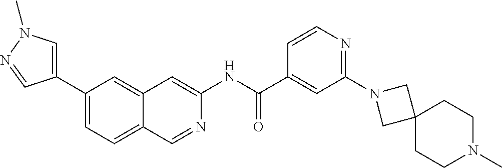 [SMILES: CN1CCC2(CC1)CN(c1cc(C(=O)Nc3cc4cc(-c5cnn(C)c5)ccc4cn3)ccn1)C2] | 71 |
| 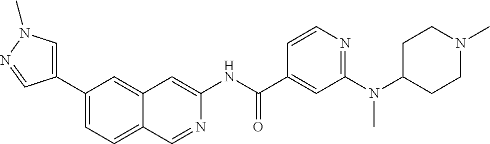 [SMILES: CN1CCC(N(C)c2cc(C(=O)Nc3cc4cc(-c5cnn(C)c5)ccc4cn3)ccn2)CC1] | 72 |
| 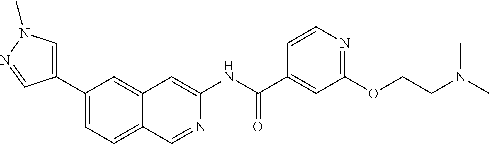 [SMILES: CN(C)CCOc1cc(C(=O)Nc2cc3cc(-c4cnn(C)c4)ccc3cn2)ccn1] | 73 |
| 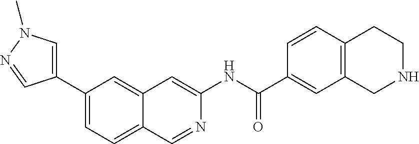 [SMILES: Cn1cc(-c2ccc3cnc(NC(=O)c4ccc5c(c4)CNCC5)cc3c2)cn1] | 74 |
| 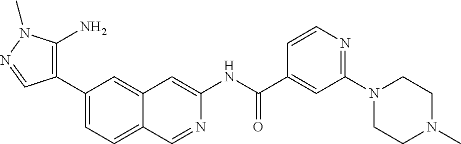 [SMILES: CN1CCN(c2cc(C(=O)Nc3cc4cc(-c5cnn(C)c5N)ccc4cn3)ccn2)CC1] | 75 |
| 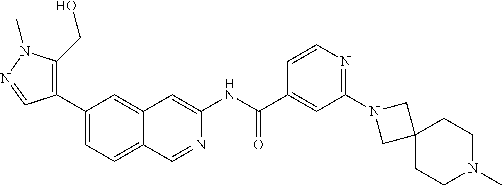 [SMILES: CN1CCC2(CC1)CN(c1cc(C(=O)Nc3cc4cc(-c5cnn(C)c5CO)ccc4cn3)ccn1)C2] | 76 |
| 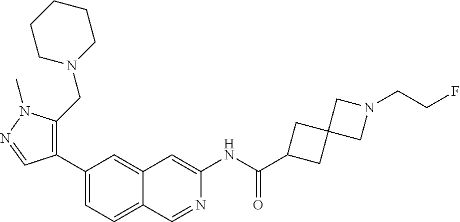 [SMILES: Cn1ncc(-c2ccc3cnc(NC(=O)C4CC5(C4)CN(CCF)C5)cc3c2)c1CN1CCCCC1] | 77 |
| 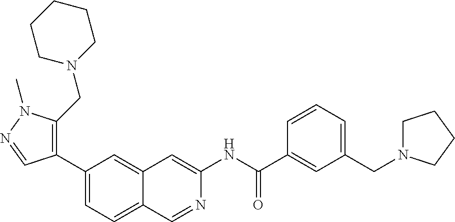 [SMILES: Cn1ncc(-c2ccc3cnc(NC(=O)c4cccc(CN5CCCC5)c4)cc3c2)c1CN1CCCCC1] | 78 |
| 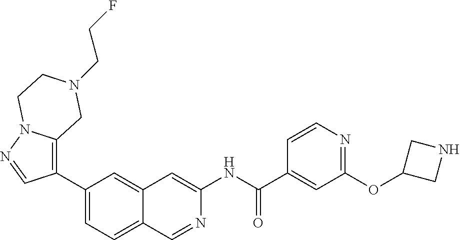 [SMILES: O=C(Nc1cc2cc(-c3cnn4c3CN(CCF)CC4)ccc2cn1)c1ccnc(OC2CNC2)c1] | 79 |
| 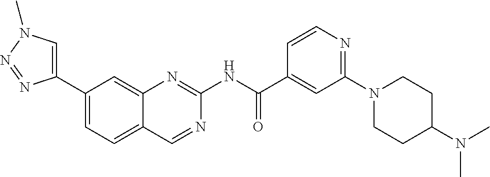 [SMILES: CN(C)C1CCN(c2cc(C(=O)Nc3ncc4ccc(-c5cn(C)nn5)cc4n3)ccn2)CC1] | 80 |
| 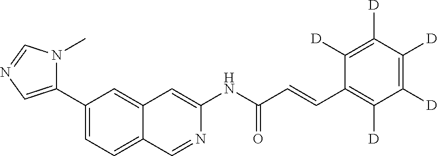 [SMILES: [2H]c1c([2H])c([2H])c(/C=C/C(=O)Nc2cc3cc(-c4cncn4C)ccc3cn2)c([2H])c1[2H]] | 81 |
| 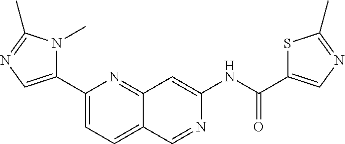 [SMILES: Cc1ncc(C(=O)Nc2cc3nc(-c4cnc(C)n4C)ccc3cn2)s1] | 82 |
| 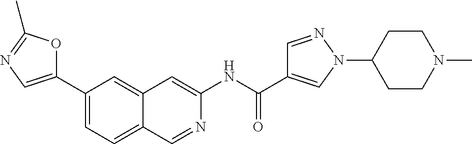 [SMILES: Cc1ncc(-c2ccc3cnc(NC(=O)c4cnn(C5CCN(C)CC5)c4)cc3c2)o1] | 83 |
| 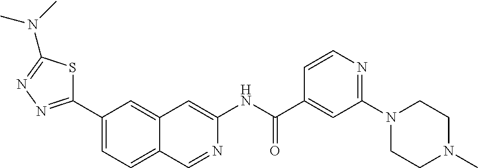 | 84 |
| 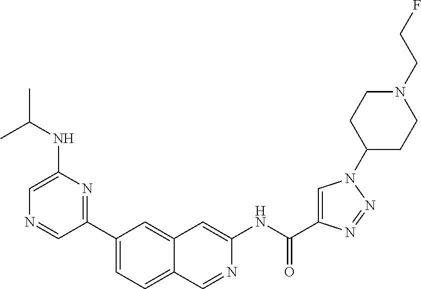 [SMILES: CC(C)Nc1cncc(-c2ccc3cnc(NC(=O)c4cn(C5CCN(CCF)CC5)nn4)cc3c2)n1] | 85 |
| 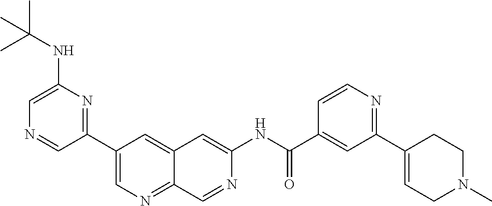 [SMILES: CN1CC=C(c2cc(C(=O)Nc3cc4cc(-c5cncc(NC(C)(C)C)n5)cnc4cn3)ccn2)CC1] | 86 |
| 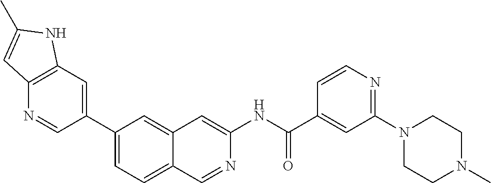 | 87 |
| 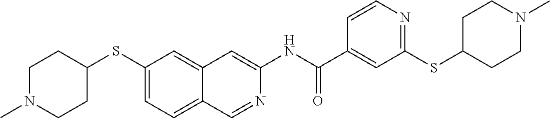 | 88 |
| 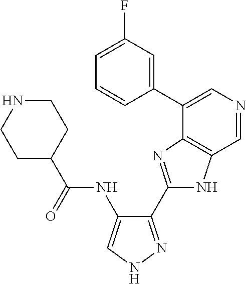 [SMILES: O=C(Nc1c[nH]nc1-c1nc2c(-c3cccc(F)c3)cncc2[nH]1)C1CCNCC1] | 89 |
| 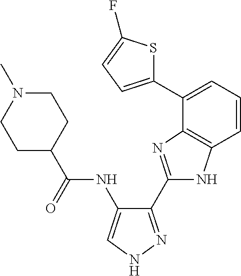 [SMILES: CN1CCC(C(=O)Nc2c[nH]nc2-c2nc3c(-c4ccc(F)s4)cccc3[nH]2)CC1] | 90 |
| 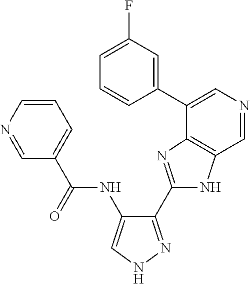 [SMILES: O=C(Nc1c[nH]nc1-c1nc2c(-c3cccc(F)c3)cncc2[nH]1)c1cccnc1] | 91 |
| 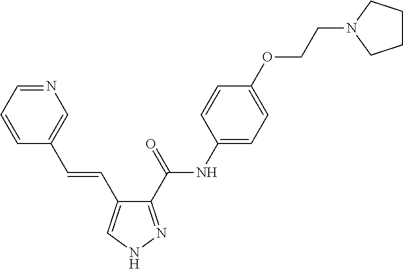 [SMILES: O=C(Nc1ccc(OCCN2CCCC2)cc1)c1n[nH]cc1/C=C/c1cccnc1] | 92 |
| 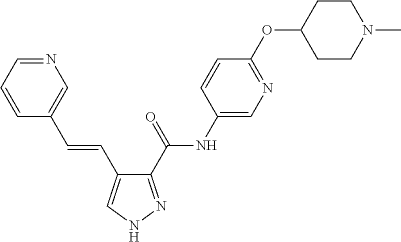 [SMILES: CN1CCC(Oc2ccc(NC(=O)c3n[nH]cc3/C=C/c3cccnc3)cn2)CC1] | 93 |

### Compositions and Kits

Also provided herein are compositions (e.g., pharmaceutical compositions) that include at least one CLK inhibitor (e.g., any of the exemplary CLK inhibitors described herein or known in the art), and instructions for performing any of the methods described herein. In some embodiments, the compositions (e.g., pharmaceutical compositions) can be disposed in a sterile vial or a pre-loaded syringe.

Administration of the compounds disclosed herein or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

In some embodiments, the compositions (e.g., pharmaceutical compositions) are formulated for different routes of administration (e.g., intravenous, intramuscular, subcutaneous, or intracranial). In some embodiments, the compositions (e.g., pharmaceutical compositions) can include a pharmaceutically acceptable salt (e.g., phosphate buffered saline). In some embodiments, the compositions (e.g., pharmaceutical compositions) can include an enantiomer, a diastereoisomer or a tautomer. Single or multiple administrations of any of the pharmaceutical compositions described herein can be given to a subject depending on, for example: the dosage and frequency as required and tolerated by the patient. A dosage of the pharmaceutical composition should provide a sufficient quantity of the CLK inhibitor (e.g., any of the CLK inhibitors described herein), or pharmaceutically acceptable salt thereof, to effectively treat or ameliorate conditions, diseases or symptoms of cancer.

The compounds provided herein may also be useful in combination (administered together or sequentially) with one another or other known agents.

Non-limiting examples of diseases which can be treated with a combination of a compound of Formulas (I)-(XII) and another active agent are colorectal cancer and ovarian cancer. For example, a compound of Formulas (I)-(XII) can be combined with one or more chemotherapeutic compounds.

In some embodiments, colorectal cancer can be treated with a combination of a compound of Formulas (I)-(XII) and one or more of the following drugs: 5-Fluorouracil (5-FU), which can be administered with the vitamin-like drug leucovorin (also called folinic acid); capecitabine (XELODA©), irinotecan (CAMPOSTAR©), oxaliplatin (ELOXATIN©). Examples of combinations of these drugs which could be further combined with a compound of Formulas (I)-(XII) are FOLFOX (5-FU, leucovorin, and oxaliplatin), FOLFIRI (5-FU, leucovorin, and innotecan), FOLFOXIRI (leucovorin, 5-FU, oxaliplatin, and irinotecan) and CapeOx (Capecitabine and oxaliplatin). For rectal cancer, chemo with 5-FU or capecitabine combined with radiation may be given before surgery (neoadjuvant treatment).

In some embodiments, ovarian cancer can be treated with a combination of a compound of Formulas (I)-(XII) and one or more of the following drugs: Topotecan, Liposomal doxorubicin (DOXIL®), Gemcitabine (GEMZAR©), Cyclophosphamide (CYTOXAN®), Vinorelbine (NAVELBINE®), Ifosfamide (IFEX®), Etoposide (VP-16), Altretamine (HEXALEN®), Capecitabine (XELODA®), Irinotecan (CPT-11, CAMPTOSAR®), Melphalan, Pemetrexed (ALIMTA®) and Albumin bound paclitaxel (nab-paclitaxel, ABRAXANE®). Examples of combinations of these drugs which could be further combined with a compound of Formulas (I)-(XII) are TIP (paclitaxel [Taxol], ifosfamide, and cisplatin), VeIP (vinblastine, ifosfamide, and cisplatin) and VIP (etoposide [VP-16], ifosfamide, and cisplatin).

In some embodiments, a compound of Formulas (I)-(XII) can be used to treat cancer in combination with any of the following methods: (a) Hormone therapy such as aromatase inhibitors, LHRH [luteinizing hormone-releasing hormone] analogs and inhibitors, and others; (b) Ablation or embolization procedures such as radiofrequency ablation (RFA), ethanol (alcohol) ablation, microwave thermotherapy and cryosurgery (cryotherapy); (c) Chemotherapy using alkylating agents such as cisplatin and carboplatin, oxaliplatin, mechlorethamine, cyclophosphamide, chlorambucil and ifosfamide; (d) Chemotherapy using anti-metabolites such as azathioprine and mercaptopurine; (e) Chemotherapy using plant alkaloids and terpenoids such as vinca alkaloids (i.e. Vincristine, Vinblastine, Vinorelbine and Vindesine) and taxanes; (f) Chemotherapy using podophyllotoxin, etoposide, teniposide and docetaxel; (g) Chemotherapy using topoisomerase inhibitors such as irinotecan, topotecan, amsacrine, etoposide, etoposide phosphate, and teniposide; (h) Chemotherapy using cytotoxic antibiotics such as actinomycin, anthracyclines, doxorubicin, daunorubicin, valrubicin, idarubicin, epirubicin, bleomycin, plicamycin and mitomycin; (i) Chemotherapy using tyrosine-kinase inhibitors such as Imatinib mesylate (GLEEVEC®, also known as STI-571), Gefitinib (Iressa, also known as ZD1839), Erlotinib (marketed as TARCEVA®), Bortezomib (VELCADE®), tamoxifen, tofacitinib, crizotinib, Bcl-2 inhibitors (e.g. obatoclax in clinical trials, ABT-263, and Gossypol), PARP inhibitors (e.g. Iniparib, Olaparib in clinical trials), PI3K inhibitors (e.g. perifosine in a phase III trial), VEGF Receptor 2 inhibitors (e.g. Apatinib), AN-152, (AEZS-108), Braf inhibitors (e.g. vemurafenib, dabrafenib and LGX818), MEK inhibitors (e.g. trametinib and MEK162), CDK inhibitors, (e.g. PD-0332991), salinomycin and Sorafenib; (j) Chemotherapy using monoclonal antibodies such as Rituximab (marketed as MABTHERA® or RITUXAN©), Trastuzumab (Herceptin also known as ErbB2), Cetuximab (marketed as ERBITUX©), and Bevacizumab (marketed as AVASTIN©); and (k) radiation therapy.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants and the like, for prolonged and/or timed, pulsed administration at a predetermined rate.

The compounds can be administered either alone or in combination with a conventional pharmaceutical carrier, excipient or the like. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In some embodiments, the unit dosage of compounds of Formulas (I)-(XII) is about 0.25 mg/Kg to about 50 mg/Kg in humans.

In some embodiments, the unit dosage of compounds of Formulas (I)-(XII) is about 0.25 mg/Kg to about 20 mg/Kg in humans.

In some embodiments, the unit dosage of compounds of Formulas (I)-(XII) is about 0.50 mg/Kg to about 19 mg/Kg in humans.

In some embodiments, the unit dosage of compounds of Formulas (I)-(XII) is about 0.75 mg/Kg to about 18 mg/Kg in humans.

In some embodiments, the unit dosage of compounds of Formulas (I)-(XII) is about 1.0 mg/Kg to about 17 mg/Kg in humans.

In some embodiments, the unit dosage of compounds of Formulas (I)-(XII) is about 1.25 mg/Kg to about 16 mg/Kg in humans.

In some embodiments, the unit dosage of compounds of Formulas (I)-(XII) is about 1.50 mg/Kg to about 15 mg/Kg in humans.

In some embodiments, the unit dosage of compounds of Formulas (I)-(XII) is about 1.75 mg/Kg to about 14 mg/Kg in humans.

In some embodiments, the unit dosage of compounds of Formulas (I)-(XII) is about 2.0 mg/Kg to about 13 mg/Kg in humans.

In some embodiments, the unit dosage of compounds of Formulas (I)-(XII) is about 3.0 mg/Kg to about 12 mg/Kg in humans.

In some embodiments, the unit dosage of compounds of Formulas (I)-(XII) is about 4.0 mg/Kg to about 11 mg/Kg in humans.

In some embodiments, the unit dosage of compounds of Formulas (I)-(XII) is about 5.0 mg/Kg to about 10 mg/Kg in humans.

In some embodiments, the compositions are provided in unit dosage forms suitable for single administration.

In some embodiments, the compositions are provided in unit dosage forms suitable for twice a day administration.

In some embodiments, the compositions are provided in unit dosage forms suitable for three times a day administration.

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable and could be higher if the composition is a solid or suspension, which could be subsequently diluted to the above percentages.

In some embodiments, the composition will comprise about 0.1-10% of the active agent in solution.

In some embodiments, the composition will comprise about 0.1-5% of the active agent in solution.

In some embodiments, the composition will comprise about 0.1-4% of the active agent in solution.

In some embodiments, the composition will comprise about 0.15-3% of the active agent in solution.

In some embodiments, the composition will comprise about 0.2-2% of the active agent in solution.

In some embodiments, the compositions are provided in dosage forms suitable for continuous dosage by intravenous infusion over a period of about 1-96 hours.

In some embodiments, the compositions are provided in dosage forms suitable for continuous dosage by intravenous infusion over a period of about 1-72 hours.

In some embodiments, the compositions are provided in dosage forms suitable for continuous dosage by intravenous infusion over a period of about 1-48 hours.

In some embodiments, the compositions are provided in dosage forms suitable for continuous dosage by intravenous infusion over a period of about 1-24 hours.

In some embodiments, the compositions are provided in dosage forms suitable for continuous dosage by intravenous infusion over a period of about 1-12 hours.

In some embodiments, the compositions are provided in dosage forms suitable for continuous dosage by intravenous infusion over a period of about 1-6 hours.

In some embodiments, these compositions can be administered by intravenous infusion to humans at doses of about 5 mg/m² to about 300 mg/m².

In some embodiments, these compositions can be administered by intravenous infusion to humans at doses of about 5 mg/m² to about 200 mg/m².

In some embodiments, these compositions can be administered by intravenous infusion to humans at doses of about 5 mg/m² to about 100 mg/m².

In some embodiments, these compositions can be administered by intravenous infusion to humans at doses of about 10 mg/m² to about 50 mg/m².

In some embodiments, these compositions can be administered by intravenous infusion to humans at doses of about 50 mg/m² to about 200 mg/m².

In some embodiments, these compositions can be administered by intravenous infusion to humans at doses of about 75 mg/m² to about 175 mg/m².

In some embodiments, these compositions can be administered by intravenous infusion to humans at doses of about 100 mg/m² to about 150 mg/m².

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formulas (I)-(XII) disclosed herein intended for respiratory delivery (either systemic or local) can be administered as aqueous formulations, as non-aqueous solutions or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the compositions of Formulas (I)-(XII) disclosed herein can be administered to the ear by various methods. For example, a round window catheter (e.g., U.S. Pat. Nos. 6,440,102 and 6,648,873) can be used.

Alternatively, formulations can be incorporated into a wick for use between the outer and middle ear (e.g., U.S. Pat. No. 6,120,484) or absorbed to collagen sponge or other solid support (e.g., U.S. Pat. No. 4,164,559).

If desired, formulations of the disclosure can be incorporated into a gel formulation (e.g., U.S. Pat. Nos. 4,474,752 and 6,911,211).

In some embodiments, compounds of Formulas (I)-(XII) disclosed herein intended for delivery to the ear can be administered via an implanted pump and delivery system through a needle directly into the middle or inner ear (cochlea) or through a cochlear implant stylet electrode channel or alternative prepared drug delivery channel such as but not limited to a needle through temporal bone into the cochlea.

Other options include delivery via a pump through a thin film coated onto a multichannel electrode or electrode with a specially imbedded drug delivery channel (pathways) carved into the thin film for this purpose. In other embodiments the acidic or basic solid compound of Formulas (I)-(XII) can be delivered from the reservoir of an external or internal implanted pumping system.

Formulations of the disclosure also can be administered to the ear by intratympanic injection into the middle ear, inner ear, or cochlea (e.g., U.S. Pat. No. 6,377,849 and Ser. No. 11/337,815).

Intratympanic injection of therapeutic agents is the technique of injecting a therapeutic agent behind the tympanic membrane into the middle and/or inner ear. In one embodiment, the formulations described herein are administered directly onto the round window membrane via transtympanic injection. In another embodiment, the ion channel modulating agent auris-acceptable formulations described herein are administered onto the round window membrane via a non-transtympanic approach to the inner ear. In additional embodiments, the formulation described herein is administered onto the round window membrane via a surgical approach to the round window membrane comprising modification of the crista fenestrae cochleae.

In some embodiments, the compounds of Formulas (I)-(XII) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG (like PEG ointments), and the like.

Suppositories for rectal administration of the drug (either as a solution, colloid, suspension or a complex) can be prepared by mixing a compound provided herein with a suitable non-irritating excipient that is solid at ordinary temperatures but liquid at the rectal temperature and will therefore melt or erode/dissolve in the rectum and release the compound. Such materials include cocoa butter, glycerinated gelatin, hydrogenated vegetable oils, poloxamers, mixtures of polyethylene glycols of various molecular weights and fatty acid esters of polyethylene glycol. In suppository forms of the compositions, a low-melting wax such as, but not limited to, a mixture of fatty acid glycerides, optionally in combination with cocoa butter, is first melted.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

Also provided herein are kits that include one or more (e.g., 1, 2, 3, 4, 5, 6, 7, 8, 9, 10, 12, 14, 15, 16, 18, or 20) of any of the pharmaceutical compositions described herein that includes a therapeutically effective amount of any of the compounds of Formulas (I)-(XII) described herein, or a pharmaceutically acceptable salt.

In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with cancer. In another embodiment, the kit can include a compound or composition as described herein and a label that indicates that the contents are to be administered to a patient with one or more of hepatocellular carcinoma, colon cancer, leukemia, lymphoma, sarcoma, and ovarian cancer.

The kits described herein are not so limited; other variations will be apparent to one of ordinary skill in the art.

### EXAMPLES

The disclosure is further described in the following examples, which do not limit the scope of the disclosure described in the claims.

### Example 1. Wnt Activity Screening Assay

The screening assay for Wnt activity is described as follows. Reporter cell lines can be generated by stably transducing cancer cell lines (e.g., colon cancer) or primary cells (e.g., IEC-6 intestinal cells) with a lentiviral construct that includes a Wnt-responsive promoter driving expression of the firefly luciferase gene.

SW480 colon carcinoma cells were transduced with a lentiviral vector expressing luciferase with a human Sp5 promoter consisting of a sequence of eight TCF/LEF binding sites. SW480 cells stably expressing the Sp5-Luc reporter gene and a hygromycin resistance gene were selected by treatment with 150 pg/mL of hygromycin for 7 days. These stably transduced SW480 cells were expanded in cell culture and used for all further screening activities. Each compound was dissolved in DMSO as a 10 mM stock and used to prepare compound source plates. Serial dilution (1:3, 10-point dose-response curves starting from 10 μM) and compound transfer was performed using the ECHO 550 (Labcyte, Sunnyvale, Calif.) into 384-well white solid bottom assay plates (Greiner Bio-One) with appropriate DMSO backfill for a final DMSO concentration of 0.1%. For Sp5-Luc reporter gene assays, the cells were plated at 4,000 cells/well in 384-well plates with a DMEM medium containing 1% fetal bovine serum, and 1% Penicillin-Streptomycin and incubated for 36 to 48 hours at 37° C. and 5% CO₂. Following incubation, 15 μL of BriteLite Plus luminescence reagent (Perkin Elmer) was added to each well of the 384-well assay plates. The plates were placed on an orbital shaker for 2 min and then luminescence was quantified using the Envision (Perkin Elmer) plate reader. Readings were normalized to DMSO only treated cells, and normalized activities were utilized for EC₅₀ calculations using the dose-response log (inhibitor) vs. response -variable slope (four parameters) nonlinear regression feature available in GraphPad Prism 5.0 (or Dotmatics).

Table 2 shows the measured activity for representative compounds of Formulas (I)-(XII) as described herein.

TABLE 2

| | EC₅₀ |
|---|---|
| Compound | (μM) |
| 1 | 0.039 |
| 2 | 0.253 |
| 3 | 0.356 |
| 4 | 0.525 |
| 5 | 0.257 |
| 6 | 0.065 |
| 7 | 0.041 |
| 8 | 0.623 |
| 9 | 0.092 |
| 10 | 0.145 |
| 11 | 0.013 |
| 12 | 0.085 |
| 13 | 0.038 |
| 14 | 0.055 |
| 15 | 0.348 |
| 16 | 0.165 |
| 17 | 1.003 |
| 18 | 0.161 |
| 19 | 0.071 |
| 20 | 0.169 |
| 21 | 0.151 |
| 22 | 0.014 |
| 23 | 0.026 |
| 24 | 0.320 |
| 25 | 0.114 |
| 26 | 0.039 |
| 27 | 0.116 |
| 28 | 0.369 |
| 29 | 0.014 |
| 30 | 0.013 |
| 31 | 0.004 |
| 32 | 0.085 |
| 33 | 0.030 |
| 34 | 0.176 |
| 35 | 0.109 |
| 36 | 0.037 |
| 37 | 0.097 |
| 38 | 0.013 |
| 39 | 0.199 |
| 40 | 0.013 |
| 41 | 0.773 |
| 42 | 0.086 |
| 43 | 0.133 |
| 44 | 0.079 |
| 45 | >10 |
| 46 | 0.021 |
| 47 | 0.285 |
| 48 | 0.310 |
| 49 | >10 |
| 50 | 0.377 |
| 51 | >10 |
| 52 | 4.464 |
| 53 | 0.188 |
| 54 | 0.042 |
| 55 | 0.052 |
| 56 | 0.087 |
| 57 | 0.338 |
| 58 | 0.577 |
| 59 | 0.015 |
| 60 | 0.080 |
| 61 | 0.144 |
| 62 | >10 |
| 63 | 1.806 |
| 64 | 0.225 |
| 65 | 0.095 |
| 66 | 0.244 |
| 67 | 0.284 |
| 68 | 0.104 |
| 69 | 0.058 |
| 70 | 0.062 |
| 71 | 0.036 |
| 72 | 0.041 |
| 73 | 0.108 |
| 74 | 0.184 |
| 75 | 0.047 |
| 76 | 0.270 |
| 77 | 0.170 |
| 78 | 0.189 |
| 79 | 0.489 |
| 80 | 0.742 |
| 81 | 0.062 |
| 82 | 0.107 |
| 83 | 0.034 |
| 84 | 0.138 |
| 85 | 0.034 |
| 86 | 0.357 |
| 87 | 2.592 |
| 88 | 0.694 |
| 89 | 2.172 |
| 90 | 0.019 |
| 91 | 0.003 |
| 92 | 1.850 |
| 93 | 0.964 |

### Example 2. CLK2 Kinase Activity

Representative compounds were screened using the assay procedure for CLK2 kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, Calif.) into 1536-well black-walled round bottom plates (Corning).

The CLK2 kinase assay was run using the Ser/Thr 6 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant CLK2 kinase, ATP and Ser/Thr peptide 6 were prepared in 1X Kinase buffer to final concentrations of 0.43 μg/mL, 60 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 6 served as control reactions. Additionally, an 11-point dose-response curve of Staurosporine (1 uM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio X F100%)−C100%)/((C0%−C100%)+(Em ratio X (F100%−F0%)))]. Dose-response curves were generated and inhibitory concentration (IC₅₀) values were calculated using non-linear regression curve fit in the Dotmatics' Studies Software (Bishops Stortford, UK).

Table 3 shows the measured activity for representative compounds of Formulas (I)-(XII) as described herein.

TABLE 3

| | EC₅₀ |
|---|---|
| Compound | (μM) |
| 1 | 0.001 |
| 2 | 0.002 |
| 3 | 0.002 |
| 4 | 0.002 |
| 5 | 0.0003 |
| 6 | 0.001 |
| 7 | 0.001 |
| 8 | 0.001 |
| 9 | 0.010 |
| 10 | 0.011 |
| 11 | 0.001 |
| 12 | 0.001 |
| 13 | 0.001 |
| 14 | 0.002 |
| 15 | 0.001 |
| 16 | 0.001 |
| 17 | 0.002 |
| 18 | 0.002 |
| 19 | 0.002 |
| 20 | 0.001 |
| 21 | 0.001 |
| 22 | 0.002 |
| 23 | 0.002 |
| 24 | 0.003 |
| 25 | 0.010 |
| 26 | 0.001 |
| 27 | 0.005 |
| 28 | 0.003 |
| 29 | 0.005 |
| 30 | 0.001 |
| 31 | 0.001 |
| 32 | 0.001 |
| 33 | 0.001 |
| 34 | 0.001 |
| 35 | 0.001 |
| 36 | 0.001 |
| 37 | 0.001 |
| 38 | 0.001 |
| 39 | 0.004 |
| 40 | 0.003 |
| 41 | 0.002 |
| 42 | 0.001 |
| 43 | 0.004 |
| 44 | 0.018 |
| 45 | 0.0010 |
| 46 | 0.0010 |
| 47 | 0.0013 |
| 48 | 0.0001 |
| 49 | 0.0022 |
| 50 | 0.0038 |
| 51 | 0.0014 |
| 52 | 0.0019 |
| 53 | 0.0097 |
| 54 | 0.0020 |
| 55 | 0.0019 |
| 56 | 0.0033 |
| 57 | 0.0028 |
| 58 | 0.0016 |
| 59 | 0.0010 |
| 60 | 0.0009 |
| 61 | 0.0006 |
| 62 | 0.0053 |
| 63 | 0.0120 |
| 64 | 0.0017 |
| 65 | 0.0010 |
| 66 | 0.0786 |
| 67 | 0.0024 |
| 68 | 0.0006 |
| 69 | 0.0019 |
| 70 | 0.0016 |
| 71 | 0.0011 |
| 72 | 0.0013 |
| 73 | 0.0019 |
| 74 | 0.0019 |
| 75 | 0.0020 |
| 76 | 0.0026 |
| 77 | 0.0058 |
| 78 | 0.0016 |
| 79 | 0.0044 |
| 80 | 0.0089 |
| 81 | 0.0034 |
| 82 | 0.0030 |
| 83 | 0.0029 |
| 84 | 0.0029 |
| 85 | 0.0040 |
| 86 | 0.0047 |
| 87 | 0.0049 |
| 88 | 0.0045 |
| 89 | 0.0022 |
| 90 | 0.0112 |
| 91 | 0.0031 |
| 92 | 0.0060 |
| 93 | 0.0037 |

### Example 3. CLK3 Kinase Activity Assay

Representative compounds were screened using the assay procedure for CLK3 kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, Calif.) into 1536-well black-walled round bottom plates (Corning).

The CLK3 kinase assay was run using the Ser/Thr 18 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant CLK3 kinase, ATP and Ser/Thr peptide 18 were prepared in 1X Kinase buffer to final concentrations of 1.5 μg/mL, 156 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (1 uM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio X F100%)−C100%)/((C0%−C100%)+(Em ratio X (F100%−F0%)))]. Dose-response curves were generated and inhibitory concentration (IC₅₀) values were calculated using non-linear regression curve fit in the Dotmatics' Studies Software (Bishops Stortford, UK).

Table 4 shows the measured activity for representative compounds of Formulas (I)-(XII) as described herein.

TABLE 4

| | EC₅₀ |
|---|---|
| Compound | (μM) |
| 1 | 0.010 |
| 2 | 0.361 |
| 3 | 0.084 |
| 4 | 1.283 |
| 5 | 0.027 |
| 6 | 0.017 |
| 7 | 0.010 |
| 8 | 0.022 |
| 9 | 0.026 |
| 10 | 0.338 |
| 11 | 0.005 |
| 12 | 0.022 |
| 13 | 0.010 |
| 14 | 0.024 |
| 15 | 0.030 |
| 16 | 0.031 |
| 17 | 0.161 |
| 18 | 0.054 |
| 19 | 0.014 |
| 20 | 0.019 |
| 21 | 0.018 |
| 22 | 0.018 |
| 23 | 0.009 |
| 24 | 0.070 |
| 25 | 0.144 |
| 26 | 0.015 |
| 27 | 0.035 |
| 28 | 0.313 |
| 29 | 0.062 |
| 30 | 0.011 |
| 31 | 0.011 |
| 32 | 0.013 |
| 33 | 0.017 |
| 34 | 0.020 |
| 35 | 0.020 |
| 36 | 0.034 |
| 37 | 0.041 |
| 38 | 0.007 |
| 39 | 0.010 |
| 40 | 0.014 |
| 41 | 0.018 |
| 42 | 0.026 |
| 43 | 0.040 |
| 44 | 0.108 |
| 45 | 0.0130 |
| 46 | 0.0250 |
| 47 | 0.0176 |
| 48 | 0.0025 |
| 49 | 0.0193 |
| 50 | 0.0351 |
| 51 | 0.0310 |
| 52 | 0.0266 |
| 53 | 0.0275 |
| 54 | 0.0120 |
| 55 | 0.0281 |
| 56 | 0.0163 |
| 57 | 0.0263 |
| 58 | 0.0119 |
| 59 | 0.0611 |
| 60 | 0.0128 |
| 61 | 0.0491 |
| 62 | 0.0358 |
| 63 | 0.3430 |
| 64 | 0.4824 |
| 65 | 0.3090 |
| 66 | 6.7086 |
| 67 | 0.0103 |
| 68 | 0.0136 |
| 69 | 0.0212 |
| 70 | 0.0245 |
| 71 | 0.0162 |
| 72 | 0.0199 |
| 73 | 0.0312 |
| 74 | 0.0393 |
| 75 | 0.0339 |
| 76 | 0.0438 |
| 77 | 0.0500 |
| 78 | 0.0146 |
| 79 | 0.0443 |
| 80 | 0.0386 |
| 81 | 0.0154 |
| 82 | 0.0313 |
| 83 | 0.0136 |
| 84 | 0.0272 |
| 85 | 0.0120 |
| 86 | 0.0115 |
| 87 | 0.0382 |
| 88 | 0.0214 |
| 89 | 0.0305 |
| 90 | 0.0217 |
| 91 | 0.0225 |
| 92 | 0.9673 |
| 93 | 0.7333 |

Representative compounds were screened using the assay procedure for gene expression as described below (CLK4 IC₅₀=0.001 μM; CLK1 IC₅₀=0.008 μM).

Each compound was dissolved in DMSO as a 10 mM stock. SW480 colorectal cancer cells were plated at 1×10⁴ cells per well into 96-well plates (Olympus). Compounds were diluted in cell culture media and added to the cells at a final concentration of 3 μM. Cells were treated with vehicle (DMSO) and compounds for 24 hours. N=3 biological replicates per conditions.

Following treatment, cells were lysed, and cDNA was generated using the Fastlane Cell cDNA Kit (Qiagen).

384-well PCR plates with pre-spotted primers for CLK1, CLK2, CLK3, DVL2, LRP5, SRSF1, SRSF3, SRSF4, SRSF5, TCF7, TCF7L2 were ordered from Bio-Rad. The generated cDNA, along with a SYBR Green qRT-PCR master mix (SsoAdvanced™ Universal SYBR® Green Supermix, Bio-Rad) was added to the PCR plates.

qRT-PCR was performed on the plates using a CFX384 Touch™ Real-Time PCR Detection System (Bio-Rad) with the manufacturer's recommended thermal cycling conditions.

Data from the qRT-PCR assay was normalized to the GAPDH and HPRT1 housekeeping genes and set gene expression fold change was set relative to vehicle-treated cells using the ΔΔCt analysis method.

Table 5 shows the gene expression fold change for representative compounds of Formulas (I)-(XII) as described herein relative to DMSO.

TABLE 5

| Compound | CLK1 | CLK2 | CLK3 | DVL2 | LRP5 | SRSF1 | SRSF3 | SRSF4 | SRSF5 | TCF7 | TCF7L2 |
|---|---|---|---|---|---|---|---|---|---|---|---|
| DMSO | 1.00 | 1.00 | 1.00 | 1.00 | 1.00 | 1.00 | 1.00 | 1.00 | 1.00 | 1.00 | 1.00 |
| 1 | 0.02 | 0.02 | 0.01 | 0.14 | 0.18 | 0.32 | 0.13 | 0.03 | 0.39 | 0.44 | 0.24 |
| 2 | 0.01 | 0.05 | 0.02 | 0.05 | 0.07 | 0.13 | 0.05 | 0.02 | 0.14 | 0.32 | 0.13 |
| 3 | 0.01 | 0.00 | 0.02 | 0.11 | 0.08 | 0.22 | 0.09 | 0.02 | 0.21 | 0.41 | 0.18 |
| 4 | 0.43 | 0.35 | 0.74 | 0.11 | 0.06 | 0.25 | 0.05 | 0.70 | 0.27 | 0.21 | 0.13 |
| 5 | 0.15 | 0.12 | 0.55 | 0.37 | 0.43 | 1.52 | 0.46 | 0.27 | 0.60 | 0.30 | 0.24 |
| 6 | 0.04 | 0.06 | 0.03 | 0.06 | 0.05 | 0.10 | 0.04 | 0.01 | 0.10 | 0.31 | 0.07 |
| 9 | 0.16 | 0.06 | 0.51 | 0.21 | 0.18 | 1.91 | 0.62 | 0.62 | 0.56 | 0.20 | 0.29 |
| 10 | 0.12 | 0.06 | 0.33 | 0.34 | 0.27 | 0.84 | 0.44 | 0.30 | 0.46 | 0.39 | 0.71 |
| 11 | 0.02 | 0.04 | 0.09 | 0.11 | 0.09 | 0.74 | 0.17 | 0.15 | 0.24 | 0.19 | 0.17 |
| 12 | 0.07 | 0.04 | 0.29 | 0.19 | 0.16 | 1.62 | 0.52 | 0.44 | 0.43 | 0.26 | 0.21 |
| 13 | 0.02 | 0.07 | 0.12 | 0.17 | 0.15 | 0.97 | 0.21 | 0.17 | 0.30 | 0.25 | 0.30 |
| 14 | 0.05 | 0.04 | 0.26 | 0.38 | 0.45 | 1.37 | 0.49 | 0.32 | 0.63 | 0.36 | 0.57 |
| 15 | 0.04 | 0.05 | 0.12 | 0.11 | 0.12 | 0.96 | 0.19 | 0.17 | 0.31 | 0.23 | 0.15 |
| 16 | 0.22 | 0.06 | 0.59 | 0.18 | 0.17 | 1.92 | 0.61 | 0.66 | 0.54 | 0.21 | 0.14 |
| 18 | 0.41 | 0.10 | 0.88 | 0.16 | 0.29 | 1.99 | 0.94 | 0.77 | 0.48 | 0.25 | 0.29 |
| 19 | 0.10 | 0.02 | 0.34 | 0.27 | 0.38 | 1.57 | 0.55 | 0.37 | 0.52 | 0.31 | 0.35 |
| 20 | 0.07 | 0.08 | 0.35 | 0.42 | 0.37 | 1.56 | 0.50 | 0.33 | 0.63 | 0.47 | 0.60 |
| 21 | 0.02 | 0.03 | 0.05 | 0.15 | 0.07 | 0.22 | 0.07 | 0.03 | 0.16 | 0.48 | 0.10 |
| 22 | 0.04 | 0.04 | 0.04 | 0.41 | 0.21 | 0.53 | 0.18 | 0.04 | 0.36 | 0.75 | 0.68 |
| 23 | 0.02 | 0.03 | 0.02 | 0.11 | 0.04 | 0.30 | 0.09 | 0.02 | 0.24 | 0.25 | 0.12 |
| 24 | 0.35 | 0.19 | 0.19 | 0.39 | 0.34 | 0.46 | 0.31 | 0.37 | 0.49 | 0.49 | 0.33 |
| 25 | 0.07 | 0.09 | 0.08 | 0.21 | 0.09 | 0.20 | 0.10 | 0.09 | 0.19 | 0.21 | 0.11 |
| 26 | 0.02 | 0.05 | 0.02 | 0.06 | 0.15 | 0.16 | 0.04 | 0.02 | 0.09 | 0.37 | 0.09 |
| 27 | 0.03 | 0.06 | 0.22 | 0.21 | 0.22 | 1.88 | 0.43 | 0.29 | 0.54 | 0.32 | 0.20 |

### Example 4. Compound 12 is a CLK Kinase Inhibitor, Impacts Both Wnt Pathway Activity and mRNA Splicing Activity in Cancer Cells

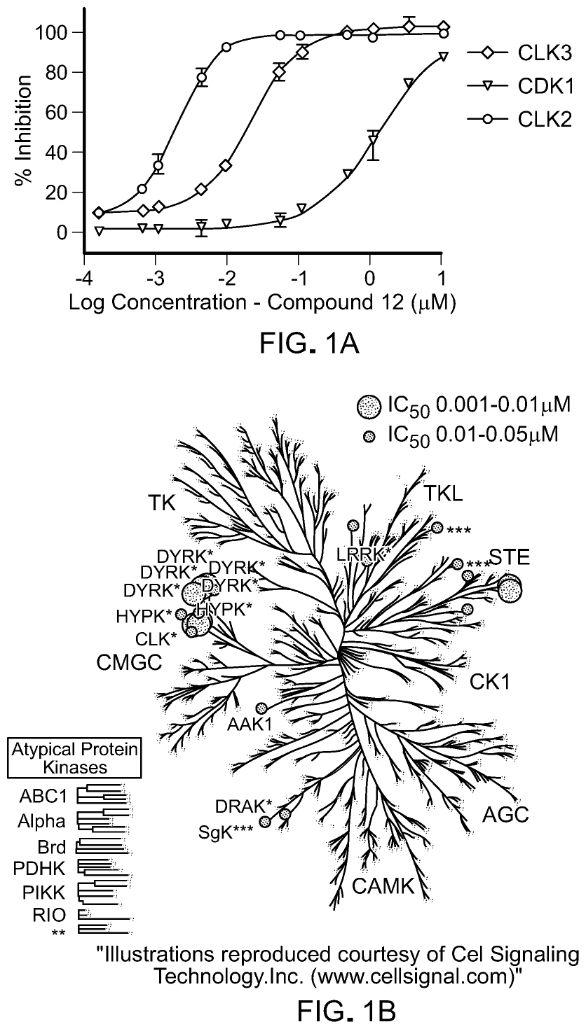

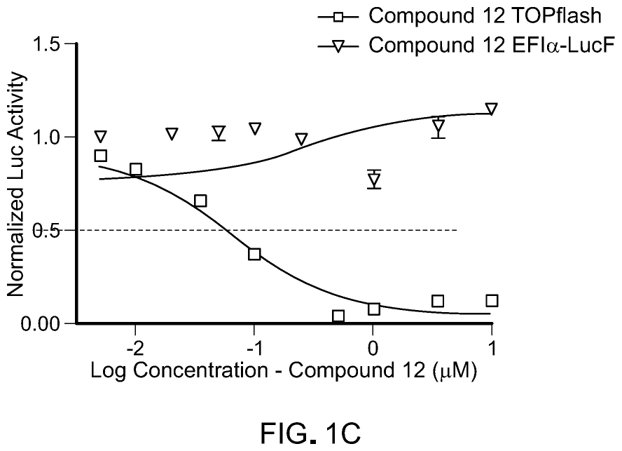

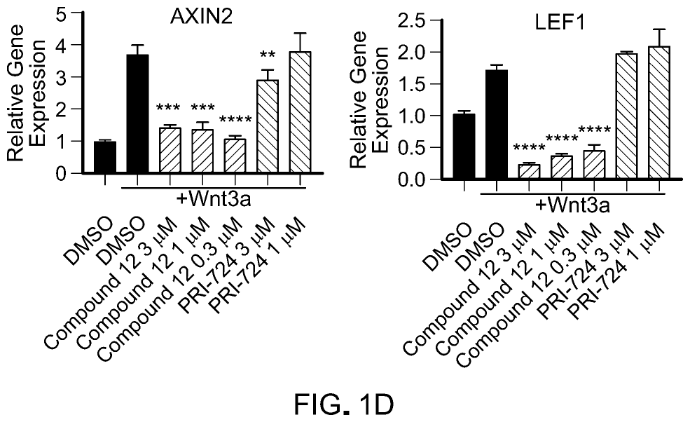

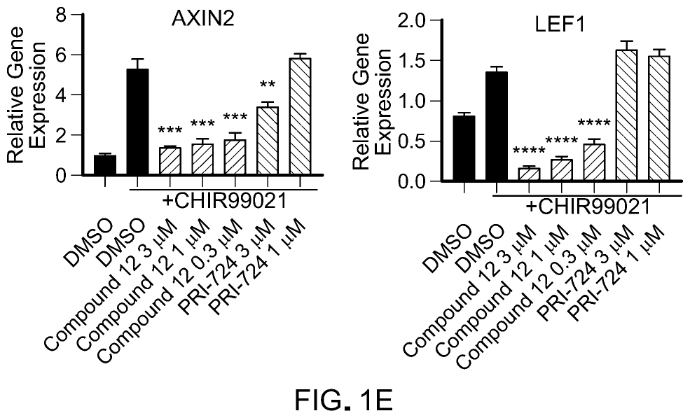

In an iterative screening campaign which involved >1,500 compounds, Compound 12 was developed as a small molecule CLK kinase inhibitor demonstrating IC₅₀ values of 0.00 μM for CLK2 and 0.022 μM for CLK3. Compared to CLK2 and CLK3 inhibitory activity, Compound 12 demonstrated ˜550-fold and ˜50-fold selectivity, respectively, against cyclin-dependent kinase 1 (CDK1) (IC₅₀=1.1 μM) (FIG. 1A). To further characterize the target profile of Compound 12, an independent kinase screen was performed using ThermoFisher SelectScreen (466 kinases tested at 1 μM). Those kinases which demonstrated >80% inhibition at 1 μM, were followed up to determine their IC₅₀ (Table 6).

TABLE 6
IC₅₀ of Additional Kinases
Inhibited by Compound 12.

| | | Fold difference |
|---|---|---|
| | | against |
| Kinase Name | IC₅₀ (nM) | CLK2 IC₅₀ |
| CLK4 | 0.001 | 0.5 |
| CLK2 | 0.002 | 1 |
| DYRK1A | 0.002 | 1 |
| DYRK1B | 0.002 | 1 |
| DYRK2 | 0.003 | 1.5 |
| MAP4K4 (HGK) | 0.007 | 3.5 |
| DYRK3 | 0.008 | 3.8 |
| CLK1 | 0.008 | 4 |
| MINK1 | 0.008 | 4 |
| DYRK4 | 0.013 | 6.5 |
| LRRK2 | 0.017 | 8.5 |
| LRRK2 FL | 0.019 | 9.5 |
| IRAK1 | 0.02 | 10 |
| CLK3 | 0.022 | 11 |
| AAK1 | 0.023 | 11.5 |
| MAP4K1 (HPK1) | 0.029 | 14.5 |
| MYLK4 | 0.03 | 15 |
| TAOK1 | 0.031 | 15.5 |
| BMPR1B (ALK6) | 0.033 | 16.5 |
| MAP3K7/MAP3K7IP1 | 0.037 | 18.5 |
| (TAK1-TAB1) | | |
| HIPK3 (YAK1) | 0.042 | 21 |
| STK17A (DRAK1) | 0.044 | 22 |
| HIPK2 | 0.046 | 23 |

Dose response curves were generated and inhibitory concentration (IC₅₀) values were calculated using non-linear regression curve Prism ® software (GraphPad). IC₅₀ determination for the other kinases were performed by Thermo Fisher Scientific SelectScreen ™ service.

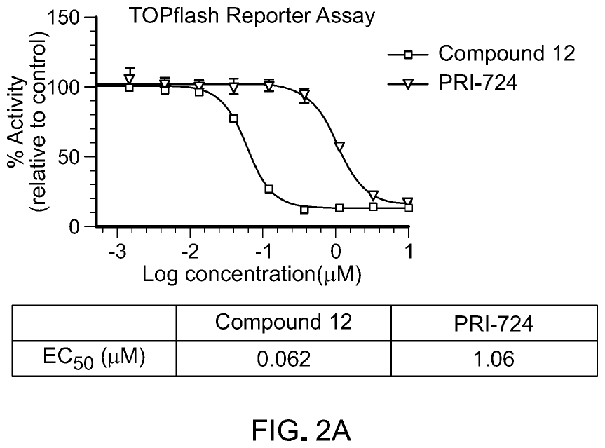

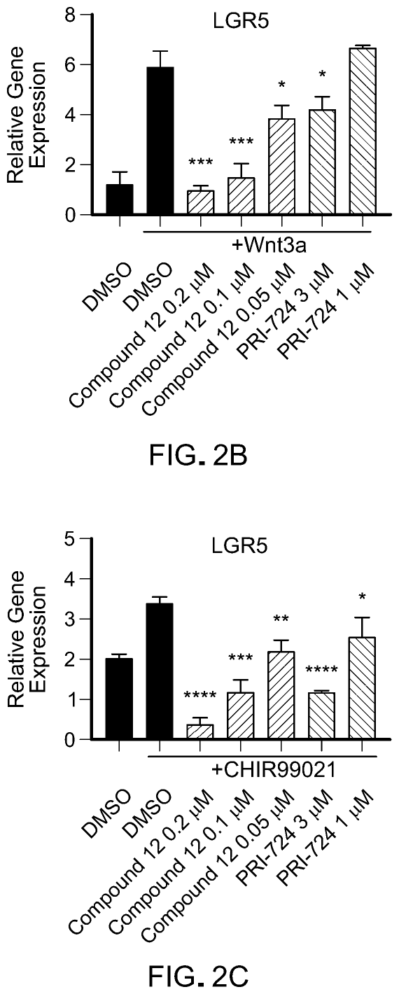

Those IC₅₀s which were ≤0.05 μM or lying within 25-fold of the CLK2 IC₅₀ of 0.002 μM reflect other potential proximal targets represented in a kinase dendrogram (FIG. 1B). As demonstrated, CLK1 and CLK4 were also inhibited at IC₅₀, 0.008 μM and 0.001 μM, respectively. Additionally, DYRK kinases (DYRK1A, -1B, -2 and -4) which are in the same part of CMGC phylogenetic tree as CLKs were inhibited at ranges 0.002-0.013 μM. Of note, as seen with CDK1, no other CDKs were inhibited by Compound 12 at IC₅₀, <0.05 μM which significantly reduces potential of confounding anti-proliferative effects mediated by CDK inhibition. Overall, Compound 12 demonstrated good selectivity against wild type kinases with 19 kinases inhibited besides CLKs, representing 4% of the 466 kinases evaluated. Compound 12 demonstrated strong Wnt pathway inhibitory activity in the TOPflash p-catenin/TCF-responsive reporter assay in SW480 colon cancer cells bearing a mutation in the APC protein, which leads to constitutively active canonical Wnt signaling (EC₅₀=0.046 μM) (Kawahara et al., J Biol. Chem. 275:8369-8374, 2000). Compound 12 demonstrated >10-fold more potency than PRI-724, a known Wnt pathway inhibitor (EC₅₀=1.06 μM) (FIG. 2A) (Emami et al., Proc. Natl. Acad. Sci. U.S.A. 101:12682-12687, 2004; Lenz et al., Cancer Sci. 105:1087-1092, 2014) and in control experiments, Compound 12 did not inhibit the activity of a non-specific EF1α-luciferase reporter (EF1α-LucF) (EC₅₀=>10 μM) (FIG. 1C). Furthermore, Compound 12 (0.3-3 μM) inhibited Wnt3a and CHIR9902-stimulated gene expression of Wnt-target genes, AXIN2 and LEF1 (FIGS. 1D & 1E). Compound 12 also inhibited the activation of Wnt/β-catenin signaling pathway in a non-transformed rat small intestinal crypt epithelial cell line, IEC-6 compared to stimulated DMSO controls. IEC-6 cells treated with Compound 12 was more potent than PRI-724 in preventing the increase of the Wnt target gene and stem cell marker LGR5 gene expression induced by Wnt3a or CHIR99021 (FIGS. 2B and 2C).

Seventeen human CRC cell lines carrying one or more of the genomic mutations in APC, CTAWB1, BRAF, and KRAS were tested to determine the effects of Compound 12 on cellular proliferation, as evaluated using the CellTiter-Blue® Viability Assay. As summarized in Table 7, all cell lines were responsive to Compound 12 with all EC₅₀ values reported as <0.5 μM across all CRC cell lines tested and a total average EC₅₀ of 0.177 μM. When considering the implications of carrying a KRAS mutation on the anti-proliferative ability of Compound 12, there appeared to be very little difference between the eight cancer cell lines which are wild type KRAS (average EC₅₀=0.150 μM) and the nine cancer cell lines positive for the KRAS mutation (average EC₅₀=0.201 μM). As expected, the majority of CRC cancer cell lines were reported to have an APC mutation (72% or 13 out of the 17 cell lines) and the four cell lines carrying a CTNNB1 mutation (SW48, HuTu80, LS513, HCT 116) did not demonstrate overtly lower EC₅₀ with values ranging from 0.091 μM to 0.321 μM. Overall, Compound 12 showed promising inhibition of CRC cell growth across all investigated mutation types.

TABLE 7
Cell Viability EC₅₀ of Compound 12
in colorectal cancer cell lines

| | KRAS | Common | EC₅₀ | KRAS Status |
|---|---|---|---|---|
| Cell Line | status | Mutations | (μM) | EC₅₀ (μM) |
| COLO 320HSR | KRASwt | APCmut | 0.110 | 0.150 |
| C2BBel | | APCmut | 0.078 | |
| SW48 | | APCmut, | 0.091 | |
| | | CTNNB1mut | | |
| HuTu 80 | | CTNNB1mut | 0.178 | |
| COLO 205 | | BRAFmut, | 0.150 | |
| | | APCmut | | |
| SW1417 | | BRAFmut, | 0.282 | |
| | | APCmut | | |
| HT29 | | BRAFmut, | 0.189 | |
| | | APCmut | | |
| RKO | | BRAFmut | 0.120 | |
| HCT 15 | KRASmut | APCmut | 0.120 | 0.201 |
| SW620 | | APCmut | 0.087 | |
| DLD-1 | | APCmut | 0.303 | |
| LoVo | | APCmut | 0.194 | |
| LS123 | | APCmut | 0.472 | |
| T84 | | APCmut | 0.127 | |
| SW480 | | APCmut | 0.085 | |
| LS513 | | CTNNB1mut, | 0.321 | |
| | | BRAFmut | | |
| HCT 116 | | CTNNB1mut | 0.103 | |

| | |
|---|---|
| Average EC50 of all cell lines (uM) | 0.177 |

Effect of Compound 12 on cellular proliferation was measured using the CellTiter-Blue ® Cell Viability Assay. The effect of an 8-point dose titration of Compound 12 was assessed after 4-days of treatment. Each EC₅₀ represents 2-4 independent experiments performed in duplicate.

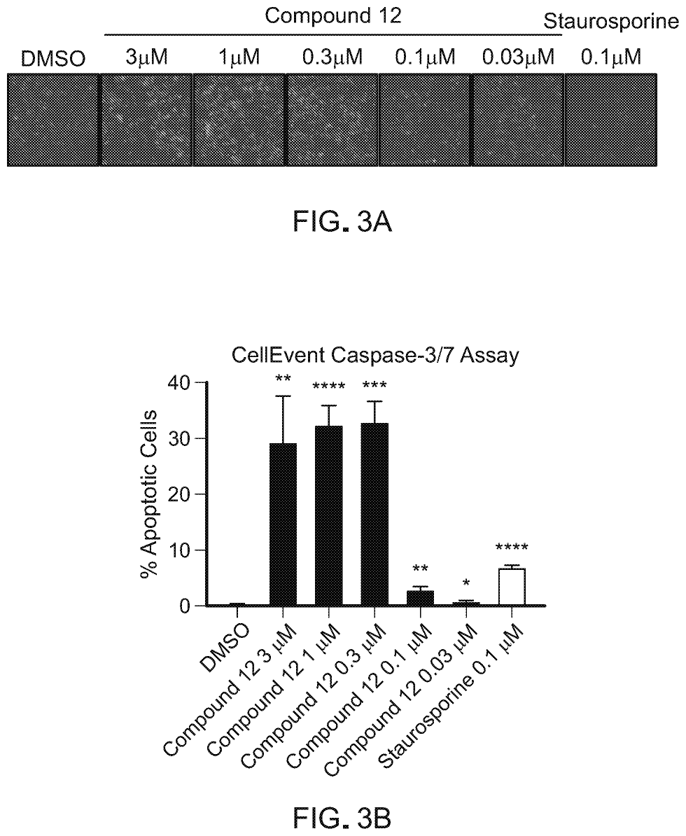

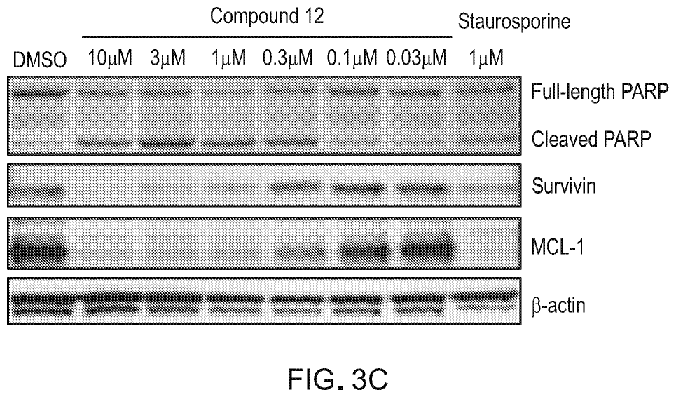

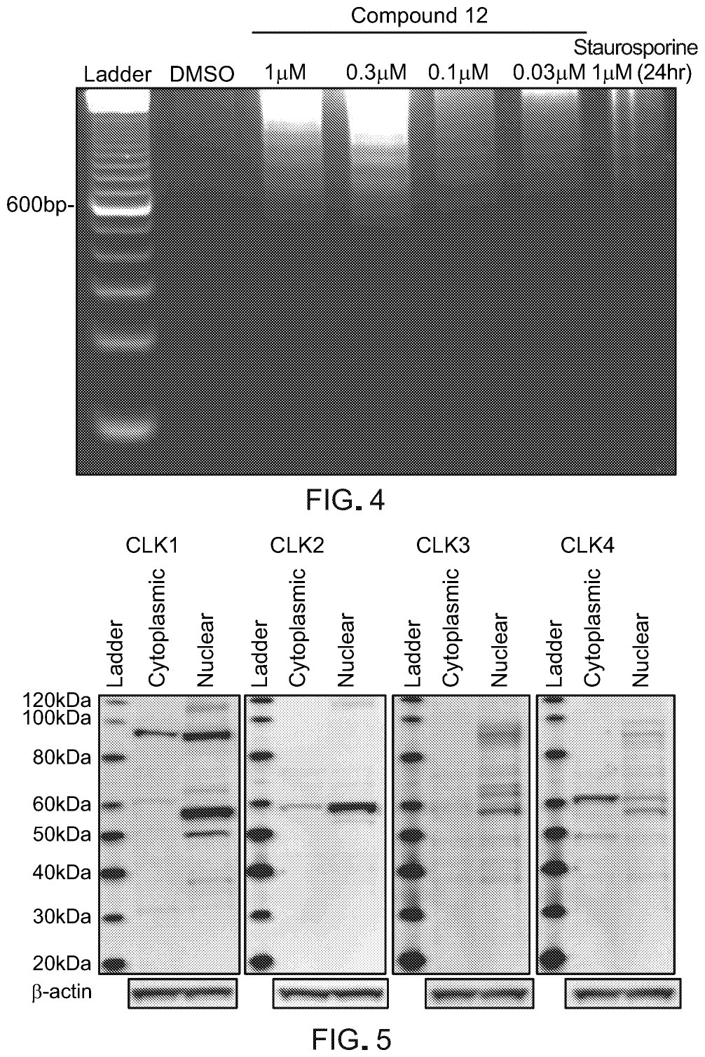

Induction of apoptosis or programmed cell death is an important mechanism by which anti-cancer drugs can exert activity. The ability of Compound 12 to regulate expression of anti-apoptotic proteins and induce apoptosis in SW480 CRC cells was also assessed. It was demonstrated that Compound 12 was a potent inducer of apoptosis as determined by assays to detect elevated activated caspase 3/7 (FIG. 3A, 3B), cleaved PARP (FIG. 3C), and DNA fragmentation (FIG. 4) in SW480 colorectal cancer cells. Additionally, Compound 12 appeared to inhibit protein expression of Survivin and MCL-1 which are both important inhibitors of apoptosis, with Survivin reported as a β-catenin target gene (FIG. 3C) (Ma et al., Oncogene 24:3019-3631, 2005; Yang et al., BMC Cancer 14:124, 2014). Taken together, this data suggests that Compound 12 has the potential to exert anti-tumor effects by inducing apoptosis in cancer cells.

To confirm that Compound 12 was functional on its primary targets, the effect of Compound 12 on SRSF phosphorylation in SW480 cells was evaluated. First, it was determined that CLK1, CLK2, and CLK3 localized into the nucleus, while CLK4 was predominantly detected in the cytoplasm (FIG. 5). The effects of Compound 12 on SR phosphorylation were then compared to Harmine, a selective DYRK1 kinase inhibitor (Gôckler et al., FEBS J 276, 6324-6337, 2009) and CC-671, a recently described CLK2/TTK kinase inhibitor that is being developed for the treatment of cancer (Riggs et al., J Med Chem 60, 8989-9002, 2017). Harmine was included to assess if DYRK1 inhibition contributes to SR phosphorylation since Compound 12 can also inhibit DYRK1 kinase activity (Table 8) and DYRK has the potential to also phosphorylate SRSF proteins (Wang et al., Nat Med 21, 383-388, 2015; Zhou et al., Chromosoma 122, 191-207, 2013).

TABLE 8
IC₅₀ of Additional Kinases
Inhibited by Compound 12

| | | Fold difference |
|---|---|---|
| Kinase Name | IC₅₀ (uM) | against CLK2 IC₅₀ |
| CLK4 | 0.001 | 0.5 |
| CLK2 | 0.002 | 1 |
| DYRK1A | 0.002 | 1 |
| DYRK1B | 0.002 | 1 |
| DYRK2 | 0.003 | 1.5 |
| MAP4K4 (HGK) | 0.007 | 3.5 |
| DYRK3 | 0.008 | 3.8 |
| CLK1 | 0.008 | 4 |
| MINK1 | 0.008 | 4 |
| DYRK4 | 0.013 | 6.5 |
| LRRK2 | 0.017 | 8.5 |
| LRRK2 FL | 0.019 | 9.5 |
| IRAK1 | 0.02 | 10 |
| CLK3 | 0.022 | 11 |
| AAK1 | 0.023 | 11.5 |
| MAP4K1 (HPK1) | 0.029 | 14.5 |
| MYLK4 | 0.03 | 15 |
| TAOK1 | 0.031 | 15.5 |
| BMPR1B (ALK6) | 0.033 | 16.5 |
| MAP3K7/MAP3K7IP1 | 0.037 | 18.5 |
| (TAK1-TAB1) | | |
| HIPK3 (YAK1) | 0.042 | 21 |
| STK17A (DRAK1) | 0.044 | 22 |
| HIPK2 | 0.046 | 23 |

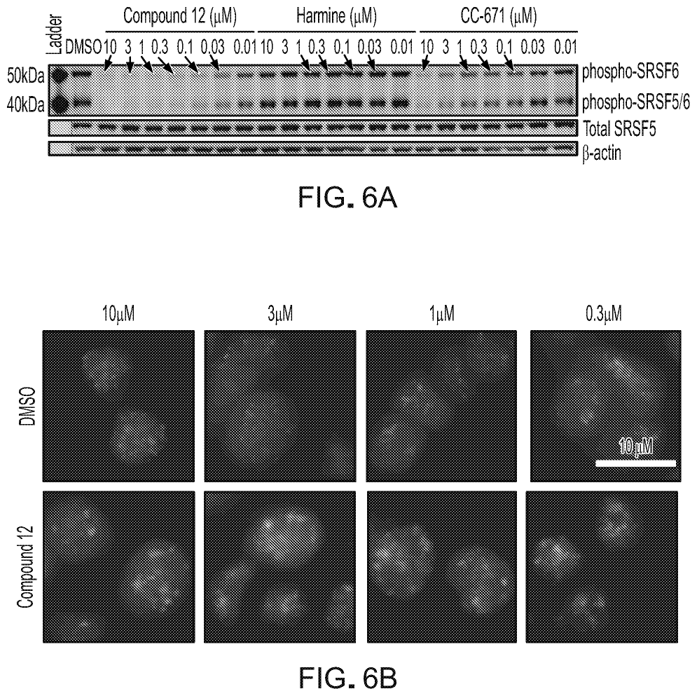

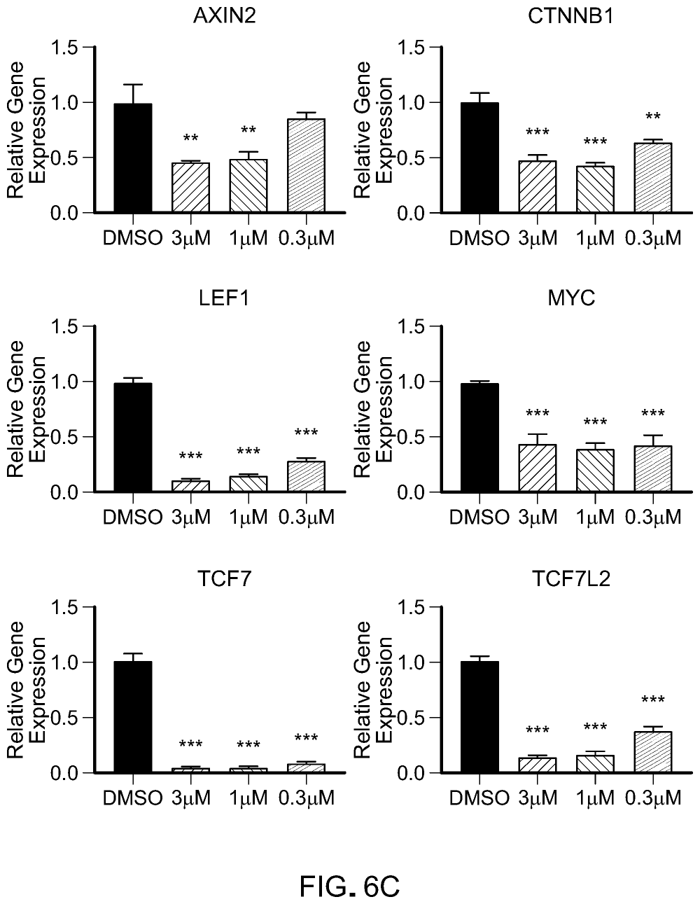

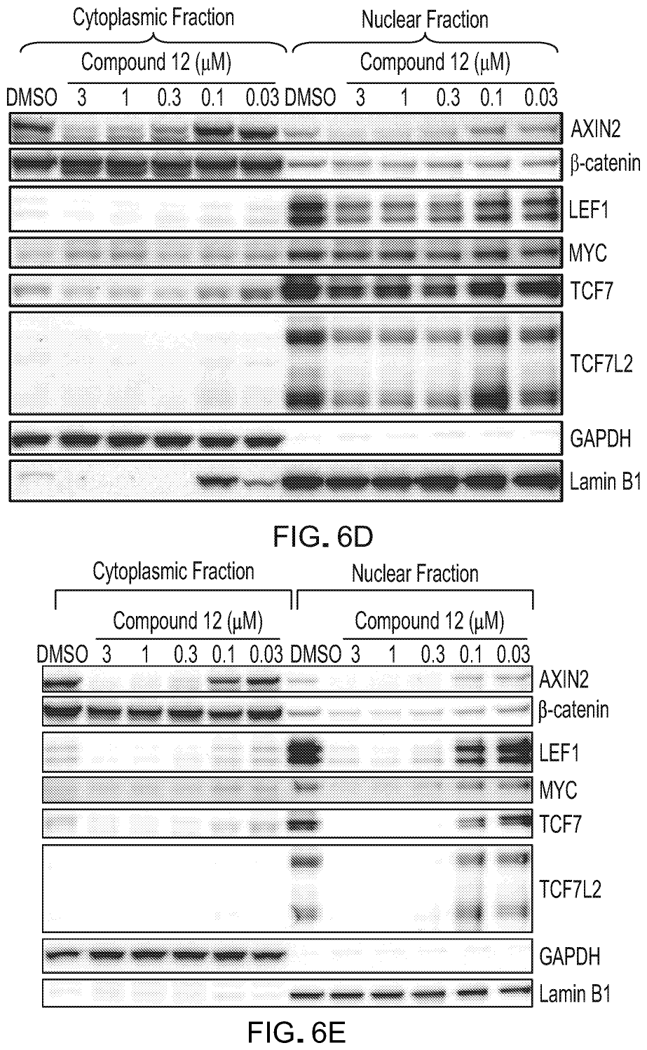

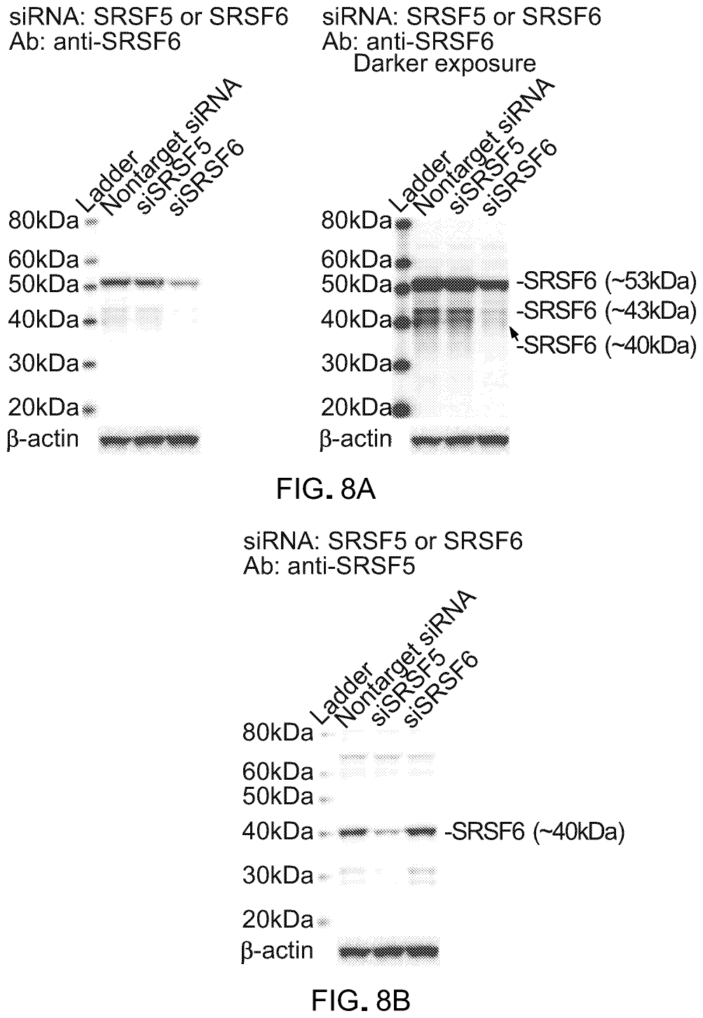

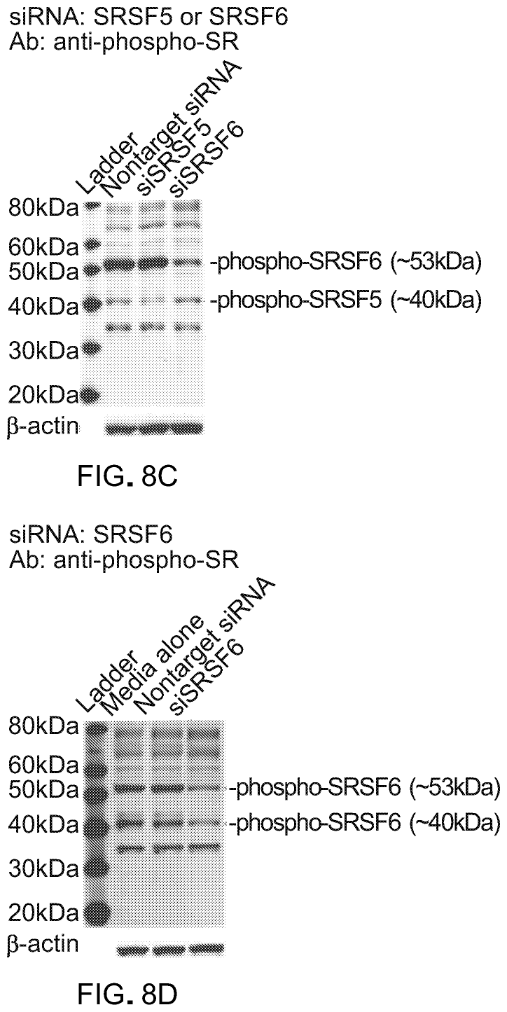

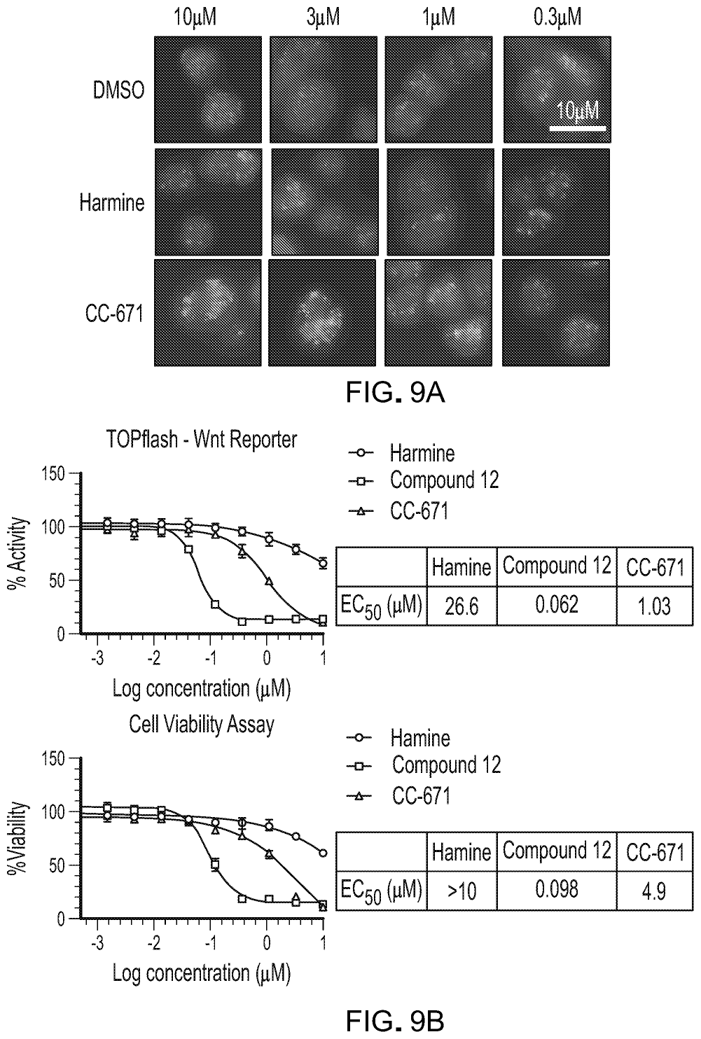

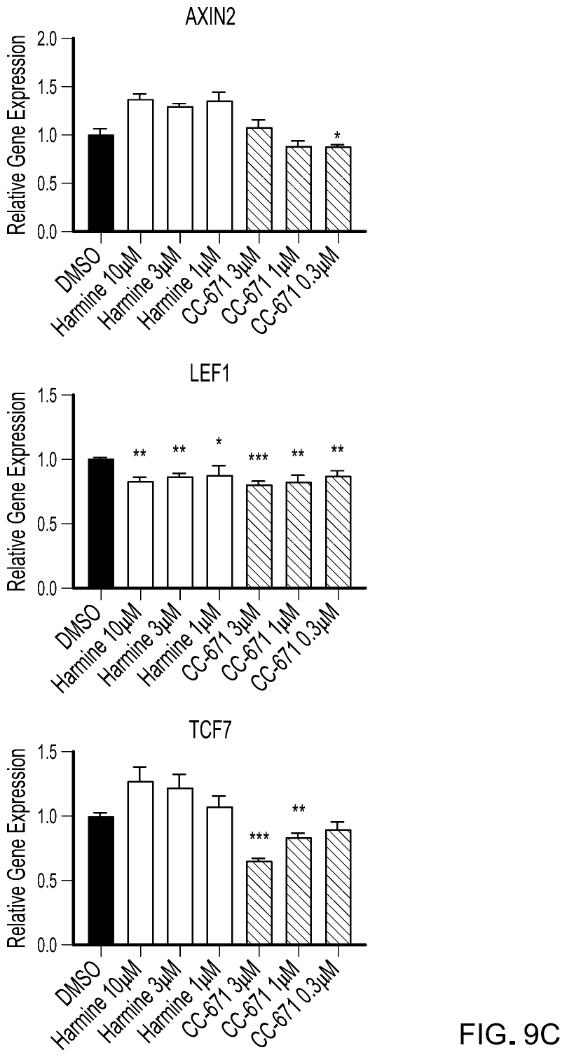

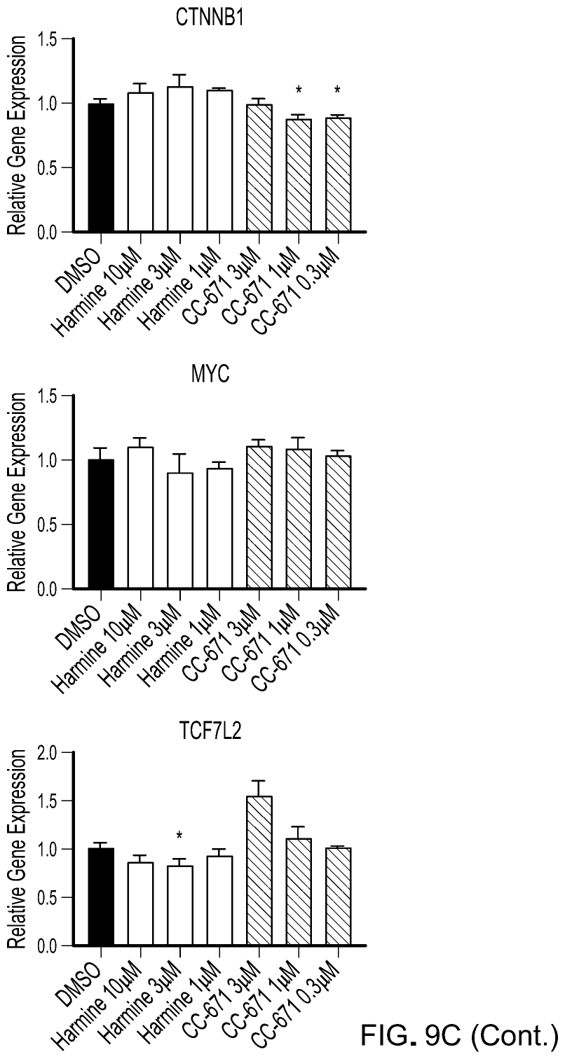

CC-671 represents a more selective CLK2 molecule that is reported not to inhibit CLK3 (Riggs et al., J Med Chem 60, 8989-9002, 2017). As shown in FIG. 6A, Compound 12 potently inhibited the phosphorylation of SRSF6 (also known as SRp55; top band at ˜53 kDa) and a lower band at ˜40 kDa. Additional siRNA studies suggest that this 40 kDa band could either SRSF5 (also known as SRp40) or a 40 kDa form of SRSF6 (FIG. 8A). In comparison, Harmine, a DYRK1 selective inhibitor was not active, while CC-671, a CLK2-selective inhibitor was not as effective as Compound 12 at inhibiting SR phosphorylation. Similar results were noted when effects on interchromatin granule clusters (IGCs), or ‘nuclear speckles’ were tested. These represent the predominant nuclear residence of SRSF proteins and can be identified as punctate, non-diffuse structures detected by immunofluorescence staining of SC35 (Lamond et al., Nat Rev Mol Cell Biol 4, 605-612, 2003). Functionally, speckles have a role in housing and supplying splicing factors to sites of active transcription which are adjacent to nuclear speckles. When transcription is blocked, splicing factors accumulate in the speckles and they appear enlarged O'Keefe et al., J Cell Biol 124, 249-260, 1994; Herbst et al., BMC Genomics 15, 74, 2014( ). After treatment with Compound 12, increased enlargement of nuclear speckles was observed at a range of doses (0.3-10 μM) (FIG. 6B), which was not observed with Harmine and only at higher doses of 10 μM and 3 μM with CC-671 (FIG. 9A). Additionally, Harmine was inactive in TOPflash Wnt reporter and cell viability assays in SW480 cells (EC₅₀=>10 μM), while CC-671 was 17-fold and 50-fold less potent than Compound 12 in the TOPflash reporter assay and cell viability assays, respectively (FIG. 9B). Inhibition of important Wnt pathway gene and protein expression was confirmed with Compound 12. Compared to DMSO-treated cells, there was a dose-dependent decrease in the gene expression of known Wnt target genes (AXIN2, LEF1, MYC, TCF7) (Herbst et al., BMC Genomics 15, 74, 2014) and other key genes such as CTNNB1 and TCF7L2 (FIG. 6C). There was minimal effect on these genes when treated with Harmine or CC-671 (FIG. 9C). Subsequent inhibition of protein expression by Compound 12was demonstrated in cytoplasmic or nuclear fractions after 24 (FIG. 6D) or 48 hours of treatment (FIG. 6E) for all tested proteins except for β-catenin. Together, these data suggested that Compound 12-mediated inhibition of additional CLK kinases such as CLK3 resulted in stronger inhibition of SR-phosphorylation, Wnt reporter activity and Wnt pathway gene expression, compared to CC-671, a CLK2-selective small molecule kinase inhibitor.

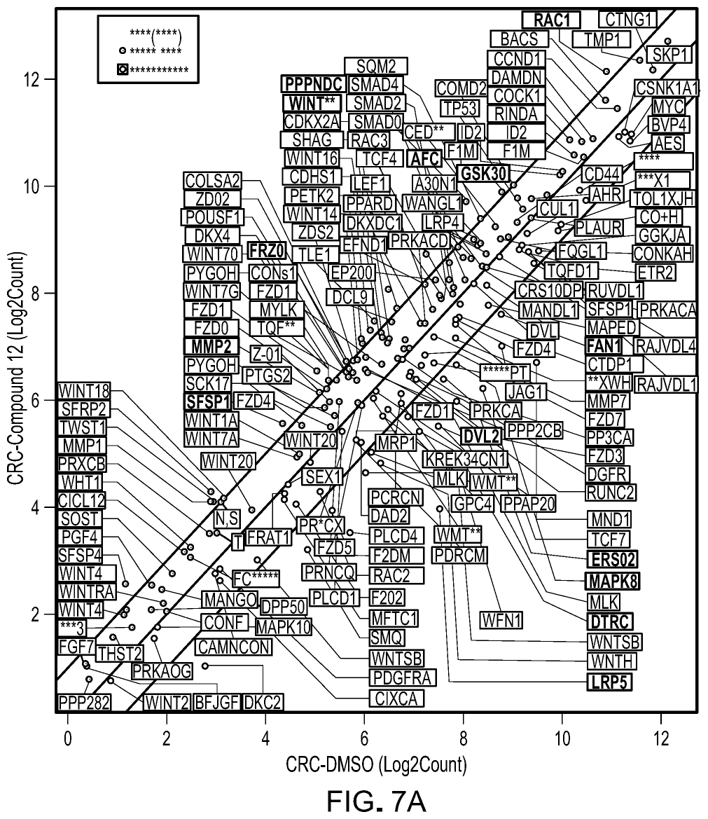

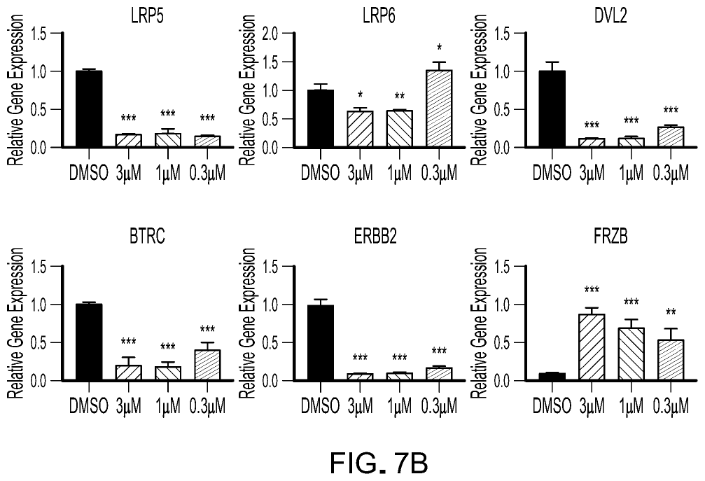

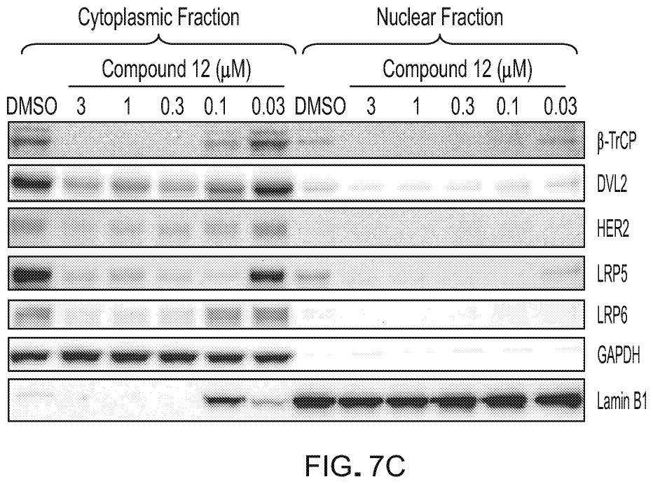

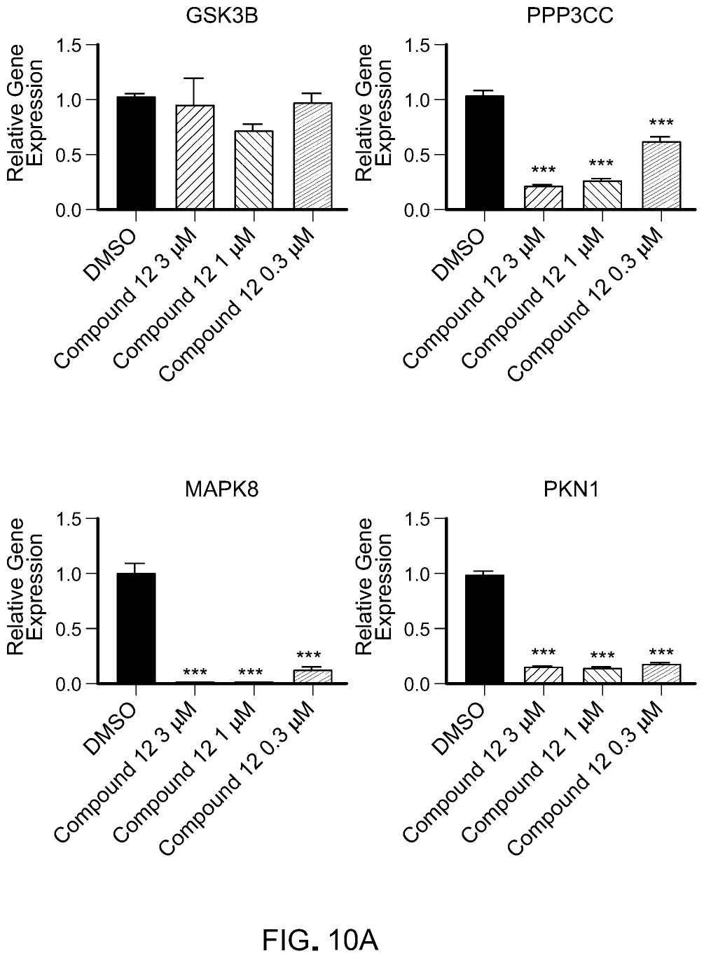

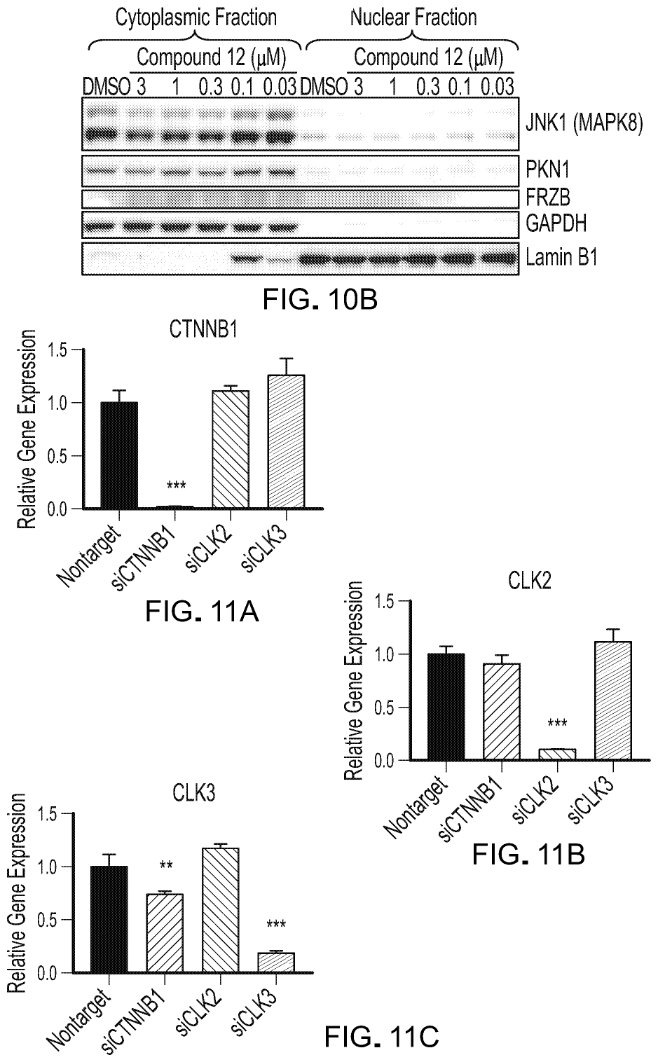

The effects of 24 hr treatment with Compound 12 (1 μM) on 180 Wnt pathway genes represented in Nanostring's nCounter® Vantage 3D™ Wnt Pathways across a panel of 17 CRC cell lines (from Table 1) was evaluated. Those genes which demonstrated greater than 2-fold statistically significant changes from baseline were then tested in SW480 cells (highlighted green in FIG. 7A). The gene expression of LRP5, DVL2, BTRC, ERRB2, MAPK8, PKN1 were significantly downregulated compared to DMSO-treated controls (FIG. 7B & FIG. 10A). There was no effect detected in GSK3/p and an inhibitory effect, instead of an increase was noted for PPP3CC expression in SW480 cells (FIG. 10A). SFRP1 and WNT9B expression levels were too low to detect in SW480 cells. Upregulation of FRZB gene expression a described inhibitor of Wnt signaling (Clevers et al., Cell 149, 1192-1205, 2012) was confirmed, but protein expression was not detectable by immunoblotting (FIG. 10). Since LRP5 expression was downregulated, the effect on LRP6, another important co-receptor of Wnt ligands, was tested. The gene and protein expression of LRP6 were indeed inhibited, but not as potently as LRP5 (FIG. 7B & FIG. 7C). Relative to DMSO-treated cells, pronounced dose-dependent inhibition of protein expression was confirmed for LRP5, LRP6, DVL2, R-TrcP and HER2, with LRP5 appearing to be the most sensitive with complete inhibition observed at 0.1 μM (FIG. 7C). However, inhibitory effects of Compound 12 on MAPK8 and PKN1 protein expression compared to vehicle-treated cells was less profound (FIG. 10B).

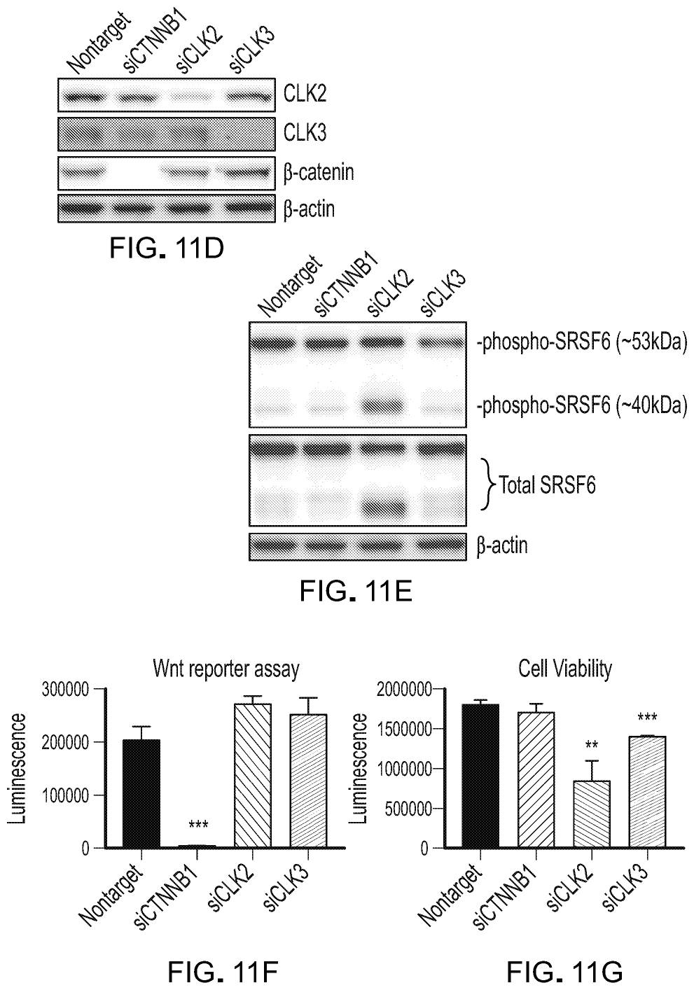

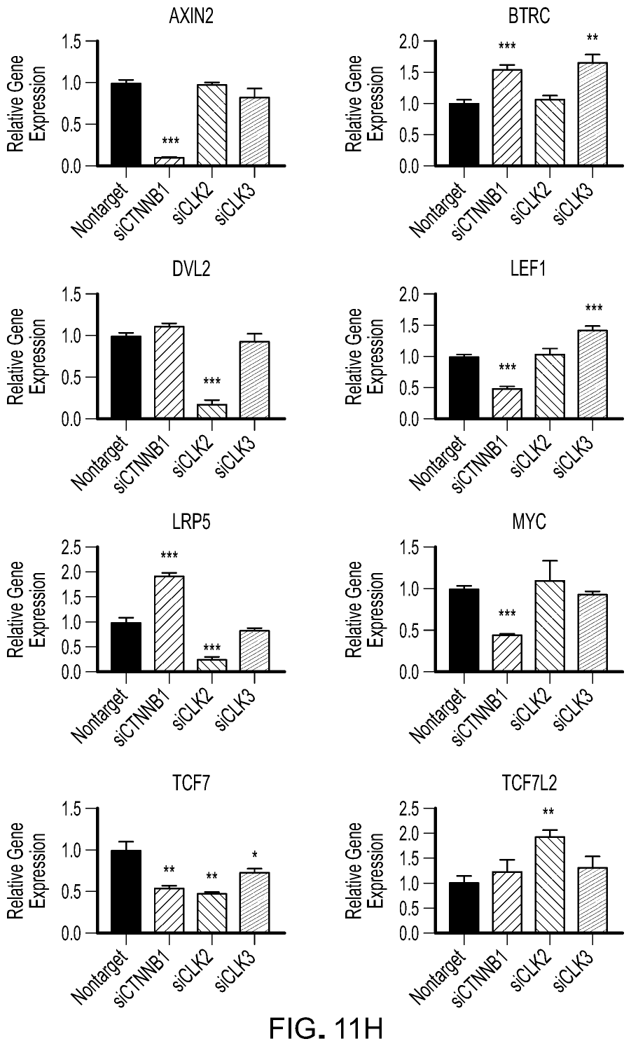

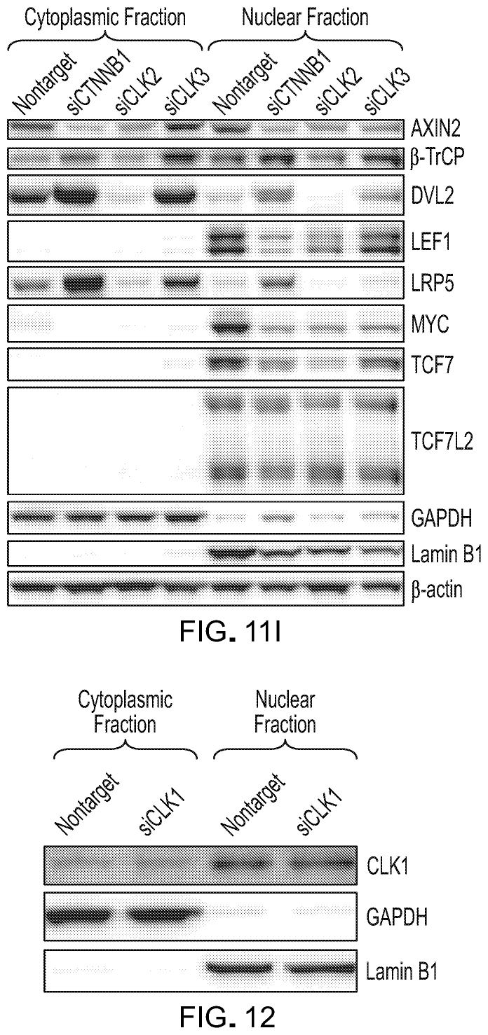

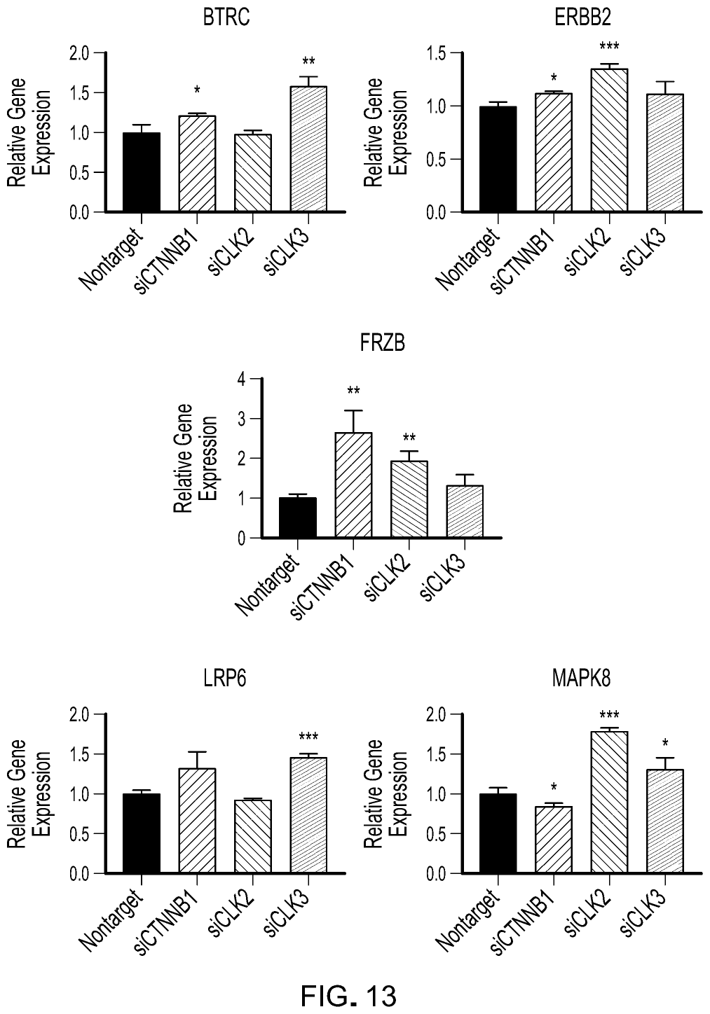

To characterize the effects of individual knockdown, the effects of CTNNB1, CLK2 and CLK3 knockdown on Wnt reporter activity, cell viability, SR-phosphorylation and Wnt pathway gene expression in SW480 cells were compared. CLK1 knockdown was attempted utilizing different siRNAs but proved unsuccessful (FIG. 12). In contrast, >80% knockdown of CTNNB1, CLK2 and CLK3 was achieved (FIG. 11A, 11B & FIG. 11C), which corresponded with a loss of the target protein expressions (FIG. 11D). Upon examination of the effects on SR phosphorylation, there was minimal impact on phospho-SRSF6 with knockdown of CTNNB1 compared to nontarget controls. Upon silencing of CLK2, an increased detection of phosphorylated of ˜40 kDa form of SRSF6 was observed, which is suggestive of the ability of other CLK isoforms to maintain SR phosphorylation in the absence of CLK2 (Prasad et al., Mol Cell Biol 19, 6991-7000, 1999). Evaluation of the total SRSF6 immunoblot detected presence of the unphosphorylated ˜40 kDa isoform compared which is not present in the nontarget control (FIG. 11E). This suggests that not only was there increased phosphorylation of the ˜40 kDa SRSF6 band, but it appears that its protein expression was increased. This is likely explained by the role CLK2 has in alternative splicing and that the loss of CLK2 resulted in the increased formation of the ˜40 kDa SRSF6 isoform (Duncan et al., Exp Cell Res 241, 300-308, 1998; Yoshida et al., Cancer Res 75, 1516-1526, 2015). Upon knockdown of CLK3, the increased presence of the 40 kDa form was not evident, but there was a moderate effect on the phosphorylation of the ˜53 kDa form of SRSF6. Expectedly, the knockdown of p-catenin led to near complete loss of Wnt reporter activity which was not seen with the individual silencing of CLK2 and CLK3, therefore suggestive that inhibition of more than one CLK may be necessary to exert profound effects on Wnt reporter activity in SW480 cells. Assessment of cell viability 5-days post-transfection revealed no effects from CTNNB1 knockdown and modest effects from silencing CLK2 and CLK3 (FIG. 11G). The lack of effects of CTNNB1 knockdown on cell viability has been observed previously in other CRC lines where additional mutational burden besides activated Wnt signaling contributed to cancer cell viability (Kim et al., Mol Cancer Ther 1, 1355-1359, 2002). Upon analysis of Wnt pathway gene expression, CTNNB1 knockdown had an effect on known Wnt-target genes such as AXIN2, LEF1, MYC and TCF7 (Nusse and Varmus, EMBO J 31, 2670-2684, 2012; Herbst et al., BMC Genomics 15, 74, 2014) (FIG. 11H). However, knockdown of CLK2 and CLK3 did not recapitulate the downregulation of AXIN2, MYC or LEF1 expression which was similar to that observed in the TOPflash assay. For TCF7, there appeared to be a direct effect on gene expression under all conditions. In contrast, none of the knockdowns affected TCF7L2 expression, while CLK2 silencing resulted in the inhibition of LRP5 and DVL2. In contrast, loss of CTNNB1 resulted an apparent increase in LRP5 and DVL2 protein levels. CLK3 silencing did not appear to affect DVL or LRP5 protein levels, while TCF7 was moderately inhibited, similar to what was observed in cells depleted of CTNNB1. Protein expression of cytoplasmic AXIN2 and nuclear LEF1 were not only inhibited with CTNNB1 siRNA, but appeared downregulated under CLK2 and knockdown, suggesting potential regulation at the post-transcriptional level mediated by CLK2 which is lost upon silencing. There was an apparent loss of MYC protein under both CLK2 and CLK3 silencing which was comparable to the decrease observed under CTNNB1 knockdown conditions. No inhibitory effects on expression of other Wnt pathway genes such as BTRC, ERBB2, FRZB, LRP6 and MAPK8 were observed (FIG. 13).

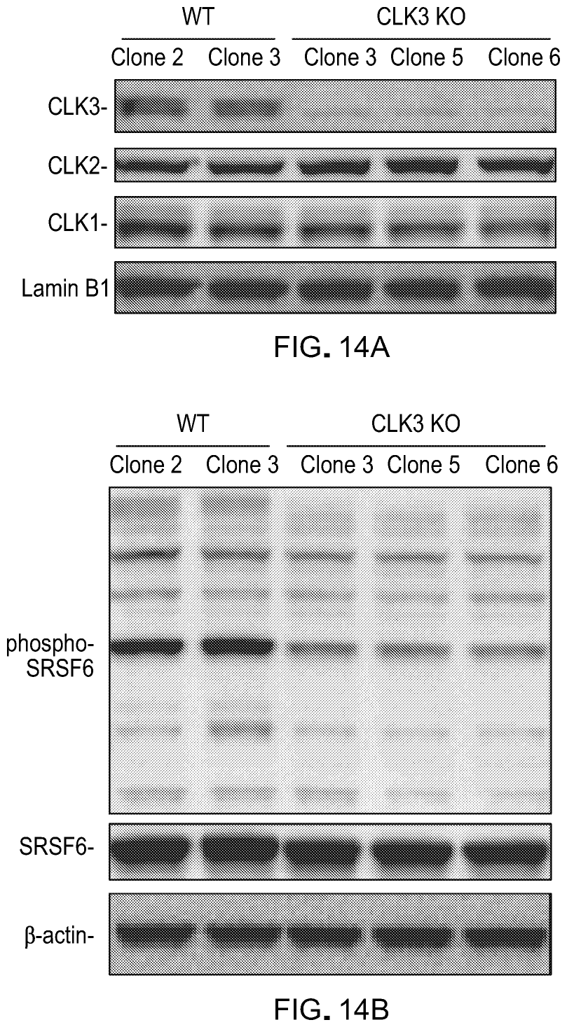

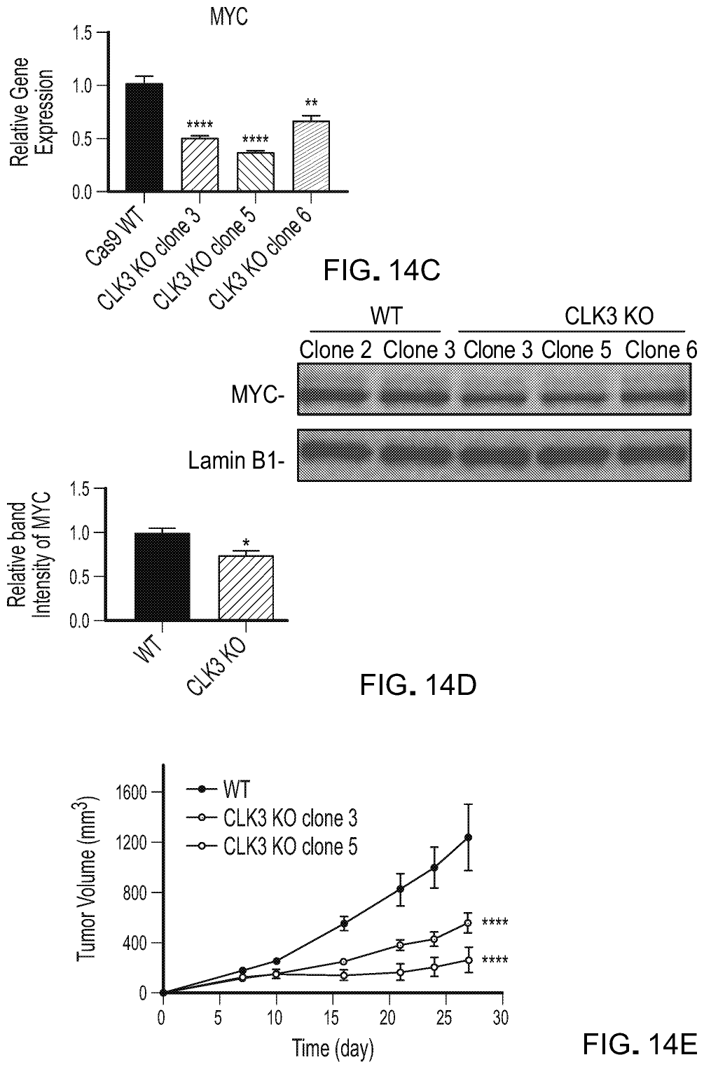

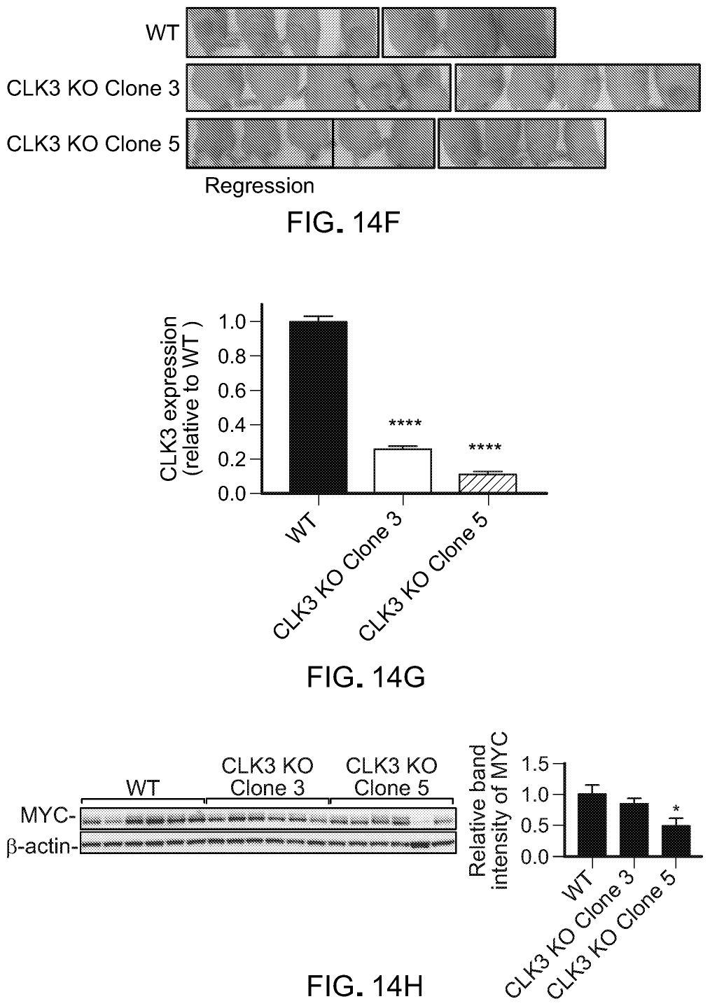

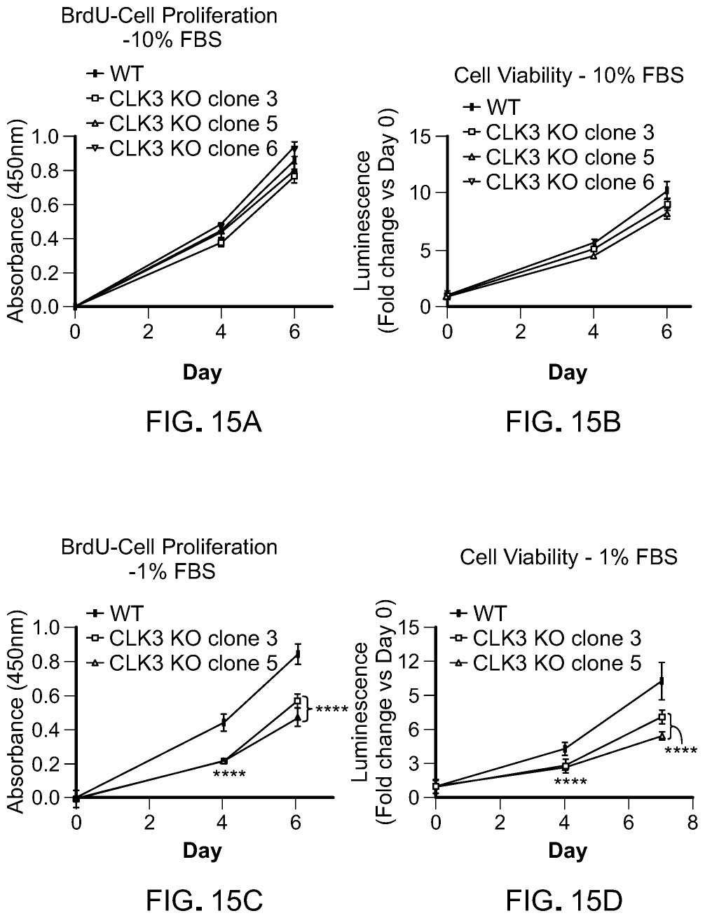

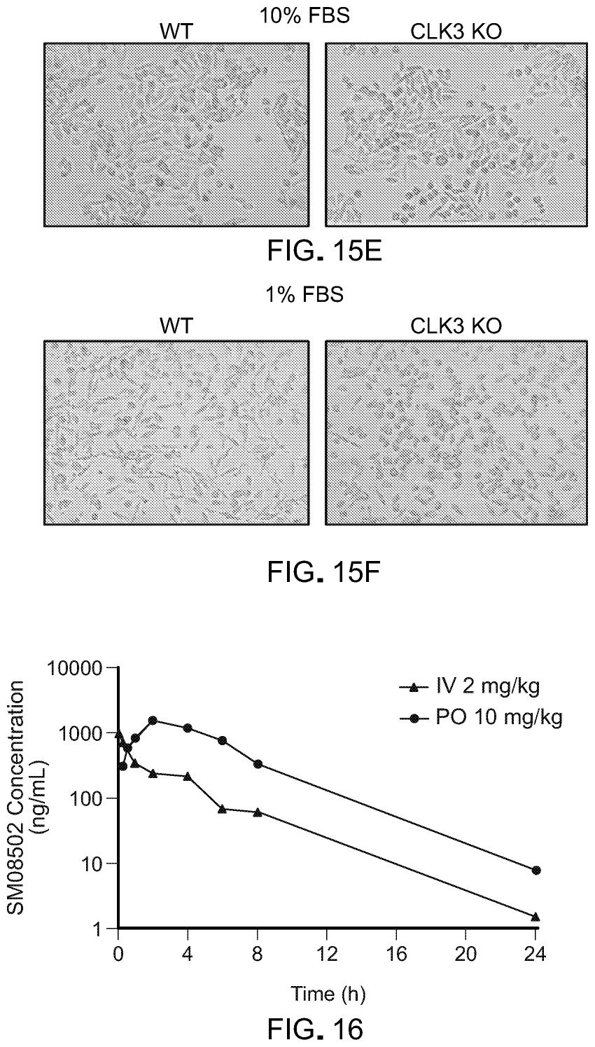

To investigate the therapeutic importance of CLK3 as an oncology target in colon cancer, a stable CLK3 knockout (KO) SW480 cancer cell line by CRISPR (Jinek et al., Science 337, 816-822, 2012) was generated. As shown in FIG. 14, there were two wild type (WT) clones (clone 2 and clone 3) and three CLK3 KO clones (clone 3, clone 5 and clone 6) generated. Compared to the WT clones, CLK3 clones, demonstrated significant loss of CLK3 protein, with no observed effect on CLK2 or CLK1 protein levels (FIG. 14A FIG. 14B). Examination of the effects on SR phosphorylation demonstrated inhibition of SRSF6 (˜53 kDa), suggesting CLK3 may have a role in preferentially phosphorylating this SRSF6 isoform as was seen in the siRNA CLK3 knockdown studies. A significant decrease in MYC gene expression in CLK3 knockout clones (FIG. 14C) was observed, which correlated with a moderate decrease in protein levels compared to WT clones (FIG. 14D). The effects of the CLK3 KO appeared to be minimal on cell proliferation when cultured in 10% FBS (FIG. 15A, FIG. 15B FIG. 15E), but a more pronounced effect became evident when the cells were cultured in low serum conditions mimicking the low nutrient conditions characterized by tumors (Heinecke et al., Proc Natd Acad Sci USA 111, 6323-6328, 2014) (FIG. 15C, FIG. 15D & FIG. 15F). This also appeared to be the case when CLK3 KO cells were implanted to assess the effect on in vivo tumor growth. As shown in FIG. 14E & FIG. 14F, the growth of CLK3 KO tumors were significantly impacted compared to wild type (55% inhibition in CLK3 KO clone 3 and 80% inhibition in CLK3 KO clone 5), with tumor regressions observed for 3 out of 9 of mice bearing CLK3 KO clone 5 (FIG. 14F). At the end of study (day 28 post-implant), CLK3 gene expression remained inhibited in the tumors with a higher percent knockdown observed in CLK3 KO clone 5 (90% inhibition) compared to CLK3 KO clone 3 (75% inhibition). This appeared to correlate the degree of tumor growth inhibition observed for each clone (FIG. 14G). Analysis of MYC protein demonstrated a modest decrease in CLK3 KO clone 3 with a more obvious and significant decrease in protein expression in CLK3 KO clone 5, as determined by densitometry (FIG. 14H). Together, these data supported the therapeutic potential of targeting CLK3 as an oncology target, and its importance in phosphorylating SRSF6. Additional work is required to assess the potential impact on other signaling pathways.

Prior to in vivo efficacy studies, pharmacokinetic studies were performed with Compound 12. After oral administration of a single dose, Compound 12 (10 mg/kg) exhibited low clearance and an estimated oral bioavailability of 91% (FIG. 16, Table 9).

TABLE 9
Mean Plasma Pharmacokinetic Parameters Following a Single Intravenous (IV)
Bolus or Oral (PO) Dose of Compound 12 to Male Balb/c Mice

| | | | | C₀ or | | | | | | |
|---|---|---|---|---|---|---|---|---|---|---|
| | Dose | t_{1/2} | tₘₐₓ | Cₘₐₓ | tₗₐₛₜ | AUC₍₀₋ₗₐₛₜ₎ | AUC_{(0-inf)} | CL | V_{SS} | |
| Route | (mg/kg) | (h) | (h) | (ng/mL) | (h) | (h · ng/mL ) | (h · ng/mL) | (mL/min/kg) | (L/kg) | % F |
| IV | 2 | 3.15 | NA | 1122 | 24.0 | 2270 | 2276 | 14.6 | 3.29 | NA |
| PO | 10 | 2.76 | 2.00 | 1547 | 24.0 | 10303 | 10334 | NA | NA | 91 |

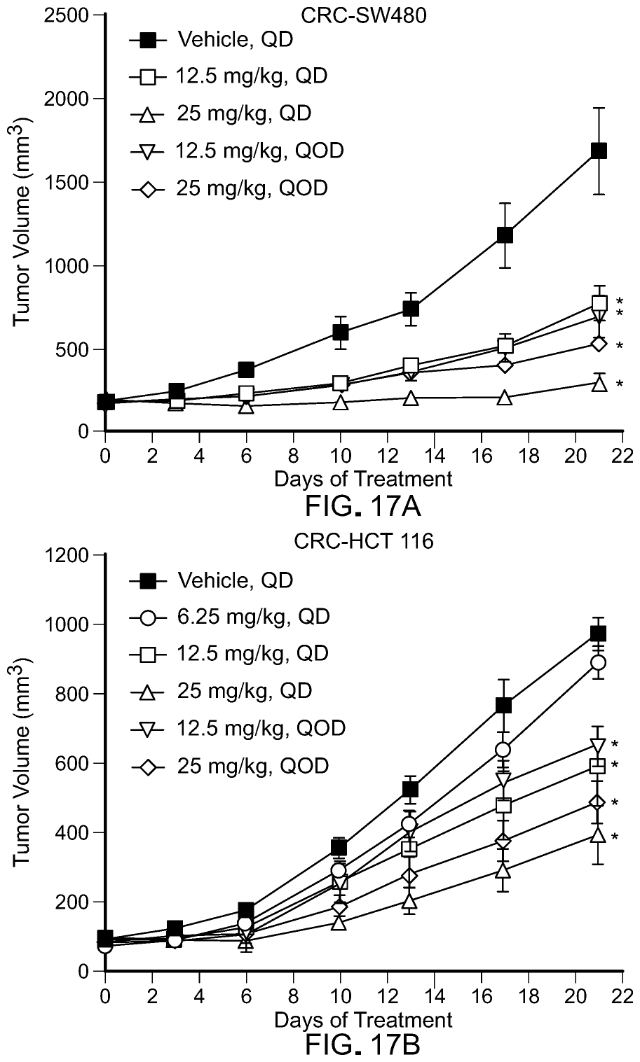

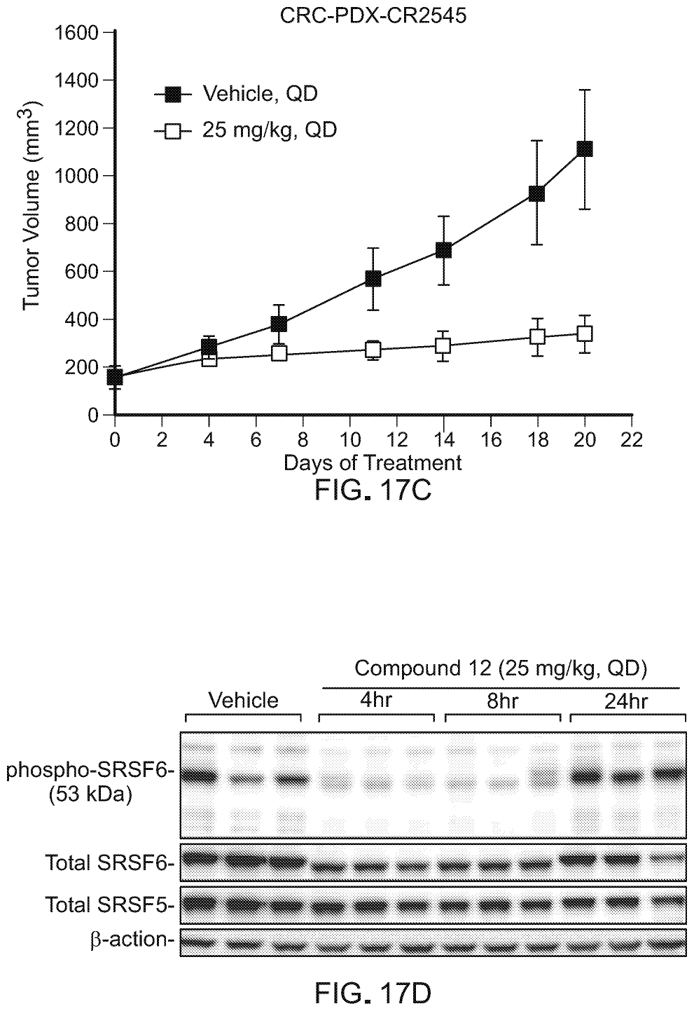

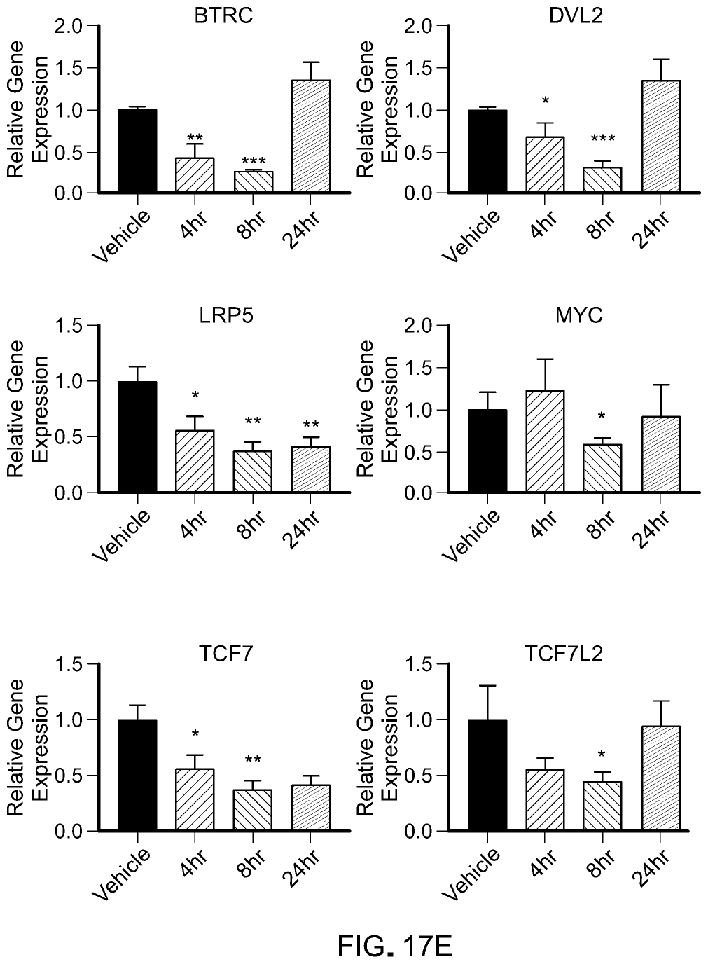

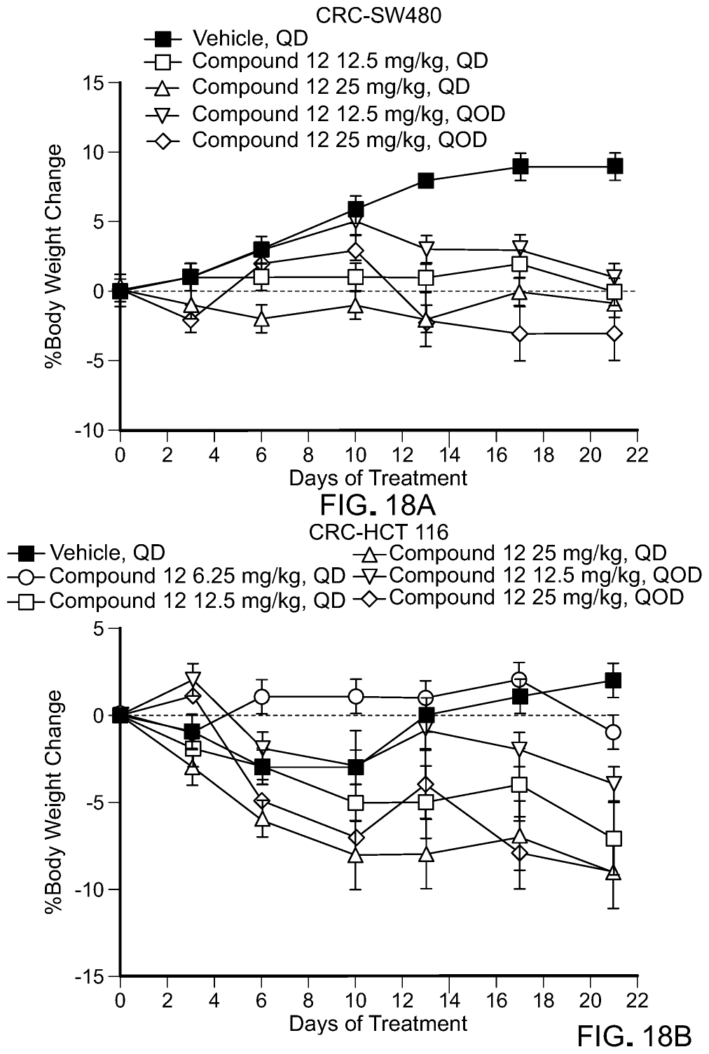

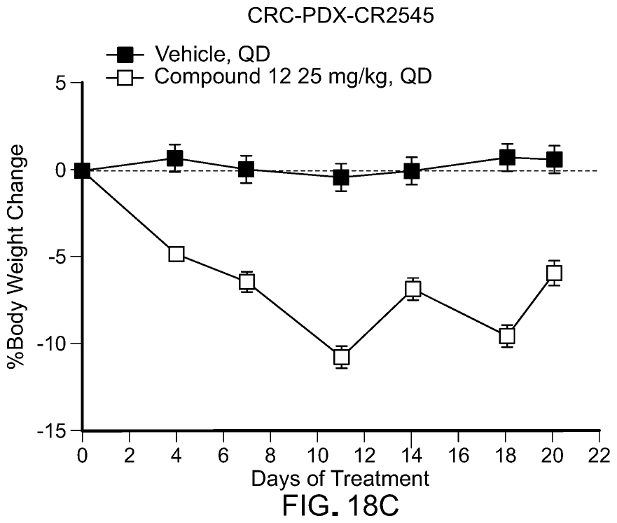

Subsequently, the effect of Compound 12 on SW480 tumor-bearing athymic nude mice was evaluated. Oral administration of Compound 12 at indicated frequencies was initiated when tumors were approximately 100-200 mm³ (FIG. 17A-E). When dosed daily at 25 mg/kg, QD, Compound 12 achieved 83% tumor growth inhibition (TGI) of SW480 tumors relative to the vehicle group (FIG. 17A). The remaining dose groups also demonstrated significant tumor growth inhibition responses, all which were >50% TGI compared to vehicle-treated mice. When dosed every other day at 25 mg/kg QOD, Compound 12 mediated tumor growth inhibition with 69% TGI, which suggested that an intermittent dosing regimen with Compound 12 had the potential to be efficacious. In comparison, the 12.5 mg/kg QD group achieved a lower TGI of 55%. Despite being dosed every other day, 12.5 mg/kg QOD dose demonstrated similar efficacy to the 12.5 mg/kg QD group with a relative TGI of 60%. Similar observations seen with the Compound 12-treated HCT 116 tumor-bearing mice, except that a 6.25 mg/kg, QD group was also included in this study and did not show any significant TGI relative to vehicle. The effect of Compound 12 was also evaluated in a patient-derived xenograft (PDX) model of CRC carrying a CTNNB1 mutation (Crown Biosciences HuPrime® Model #CR2545). As shown in FIG. 17C, Compound 12, produced significant anti-tumor response (69.8% TGI) compared with vehicle treatment. For all studies, all doses were well-tolerated, with acceptable body weight changes (<−10%) compared to baseline by end of the studies (FIGS. 18A-C).

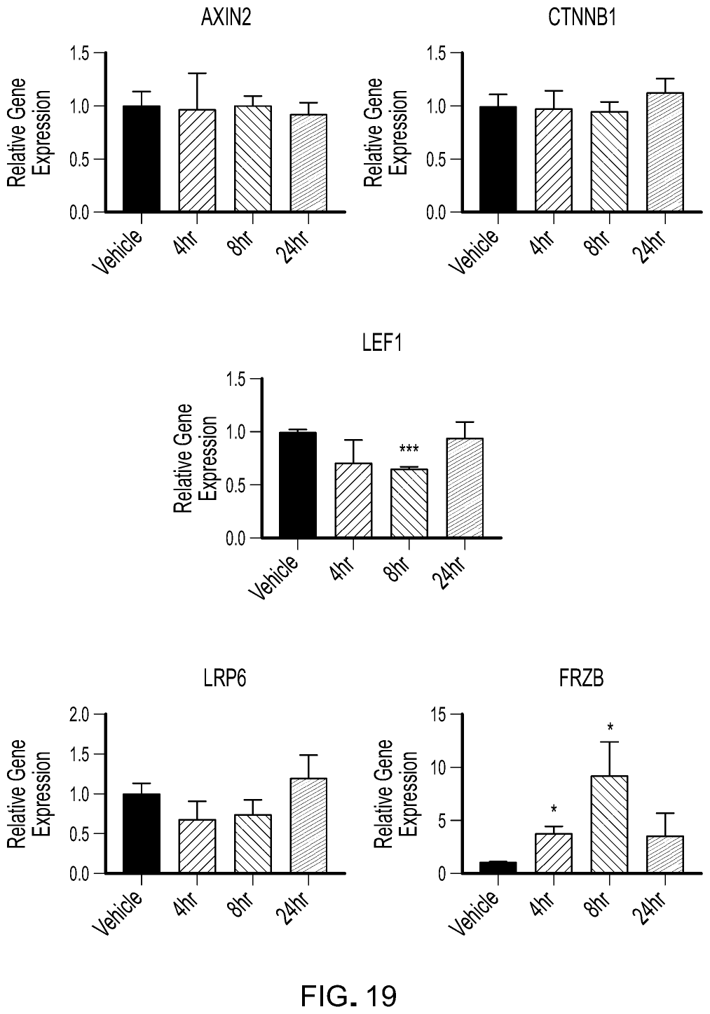

A tumor pharmacodynamic study was performed in athymic nude mice bearing SW480 tumors. After a single dose of Compound 12, tumors were harvested at 4, 8, and 24 hours after dosing. As shown in FIG. 17D, Compound 12 inhibited SR phosphorylation at 4 and 8 hours compared to vehicle treated tumors. By 24 hours, SR phosphorylation was comparable to vehicle, which is explained by the clearance of the compound by this timepoint (FIG. 16, Table 4). Analysis using qRT-PCR of Wnt pathways genes was performed on RNA extracted from the SW480 tumors and confirmed Compound 12 caused significant inhibition of certain Wnt pathway genes compared to vehicle over the time course (TCF7L2, TCF7, MYC, LRP5, DVL2 and BTRC) (FIG. 17E). There were no apparent changes in AXIN2, CTNNB1, LEF, LRP6 gene expression, while an increase in the Wnt pathway inhibitor, FRZB was observed at 4 hr and 8 hr (FIG. 19).

The effect of Compound 12 on cell proliferation were also determined in six gastric cancer (GC) cell lines carrying different mutations (Table 10).

TABLE 10
EC₅₀ of Compound 12 in Gastric Cancer Cell
Lines Carrying Different Mutations

| Cell Line | Mutation | EC₅₀(μM) | SEM |
|---|---|---|---|
| KATO III | TP53 | 0.447 | 0.003 |
| NCI-N87 | TP53, SMAD4, | 0.017 | 0.001 |
| | HER2 amplification | | |
| SNU-16 | TP53, CDKN2A | 0.109 | 0.0002 |
| SNU-5 | TP53, CDKN2A, CDH1 | 0.280 | 0.046 |
| AGS | CDH1, CTNNB1, | 0.185 | 0.034 |
| | KRAS, PIK3CA | | |
| SNU-1 | KRAS, MLH1 | 0.031 | 0.008 |

| | |
|---|---|
| Average EC50 of all cell lines | 0.178 |

This panel of gastric cancer cell lines were obtained from ATCC:
www.atcc.org/~/media/4B8544B854484A098F301F27E6E3B628.ashx
Effect of Compound 12 on cellular proliferation was measured using the CellTiter-Blue ® Cell Viability Assay. The effect of an 8-point dose titration of Compound 12 was assessed after 4-days of treatment. Each EC₅₀ represents 2-4 independent experiments performed in duplicate.

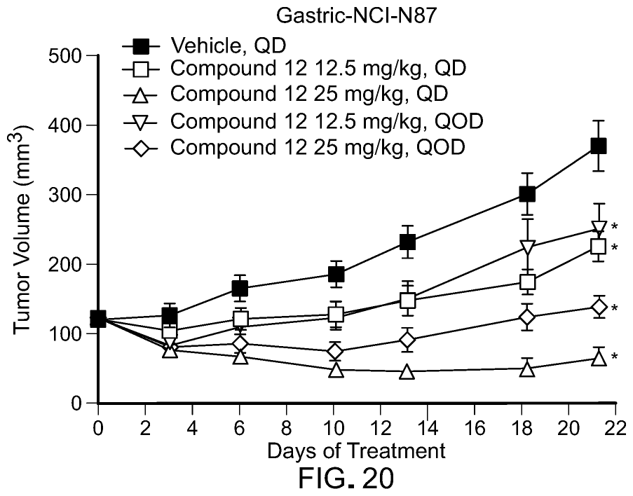

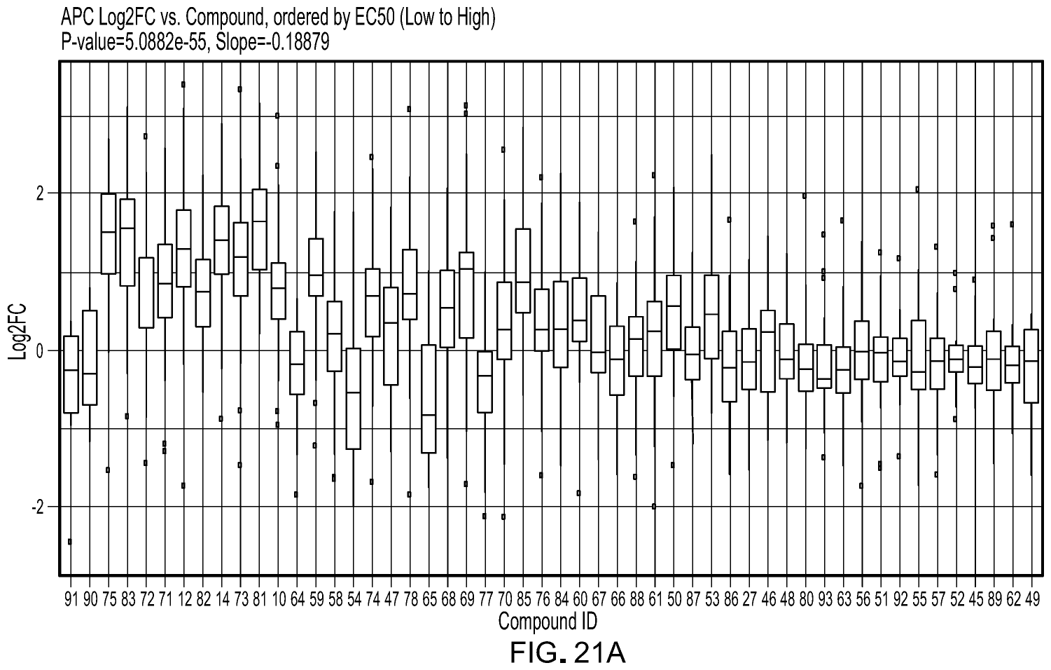

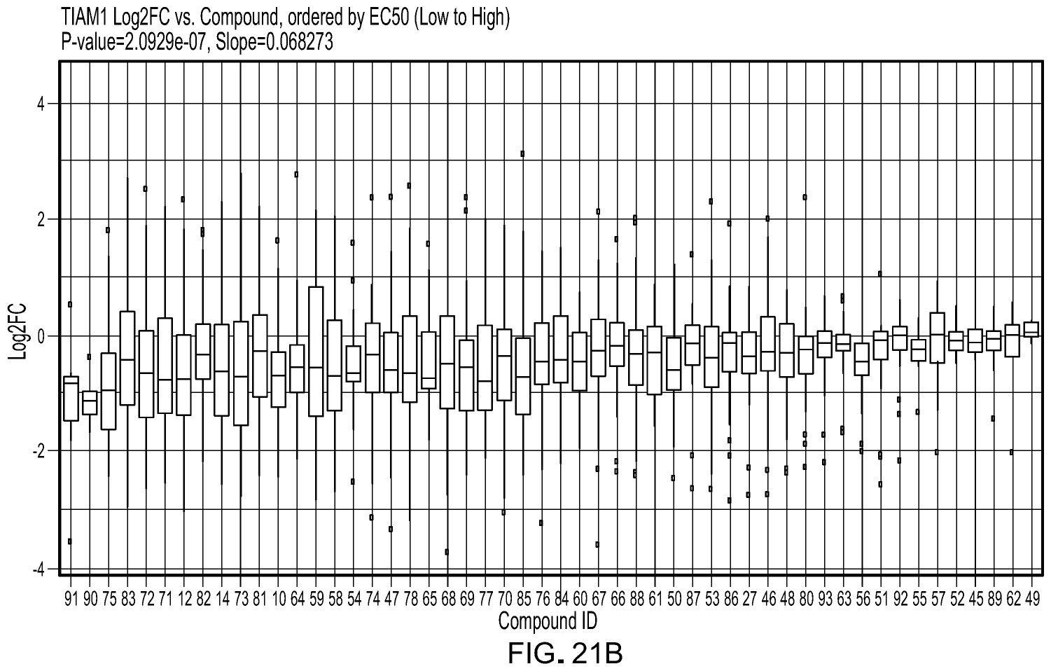

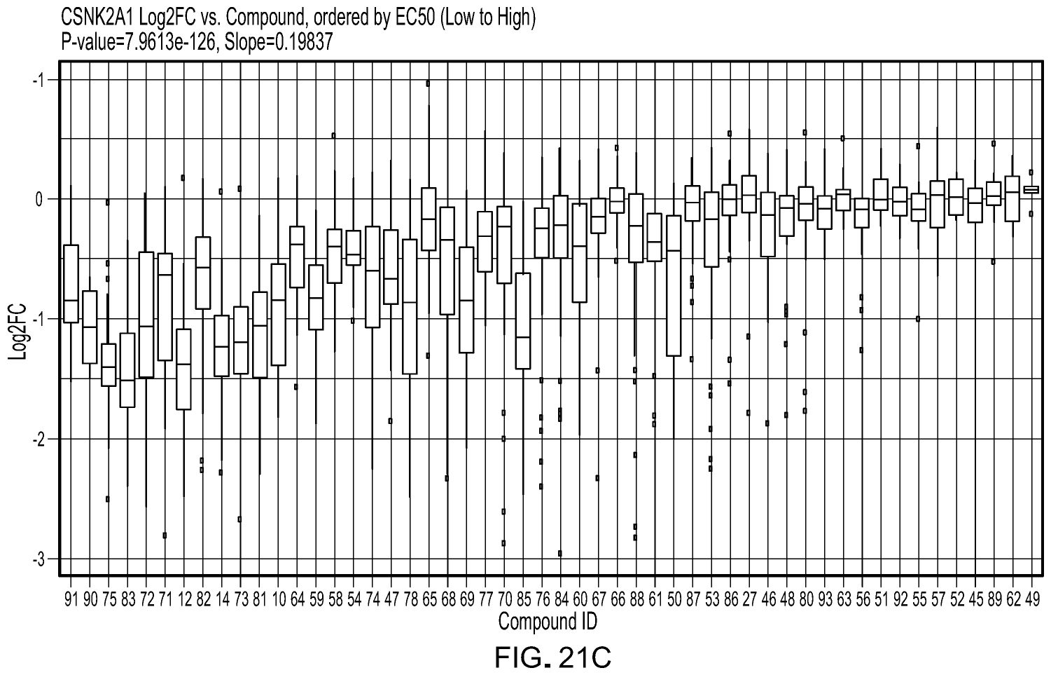

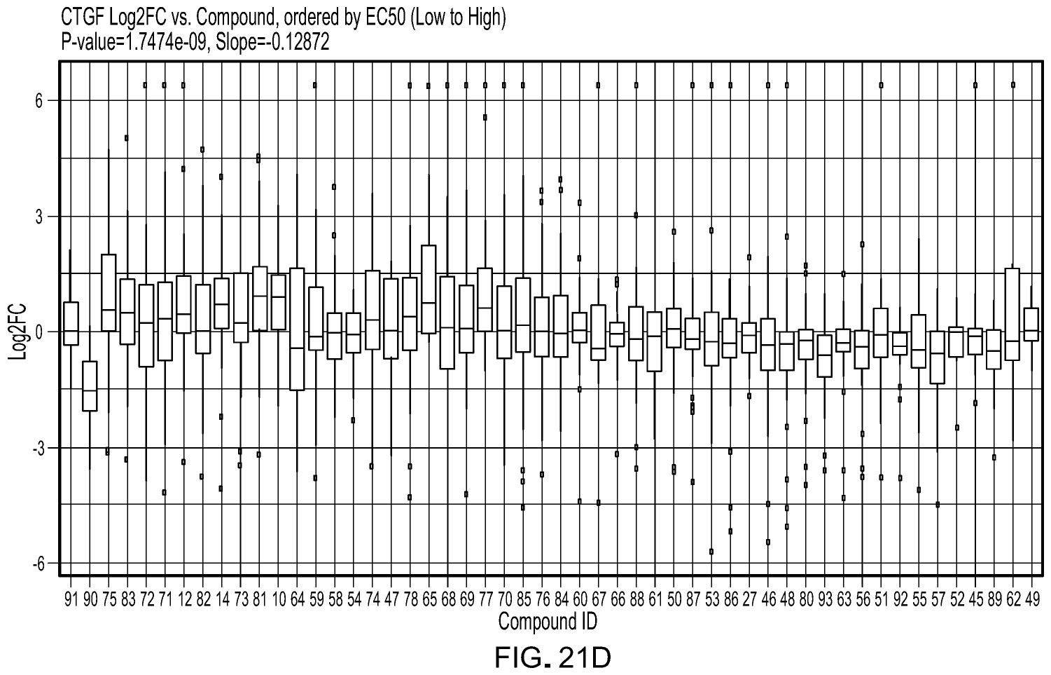

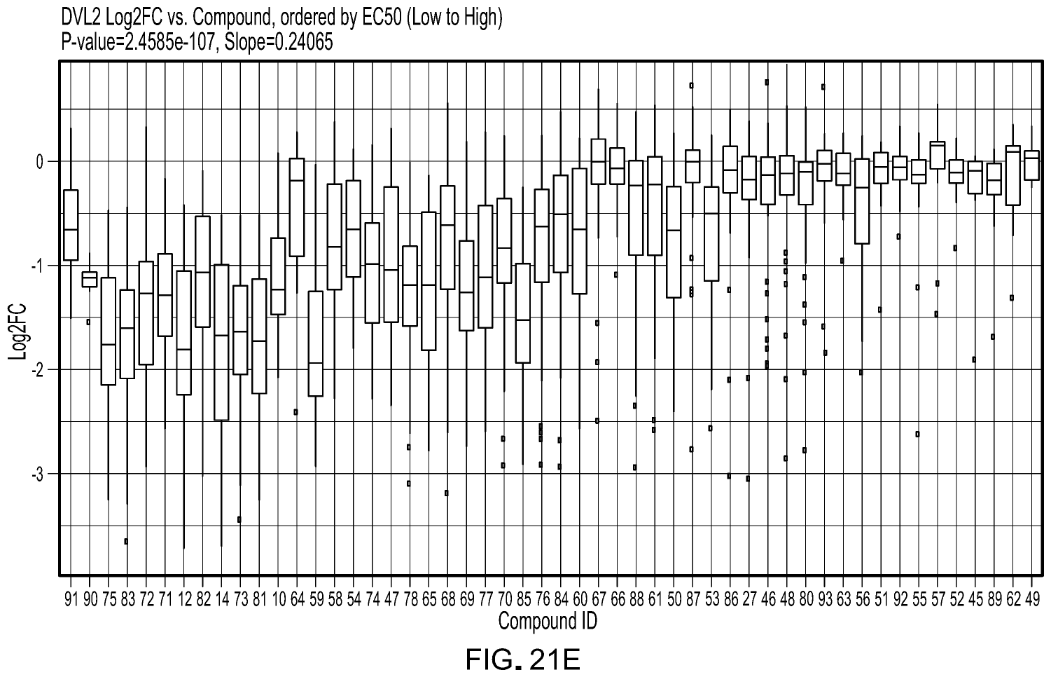

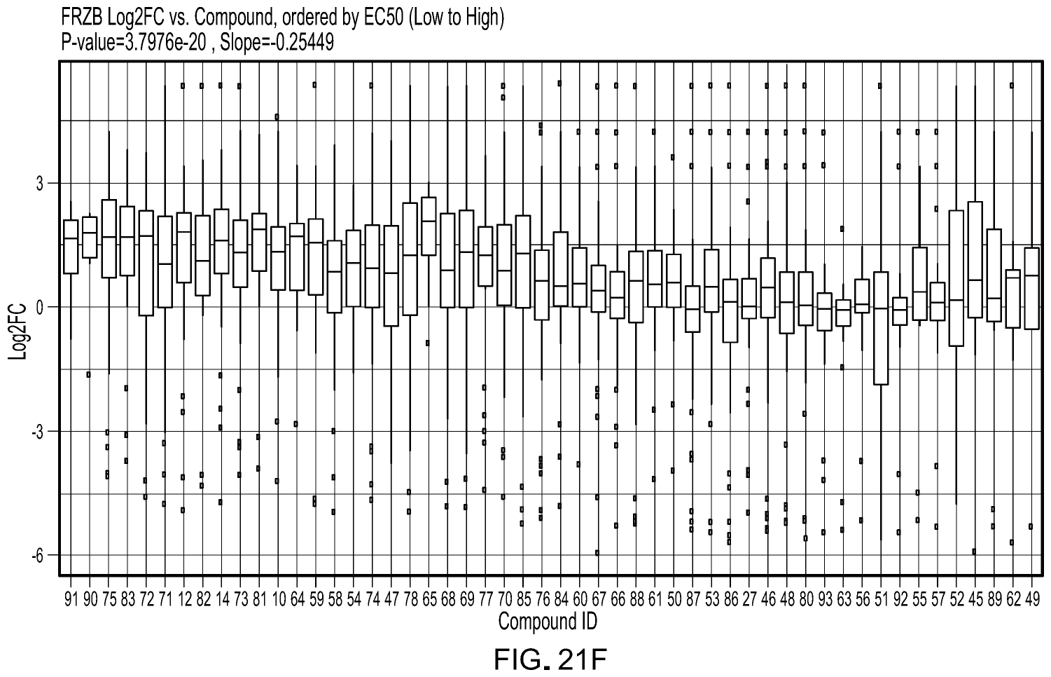

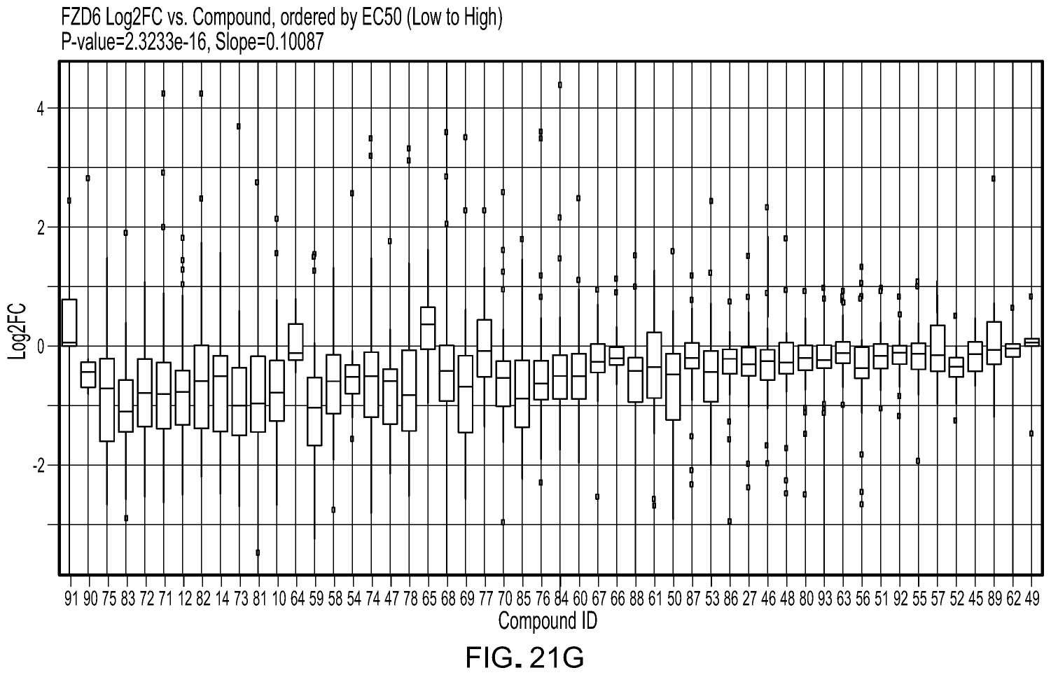

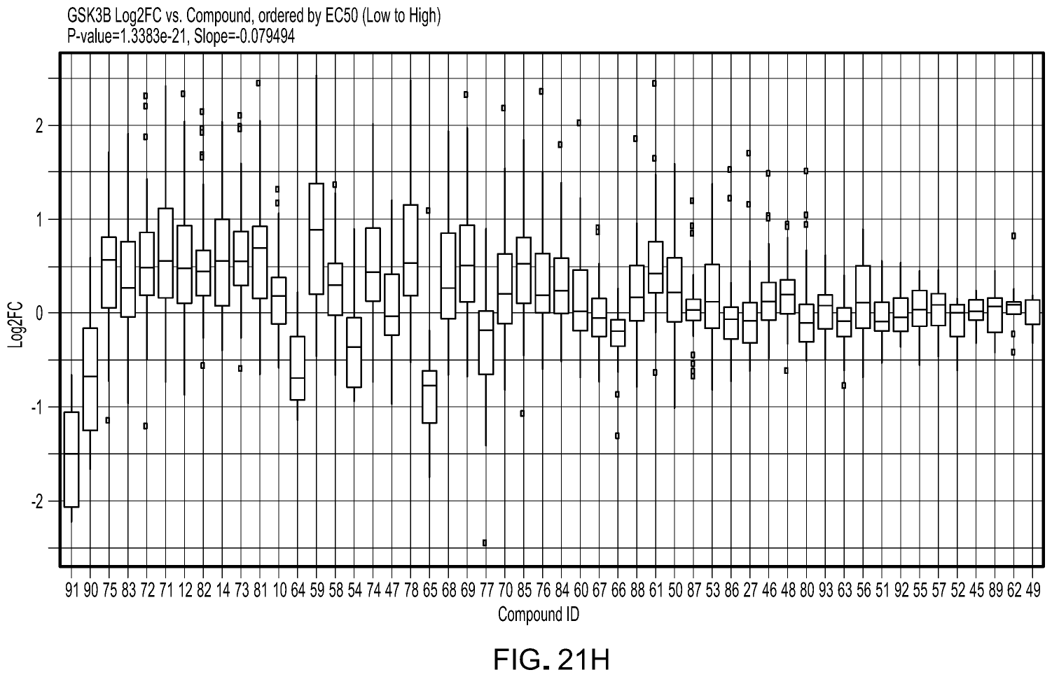

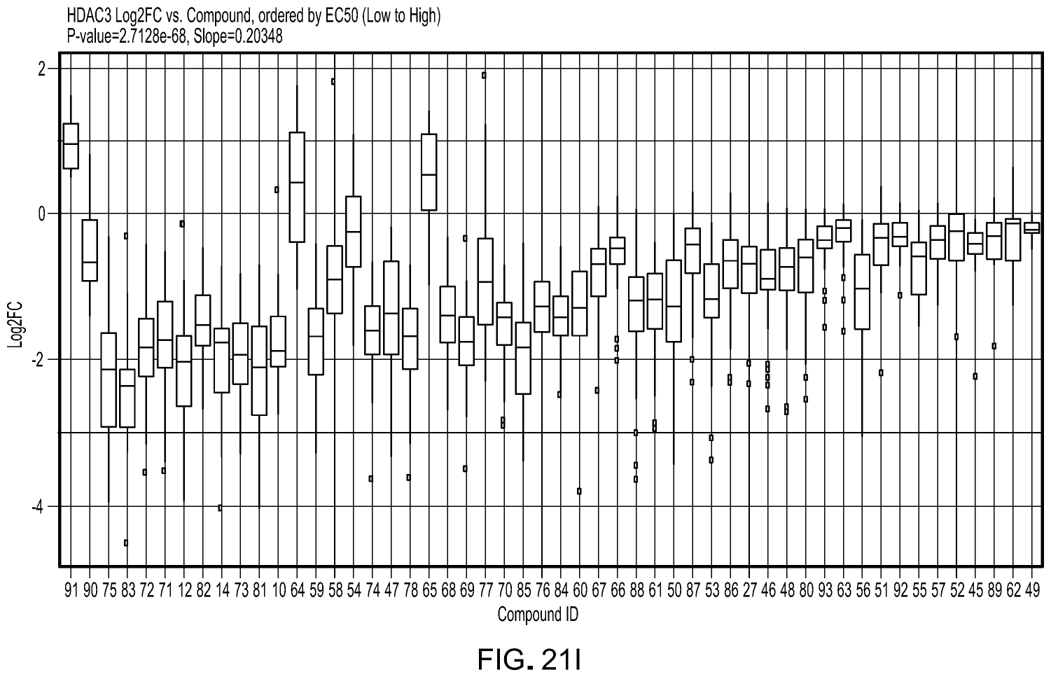

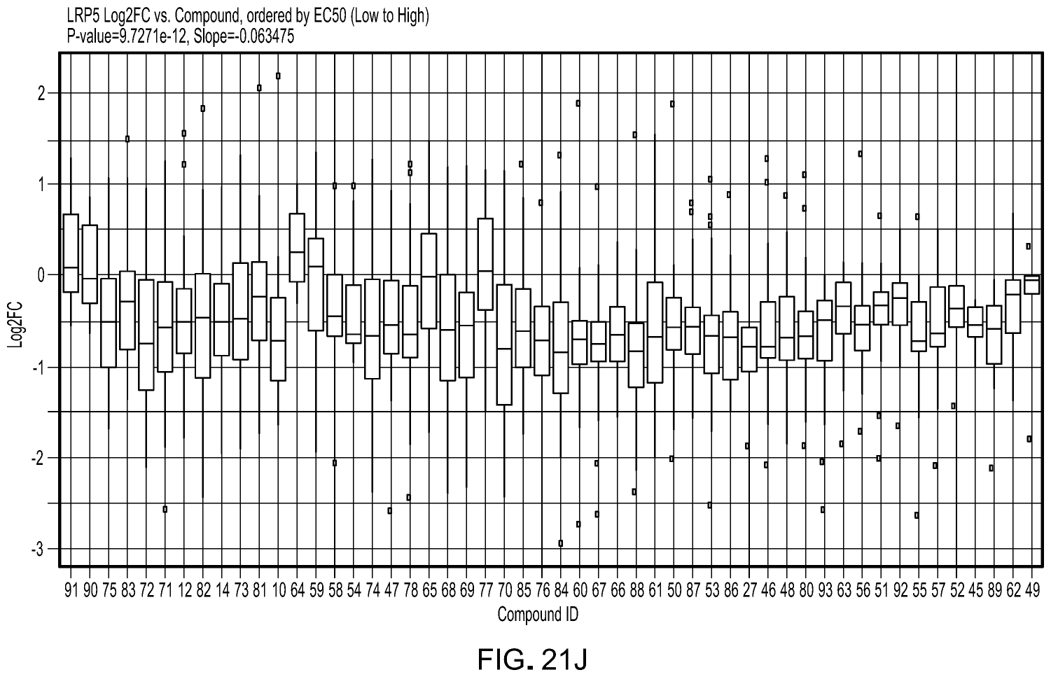

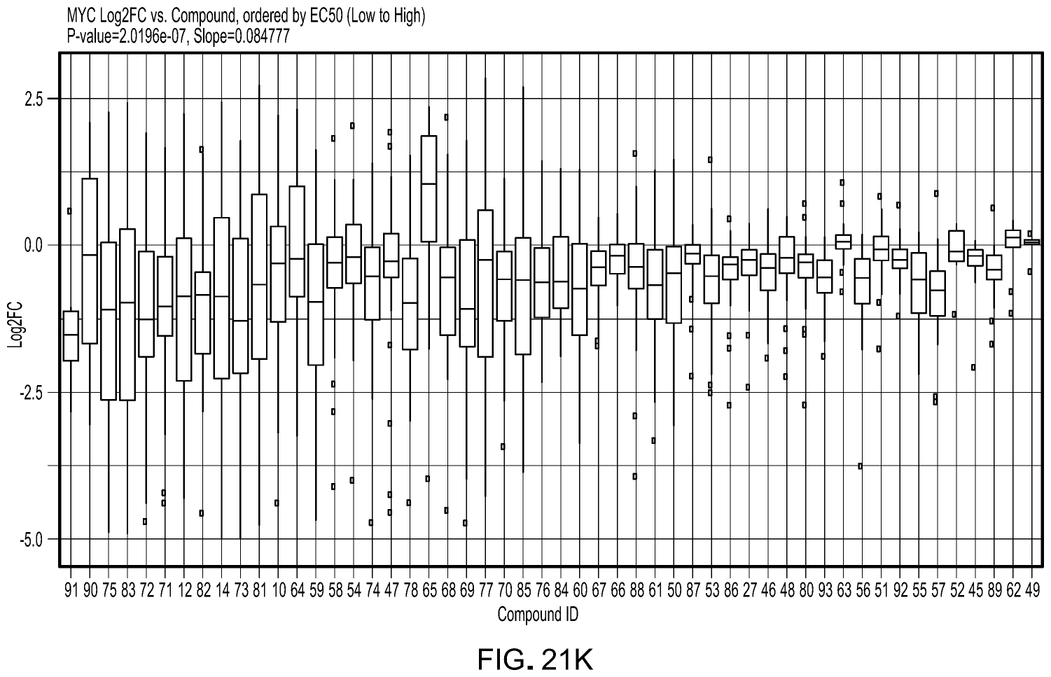

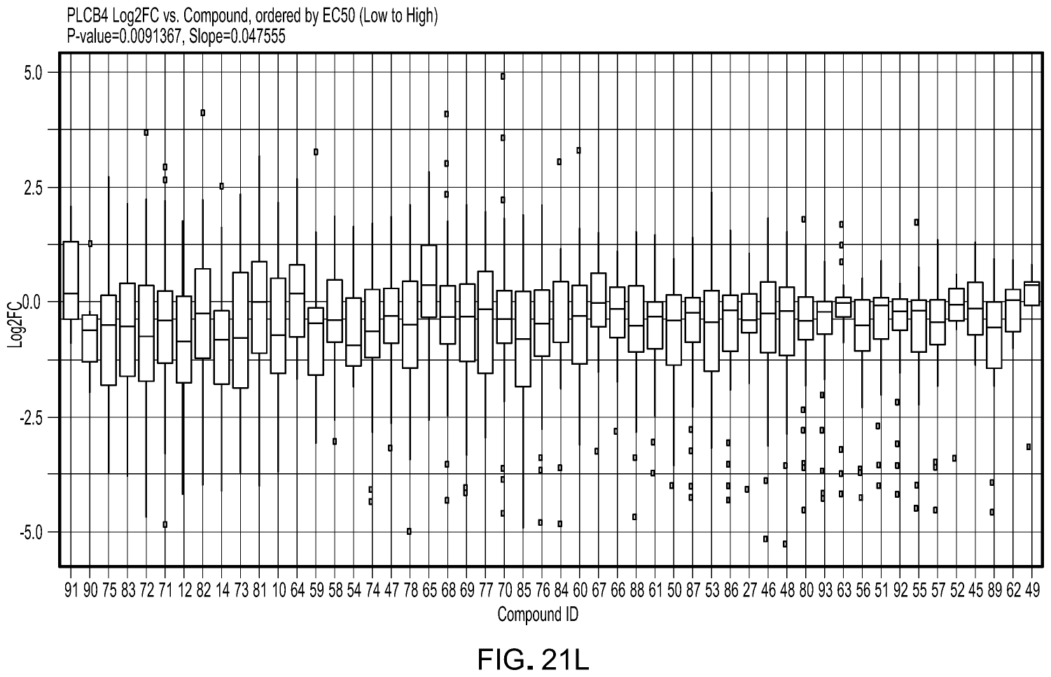

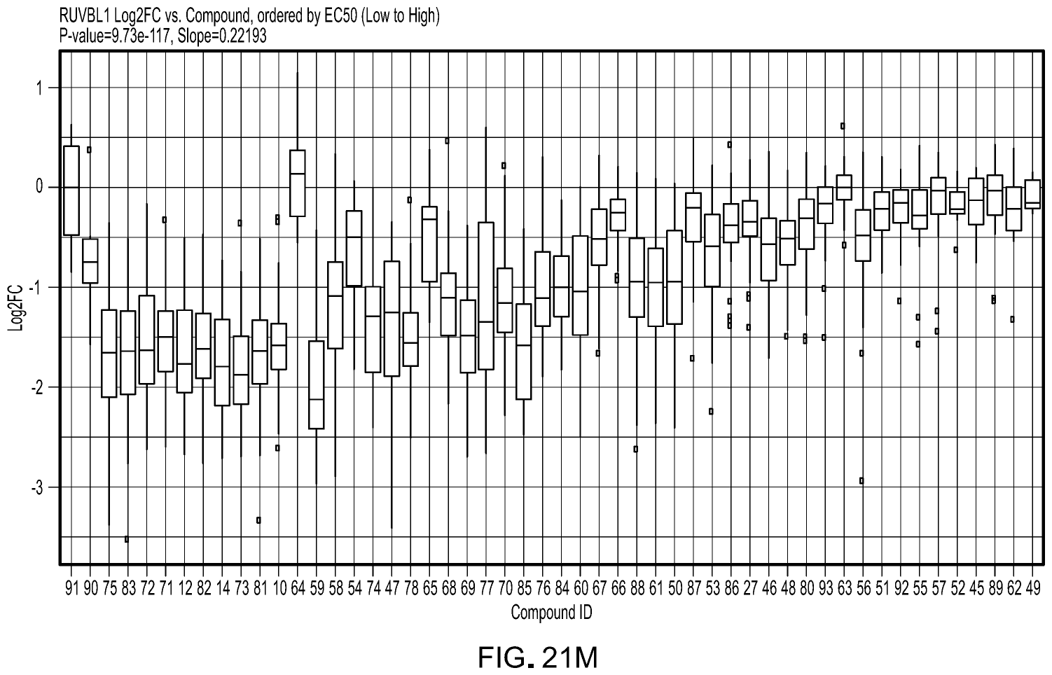

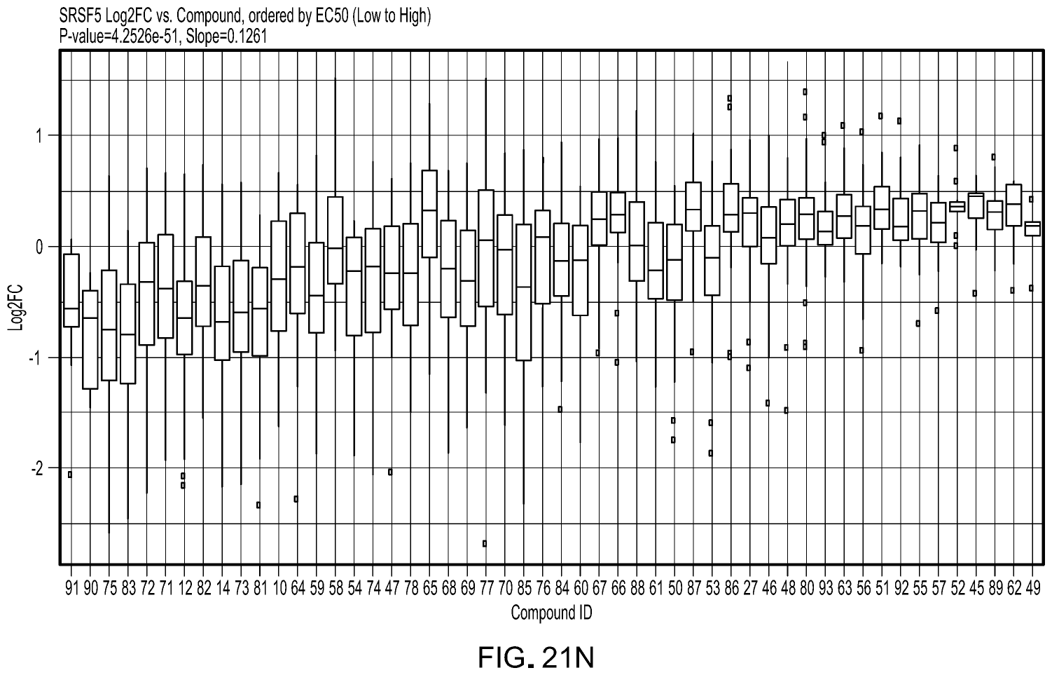

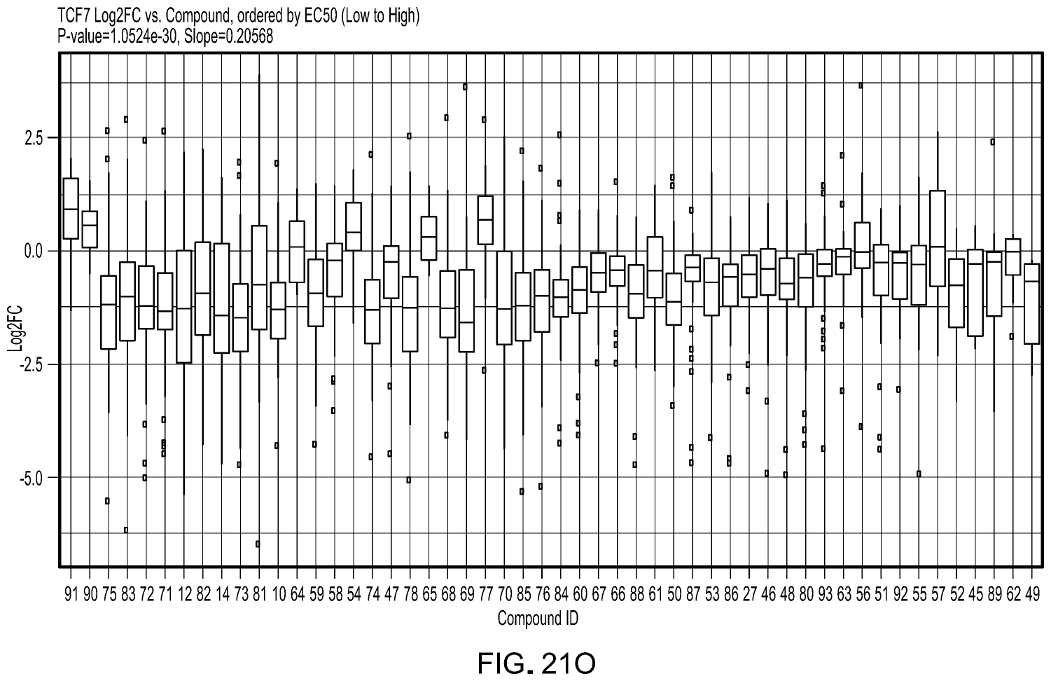

KATO III, which is characterized to have a TP53 mutation, demonstrated the highest EC₅₀ of 0.447 μM. Compound 12 was most potent in NCI-N87 (EC₅₀=0.017 μM) which is a cell line with TP53 and SMAD4 mutations but is also reported to overexpress HER2 (Weinberg et al., Clin Cancer Res 16, 1509-1519, 2010). The proliferation of the remaining cell lines was also inhibited demonstrating EC₅₀<0.3 μM. Additionally, Compound 12 exhibited anti-tumor responses in the NCI-N87 human tumor xenograft model of GC (FIG. 20). Compound 12 dosed at 25 mg/kg/day QD achieved 82% TGI relative to the vehicle group with signs of tumor regressions in 5 of 7 animals at the end study. When Compound 12 was dosed at 25 mg/kg QOD, a significant 63% TGI was achieved, which supports the therapeutic potential of an intermittent dosing strategy with Compound 12. Furthermore, 39% TGI was achieved by the lowest 12.5 mg/kg/day QD group. Overall, Compound 12 demonstrated excellent anti-tumor activity in GI tumor xenograft models at doses which were well-tolerated.

It is notable that single knockdowns of CLK2 or CLK3 could not recapitulate the inhibition of SW480 TOPflash Wnt reporter activity which was demonstrated by Compound 12. These observations support the notion that as a small molecule kinase inhibitor, Compound 12's ability to inhibit the activities of multiple CLKs, particularly that of CLK2 and CLK3 allows for stronger inhibition of SR phosphorylation.

In addition, when comparing the abilities of Compound 12with CC-671, a more selective CLK2 inhibitor which does not inhibit CLK3 (Riggs et al., J Med Chem 60, 8989-9002, 2017), to inhibit SR phosphorylation, Compound 12 was much more potent at inhibiting SRSF6 and SRSF5 phosphorylation in SW480 cells. The weaker phenotype exhibited by the more CLK2-selective inhibitor was reflected in the 17-fold, and 50-fold less potent EC₅₀ₛ demonstrated by CC-671 in the TOPflash reporter and cell viability assays, respectively. As a result, there was minimal effect of CC-671 in inhibiting expression of Wnt pathway genes such as AXIN2, CTNNB1, LEF1, MYC, TCF7 and TCF7L2 in SW480 cells. Inhibition of protein expression of these key Wnt pathway genes by Compound 12 was confirmed for all genes except for CTNNB1, where cytoplasmic and nuclear protein levels of β-catenin appeared unaffected by Compound 12. This finding along with the observation that Compound 12 can inhibit the expression of genes which are not directly regulated by β-catenin such as TCF7L2, BTRC and DVL2 suggest that Compound 12 regulates these Wnt pathway genes via a β-catenin independent mechanism in CRC cells (Herbest et al., BMC Genomics 15, 74, 2014). These observations subsequent to profound inhibition of SR phosphorylation underscore the putative role of SR proteins at the interface of alternative splicing and regulation of gene expression (Long et al., Biochem J 417, 15-27, 2009; Ånkö, Semin Cell Dev Biol 32, 11-212, 2014; Zhou et al., Chromosoma 122, 191-207, 2013). SR proteins play an important function in pre-mRNA splicing by facilitating recruitment of spliceosome proteins, splicing site selection and reported to facilitate mRNA export (Ånkö, Semin Cell Dev Biol 32, 11-212, 2014; Huang et al., Proc Natl Acad Sci USA 101, 9666-9670, 2004). Disruption of alternative splicing by pharmacological inhibition of SR phosphorylation can lead to generation of premature termination codons (PTCs) which is part of nonsense-mediated mRNA decay (NMD) pathway to eliminate unstable transcripts (Isken et al., Nat Rev Genet 9, 699-712, 2008; Araki et al., PLoS One 10, 1-18, 2015; Funnell et al., Nat Commun 8, 1-15, 2017). This supports Compound 12's mechanism of action by which strong inhibition of CLKs, particularly that of CLK2 and CLK3 mediated inhibition of Wnt signaling and Wnt pathway gene expression. Further studies are required to understand if there are dominant negative spliced forms such as those described for TCF7L2 (Arce et al., Oncogene 25, 7492-7504, 2006; Vacik et al., Cell Cycle 10, 4199-4200, 2011) which could be contributing Compound 12's ability to inhibit the Wnt pathway. Though Compound 12 can also inhibit DYRK kinase activity, the inability of a DYRK-selective small molecule kinase inhibitor, Harmine (Riggs et al., J Med Chem 60, 8989-9002, 2017; Zhou et al., Chromosoma 122, 191-207, 2013), to have any effect on SR phosphorylation, nuclear speckle size and Wnt pathway gene expression suggests that there is limited role for DYRK inhibition in Compound 12's main mechanism of action.

Analysis of the effect of 1 μM Compound 12 on 180 Wnt pathway genes across seventeen CRC cells revealed that LRP5, DVL2 and βTRC appeared to be commonly regulated by Compound 12. This novel and apparently, direct relationship between the expressions of LRP5, DVL2 and TCF7 and CLK2 was confirmed by siRNA knockdown studies, whereby the silencing of CLK2 led to loss of gene and protein expression. This fits with the hypothesis that the loss of CLK2 is impacting pre-mRNA gene processing, leading to the formation of unstable transcripts which are destroyed and therefore exerting an overall inhibitory effect on gene expression (Ånkö, Semin Cell Dev Biol 32, 11-21; Smith et al., J Cell Biol 144, 617-629, 1999). There was a group of genes, AXIN2, MYC, LEF1 and BTRC which were not affected at the gene expression level but were inhibited at the protein expression levels. For these genes, these data suggest a potential post-transcriptional regulation by CLK2 or possibly CLK2/SRSF-mediated events affecting translation or RNA export (Huang et al., Proc Natl Acad Sci USA 101, 9666-9670, 2004; Smith et al., J Cell Biol 144, 617-629, 1999). However, there was evidence of an alternatively spliced form was detected at the protein level. Upon silencing of CLK2, there appeared to be an effect on SRSF6 protein expression whereby the formation of the 40 kDa isoform was increased. This suggests that there was an impact on alternative splicing due to the loss of CLK2, which led to increased transcription and subsequent translation of this isoform compared to nontarget controls. However, the phosphorylation of the 53 kDa and 40 kDa SRSF6 isoforms did not appear affected by CLK2 knockdown, supporting the ability of other CLKs to compensate and maintain phosphorylation of SR proteins (Stojdl and Bell, Biochem Cell Biol 77, 293-298, 1999). In contrast, upon siRNA knockdown of CLK3, there was a moderate decrease of SRSF6 phosphorylation which was stronger in the CRISPR CLK3 knockout, suggesting preferential phosphorylation of SRSF6 by CLK3. In common with the effects of CLK2 knockdown, there was a decrease in TCF7 and MYC protein expression upon CLK3 loss, which reinforces an importance of CLK interaction in the regulation of these Wnt pathway genes. It also supports recent reports that MYC oncogene requires an intact spliceosome to maintain cancer cell survival (Hsu et al., Nature 525, 384-388, 2015). Decrease of MYC may also have contributed to the profound inhibition of in vivo tumor growth and tumor regressions observed by CRISPR-generated CLK3 KO SW480 clones. The pronounced effect of the CLK3 KO on in vivo tumor growth also supports its role as an oncogenic kinase which has been described for CLK2 in breast cancer cells (Yoshida et al., Cancer Res 75, 1516-1526, 2015).

In additional to strong biological activity, Compound 12 was also optimized to for drug properties as evidenced by excellent bioavailability when administered orally in mice. The anti-tumor effect exerted by Compound 12 was on-target as demonstrated by strong inhibition of SR phosphorylation in SW480 CRC tumors. This data also suggests Compound 12 was able to permeate the nucleus and inhibit CLK activity, but that inhibition was reversible as seen by the return of SR phosphorylation to control levels 24 hours post-dose. When administered orally once a day, Compound 12demonstrated convincing tumor growth inhibition (>50% TGI) in both CRC and gastric tumor xenograft models. This activity was also seen a human PDX model of CRC. PDX tumors derived directly from the patient are reported to retain more of the complexities of tumor architecture and heterogeneity compared to cell line xenograft studies (Izumchenko et al., Clin Pharmacol Ther 99, 612-621, 2016). These studies complement traditional cell line xenograft efficacy models and provide additional insight on the potential cancer types and anticipated clinical response to Compound 12 as a potential cancer therapeutic.

### Example 5. Cell Viability Activity Assay

Fifty-one human cancer cell lines (breast cancer (8 cell lines), colorectal cancer (6 cell lines), haematopoietic & lymphoid tumors (13 cell lines), liver cancer (3 cell lines), lung cancer (4 cell lines), ovarian cancer (4 cell lines), pancreatic cancer (8 cell lines), and prostate cancer (5 cell lines)) were tested to determine the effects of representative compounds of Formulas (I)-(XII) on cellular proliferation, as evaluated using the CellTiter-Glo® Viability Assay.

Representative compounds were screened using the assay procedure to assess the effect on cell proliferation as described below.

Tables 11-17 shows the measured EC₅₀ for inhibition of cancer cell proliferation for representative compounds of Formulas (I)-(XII) as described herein in different cancer cell lines.

TABLE 11

| | Breast cancer, EC₅₀ (μM) | | | | | | | |
|---|---|---|---|---|---|---|---|---|
| Compound | HCC1599 | DU4475 | CAMA1 | MDA-MB-231-Luc | T47D | MCF7 | BT-549 | ZR-75-1 |
| 10 | 0.275 | 0.101 | 0.666 | 0.625 | 1.445 | 1.723 | 0.283 | 0.620 |
| 12 | 0.103 | 0.182 | 0.347 | 0.304 | 0.427 | 0.600 | 0.231 | 0.361 |
| 14 | 0.165 | 0.137 | 0.329 | 0.360 | 0.503 | 0.725 | 0.339 | 0.410 |
| 27 | 8.037 | 1.987 | 4.250 | 3.648 | 4.100 | 3.967 | 3.614 | 3.907 |
| 46 | 1.171 | 4.012 | 2.735 | 2.094 | 2.355 | >10 | 1.168 | 1.681 |
| 47 | 0.075 | 0.077 | 0.210 | 0.407 | 0.468 | 1.962 | 0.409 | 0.358 |
| 48 | 0.146 | 1.001 | 1.907 | 2.857 | >3.5 | >3.5 | 1.306 | 3.413 |
| 50 | 0.828 | 0.750 | 0.919 | 1.195 | 1.735 | 3.521 | 1.529 | 1.106 |
| 51 | 0.407 | 7.194 | >10 | >10 | >10 | >10 | >10 | >10 |
| 53 | 0.209 | 3.127 | 0.340 | 1.496 | 0.455 | 6.394 | 0.761 | 1.279 |
| 54 | 0.085 | 0.057 | 0.167 | 0.153 | 0.167 | 0.794 | 0.075 | 0.145 |
| 55 | 0.860 | 2.801 | >3.5 | >3.5 | >3.5 | >3.5 | 0.812 | >3.5 |
| 56 | 0.248 | 0.042 | 1.526 | 6.628 | 3.223 | >10 | 0.328 | 6.905 |
| 57 | 2.196 | 1.661 | >10 | >10 | >10 | >10 | 5.812 | >10 |
| 58 | 0.379 | 0.131 | 1.018 | 0.505 | 1.243 | 1.811 | 0.480 | 0.519 |
| 59 | 0.286 | 0.178 | 0.345 | 0.636 | 0.730 | 0.853 | 0.473 | 0.653 |
| 60 | 0.057 | 0.169 | 0.252 | 0.557 | 0.296 | 1.601 | 0.734 | 0.738 |
| 61 | 0.367 | 0.411 | 3.847 | 2.039 | 3.547 | 3.387 | 0.786 | 1.843 |
| 62 | 0.026 | 0.387 | >10 | >10 | >10 | >10 | >10 | >10 |
| 63 | 2.333 | 3.871 | 5.209 | 6.470 | 8.473 | >10 | 4.744 | 6.813 |
| 64 | 0.262 | 0.121 | 0.473 | 0.451 | 0.465 | 0.633 | 0.228 | 0.211 |
| 65 | 0.209 | 0.114 | 0.457 | 0.106 | 0.886 | 0.989 | 0.414 | 0.617 |
| 66 | 0.214 | 0.825 | 0.765 | 2.136 | 3.576 | 6.413 | 1.442 | 1.346 |
| 67 | 0.038 | 0.247 | 1.342 | 2.698 | 4.956 | 3.701 | 0.731 | 2.919 |
| 68 | 0.224 | 0.487 | 0.901 | 1.109 | 1.179 | 1.821 | 0.421 | 1.366 |
| 69 | 0.648 | 0.374 | 1.227 | 0.312 | 1.291 | 3.344 | 0.718 | 0.412 |
| 70 | 0.510 | 0.624 | 0.788 | 0.891 | 1.353 | 2.151 | 0.496 | 1.124 |
| 71 | 0.177 | 0.140 | 0.307 | 0.292 | 0.493 | 0.860 | 0.215 | 0.365 |
| 72 | 0.147 | 0.121 | 0.294 | 0.230 | 0.506 | 0.670 | 0.186 | 0.284 |
| 73 | 0.073 | 0.163 | 0.379 | 0.374 | 0.534 | 0.914 | 0.272 | 0.467 |
| 74 | 0.168 | 0.302 | 0.694 | 0.750 | 0.997 | 1.898 | 0.346 | 0.971 |
| 75 | 0.091 | 0.123 | 0.216 | 0.188 | 0.246 | 0.483 | 0.169 | 0.177 |
| 76 | 0.673 | 0.468 | 0.640 | 0.798 | 1.221 | 3.364 | 1.053 | 0.950 |
| 77 | 0.606 | 0.452 | 1.852 | 0.890 | 1.018 | 1.279 | 0.549 | 0.826 |
| 78 | 0.512 | 0.381 | 0.561 | 0.677 | 0.888 | 1.419 | 0.655 | 0.711 |
| 80 | 2.192 | 3.215 | 3.090 | 4.064 | 4.623 | 6.642 | 4.505 | 4.031 |
| 81 | 0.165 | 0.152 | 0.429 | 0.338 | 0.471 | 0.505 | 0.273 | 0.441 |
| 82 | 0.154 | 0.148 | 0.244 | 0.462 | 0.451 | 0.942 | 0.277 | 0.440 |
| 83 | 0.099 | 0.145 | 0.304 | 0.193 | 0.253 | 0.491 | 0.176 | 0.220 |
| 84 | 1.126 | 0.640 | 1.215 | 1.103 | 1.434 | 2.555 | 1.242 | 1.412 |
| 85 | 0.094 | 0.420 | 0.399 | >10 | 0.635 | 0.683 | 0.210 | 0.506 |
| 86 | >10 | 2.504 | 2.133 | 3.791 | >10 | >10 | 2.887 | 4.175 |
| 87 | 1.048 | 1.372 | 2.646 | 3.047 | 7.090 | 4.117 | 1.550 | 2.964 |
| 88 | 2.305 | 0.540 | 1.407 | 1.220 | 7.190 | 1.902 | 1.028 | 1.435 |
| 90 | 0.125 | 0.095 | 0.433 | 0.202 | 0.281 | 0.360 | 0.116 | 0.216 |
| 91 | 0.008 | 0.046 | 0.088 | 0.118 | 0.402 | 0.336 | 0.024 | 0.113 |
| 92 | 0.312 | 6.041 | 8.715 | >10 | 0.867 | >10 | 5.513 | >10 |
| 93 | 0.240 | 1.024 | 7.756 | 6.787 | 0.633 | >10 | 3.756 | 6.794 |

TABLE 12

| Com- | Colorectal cancer, EC₅₀ (μM) | | | | | |
|---|---|---|---|---|---|---|
| pound | SW480 | SW48 | SW620 | HCT116 | HT29 | DLD-1 |
| 10 | 0.272 | 0.006 | 0.010 | 0.009 | 0.127 | 0.013 |
| 12 | 0.159 | 0.090 | 0.114 | 0.138 | 0.291 | 0.183 |
| 14 | 0.191 | 0.096 | 0.113 | 0.140 | 0.295 | 0.161 |
| 27 | 1.078 | 0.675 | 0.970 | 1.076 | 2.377 | 1.654 |
| 46 | 0.491 | 0.469 | 0.473 | 0.860 | 8.893 | 2.347 |
| 47 | 0.220 | 0.164 | 0.164 | 0.402 | 1.432 | 0.658 |
| 48 | 1.574 | 0.630 | 0.886 | 1.173 | 1.538 | 2.205 |
| 50 | 0.274 | 0.312 | 0.239 | 0.522 | 2.798 | 1.172 |
| 51 | >10 | 3.765 | 2.343 | 3.447 | >10 | 1.637 |
| 53 | 0.305 | 0.861 | 0.385 | 0.749 | 8.885 | 3.566 |
| 54 | 0.042 | 0.049 | 0.041 | 0.056 | 1.282 | 0.136 |
| 55 | >3.5 | >3.5 | 1.965 | >3.5 | >3.5 | >3.5 |
| 56 | 1.380 | 5.050 | 1.092 | 3.812 | >10 | 9.993 |
| 57 | >10 | 2.031 | 7.704 | >10 | >10 | >10 |
| 58 | 0.165 | 0.057 | 0.066 | 0.150 | 0.455 | 0.132 |
| 59 | 0.353 | 0.132 | 0.187 | 0.193 | 0.666 | 0.376 |
| 60 | 0.486 | 0.174 | 0.455 | 0.639 | 2.519 | 1.757 |
| 61 | 0.769 | 0.151 | 0.418 | 0.611 | 1.703 | 0.644 |
| 62 | >10 | 0.393 | >10 | >10 | >10 | >10 |
| 63 | 3.239 | 1.492 | 1.818 | 2.934 | 6.161 | 5.010 |
| 64 | 0.123 | N/A | N/A | N/A | N/A | N/A |
| 65 | 0.456 | N/A | N/A | N/A | N/A | N/A |
| 66 | 1.467 | 0.076 | 0.178 | 0.061 | 0.345 | 0.063 |
| 67 | 0.644 | 0.147 | 0.156 | 0.318 | 1.202 | 0.264 |
| 68 | 0.546 | 0.190 | 0.206 | 0.389 | 0.774 | 0.350 |
| 69 | 0.190 | 0.131 | 0.125 | 0.156 | 0.318 | 0.265 |
| 70 | 0.739 | 0.285 | 0.352 | 0.465 | 1.086 | 0.481 |
| 71 | 0.145 | 0.052 | 0.068 | 0.105 | 0.247 | 0.137 |
| 72 | 0.129 | 0.057 | 0.072 | 0.103 | 0.257 | 0.157 |
| 73 | 0.160 | 0.105 | 0.130 | 0.155 | 0.385 | 0.241 |
| 74 | 0.313 | 0.151 | 0.163 | 0.239 | 0.520 | 0.215 |
| 75 | 0.143 | 0.051 | 0.067 | 0.102 | 0.187 | 0.141 |
| 76 | 0.384 | 0.160 | 0.241 | 0.455 | 1.407 | 1.530 |
| 77 | 0.383 | 0.120 | 0.187 | 0.294 | 0.537 | 0.229 |
| 78 | 0.404 | 0.182 | 0.198 | 0.380 | 0.674 | 0.499 |
| 80 | 1.510 | 0.926 | 1.403 | 2.063 | 6.220 | 7.217 |
| 81 | 0.132 | 0.120 | 0.135 | 0.147 | 0.433 | 0.199 |
| 82 | 0.339 | 0.122 | 0.143 | 0.168 | 0.252 | 0.193 |
| 83 | 0.117 | 0.058 | 0.065 | 0.102 | 0.182 | 0.145 |
| 84 | 0.551 | 0.185 | 0.260 | 0.396 | 1.133 | 0.658 |
| 85 | 0.136 | 0.059 | 0.067 | 0.077 | 0.198 | 0.173 |
| 86 | 1.463 | 0.454 | 0.490 | 0.718 | 1.172 | 0.848 |
| 87 | 1.702 | 0.394 | 0.798 | 0.904 | 1.316 | 1.098 |
| 88 | 0.796 | 0.153 | 0.465 | 0.497 | 0.574 | 0.677 |
| 90 | 0.061 | 0.023 | 0.047 | 0.060 | 0.171 | 0.131 |
| 91 | 0.078 | 0.082 | 0.044 | 0.050 | 0.211 | 0.095 |
| 92 | >10 | 1.871 | 5.351 | 6.219 | >10 | 2.529 |
| 93 | 3.505 | 0.577 | 1.364 | 1.881 | 5.478 | 1.349 |

N/A = compound not tested

TABLE 13

| | Haematopoietic & Lymphoid tumors, EC₅₀ (μM) | | | | | | |
|---|---|---|---|---|---|---|---|
| Com- | JeKo- | | | MV- | DND- | | KASUMI- |
| pound | 1 | REC-1 | TOLEDO | 4-11 | 41 | TF-1 | 1 |
| 10 | 0.010 | 0.092 | 0.015 | 0.009 | 0.182 | 0.031 | 0.025 |
| 12 | 0.081 | 0.075 | 0.101 | 0.082 | 0.208 | 0.386 | 0.156 |
| 14 | 0.073 | 0.080 | 0.104 | 0.101 | 0.216 | 0.367 | 0.174 |
| 27 | 1.216 | 1.005 | 1.096 | 0.275 | 1.776 | 3.414 | 2.100 |
| 46 | 0.182 | 2.781 | 0.170 | 0.039 | 0.432 | 6.087 | 0.801 |
| 47 | 0.048 | 0.453 | 0.077 | 0.070 | 0.129 | 0.861 | 0.250 |
| 48 | 0.455 | 2.315 | 0.317 | 0.147 | 0.894 | 2.920 | 2.007 |
| 50 | 0.159 | 3.621 | 0.441 | 0.122 | 0.258 | 2.731 | 1.267 |
| 51 | 0.713 | 0.527 | 0.302 | 0.406 | 0.853 | 7.922 | 5.066 |
| 53 | 0.088 | 7.199 | 0.249 | 0.046 | 0.176 | 4.701 | 0.804 |
| 54 | 0.017 | 0.109 | 0.022 | 0.005 | 0.031 | 0.108 | 0.058 |
| 55 | 0.357 | >3.5 | 0.869 | 0.094 | 0.776 | >3.5 | 0.711 |
| 56 | 0.390 | 5.512 | 0.402 | 0.121 | 0.959 | >10 | 0.767 |
| 57 | 0.917 | 6.851 | 1.846 | 0.188 | 2.377 | 4.317 | 9.461 |
| 58 | 0.067 | 0.089 | 0.065 | 0.013 | 0.106 | 0.921 | 0.185 |
| 59 | 0.141 | 0.133 | 0.121 | 0.077 | 0.529 | 0.410 | 0.157 |
| 60 | 0.039 | 1.454 | 0.219 | 0.088 | 0.154 | 3.054 | 0.685 |
| 61 | 0.201 | 0.261 | 0.182 | 0.057 | 0.798 | 1.315 | 0.273 |
| 62 | 0.452 | >10 | >10 | 0.190 | 7.798 | >10 | >10 |
| 63 | 1.489 | 3.523 | 2.211 | 3.336 | 7.095 | 5.999 | 3.185 |
| 64 | 0.140 | 0.812 | 0.269 | 0.071 | 0.186 | 0.920 | 0.275 |
| 65 | 0.243 | 0.085 | 0.141 | 0.008 | 0.635 | 0.392 | 0.184 |
| 66 | 0.149 | 0.226 | 0.116 | 0.127 | 0.948 | 0.215 | 0.256 |
| 67 | 0.139 | 0.149 | 0.108 | 0.032 | 0.221 | 0.789 | 0.110 |
| 68 | 0.176 | 0.162 | 0.183 | 0.070 | 0.571 | 0.649 | 0.257 |
| 69 | 0.458 | 0.110 | 0.132 | 0.101 | 0.225 | 0.350 | 1.003 |
| 70 | 0.347 | 0.189 | 0.250 | 0.193 | 0.774 | 0.785 | 0.555 |
| 71 | 0.062 | 0.061 | 0.073 | 0.056 | 0.191 | 0.368 | 0.129 |
| 72 | 0.054 | 0.049 | 0.057 | 0.049 | 0.167 | 0.314 | 0.130 |
| 73 | 0.115 | 0.099 | 0.122 | 0.098 | 0.310 | 0.476 | 0.179 |
| 74 | 0.157 | 0.134 | 0.134 | 0.076 | 0.491 | 0.673 | 0.203 |
| 75 | 0.046 | 0.045 | 0.046 | 0.042 | 0.154 | 0.225 | 0.119 |
| 76 | 0.142 | 1.771 | 0.373 | 0.123 | 0.547 | 2.391 | 0.547 |
| 77 | 0.469 | 0.361 | 0.584 | 0.069 | 0.854 | 0.944 | 0.197 |
| 78 | 0.228 | 0.164 | 0.156 | 0.099 | 0.513 | 0.897 | 0.363 |
| 80 | 0.731 | 2.450 | 2.802 | 0.879 | 2.113 | >10 | 3.050 |
| 81 | 0.136 | 0.106 | 0.130 | 0.025 | 0.213 | 0.391 | 0.206 |
| 82 | 0.125 | 0.103 | 0.117 | 0.081 | 0.288 | 0.316 | 0.163 |
| 83 | 0.064 | 0.065 | 0.087 | 0.033 | 0.185 | 0.256 | 0.129 |
| 84 | 0.319 | 0.230 | 0.300 | 0.316 | 0.914 | 1.313 | 0.842 |
| 85 | 0.109 | 0.082 | 0.122 | 0.067 | 0.207 | 0.226 | 0.209 |
| 86 | 0.884 | 0.663 | 0.811 | 0.292 | 1.374 | 2.106 | 1.222 |
| 87 | 0.259 | 0.203 | 0.337 | 0.542 | 0.538 | 3.928 | 1.159 |
| 88 | 0.320 | 0.192 | 0.274 | 0.204 | 1.000 | 2.668 | 0.517 |
| 90 | 0.040 | 0.022 | 0.066 | 0.008 | 0.106 | 0.080 | 0.053 |
| 91 | 0.008 | 0.024 | 0.019 | 0.007 | 0.014 | 0.008 | 0.010 |
| 92 | 2.181 | 2.249 | 2.507 | 0.675 | >10 | 6.966 | 1.627 |
| 93 | 0.706 | 1.259 | 0.886 | 0.273 | 4.783 | 3.590 | 0.479 |

TABLE 14

| | Haematopoietic & Lymphoid tumors, EC₅₀ (μM) | | | | | |
|---|---|---|---|---|---|---|
| Comp- | MOLT- | JURKAT, | | | | |
| ound | 4 | Clone E6-1 | HL-60 | Loucy | SUDHL4 | JM1 |
| 10 | 0.008 | 0.008 | 0.017 | 0.014 | 0.120 | 0.041 |
| 12 | 0.302 | 0.412 | 0.518 | 0.155 | 0.335 | 0.095 |
| 14 | 0.327 | 0.511 | 0.719 | 0.192 | 0.317 | 0.093 |
| 27 | 2.392 | 3.852 | 4.975 | 1.488 | 2.535 | 0.982 |
| 46 | 2.962 | 3.174 | 1.419 | 0.466 | 0.398 | 0.176 |
| 47 | 0.404 | 0.466 | 0.769 | 0.136 | 0.140 | 0.075 |
| 48 | 2.523 | 1.768 | 3.061 | 0.952 | 1.227 | 0.409 |
| 50 | 1.287 | 1.067 | 0.767 | 0.174 | 0.419 | 0.256 |
| 51 | 7.256 | 2.788 | 9.458 | 1.278 | 0.501 | 0.195 |
| 53 | 2.021 | 2.441 | 1.017 | 0.150 | 0.477 | 0.222 |
| 54 | 0.254 | 0.170 | 0.121 | 0.041 | 0.036 | 0.026 |
| 55 | >3.5 | >3.5 | >3.5 | 1.357 | 0.983 | 0.952 |
| 56 | >10 | >10 | 3.212 | 0.485 | 0.365 | 0.394 |
| 57 | >10 | >10 | 3.130 | 1.707 | 0.953 | 1.676 |
| 58 | 0.314 | 0.271 | 1.134 | 0.145 | 0.074 | 0.032 |
| 59 | 0.689 | 1.020 | 1.241 | 0.304 | 0.525 | 0.115 |
| 60 | 0.503 | 1.424 | 3.244 | 0.207 | 0.641 | 0.121 |
| 61 | 0.999 | 0.880 | 1.638 | 0.581 | 0.779 | 0.237 |
| 62 | >10 | >10 | >10 | >10 | >10 | >10 |
| 63 | 7.065 | >10 | 8.695 | 3.716 | 3.459 | 1.689 |
| 64 | 0.383 | 0.624 | 0.488 | 0.217 | 0.138 | 0.175 |
| 65 | 0.715 | 0.780 | 1.011 | 0.576 | 0.309 | 0.319 |
| 66 | 0.062 | 0.097 | 0.211 | 0.300 | 0.896 | 0.417 |
| 67 | 0.381 | 0.298 | 1.540 | 0.280 | 0.241 | 0.062 |
| 68 | 0.546 | 0.561 | 0.958 | 0.317 | 0.523 | 0.165 |
| 69 | 0.343 | 0.500 | 0.522 | 0.385 | 0.498 | 0.122 |
| 70 | 0.643 | 0.863 | 1.241 | 0.617 | 0.899 | 0.273 |
| 71 | 0.200 | 0.431 | 0.432 | 0.146 | 0.208 | 0.054 |
| 72 | 0.189 | 0.337 | 0.453 | 0.139 | 0.229 | 0.047 |
| 73 | 0.263 | 0.549 | 0.592 | 0.182 | 0.305 | 0.103 |
| 74 | 0.465 | 0.464 | 0.760 | 0.241 | 0.376 | 0.135 |
| 75 | 0.162 | 0.333 | 0.449 | 0.142 | 0.155 | 0.044 |
| 76 | 0.734 | 1.334 | 1.632 | 0.385 | 0.533 | 0.167 |
| 77 | 1.216 | 0.699 | 1.775 | 0.368 | 0.424 | 0.375 |
| 78 | 0.562 | 0.976 | 1.291 | 0.522 | 0.620 | 0.177 |
| 80 | 3.612 | 4.053 | 3.252 | 1.605 | 2.269 | 1.192 |
| 81 | 0.186 | 0.305 | 0.235 | 0.374 | 0.407 | 0.120 |
| 82 | 0.233 | 0.169 | 0.221 | 0.206 | 0.292 | 0.137 |
| 83 | 0.196 | 0.353 | 0.409 | 0.135 | 0.151 | 0.064 |
| 84 | 0.965 | 1.618 | 2.589 | 0.780 | 0.972 | 0.216 |
| 85 | 0.130 | 0.466 | 0.252 | 0.241 | 0.491 | 0.175 |
| 86 | 1.456 | 2.136 | 2.845 | 1.074 | 1.113 | 0.645 |
| 87 | 1.350 | 1.263 | 2.456 | 0.916 | 0.514 | 0.210 |
| 88 | 1.448 | 1.191 | 1.275 | 0.614 | 0.527 | 0.158 |
| 90 | 0.125 | 0.226 | 0.246 | 0.105 | 0.079 | 0.060 |
| 91 | 0.045 | 0.031 | 0.034 | 0.013 | 0.009 | 0.010 |
| 92 | 6.758 | 3.806 | 7.973 | 3.963 | 4.870 | 3.239 |
| 93 | 5.556 | 3.725 | 4.594 | 1.955 | 2.562 | 0.765 |

TABLE 15

| | Lung cancer, EC₅₀ (μM) | | | | Ovarian cancer, EC₅₀ (μM) | | | |
|---|---|---|---|---|---|---|---|---|
| Compound | NCI-H522 | A427 | NCI-H460 | HCC-78 | OVCAR-3 | PA1 | TOV-112D | OV-90 |
| 10 | 0.185 | 0.027 | 0.179 | 0.620 | 0.196 | 0.009 | 0.017 | 0.600 |
| 12 | 0.133 | 0.277 | 0.261 | 0.326 | 0.113 | 0.172 | 0.152 | 0.456 |
| 14 | 0.142 | 0.303 | 0.268 | 0.467 | 0.116 | 0.186 | 0.171 | 0.464 |
| 27 | 1.988 | 3.075 | 3.537 | 3.721 | 1.306 | 1.581 | 2.318 | 3.871 |
| 46 | 0.417 | 0.591 | >10 | 4.680 | 0.302 | 0.795 | 0.713 | 5.694 |
| 47 | 0.157 | 0.140 | 2.643 | 0.666 | 0.179 | 0.403 | 0.192 | 0.567 |
| 48 | 1.172 | 0.875 | >3.5 | >3.5 | 0.890 | 1.286 | 1.683 | >3.5 |
| 50 | 0.310 | 0.489 | 9.680 | 0.920 | 0.670 | 1.086 | 0.419 | 1.339 |
| 51 | 5.830 | >10 | >10 | >10 | 3.217 | 8.983 | >10 | >10 |
| 53 | 0.327 | 0.230 | >10 | 0.961 | 0.145 | 2.295 | 0.446 | 1.604 |
| 54 | 0.040 | 0.040 | >3.5 | 0.225 | 0.037 | 0.147 | 0.065 | 0.532 |
| 55 | 2.469 | 3.096 | >3.5 | >3.5 | 0.766 | >3.5 | >3.5 | >3.5 |
| 56 | 0.224 | 1.311 | >10 | >10 | 0.436 | 0.767 | 1.451 | >10 |
| 57 | 7.580 | 7.795 | >10 | >10 | 3.887 | >10 | >10 | >10 |
| 58 | 0.103 | 0.177 | 0.454 | 0.900 | 0.162 | 0.147 | 0.292 | 0.847 |
| 59 | 0.190 | 0.443 | 0.320 | 0.997 | 0.100 | 0.270 | 0.264 | 0.936 |
| 60 | 0.191 | 0.350 | 2.651 | 1.694 | 0.116 | 0.405 | 0.475 | 1.083 |
| 61 | 0.570 | 0.619 | 0.856 | 3.023 | 0.568 | 0.819 | 0.533 | 1.979 |
| 62 | >10 | >10 | >10 | >10 | 0.554 | >10 | >10 | >10 |
| 63 | 3.302 | 4.178 | 7.406 | 8.179 | 2.621 | 3.036 | 3.631 | 7.966 |
| 64 | 0.396 | 0.243 | 0.759 | 0.725 | 0.127 | 0.200 | 0.155 | 0.179 |
| 65 | 0.379 | 0.260 | 0.810 | 1.190 | 0.645 | 0.146 | 0.437 | 0.918 |
| 66 | 0.830 | 0.205 | 0.234 | 4.113 | 1.351 | 0.088 | 0.208 | 0.529 |
| 67 | 0.097 | 0.502 | 0.919 | 1.437 | 0.291 | 0.768 | 0.421 | 2.864 |
| 68 | 0.277 | 0.506 | 0.709 | 1.136 | 0.232 | 0.537 | 0.327 | 1.483 |
| 69 | 0.430 | 0.756 | 0.922 | 0.666 | 0.406 | 0.195 | 0.233 | 0.901 |
| 70 | 0.389 | 0.605 | 1.133 | 1.122 | 0.370 | 0.585 | 0.593 | 1.451 |
| 71 | 0.095 | 0.198 | 0.248 | 0.304 | 0.092 | 0.209 | 0.117 | 0.334 |
| 72 | 0.119 | 0.136 | 0.207 | 0.211 | 0.095 | 0.154 | 0.106 | 0.317 |
| 73 | 0.144 | 0.290 | 0.396 | 0.504 | 0.123 | 0.325 | 0.165 | 0.486 |
| 74 | 0.188 | 0.355 | 0.574 | 0.835 | 0.195 | 0.495 | 0.229 | 1.491 |
| 75 | 0.079 | 0.114 | 0.196 | 0.190 | 0.067 | 0.120 | 0.108 | 0.256 |
| 76 | 0.246 | 0.481 | 2.054 | 1.074 | 0.194 | 0.725 | 0.277 | 1.072 |
| 77 | 0.312 | 0.973 | 0.649 | 1.276 | 0.204 | 0.710 | 0.457 | 0.806 |
| 78 | 0.395 | 0.672 | 0.856 | 1.104 | 0.285 | 0.432 | 0.327 | 1.041 |
| 80 | 1.594 | 2.815 | >10 | 5.457 | 1.408 | 3.181 | 1.966 | 5.601 |
| 81 | 0.216 | 0.216 | 0.329 | 0.396 | 0.315 | 0.218 | 0.228 | 0.838 |
| 82 | 0.190 | 0.236 | 0.257 | 0.308 | 0.073 | 0.175 | 0.147 | 0.628 |
| 83 | 0.105 | 0.275 | 0.197 | 0.173 | 0.110 | 0.170 | 0.121 | 0.234 |
| 84 | 0.553 | 0.856 | 1.261 | 1.467 | 0.446 | 0.643 | 0.610 | 1.638 |
| 85 | 0.412 | 0.452 | 0.564 | 0.640 | 0.171 | >10 | 0.143 | 0.562 |
| 86 | 0.895 | 1.500 | 2.719 | 6.856 | 1.198 | 1.247 | 1.225 | 6.930 |
| 87 | 0.522 | 1.122 | 1.169 | 3.407 | 0.866 | 1.365 | 1.434 | 3.645 |
| 88 | 0.330 | 0.841 | 0.697 | 1.290 | 0.569 | 1.054 | 0.873 | 1.877 |
| 90 | 0.091 | 0.130 | 0.243 | 0.136 | 0.111 | 0.230 | 0.101 | 0.361 |
| 91 | 0.060 | 0.064 | 0.476 | 0.100 | 0.025 | 0.057 | 0.102 | 0.255 |
| 92 | 7.146 | 8.562 | >10 | >10 | 8.423 | 9.917 | 7.048 | >10 |
| 93 | 1.418 | 4.121 | 5.174 | >10 | 2.538 | 5.949 | 1.547 | >10 |

TABLE 16

| | Liver cancer, EC₅₀ (μM) | | | | Prostate cancer EC₅₀ (μM) | | | |
|---|---|---|---|---|---|---|---|---|
| | | | | | | VCaP | LNCap | |
| Compound | SNU398 | HEPG2 | PLC/PRF/5 | PC3 | Du-145 | (Sigma) | clone FGC | 22Rv1 |
| 10 | 0.064 | 0.546 | 0.538 | 0.214 | 0.059 | 2.527 | 1.139 | 0.017 |
| 12 | 0.062 | 0.309 | 0.465 | 0.237 | 0.377 | 0.462 | 0.329 | 0.191 |
| 14 | 0.069 | 0.431 | 0.541 | 0.240 | 0.325 | 0.588 | 0.422 | 0.263 |
| 27 | 0.917 | 3.342 | 4.022 | 2.578 | 3.385 | 7.472 | 4.178 | 2.907 |
| 46 | 0.174 | 3.591 | >10 | 1.185 | 2.567 | 4.541 | 7.777 | 0.968 |
| 47 | 0.057 | 0.371 | 1.818 | 0.322 | 0.459 | 0.574 | 0.965 | 0.283 |
| 48 | 0.381 | 2.204 | >3.5 | 1.027 | 1.873 | 1.995 | >3.5 | 1.145 |
| 50 | 0.091 | 1.464 | 3.543 | 0.759 | 0.955 | 1.786 | 1.635 | 0.833 |
| 51 | 0.232 | >10 | >10 | 7.866 | >10 | >10 | >10 | 0.859 |
| 53 | 0.079 | 2.086 | >10 | 0.473 | 1.164 | 2.463 | 2.818 | 0.563 |
| 54 | 0.021 | 0.377 | 1.773 | 0.055 | 0.098 | 0.162 | 0.234 | 0.117 |
| 55 | 0.605 | >3.5 | >3.5 | 3.362 | >3.5 | >3.5 | >3.5 | >3.5 |
| 56 | 0.160 | >10 | >10 | 3.373 | >10 | >10 | >10 | 7.898 |
| 57 | 3.378 | >10 | >10 | 8.343 | >10 | >10 | >10 | 6.978 |
| 58 | 0.031 | 2.108 | 1.601 | 0.313 | 0.179 | 0.350 | 1.062 | 0.121 |
| 59 | 0.161 | 0.428 | 1.032 | 0.394 | 0.563 | 0.612 | 0.553 | 0.253 |
| 60 | 0.120 | 0.452 | 2.029 | 0.831 | 1.194 | 3.472 | 4.237 | 0.360 |
| 61 | 0.450 | 1.460 | 3.156 | 1.231 | 1.259 | 1.481 | 0.779 | 0.672 |
| 62 | 0.473 | >10 | >10 | >10 | >10 | >10 | >10 | 7.428 |
| 63 | 1.367 | 3.441 | 9.521 | 4.417 | 6.487 | 6.662 | 6.846 | 3.981 |
| 64 | 0.059 | 0.402 | 1.492 | 0.294 | 0.543 | 0.616 | 0.636 | 0.379 |
| 65 | 0.132 | 0.667 | 1.255 | 0.557 | 0.665 | 2.937 | 0.508 | 0.803 |
| 66 | 0.335 | 3.523 | 0.686 | 1.089 | 0.379 | 8.051 | 2.603 | 0.465 |
| 67 | 0.071 | 3.290 | 3.347 | 0.860 | 0.654 | 0.756 | 4.687 | 0.348 |
| 68 | 0.134 | 0.597 | 1.272 | 0.602 | 0.652 | 1.375 | 0.651 | 0.449 |
| 69 | 0.166 | 0.372 | 0.864 | 0.866 | 0.748 | 3.396 | 0.426 | 0.316 |
| 70 | 0.205 | 0.750 | 1.468 | 0.701 | 0.806 | 1.063 | 1.085 | 0.612 |
| 71 | 0.048 | 0.165 | 0.459 | 0.205 | 0.252 | 0.466 | 0.333 | 0.232 |
| 72 | 0.051 | 0.136 | 0.386 | 0.174 | 0.232 | 0.509 | 0.257 | 0.142 |
| 73 | 0.088 | 0.383 | 0.674 | 0.302 | 0.308 | 0.430 | 0.526 | 0.194 |
| 74 | 0.121 | 0.716 | 1.103 | 0.438 | 0.549 | 0.823 | 0.587 | 0.347 |
| 75 | 0.034 | 0.130 | 0.301 | 0.159 | 0.208 | 0.333 | 0.305 | 0.113 |
| 76 | 0.120 | 0.654 | 2.550 | 0.546 | 0.941 | 1.703 | 1.383 | 0.397 |
| 77 | 0.339 | 0.766 | 2.191 | 0.901 | 0.734 | 1.220 | 0.432 | 0.814 |
| 78 | 0.132 | 0.845 | 1.291 | 0.674 | 0.713 | 0.546 | 0.966 | 0.525 |
| 80 | 0.631 | 3.684 | >10 | 3.980 | 5.778 | 6.022 | 9.118 | 2.849 |
| 81 | 0.112 | 0.470 | 0.466 | 0.287 | 0.429 | 0.497 | 0.444 | 0.329 |
| 82 | 0.123 | 0.485 | 0.613 | 0.287 | 0.360 | 0.770 | 0.293 | 0.218 |
| 83 | 0.050 | 0.107 | 0.314 | 0.195 | 0.199 | 0.255 | 0.330 | 0.140 |
| 84 | 0.234 | 0.734 | 1.926 | 0.848 | 1.043 | 3.502 | 1.536 | 0.847 |
| 85 | 0.096 | >10 | 0.350 | 0.250 | 0.239 | 0.505 | 0.509 | 0.414 |
| 86 | 0.459 | 3.856 | >10 | 3.349 | 2.686 | 2.246 | 5.332 | 1.211 |
| 87 | 0.177 | 6.941 | 4.289 | 1.644 | 1.479 | 0.525 | 7.841 | 0.643 |
| 88 | 0.167 | 6.395 | 1.560 | 0.907 | 0.772 | 0.728 | 1.193 | 0.532 |
| 90 | 0.055 | 0.164 | 0.371 | 0.093 | 0.123 | 0.098 | 0.332 | 0.102 |
| 91 | 0.038 | 0.375 | 0.444 | 0.031 | 0.091 | 0.009 | 0.050 | 0.021 |
| 92 | 4.915 | >10 | >10 | >10 | >10 | 5.583 | 9.148 | 0.867 |
| 93 | 2.040 | 3.624 | 9.898 | 9.343 | 9.087 | 2.022 | 8.652 | 1.061 |

TABLE 17

| | Pancreatic cancer, EC₅₀ (μM) | | | | | | |
|---|---|---|---|---|---|---|---|
| Compound | MIA PaCa-2 | UPAFII | PANC-1 | BxPC3 | Capan1 | Capan2 | PANC 05.04 |
| 10 | 0.011 | 0.194 | 0.946 | 0.538 | 0.350 | 0.673 | 0.198 |
| 12 | 0.131 | 0.153 | 0.294 | 0.420 | 0.343 | 0.415 | 0.340 |
| 14 | 0.134 | 0.157 | 0.429 | 0.484 | 0.225 | 0.464 | 0.323 |
| 27 | 1.440 | 1.870 | 4.642 | 4.612 | 3.467 | 3.772 | 3.636 |
| 46 | 0.414 | 1.971 | >10 | 6.079 | 2.371 | 4.983 | 5.402 |
| 47 | 0.112 | 0.357 | 1.781 | 0.770 | 0.761 | 0.540 | 0.791 |
| 48 | 1.005 | 1.796 | >3.5 | 3.300 | 2.760 | >3.5 | >3.5 |
| 50 | 0.381 | 1.723 | >10 | 3.410 | 3.513 | 2.210 | 2.924 |
| 51 | 3.525 | 8.924 | >10 | >10 | >10 | >10 | >10 |
| 53 | 0.220 | 1.290 | >10 | 3.545 | 1.808 | 3.002 | 2.120 |
| 54 | 0.041 | 0.259 | 2.677 | 0.361 | 0.569 | 0.094 | 0.453 |
| 55 | 1.689 | >3.5 | >3.5 | >3.5 | >3.5 | >3.5 | >3.5 |
| 56 | 0.697 | >10 | >10 | >10 | 3.621 | >10 | >10 |
| 57 | 6.720 | >10 | >10 | 7.245 | >10 | >10 | >10 |
| 58 | 0.120 | 0.190 | 0.578 | 0.462 | 0.323 | 0.249 | 0.752 |
| 59 | 0.175 | 0.282 | 0.666 | 1.050 | 0.228 | 0.055 | 0.226 |
| 60 | 0.331 | 0.397 | 3.854 | 2.675 | 1.897 | 1.284 | 1.169 |
| 61 | 0.438 | 1.029 | 2.157 | 2.027 | 2.091 | 2.715 | 2.258 |
| 62 | >10 | >10 | >10 | >10 | >10 | >10 | >10 |
| 63 | 1.587 | 3.819 | 5.643 | 9.161 | 4.842 | 8.872 | 5.514 |
| 64 | 0.157 | 0.403 | 0.815 | 0.365 | 0.498 | 0.361 | 0.427 |
| 65 | 0.539 | 0.594 | 1.493 | 0.678 | 0.353 | 0.760 | 1.281 |
| 66 | 0.169 | 0.586 | 1.380 | 0.991 | 0.883 | 0.920 | 1.098 |
| 67 | 0.267 | 0.511 | 1.904 | 0.660 | 0.583 | 1.120 | 3.474 |
| 68 | 0.279 | 0.435 | 0.903 | 0.443 | 0.811 | 1.650 | 1.041 |
| 69 | 0.433 | 0.281 | 1.159 | 0.918 | 1.024 | 1.418 | 0.664 |
| 70 | 0.461 | 0.385 | 1.221 | 0.940 | 0.702 | 1.142 | 0.713 |
| 71 | 0.090 | 0.140 | 0.352 | 0.383 | 0.225 | 0.344 | 0.289 |
| 72 | 0.088 | 0.125 | 0.276 | 0.310 | 0.215 | 0.409 | 0.226 |
| 73 | 0.141 | 0.203 | 0.401 | 0.451 | 0.249 | 0.555 | 0.232 |
| 74 | 0.192 | 0.295 | 0.907 | 0.490 | 0.446 | 1.048 | 0.733 |
| 75 | 0.084 | 0.083 | 0.251 | 0.284 | 0.152 | 0.289 | 0.138 |
| 76 | 0.219 | 0.635 | 1.663 | 2.069 | 0.810 | 1.188 | 1.064 |
| 77 | 0.318 | 0.660 | 0.659 | 0.422 | 0.673 | 0.495 | 0.635 |
| 78 | 0.272 | 0.381 | 0.736 | 1.110 | 0.627 | 1.043 | 0.818 |
| 80 | 1.106 | 3.511 | 8.912 | 9.580 | 3.721 | 7.505 | 5.107 |
| 81 | 0.261 | 0.348 | 0.600 | 0.848 | 0.372 | 0.620 | 0.564 |
| 82 | 0.159 | 0.169 | 0.514 | 0.199 | 0.269 | 0.579 | 0.279 |
| 83 | 0.091 | 0.140 | 0.291 | 0.254 | 0.198 | 0.321 | 0.169 |
| 84 | 0.461 | 0.593 | 1.830 | 2.068 | 1.118 | 1.287 | 1.191 |
| 85 | 0.148 | 0.135 | 0.488 | 0.527 | 0.224 | 0.295 | 0.419 |
| 86 | 1.014 | 0.983 | 5.421 | 1.714 | 1.892 | 2.626 | 2.894 |
| 87 | 0.759 | 1.028 | 2.628 | 1.253 | 0.950 | 1.163 | 2.882 |
| 88 | 0.296 | 0.547 | 0.808 | 1.226 | 1.027 | 0.937 | 2.108 |
| 90 | 0.060 | 0.106 | 0.217 | 0.046 | 0.114 | 0.151 | 0.140 |
| 91 | 0.040 | 0.136 | 0.413 | 0.022 | 0.103 | 0.074 | 0.060 |
| 92 | 5.864 | >10 | >10 | >10 | >10 | >10 | >10 |
| 93 | 2.018 | 6.876 | >10 | 9.042 | >10 | >10 | >10 |

Table 18 shows the average EC₅₀ for inhibition of cancer cell proliferation (across all 50 cell lines) for representative compounds of Formulas (I)-(XII) as described herein. Compounds are showed ordered from most active to least active.

TABLE 18

| Com- | EC₅₀ | Com- | EC₅₀ | Com- | EC₅₀ | Com- | EC₅₀ |
|---|---|---|---|---|---|---|---|
| pound | (μM) | pound | (μM) | pound | (μM) | pound | (μM) |
| 91 | 0.099 | 58 | 0.441 | 67 | 1.071 | 56 | 5.055 |
| 90 | 0.142 | 54 | 0.453 | 66 | 1.073 | 51 | 6.510 |
| 75 | 0.167 | 74 | 0.504 | 88 | 1.146 | 92 | 6.834 |
| 83 | 0.182 | 47 | 0.516 | 61 | 1.218 | 55 | 6.890 |
| 72 | 0.206 | 78 | 0.610 | 50 | 1.592 | 57 | 7.295 |
| 71 | 0.232 | 65 | 0.615 | 87 | 1.829 | 52 | 8.464 |
| 12 | 0.257 | 68 | 0.632 | 53 | 2.121 | 45 | 8.487 |
| 82 | 0.282 | 69 | 0.636 | 86 | 2.710 | 89 | 8.500 |
| 14 | 0.289 | 77 | 0.684 | 27 | 2.842 | 62 | 8.554 |
| 73 | 0.306 | 70 | 0.750 | 46 | 2.861 | 49 | 9.722 |
| 81 | 0.324 | 85 | 0.880 | 48 | 3.221 | 79 | >10 |
| 10 | 0.332 | 76 | 0.922 | 80 | 4.065 | | |
| 64 | 0.398 | 84 | 1.040 | 93 | 4.534 | | |
| 59 | 0.434 | 60 | 1.071 | 63 | 5.050 | | |

### Example 6. Drug Response Gene Expression Biomarkers

Representative compounds were screened using the assay procedure to assess the effect on gene expression as described below. In total, 50 cell lines were treated with 54 compounds and run in triplicates.

Each compound was dissolved in DMSO as a 10 mM stock and used to prepare compound source plates. Compound transfer was performed using the ECHO 550 (Labcyte, Sunnyvale, Calif.) into 384-well white solid bottom assay plates (Greiner Bio-One) with appropriate DMSO backfill for a final DMSO concentration of 0.1%.

Cells were plated at 5,000-15,000 cells/well in 384-well plates in corresponding culture media and incubated for 24 hours at 37° C. and 5% CO₂. The resulting compound concentration was 3 μM. There were three biological replicates per compound and six biological replicates for the DMSO control.

After incubation, media was aspirated off the cells and wells were washed with PBS using an EL406 liquid handler (BioTek). PBS was removed and cells were lysed using the MagMAX™ Lysis/Binding Solution (Thermo Fisher Scientific). Total RNA was purified from the cell lysates using the MagMAX™-96 Total RNA Isolation Kit (Thermo Fisher Scientific) along with a S2 Pipettor liquid handler system (Apricot Designs)

Purified RNA was quantified using the Qubit™ RNA HS Assay Kit (Thermo Fisher Scientific) and fluorescence readings were taken using a Cytation3 plate reader (BioTek). RNA samples were normalized to 5 ng of total RNA using a Mantis liquid handler (Formulatrix). Libraries were generated from the normalized RNA samples using the QIAseq UPX 3′ Targeted RNA Panels (QIAGEN) with custom primers targeting genes of interest.

The generated libraries were then sequenced on a HiSeq 4000 instrument (Illumina) at the UCSD Institute of Genomic Medicine. Data files from the sequencing run were demultiplexed and gene counts were generated using QIAGEN GeneGlobe Data Analysis Center. A pseudocount of 1 was added to all raw counts and then normalized across all samples using the geometric mean of 20 housekeeping genes. Samples with low geometric means (<40) were excluded from downstream analysis. Treated samples were compared to untreated controls to determine relative fold changes. To correlate compound efficacy with gene expression changes, a linear regression was run against a compound's efficacy (EC50) and the corresponding gene expression change (log 2FC). Boxplots were generated to visualize the regression trends (R v. 3.6.0, ggplot2 v. 3.2.0).

### Materials and Methods

### Kinase Assays

Compounds were acoustically transferred on 1536-well plates (Echo 550, LabCyte) instrument. Kinase, peptide, and ATP reagents from the Z′-LYTE Kinase Assay Kits (Thermo Fisher) were dispensed onto the compound plates using an EL406 liquid dispenser (BioTek). Plates were incubated in the dark at room temperature for 1 hr. Development reagent was added to the plates and then incubated in the dark at room temperature for 1 hr. Fluorescence signal form the plate was then read using an EnVision Multilabel Plate Reader (Perkin Elmer). To assess the target profile of Compound 12, a full kinome screen of 466 kinases at 1 μM was performed (Thermo Fisher). IC₅₀ determinations were followed up for hits demonstrating >80% inhibition (Thermo Fisher).

### Cell Reporter Assays

Human colorectal cancer cell line SW480, stably expressing the Wnt responsive TOPflash promoter linked to luciferase gene (TOPflash) was used along with SW480 stably expressing a control EF1a-Luciferase reporter gene (GenTarget #LVP434) as a counterscreen. DMSO (vehicle control) and Compound 12 with an 8-point dose response following a 3-fold serial dilution starting at 10 μM were transferred to a 96-well assay plate (Echo 550, Labcyte Inc) in a duplicate or triplicate format. Cells were plated at ˜10,000 cells/well and incubated for 40 hours. Luminescence was detected using Bright-Glo (Promega Corp.). The effective concentration inhibiting 50% of cell reporter luminescence (EC₅₀) was determined using the sigmoidal dose-response equation using Prism7 software (GraphPad).

### Cell Lines and Assays

The rat IEC-6 small intestine cell line and 67 different human cancer cell lines selected from 9 different human tissues were cultured in appropriate tissue culture medium (ATCC), 1000 fetal bovine serum (Thermo Fisher) and 1% Penicillin Streptomycin (Thermo Fisher). All cells were grown under 37° C. and 5% CO₂ conditions (additional information provided in below).

| | | | Catalog | |
|---|---|---|---|---|
| Cancer Type | Cell Line | Vendor | Number | Culture Media |
| Breast | BT-549 | ATCC | HTB-122 | RPMI-1640 + 10% FBS |
| Carcinoma | CAMA1 | ATCC | HTB-21 | EMEM + 10% FBS |
| | DU4475 | ATCC | HTB-123 | RPMI-1640 + 10% FBS |
| | HCC1599 | ATCC | CRL-2331 | RPMI-1640 + 10% FBS |
| | MCF7 | ATCC | HTB-22 | EMEM + 0.01 mg/ml human |
| | | | | recombinant insulin + 10% FBS |
| | MDA-MB-231- | ATCC | HTB-26 | DMEM +10% FBS |
| | Luc | | | |
| | T47D | ATCC | HTB-133 | RPMI-1640 + 0.2 Units/ml bovine |
| | | | | insulin + 10% FBS |
| | ZR-75-1 | ATCC | CEL-1500 | RPMI-1640 + 10% FBS |
| Colorectal | C2BBel | ATCC | CRL-2102 | DMEM + 10% FBS + .01 mg/mL |
| Carcinoma | | | | human transferrin |
| | COLO 205 | ATCC | CCL-222 | RPMI-1640 + 10% FBS |
| | COLO 320HSR | ATCC | CRL-220.1 | RPMI-1640 + 10% FBS |
| | DLD-1 | ATCC | CCL-221 | RPMI-1640 + 10% FBS |
| | HCT 116 | ATCC | CCL-247 | RPMI-1640 + 10% FBS |
| | HCT 15 | ATCC | CCL-225 | RPMI-1640 + 10% FBS |
| | HT-29 | ATCC | HTB-38 | McCoy's 5A Medium + 10% FBS |
| | HuTu 80 | ATCC | HTB-40 | Eagle's MEM + 10% FBS |
| | LoVo | ATCC | CCL-229 | F-12K + 10% FBS |
| | LS123 | ATCC | CCL-255 | Eagle's MEM + 10% FBS |
| | LS513 | ATCC | CRL-2134 | RPMI-1640 + 10% FBS |
| | RKO | ATCC | CRL-2577 | DMEM + 10% FBS |
| | SW1417 | ATCC | CCL-238 | RPMI-1640 + 10% FBS |
| | SW48 | ATCC | CCL-231 | DMEM + 10% FBS |
| | SW480 | ATCC | CCL-228 | DMEM + 10% FBS |
| | SW620 | ATCC | CCL-227 | DMEM + 10% FBS |
| | T84 | ATCC | CCL-248 | DMEM: F12 Medium + 10% FBS |
| Gastric | KATO III | ATCC | HTB-103 | IMDM + 20% FBS |
| Carcinoma | NCI-N87 | ATCC | CRL-5822 | RPMI-1640 + 10% FBS |
| | SNU-16 | ATCC | CRL-5974 | RPMI-1640 + 10% FBS |
| | SNU-5 | ATCC | CRL-5973 | IMDM + 20% FBS |
| | AGS | ATCC | CRL-1739 | F-12K + 10% FBS |
| | SNU-1 | ATCC | CRL-5971 | RPMI-1640 + 10% FBS |
| Haematopoietic | DND-41 | ATCC | ACC-525 | RPMI1640 + 10% FBS |
| & Lymphoid | HL-60 | ATCC | CCL-240 | IMDM + 20% FBS |
| | JeKo-1 | ATCC | CRL-3006 | RPMI1640 + 20% FBS |
| | JM1 | ATCC | CRL-2957 | RPMI1640 + 10% FBS |
| | JURKAT, | ATCC | TIB-152 | RPMI1640 + 10% FBS |
| | Clone E6-1 | | | |
| | KASUMI-1 | ATCC | CRL-2724 | RPMI1640 + 20% FBS |
| | Loncy | ATCC | CRL-2629 | RPMI1640 + 10% FBS |
| | MOLT-4 | ATCC | CRL-1582 | RPMI-1640 + 10% FBS |
| | MV-4-11 | ATCC | CRL-9591 | IMDM + 10% FBS |
| | REC-1 | ATCC | CRL-3004 | RPMI1640 + 10% FBS |
| | SUDHL4 | ATCC | CRL-10423 | RPMI1640 + 10% FBS |
| | TF-1 | ATCC | CRL-2003 | RPMI1640 + 10% FBS + 2 ng/ml |
| | | | | recombinant human GM-CSF |
| | TOLEDO | ATCC | CRL-2631 | RPMI1640 + 10% FBS |
| Liver Cancer | HEPG2 | ATCC | HB-8065 | DMEM + 10% FBS |
| | PLC/PRF/5 | ATCC | CRL-8024 | EMEM + 10% FBS |
| | SNU398 | ATCC | CRL-2233 | RPMI-1640 + 10% FBS |
| Lung Cancer | A427 | ATCC | HTB-53 | EMEM + 10% FBS |
| | HCC-78 | ATCC | ACC-563 | RPMI-1640 + 10% FBS |
| | NCI-H460 | ATCC | HTB-177 | RPMI-1640 + 10% FBS |
| | NCI-H522 | ATCC | CRL-5810 | RPMI-1640 + 10% FBS |
| Ovarian Cancer | OV-90 | ATCC | CRL-11732 | 1:1 (MCDB + 1.5 g/L sodium |
| | | | | bicarbonate & Medium 199 + 2.2 g/L |
| | | | | sodium bicarbonate) + 15% FBS |
| | OVCAR-3 | ATCC | HTB-161 | RPMI-1640 + 0.01 mg/ml bovine |
| | | | | insulin + 20% FBS |
| | PA1 | ATCC | CRL-1572 | EMEM + 10% FBS |
| | TOV-112D | ATCC | CRL-11731 | 1:1 (MCDB + 1.5 g/L sodium |
| | | | | bicarbonate & Medium 199 + 2.2 g/L |
| | | | | sodium bicarbonate) + 15% FBS |
| Pancreatic | BxPC3 | ATCC | CRL4687 | RPMI-1640 + 10% FBS |
| Cancer | Capan1 | ATCC | HTB-79 | IMDM + 20% FBS |
| | Capan2 | ATCC | HTB-80 | McCoy's 5a Medium |
| | | | | Modified + 10% FBS |
| | HPAFII | ATCC | CRL-1997 | EMEM + 10% FBS |
| | MIA PaCa-2 | ATCC | CRM-CRL- | DMEM + 10% FBS + 2.5% FBS |
| | | | 1420 | |
| | PANC 05.04 | ATCC | CRL-2557 | RPMI-1640 + 15% FBS + 20 units/mL |
| | | | | human recombinant insulin |
| | PANC-1 | ATCC | CRL-1469 | DMEM + 10% FBS |
| Prostate Cancer | PC3 | ATCC | CRL-1435 | F-12F + 10% FBS |
| | Du-145 | ATCC | HTB-81 | EMEM + 10% FBS |
| | VCaP (Sigma) | ATCC | CRL-2876 | DMEM + 10% FBS (DMEM:F12 |
| | | | | 2 mM Glutamine + 10% FBS??) |
| | LNCap clone | ATCC | CRL-1740 | RPMI-1640 + 10% FBS |
| | FGC | | | |
| | 22Rv1 | ATCC | CRL-2505 | RPMI-1640 + 10% FBS |
| Rat Small | IEC-6 | ATCC | CRL-1592 | DMEM + 10% FBS + 0.1 Unit/mL |
| Intestine Cell | | | | bovine insulin |
| Line | | | | |

Effect on cell proliferation was performed using the CellTiter-Blue*® viability assay or the CellTiter-Glo© Viability Assay as recommended by the manufacturer (Promega).

### CellTiter-Blue® Viability Assay

Cells were plated in a black-walled, clear-bottomed 96-well plates with ˜1.5-3.0×10³ cells/well in appropriate medium containing 10% FBS. Cells were subsequently treated with or without Compound 12 following a 3-fold serial dilution starting at 10 μM and incubated for 4 days. Fluorescence signal was measured at 560ₑₓ/590ₑₘ nm using the Cytation3 multimodal plate reader (BioTek).

For apoptosis assays, SW480 cells were plated at 7500 cells/well in a black-walled clear-bottom 96-well plate (Corning). Following an overnight incubation, cells were treated with DMSO (vehicle control), Compound 12 following 3-fold titration starting at 3 μM or Staurosporine (0.1 μM) at 37° C. for 48 hours. After the 48-hour treatment timepoint, CellEvent™ Caspase 3/7 Green Detection Reagent (Thermo Fisher) was incubated at 37° C. for 30 minutes, followed by Hoechst 33342 nuclear staining (Thermo Fisher). Imaging and quantitation was performed using CellInsight™ CX5 high content imager (Thermo Fisher). For each well, the percentage of apoptotic cells was calculated as a ratio of the total number of cells stained positive for CellEvent Caspase 3/7 reagent to the total number of nuclei. Average of the three replicate wells per condition are presented.

For nuclear speckle staining, 2×10⁵ SW480 cells were seeded per well on glass cover slips in 12-well plates and treated with the indicated concentrations of compounds Compound 12, Harmine (Abcam), or CC-671 (Riggs et al., J Med Chem 60, 8989-9002, 2017). After approximately 6 hr, cells were fixed and stained a phospho-SC35 antibody (Santa Cruz Biotechnology). Cells were then labelled with an Alexa-Fluor 488 secondary antibody (Thermo Fisher) containing DAPI (Thermo Fisher). Cells were imaged at 100× magnification.

For Hek-293T experiments, cells were treated with DMSO or Compound 12 (3 μM, 1 μM and 0.3 μM) or PRI-724 (3 μM and 1 μM) for 1 hr before stimulation with 200 ng/ml of recombinant murine Wnt3a (Peprotech) or 4 μM of CHIR99021 (Selleckchem). Cells were collected 20 hr after stimulation for RNA extraction followed by gene expression analysis by qRT-PCR.

### CellTiter-Glo® Viability Assay

Each compound was dissolved in DMSO as a 10 mM stock and used to prepare compound source plates. Serial dilution (1:3, 10-point dose-response curves from 10 μM to 0.00035 μM) and compound transfer was performed using the Echo® 550 Liquid Handler (Labcyte, Sunnyvale, Calif.) into 384-well white solid bottom assay plates (Greiner Bio-One) with appropriate DMSO backfill for a final DMSO concentration of 0.1%.

For the Cell Viability Assays, cells were plated at 300-3,000 cells/well in 384-well plates in their respective media containing 1% Penicillin-Streptomycin and incubated for four days at 37° C. and 5% CO₂. Twenty-eight replicates of DMSO-treated cells served as controls and cells treated with compound were performed in duplicate.

After incubation, 10 μL of CellTiter-Glo® (Promega) was added to each well allowed to incubate for approximately 12 minutes. This reagent results in cell lysis and generation of a luminescent signal proportional to the amount of ATP present. The amount of ATP is directly proportional to the number of metabolically active, viable cells present in culture. The CellTiter-Glo® Assay generates a luminescent signal, produced by the luciferase reaction (Promega.com).

After incubation, the luminescence signal was read using an EnVision™ Multilabel Plate Reader (Perkin Elmer). Dose-response curves were generated and EC₅₀ concentration values were calculated using Dotmatics' Studies Software (Bishops Stortford, UK).

### Immunoblotting

For indicated experiments, cells were pelleted by centrifugation and washed with PBS and protein from the cell pellet was fractionated into cytoplasmic and nuclear fractions using a NE-PER™Nuclear and Cytoplasmic Extraction Reagents kit containing Halt™ protease and phosphatase inhibitors (Thermo Fisher). Protein concentrations of the samples were quantified using the Pierce Micro BCA protein assay kit (Thermo Fisher). Reduced protein samples were resolved on NuAGE 4-12% Bis-Tris gels and transferred onto nitrocellulose membranes (Thermo Fisher). Primary antibodies were incubated overnight at 4° C., with GAPDH, Lamin B1 or β-actin being used as loading controls (refer to Table 19 for primary antibodies and dilutions used).

TABLE 19
List of primary antibodies

| Antibody | Vendor | Catalog Number |
|---|---|---|
| AXIN2 | Cell Signaling Technologies | 2151 |
| PARP | Cell Signaling Technologies | 9542 |
| CLK1 | abcam | ab74044 |
| CLK2 | abcam | ab65082 |
| CLK3 | Cell Signaling Technologies | 3256 |
| CLK4 | abcam | ab67936 |
| c-Myc | Cell Signaling Technologies | 5605 |
| DVL2 | Cell Signaling Technologies | 3224 |
| FRZB | Sigma | SAB1412258 |
| GAPDH | Cell Signaling Technologies | 8884 |
| HER2/ErbB2 | Cell Signaling Technologies | 4290 |
| Lamin B1 | abcam | ab194109 |
| LEF1 | Cell Signaling Technologies | 2230 |
| LRP5 | Cell Signaling Technologies | 5731 |
| LRP6 | Cell Signaling Technologies | 2560 |
| MAPK8/JNK1 | Cell Signaling Technologies | 3708 |
| MCL-1 | Cell Signaling Technologies | 5453 |
| phospho-SR | EMD Millipore | MABE50 |
| PKN1 | abcam | ab195264 |
| PPP3CC | abcam | ab154863 |
| SRSF5 | Sigma | HPA043484 |
| SRSF6 | LSBio | LS-C290327 |
| Survivin | Cell Signaling Technologies | 2802 |
| TCF7 | Cell Signaling Technologies | 2203 |
| TCF7L2 | Cell Signaling Technologies | 2569 |
| β-actin | Santa Cruz Biotechnology | sc-47778 |
| β-catenin | Cell Signaling Technologies | 8480 |
| β-TrCP | Cell Signaling Technologies | 4394 |

Mouse and rabbit horseradish peroxidase (HRP)-conjugated secondary antibodies were diluted in 5% blocking buffer in TBS-T. Protein-antibody complexes were detected by chemiluminescence using the SuperSignal West Femto Chemiluminescent Substrate (Thermo Fisher) and images were captured with a ChemiDocIt2 camera system (UVP).

qRT-PCR

For IEC-6 studies, cells were treated with DMSO, Compound 12 (0.2 μM, 0.1 μM and 0.05 μM) or PRI-724 (3 μM and 1 μM) 1 hr before stimulation with 200 ng/mL of recombinant murine Wnt3a (Peprotech) or 4 μM of CHIR99021 (Selleckchem). Cells were collected 16 hr after stimulation for RNA extraction followed by gene expression analysis by qRT-PCR.

Total RNA was isolated using RNeasy Plus mini kit (Qiagen) or MagMAX Total RNA kit (Thermo Fisher) as per the manufacturers' protocol. cDNA was synthesized using the iScript cDNA Synthesis Kit (Bio-Rad), followed before performing qRT-PCR. Reactions were then run on a real-time PCR system (CFX384; Bio-Rad). A list of TaqMan™ primers (Thermo Fisher) or with custom oligos is provided in the table below and in Table 20. Relative gene expression was determined by normalizing to GAPDH using the ΔΔCt method.

| Target gene | Target sequence FW | Target sequence Rev |
|---|---|---|
| LRG5 | 5′GCTGCCAAATTGTTGGTTTT 3′ | 5′CAGGCTAGAAAGGGGAGCTT 3′ |
| | (SEQ ID NO: 29) | (SEQ ID NO: 30) |
| B2MG | 5′ ACATCCTGGCTCACACTGAA 3′ | 5′ ATGTCTCGGTCCCAGGTG 3′ |
| | (SEQ ID NO: 31) | (SEQ ID NO: 32) |

TABLE 20
List of primers

| Primer Target | Vendor | Catalog Number |
|---|---|---|
| AXIN2 | Thermo Fisher Scientific | Hs00610344_m1 |
| BTRC | Thermo Fisher Scientific | Hs00182707_m1 |
| CLK1 | Thermo Fisher Scientific | Hs00964634_m1 |
| CLK2 | Thermo Fisher Scientific | Hs00241874_m1 |
| CLK3 | Thermo Fisher Scientific | Hs00421111_m1 |
| CTNNB1 | Thermo Fisher Scientific | Hs00355045_m1 |
| DVL2 | Thermo Fisher Scientific | Hs01005253_m1 |
| ERBB2 | Thermo Fisher Scientific | Hs01001580_m1 |
| FRZB | Thermo Fisher Scientific | Hs00173503_m1 |
| GAPDH | Thermo Fisher Scientific | 4326317E |
| GSK3B | Thermo Fisher Scientific | Hs01047719_m1 |
| LEF1 | Thermo Fisher Scientific | Hs01547250_m1 |
| LGR5 | Thermo Fisher Scientific | Hs00969422_m1 |
| LRP5 | Thermo Fisher Scientific | Hs01124561_ml |
| LRP6 | Thermo Fisher Scientific | Hs00233945_ml |
| MAPK8 | Thermo Fisher Scientific | Hs01548508_m1 |
| MYC | Thermo Fisher Scientific | Hs00153408_m1 |
| PKN1 | Thermo Fisher Scientific | Hs00177028_m1 |
| PPP3CC | Thermo Fisher Scientific | Hs00904234_m1 |
| TCF7 | Thermo Fisher Scientific | Hs01556515_m1 |
| TCF7L2 | Thermo Fisher Scientific | Hs01009044_m1 |

### Nanostring Gene Expression Panel

Fifty nanograms of RNA was hybridized with Tagsets and probe pools from the nCounter@Vantage 3D™ Wnt Pathways Panel (NanoString Technologies) for 16 hours at 67° C. Hybridized samples were run on a nCounter© SPRINT Profiler (NanoString Technologies). Nanostring gene counts were normalized by the geometric mean of all housekeeping genes by nSolver (v3.0). P-values from normalized counts of CRC cell lines (n=17) were calculated by an independent t-test and adjusted by the false discovery rate (FDR) method (Benjamini & Hochberg) to correct for multiple comparisons using R (v3.4.2). Data was plotted using R (v3.4.2).

siRNA and CRISPR

1×10⁵ SW480 cells were seeded in 6-well plates and transfected with siRNA (GE Dharmacon) control or a pool of hairpins targeting human CTNNB1, CLK1, CLK2, CLK3, SRSF5, and SRSF6 mRNA using Lipofectamine RNAiMAX transfection reagent (ThermoFisher) (refer to Table 21 for list of siRNAs).

TABLE 21
List of siRNAs

| Target | Manufacturer | Catalog Number | Target Species |
|---|---|---|---|
| CLK1 | GE Dharmacon | L-004800-00-0010 | Human |
| CLK2 | Sigma Aldrich | SIHK0460-0.25NMOL | Human |
| CLK3 | GE Dharmacon | L-004802-00-0010 | Human |
| CTNNB1 | GE Dharmacon | L-003482-00-0005 | Human |
| Non-target | GE Dharmacon | D-001810-10-20 | Human |
| SRSF5 | GE Dharmacon | L-007279-01-0005 | Human |
| SRSF6 | GE Dharmacon | L-016067-01-0005 | Human |

Cell proliferation was analyzed with CellTiter-Glo® Assay (Promega) as described by the manufacturer. Reporter activity was analyzed with Bright-Glo™ Luminescent Cell Viability Assay (Promega) as described by the manufacturer.

The knockout of human CLK3 in SW480 cells was performed by clustered regularly interspaced short palindrome repeats (CRISPR)/Cas9 genome editing. First, Cas9 expressing SW480 cells were generated by transduction of Cas9 expressing lentiviral particles (Dharmacon, #VCAS10126) and Blasticidin selection (InvivoGen, #abt-bl-1) following a manufacturer protocol. Human CLK3 targeting synthetic crRNAs (Dharmacon, Table 22) and TracrRNA (Dharnacon, U-002000) were transfected to SW480-Cas9 cells using DharmaFECT1 transfection reagent (Dharmacon, #T-2001-02) as per manufacturer protocol.

TABLE 22
List of crRNAs

| Target | | | | Targeted |
|---|---|---|---|---|
| gene | Manufacturer | Catalog # | Target sequence | exon |
| CLK3 | GE Dharmacon | CR-004802-01-0005 | GCCGTGACAGCGATACATAC | 3 |
| | | | (SEQ ID NO: 33) | |
| CLK3 | GE Dharmacon | CR-004802-03-0005 | ACCCGTACCTGAGCTACCGA | 2 |
| | | | (SEQ ID NO: 34) | |

Approximately 48 hr after transfection, cells were harvested, and genomic DNAs were isolated to check gene editing by DNA mismatch detection assay using T7 endonuclease I (NE BioLabs) following a manufacturer protocol. After confirming gene editing, single cell clonal cell lines were generated from CLK3 crRNAs transfected SW480 cells by serial dilution. CLK3 knockout status of the clonal cell lines was assessed by Immunoblot analysis to validate sufficient knock-out of CLK3. To assess the effect of CLK3 knockdown on in vivo tumor growth, mice were injected subcutaneously with -2×10⁶ WT or CLK3 KO SW480 cells in PBS with 50% Matrigel. Tumors were measured by digital caliper twice weekly and tumor volume was calculated in mm³ using the formula: TV=0.5×a×b², where a and b are the long and short diameters of the tumor, respectively.

### In Vivo Tumor Xenograft Studies

All tumor xenograft studies are performed in accordance with approved Samumed, LLC Animal Committee protocols. Athymic nude Foxn1 mice are inoculated subcutaneously in the right flank region with ˜3×10⁶ SW480 CRC cells, ˜4×10⁶ HCT-116 CRC cells, or ˜5×10⁶ NCI-N87 GC cells/mouse. PDX studies are performed by Crown Bioscience. Tumor fragments from stock mice inoculated with selected primary human colorectal cancer tissues are harvested and used for inoculation into BALB/c nude mice. Each mouse is inoculated subcutaneously at the right flank with primary human colorectal cancer model CR2545 fragment (2-3 mm in diameter). For all studies, when tumors reached -100-200 mm³, tumors are randomized, and dosing is initiated. Tumor volume (mm³) and body weights are determined twice a week. % Tumor growth inhibition (% TGI) is calculated according to the following formula: (1−(Tᵢ/Cᵢ))×100% where Tᵢ and Cᵢ are the mean tumor volumes measured on a given day of in the treatment and vehicle control groups respectively. This value reflects the degree of tumor growth inhibition relative to the vehicle-treated group.

Tumor pharmacodynamic studies are performed in athymic nude mice bearing SW480 tumors. After a single dose of 25 mg/kg, Compound 12, tumors are harvested at 4, 8, and 24 hours after dosing, the tumor is extracted and cut into two pieces. One portion is appropriately lysed and SR phosphorylation immunoblotting with total SRSF6, SRSF5 and β-actin blots as loading controls are performed. qRT-PCR analysis of Wnt pathways genes is performed with RNA extracted from the second piece of tumor piece.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method of treating a cancer in a subject, the method comprising:
identifying a subject having a cancer cell that has an elevated level of Wnt pathway activity as compared to a reference level; and
administering to the identified subject a therapeutically effective amount of a CLK inhibitor or a pharmaceutically acceptable salt or solvate thereof.

2. A method of treating a cancer in a subject, the method comprising administering a therapeutically effective amount of a CLK inhibitor or a pharmaceutically acceptable salt or solvate thereof to a subject identified as having a cancer cell that has an elevated level of Wnt pathway activity as compared to a reference level.

3. A method of selecting a treatment for a subject, the method comprising:
identifying a subject having a cancer cell that has an elevated level of Wnt pathway activity as compared to a reference level; and
selecting for the identified subject a treatment comprising a therapeutically effective amount of a CLK inhibitor or a pharmaceutically acceptable salt or solvate thereof.

4. A method of selecting a treatment for a subject, the method comprising selecting a treatment comprising a therapeutically effective amount of a CLK inhibitor or a pharmaceutically acceptable salt or solvate thereof for a subject identified as having a cancer cell that has an elevated level of Wnt pathway activity as compared to a reference level.

5. A method of selecting a subject for treatment, the method comprising:
identifying a subject having a cancer cell that has an elevated level of Wnt pathway activity as compared to a reference level; and
selecting an identified subject for treatment with a therapeutically effective amount of a CLK inhibitor or a pharmaceutically acceptable salt or solvate thereof.

6. A method of selecting a subject for treatment, the method comprising selecting a subject identified as having a cancer cell that has an elevated level of Wnt pathway activity as compared to a reference level, for treatment with a therapeutically effective amount of a CLK inhibitor or a pharmaceutically acceptable salt or solvate thereof.

7. A method of selecting a subject for participation in a clinical trial, the method comprising:
identifying a subject having a cancer cell that has an elevated level of Wnt pathway activity as compared to a reference level; and
selecting the identified subject for participation in a clinical trial that comprises administration of a therapeutically effective amount of a CLK inhibitor or a pharmaceutically acceptable salt or solvate thereof.

8. A method of selecting a subject for participation in a clinical trial, the method comprising selecting a subject identified as having a cancer cell that has an elevated level of Wnt pathway activity as compared to a reference level for participation in a clinical trial that comprises administration of a therapeutically effective amount of a CLK inhibitor or a pharmaceutically acceptable salt or solvate thereof.

9. A method of treating a subject having a cancer, the method comprising:
(a) administering to the subject a therapeutic agent;
(b) after (a), identifying the subject as having a cancer cell that has an elevated level of Wnt pathway activity as compared to a reference level; and
(c) administering to the identified subject a treatment comprising a therapeutically effective amount of a CLK inhibitor or a pharmaceutically acceptable salt or solvate thereof.

10. A method of treating a subject having a cancer, the method comprising:
identifying a subject previously administered a therapeutic agent, as having a cancer cell that has an elevated level of Wnt pathway activity as compared to a reference level; and
administering to the identified subject a treatment comprising a therapeutically effective amount of a CLK inhibitor or a pharmaceutically acceptable salt or solvate thereof.

11. A method of treating a subject having a cancer, the method comprising administering to a subject previously administered a therapeutic agent and later identified as having an elevated level of Wnt pathway activity as compared to a reference level, a therapeutically effective amount of a CLK inhibitor or a pharmaceutically acceptable salt or solvate thereof.

12. A method of determining the efficacy of a CLK inhibitor or a pharmaceutically acceptable salt or solvate thereof in a subject, the method comprising:
(a) determining a first level of Wnt pathway activity in a cancer cell obtained from a subject at a first time point;
(b) administering to the subject after the first time a CLK inhibitor or a pharmaceutically acceptable salt or solvate thereof,
(c) determining a second level of Wnt pathway activity in a cancer cell obtained from the subject at a second time point; and
(d) determining that the CLK inhibitor is effective in a subject having a second level of Wnt pathway activity that is decreased as compared to the first level of Wnt pathway activity.

13. The method of claim 12, wherein method further comprises:
(e) after (d), administering one or more additional doses of the CLK inhibitor to the subject.

14. The method of any one of claims 1-13, wherein the level of Wnt pathway activity is the level of CLK1, CLK2, CLK3, CLK4, or β-catenin expression.

15. The method of claim 14, wherein the level of CLK1, CLK2, CLK3, CLK4, or β-catenin expression is the level of CLK1, CLK2, CLK3, CLK4, or β-catenin protein.

16. The method of any one of claims 1-13, wherein the level of Wnt pathway activity is the level of β-catenin in the nucleus.

17. The method of any one of claims 1-13, wherein the Wnt pathway activity is detection of a mutation in a Wnt pathway gene selected from the group consisting of: gain-of-function mutation in a β-catenin gene, a loss-of-function mutation in an AXIN gene, a loss-of-function mutation in an AXIN2 gene, a loss-of-function mutation in a APC gene, a loss-of-function mutation in a CTNNB1 gene, a loss-of-function mutation in a Tsc1 gene, a loss-of-function mutation in a Tsc2 gene, and a loss-of-function mutation GSK3D gene.

18. The method of any one of claims 1-13, wherein the Wnt pathway activity is detection of an elevated level of expression of one or more Wnt-upregulated genes.

19. The method of claim 18, wherein the one or more Wnt-upregulated genes are selected from the group consisting of: CCND1, CSNK2A1, CXCL12, LRP5, MMP7, MMP9, LEF1, AXIN2, MYC, TCF7L2, TCF7, LRP6, DVL2, BIRC, ERRB2, MAPK8, PKN1, AXIN2, ABCB1, ADAM1O, ALEX1, ASCL2, BAMBI, BCL2L2, BIRC5, BMI1, BMP4, CCND1, CD44, CDKN2A, CDX1, CEBPD, CLDN1, COX2, DNMT1, EDN1, EFNB1, ENC1, EPHB2, EPHB3, FGF18, FGFBP, FRA1, FSCN1, FZD6, FZD7, FZD8, GAST, HDAC3, HEF1, HES1, ID2, ITF2, JAG1, JUN, L1CAM, LAMC2, LGR5, MENA, MET, MMP14, MYB, MYCBP, NOS2, NOTCH2, NRCAM, PLAU, PLAUR, PLCB4, PPARD, RUVBL1, S100A4, S100A6, SGK1, SMC3, SOX9, SP5, SRSF3, SUZ12, TCF1, TIAM1, TIMP-1. TN-C, VEGF, WNT-5a, WNT-5b, WNT11, and YAP.

20. The method of any one of claims 1-13, wherein the Wnt-pathway activity is detection of a decreased level of expression of one or more of APC, FRZB, CTGF, and GSK3B.

21. The method of any one of claims 1-20, wherein the cancer is a small cell lung cancer, colorectal cancer, head and neck cancer, ovarian cancer, melanoma, renal cell carcinoma, pancreatic cancer, breast, prostate and hematologic cancers, and non-small cell lung cancer.

22. A method of decreasing the activity of one or more of CLK1, CLK2, CLK3, and CLK4, the method comprising contacting one or more of CLK1, CLK2, CLK3 and CLK4 with an effective amount of a CLK inhibitor or a pharmaceutically acceptable salt or solvate thereof.

23. The method of claim 22, wherein the method comprises contacting one or both of CLK2 and CLK3 with an effective amount of a CLK inhibitor or a pharmaceutically acceptable salt or solvate thereof.

24. A method of decreasing the activity of one or more of CLK1, CLK2, CLK3 and CLK4 in a mammalian cell, the method comprising contacting the mammalian cell with an effective amount of a CLK inhibitor or a pharmaceutically acceptable salt or solvate thereof.

25. The method of claim 24, wherein the mammalian cell is a cancer cell.

26. The method of claim 25, wherein the cancer cell has been identified as having an elevated level of Wnt pathway activity as compared to a reference level.

27. The method of any one of claim 24, wherein the contacting results in a decrease in the activity of one or both of CLK2 and CLK3 in the mammalian cell.

28. A method of altering mRNA splicing in a mammalian cell having aberrant mRNA splicing activity, the method comprising contacting the mammalian cell with an effective amount of a CLK inhibitor or a pharmaceutically acceptable salt or solvate thereof.

29. The method of claim 28, wherein the mammalian cell is a cancer cell.

30. The method of claim 29, wherein the cancer cell having aberrant mRNA spicing activity has one or more of:
an increased level of phosphorylated SRSF6 as compared to a reference level;
an increased level of phosphorylated SRSF5 as compared to a reference level;
a mutation in a SF3B1 gene, a SRSF1 gene, a SRSF2 gene, a U2AF1 gene, or a ZRSR2 gene; and
an increased level of SRSF1, SRSF2, SRSF3, SRSF4, SRSF5, SRSF6, and SRSF10 as compared to a reference level.

31. A method of treating a cancer in a subject, the method comprising:
identifying a subject having a cancer cell that has aberrant mRNA splicing activity as compared to a reference level; and
administering to the identified subject a therapeutically effective amount of a CLK inhibitor or a pharmaceutically acceptable salt or solvate thereof.

32. A method of treating a cancer in a subject, the method comprising administering a therapeutically effective amount of a CLK inhibitor or a pharmaceutically acceptable salt or solvate thereof to a subject identified as having a cancer cell that has aberrant mRNA splicing activity as compared to a reference level.

33. A method of selecting a treatment for a subject, the method comprising:
identifying a subject having a cancer cell that has aberrant mRNA splicing activity as compared to a reference level; and
selecting for the identified subject a treatment comprising a therapeutically effective amount of a CLK inhibitor or a pharmaceutically acceptable salt or solvate thereof.

34. A method of selecting a treatment for a subject, the method comprising selecting a treatment comprising a therapeutically effective amount of a CLK inhibitor or a pharmaceutically acceptable salt or solvate thereof for a subject identified as having a cancer cell that has aberrant mRNA splicing activity as compared to a reference level.

35. A method of selecting a subject for treatment, the method comprising:
identifying a subject having a cancer cell that has aberrant mRNA splicing activity as compared to a reference level; and
selecting an identified subject for treatment with a therapeutically effective amount of a CLK inhibitor or a pharmaceutically acceptable salt or solvate thereof.

36. A method of selecting a subject for treatment, the method comprising selecting a subject identified as having a cancer cell that has aberrant mRNA splicing activity as compared to a reference level, for treatment with a therapeutically effective amount of a CLK inhibitor or a pharmaceutically acceptable salt or solvate thereof.

37. A method of selecting a subject for participation in a clinical trial, the method comprising:
identifying a subject having a cancer cell that has aberrant mRNA splicing activity as compared to a reference level; and
selecting the identified subject for participation in a clinical trial that comprises administration of a therapeutically effective amount of a CLK inhibitor or a pharmaceutically acceptable salt or solvate thereof.

38. A method of selecting a subject for participation in a clinical trial, the method comprising selecting a subject identified as having a cancer cell that has aberrant mRNA splicing activity as compared to a reference level for participation in a clinical trial that comprises administration of a therapeutically effective amount of a CLK inhibitor or a pharmaceutically acceptable salt or solvate thereof.

39. A method of treating a subject having a cancer, the method comprising:
(a) administering to the subject a therapeutic agent;
(b) after (a), identifying the subject as having a cancer cell that has aberrant mRNA splicing activity as compared to a reference level; and
(c) administering to the identified subject a treatment comprising a therapeutically effective amount of a CLK inhibitor or a pharmaceutically acceptable salt or solvate thereof.

40. A method of treating a subject having a cancer, the method comprising:
identifying a subject previously administered a therapeutic agent, as having a cancer cell that has aberrant mRNA splicing activity as compared to a reference level; and
administering to the identified subject a treatment comprising a therapeutically effective amount of a CLK inhibitor or a pharmaceutically acceptable salt or solvate thereof.

41. A method of treating a subject having a cancer, the method comprising administering to a subject previously administered a therapeutic agent and later identified as having aberrant mRNA splicing activity as compared to a reference level, a therapeutically effective amount of a CLK inhibitor or a pharmaceutically acceptable salt or solvate thereof.

42. The method of any one of claims 31-41, wherein the level of aberrant mRNA splicing is determined by detecting:
the level of SRSF6 phosphorylation in the cell;
the level of SRSF5 phosphorylation in the cell;
the level of a ˜55 kDa isoform of SRSF6 in the cell; or
the level of ˜35 kDa isoform of SRSF1 in the cell.

43. A method of determining the efficacy of a CLK inhibitor in a subject, the method comprising:
(a) determining a first level of SRSF6 phosphorylation and/or SRSF5 phosphorylation in a cancer cell obtained from a subject at a first time point;
(b) administering to the subject after the first time a CLK inhibitor or a pharmaceutically acceptable salt or solvate thereof,
(c) determining a second level of SRSF6 phosphorylation and/or SRSF5 phosphorylation in a cancer cell obtained from the subject at a second time point; and
(d) determining that the CLK inhibitor is effective in a subject having a second level that is decreased as compared to the first level.

44. A method of determining the efficacy of a CLK inhibitor or a pharmaceutically acceptable salt or solvate thereof in a subject, the method comprising:
(a) determining a first level of a ˜55 kDa isoform of SRSF6 in a cancer cell obtained from a subject at a first time point;
(b) administering to the subject after the first time a CLK inhibitor or a pharmaceutically acceptable salt or solvate thereof,
(c) determining a second level of the ˜55 kDa isoform of SRSF6 in a cancer cell obtained from the subject at a second time point; and
(d) determining that the CLK inhibitor is effective in a subject having a second level of the ˜55 kDa isoform of SRSF6 that is increased as compared to the first level of the ˜55 kDa isoform of SRSF6.

45. A method of determining the efficacy of a compound of any one of Formulas I-XII or a pharmaceutically acceptable salt or solvate thereof in a subject, the method comprising:
(a) determining a first level of a ˜35 kDa isoform of SRSF1 in a cancer cell obtained from a subject at a first time point;
(b) administering to the subject after the first time point a compound of any one of Formulas I-XII or a pharmaceutically acceptable salt or solvate thereof,
(c) determining a second level of the ˜35 kDa isoform of SRSF1 in a cancer cell obtained from the subject at a second time point; and
(d) determining that the CLK inhibitor is effective in a subject having a second level of the ˜35 kDa isoform of SRSF1 that is increased as compared to the first level of the −35 kDa isoform of SRSF1.

46. The method of any one of claims 43-45, wherein method further comprises:
(e) after (d), administering one or more additional doses of the CLK inhibitor to the subject.

47. The method of any one of claims 1-46, wherein the CLK inhibitor is a multi-isoform CLK inhibitor.

48. The method of claim 47, wherein the multi-isoform CLK inhibitor has an IC₅₀ of between about 1 nM and about 10 μM for each of CLK2 and CLK3.

49. The method of claim 48, wherein the multi-isoform CLK inhibitor has an IC₅₀ of between about 1 nM and about 1 μM for each of CLK2 and CLK3.

50. The method of claim 49, wherein the multi-isoform CLK inhibitor has an IC₅₀ of between about 1 nM and about 100 nM for each of CLK2 and CLK3.

51. The method of any one of claims 1-50, wherein the CLK inhibitor is a compound of any one of Formulas I-XII or a pharmaceutically acceptable salt or solvate thereof.

52. The method of claim 47, wherein the multi-isoform CLK inhibitor has an IC₅₀ of between about 2 nM and about 10 μM for each of CLK1, CLK2, and CLK3.

53. The method of claim 52, wherein the multi-isoform CLK inhibitor has an IC₅₀ of between about 2 nM and about 1 μM for each of CLK1, CLK2, and CLK3.

54. The method of claim 47, wherein the multi-isoform CLK inhibitor has an IC₅₀ of between about 2 nM and about 10 μM for each of CLK1, CLK2, CLK3, and CLK4.

55. The method of claim 54, wherein the multi-isoform CLK inhibitor has an IC₅₀ of between about 2 nM and about 1 μM for each of CLK1, CLK2, CLK3, and CLK4.

56. The method of any one of claims 1-55, wherein the CLK inhibitor is a compound of Formula I 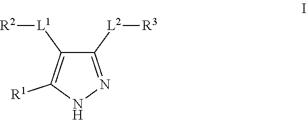
or a pharmaceutically acceptable salt or solvate thereof, wherein:
R¹ is selected from the group consisting of H, halide, and unsubstituted —(C₁₋₃ alkyl);
R² is selected from the group consisting of unsubstituted —(C₁₋₃ alkyl), unsubstituted —(C₂₋₉ alkenyl), unsubstituted —(C₁₋₉ haloalkyl), —(C₁₋₂ alkylene)ₚ(C₃₋₆ carbocyclyl) optionally substituted with 1-12 R⁴, -monocyclic heterocyclyl optionally substituted with 1-10 R, -phenyl substituted with 1-5 R⁶, -heteroaryl optionally substituted with 1-4 R⁵, —CO₂R, —OR⁹, and —(C═O)R¹⁰; wherein heteroaryl selected from the group consisting of pyridinyl, pyrimidinyl, pyrazinyl, oxazolyl, oxadiazolyl, thiazolyl, 2,3-dihydrobenzo[b]dioxinyl, 5,6,7,8-tetrahydroimidazo[1,2-a]pyrazinyl, 4,5,6,7-tetrahydro-1H-imidazo[4,5-c]pyridinyl, isoquinolinyl, and quinolinyl; wherein —(C₁₋₄ alkylene) is optionally substituted with one or more substituents as defined anywhere herein;
with the proviso that when L¹ is a bond, R² is selected from the group consisting of -phenyl substituted with 1-5 R⁶ and -heteroaryl optionally substituted with 1-4 R⁷; wherein heteroaryl selected from the group consisting of pyridinyl, oxazolyl, oxadiazolyl, thiazolyl, 2,3-dihydrobenzo[b]dioxinyl, 5,6,7,8-tetrahydroimidazo[1,2-a]pyrazinyl, 4,5,6,7-tetrahydro-1H-imidazo[4,5-c]pyridinyl, isoquinolinyl, and quinolinyl;
R³ is selected from the group consisting of -heterocyclyl substituted with 1-10 R¹¹, —(C₁₋₄ alkylene)ₚphenyl substituted with 1-5 R¹², -heteroaryl optionally substituted with 1-4 R¹³, and —(C₁₋₄ alkylene)OR¹⁴; wherein heteroaryl selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein 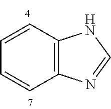
is only substituted at positions 4 and 7; wherein each —(C₁₋₄ alkylene) is, independently, optionally substituted with one or more substituents as defined anywhere herein;
with the proviso that when L² is a bond, R³ is selected from -heteroaryl optionally substituted with 1-4 R¹³; wherein heteroaryl selected from the group consisting of pyridinyl, pyrimidinyl, pyrazinyl, pyridazinyl, oxazolyl, oxadiazolyl, thiadiazolyl, indolyl, indazolyl, benzimidazolyl, imidazo[4,5-b]pyridinyl, imidazo[4,5-c]pyridinyl, 5,6,7,8-tetrahydroimidazo[1,2-a]pyrazinyl, 4,5,6,7-tetrahydro-1H-imidazo[4,5-c]pyridinyl, 1,2,3,4-tetrahydroisoquinolinyl, isoquinolinyl, and quinolinyl; wherein 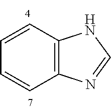
is only substituted at positions 4 and 7;
each R⁴ is halide;
each R⁵ is independently selected from the group consisting of halide, Me, and Et;
each R⁶ is independently selected from the group consisting of methyl, —CH₂F, —CHF₂, —CF₃, —OR^{15a}, and —(C₁₋₄ alkylene)ₚN(R^{16a})(R^{16b}); wherein —(C₁₋₄ alkylene) is optionally substituted with one or more substituents as defined anywhere herein;
each R⁷ is independently selected from the group consisting of F, methyl, —CH₂F, —CHF₂, —CF₃, —CF₂CH₃, —OR^{15a}, —CO₂R¹⁷, —NR¹⁸(C═O)R¹⁹, —(C₁₋₄ alkylene)ₚheterocyclyl optionally substituted with 1-10 R^{20b}, and —(C₁₋₄ alkylene)ₚN(R^{16a})(R^{16b}); wherein each —(C₁₋₄ alkylene) is, independently, optionally substituted with one or more substituents as defined anywhere herein;
R⁸ is unsubstituted —(C₁₋₉ alkyl);
R⁹ is unsubstituted —(C₁₋₉ alkyl);
R¹⁰ is -aryl optionally substituted with 1-5 R²¹;
each R¹¹ is independently selected from the group consisting of halide, methyl, and ethyl;
each R¹² is independently selected from the group consisting of —(C₁₋₄ alkylene)ₚheterocyclyl optionally substituted with 1-10 R^{20a}, -aryl optionally substituted with 1-5 R²², —(C₁₋₄ alkylene)N(R^{16a})(R^{16b}), and —OR^{23a}; wherein heterocyclyl selected from the group consisting of azetidinyl, pyrrolidinyl, piperidinyl, and piperazinyl; wherein each —(C₁₋₄ alkylene) is, independently, optionally substituted with one or more substituents as defined anywhere herein;
each R¹³ is independently selected from the group consisting of F, methyl, —CH₂F, —CHF₂, —CF₃, —(C₁₋₄ alkylene)ₚN(R^{16a})₂, —OR^{23b}, —(C₁₋₄ alkylene)ₚheterocyclyl optionally substituted with 1-10 R^{20b}, -aryl optionally substituted with 1-5 R²², and -heteroaryl substituted with 1-4 R²⁴; wherein —(C₁₋₄ alkylene) is optionally substituted with one or more substituents as defined anywhere herein;
R¹⁴ is selected from the group consisting of unsubstituted —(C₁₋₄ alkyl) and -aryl optionally substituted with 1-5 R²²;
each R^{15a} is independently selected from the group consisting of unsubstituted —(C₂₋₃ alkyl), and -heterocyclyl optionally substituted with 1-10 R^{20b};
each R^{15b} is independently selected from the group consisting of H, unsubstituted —(C₂₋₉ alkyl), and -heterocyclyl optionally substituted with 1-10 R^{20b};
each R^{16a} is independently selected from the group consisting of H and unsubstituted —(C₁₋₂ alkyl);
each R^{16b} is unsubstituted —(C₁₋₂ alkyl);
each R¹⁷ is unsubstituted —(C₁₋₉ alkyl);
each R¹⁸ is independently selected from the group consisting of H and Me;
each R¹⁹ is unsubstituted —(C₁₋₉ alkyl);
each R^{20a} is independently selected from the group consisting of halide and unsubstituted —(C₂₋₉ alkyl);
each R^{20b} is independently selected from the group consisting of halide and unsubstituted —(C₁₋₉ alkyl);
each R²¹ is independently selected from the group consisting of halide and unsubstituted —(C₁₋₉ alkyl);
each R²² is independently selected from the group consisting of halide and unsubstituted —(C₁₋₉ alkyl);
each R^{23a} is independently selected from the group consisting of unsubstituted —(C₂₋₉ alkyl), —(C₁₋₄ alkylene)OR²⁵, and —(C₁₋₄ alkylene)ₚheterocyclyl optionally substituted with 1-10 R^{20b}; wherein each —(C₁₋₄ alkylene) is, independently, optionally substituted with one or more substituents as defined anywhere herein;
each R^{23b} is independently selected from the group consisting of unsubstituted —(C₁₋₉ alkyl), —(C₁₋₄ alkylene)OR²⁵, and —(C₁₋₄ alkylene)ₚheterocyclyl optionally substituted with 1-10 R^{20b}; wherein each —(C₁₋₄ alkylene) is, independently, optionally substituted with one or more substituents as defined anywhere herein;
each R²⁴ is independently selected from the group consisting of halide and unsubstituted —(C₁₋₉ alkyl);
each R²⁵ is independently selected from the group consisting of H and unsubstituted —(C₁₋₉ alkyl);
L¹ is selected from the group consisting of a bond, —CH═CH—, 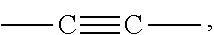
(CH₂)ₚNR¹⁸(C═O)—, —(C═O)NR¹⁸(CH₂)ₚ—, —NR¹⁸(C═O)NR¹⁸—, —NH(CH₂)ₚ—, and —(CH₂)ₚNH—;
L² is selected from the group consisting of a bond, —(C═O)NR¹⁸, —NR¹⁸ (C═O)—, —NHCH₂—, and —CH₂NH—; and
each p is independently an integer of 0 or 1.

57. The method of any one of claims 1-55, wherein the CLK inhibitor is a compound of Formula II 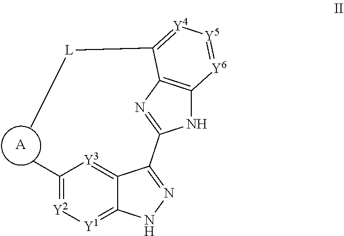
or a pharmaceutically acceptable salt or solvate thereof, wherein:
Ring A is a 5-6-membered heteroaryl optionally substituted with 1-4 R¹;
L is -L¹-L²-L³-L⁴-;
L¹ is selected from the group consisting of unsubstituted —(C₁₋₃ alkylene)-, —NR²—, —NR³(C═O)—, —(C═O)NR³—, and —O—;
L² is selected from the group consisting of unsubstituted —(C₁₋₆ alkylene)- and —NR²—;
L³ is selected from the group consisting of unsubstituted —(C₁₋₆ alkylene)-, —O—, and -carbocyclylene- optionally substituted with one or more halides;
L⁴ is selected from the group consisting of unsubstituted —(C₁₋₆ alkylene)-, —O—, —NR²—, —NR³(C═O)—, —(C═O)NR³—, -arylene- optionally substituted with 1-5 R⁴, and -heteroarylene-optionally substituted with 1-4 R⁵;
with the proviso that —NR²— and —O— are not adjacent to each other;
with the proviso that two —NR³(C═O)— and/or —(C═O)NR³—, are not adjacent to each other;
each R¹ is selected from the group consisting of halide, unsubstituted —(C₁₋₃ alkyl), unsubstituted —(C₁₋₃ haloalkyl), and —CN;
each R² is selected from the group consisting of H and unsubstituted —(C₁₋₆ alkyl);
each R³ is selected from the group consisting of H and unsubstituted —(C₁₋₆ alkyl);
each R⁴ is selected from the group consisting of halide, unsubstituted —(C₁₋₆ alkyl), unsubstituted —(C₁₋₆ haloalkyl), and —CN;
each R⁵ is selected from the group consisting of halide, unsubstituted —(C₁₋₆ alkyl), unsubstituted —(C₁₋₆ haloalkyl), and —CN;
Y¹, Y², Y³, Y⁴, Y⁵, and Y⁶ are independently selected from the group consisting of carbon and nitrogen; wherein
if Y¹ is nitrogen then Y² and Y³ are CH;
if Y² is nitrogen then Y¹ and Y³ are CH;
if Y³ is nitrogen then Y¹ and Y² are CH;
if Y⁴ is nitrogen then Y⁵ and Y⁶ are CH;
if Y⁵ is nitrogen then Y⁴ and Y⁶ are CH; and
if Y⁶ is nitrogen then Y⁴ and Y⁵ are CH.

58. The method of any one of claims 1-55, wherein the CLK inhibitor is a compound of Formula III 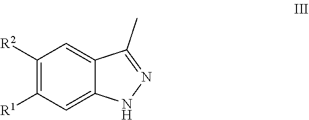
or a pharmaceutically acceptable salt or solvate thereof, wherein:
R¹ is selected from the group consisting of H and halide;
R² is a 6-membered -heteroaryl substituted with 1-4 R³;
each R³ is selected from the group consisting of —OR⁴, —NHR⁵, and —(C₁₋₄ alkylene)ₚheterocyclyl optionally substituted with 1-10 R⁶; wherein —(C₁₋₄ alkylene) is optionally substituted with one or more substituents as defined anywhere herein;
each R⁴ is independently selected from the group consisting of -heterocyclyl optionally substituted with 1-10 R⁷ and —CH₂CH(R⁸)NH₂;
each R is independently selected from the group consisting of —(C₁₋₄ alkylene)ₚheterocyclyl optionally substituted with 1-10 R⁹ and -carbocyclyl optionally substituted with 1-12 R¹⁰; wherein —(C₁₋₄ alkylene) is optionally substituted with one or more substituents as defined anywhere herein;
each R⁶ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁷ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁸ is independently selected from the group consisting of —(C₁₋₄ alkylene)aryl optionally substituted with 1-5 R¹¹ and —(C₁₋₄ alkylene)heteroaryl optionally substituted with 1-4 R¹²; wherein each —(C₁₋₄ alkylene) is, independently, optionally substituted with one or more substituents as defined anywhere herein;
each R⁹ is independently selected from the group consisting of halide, , —OH, —NH₂, unsubstituted —(C₁₋₆ alkyl), unsubstituted —(C₂₋₆ alkenyl), unsubstituted —(C₂₋₆ alkynyl), and unsubstituted —(C₁₋₆haloalkyl);
each R¹⁰ is independently selected from the group consisting of halide, —OH, —NH₂, unsubstituted —(C₁₋₆ alkyl), unsubstituted —(C₂₋₆ alkenyl), unsubstituted —(C₂₋₆ alkynyl), and unsubstituted —(C₁₋₆haloalkyl);
each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹² is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); and
each p is independently 0 or 1.

59. The method of any one of claims 1-55, wherein the CLK inhibitor is a compound of Formula IV 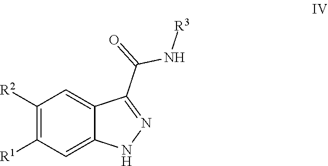
or a pharmaceutically acceptable salt or solvate thereof, wherein:
R¹ is selected from the group consisting of H and halide;
R² is a -heteroaryl optionally substituted with 1-4 R⁴;
R³ is selected from the group consisting of -aryl optionally substituted with 1-5 R⁵ and -heteroaryl optionally substituted with 1-4 R⁶;
each R⁴ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚN(R⁷)(R⁸), —NHC(═O)R⁹, —(C₁₋₄ alkylene)ₚOR¹⁰, unsubstituted -carbocyclyl, —(C₁₋₄ alkylene)ₚheterocyclyl optionally substituted with 1-10 R¹⁴, —(C₁₋₄ alkylene)ₚaryl optionally substituted with 1-5 R¹¹, and —(C₁₋₄ alkylene)ₚheteroaryl optionally substituted with 1-4 R¹²; wherein each —(C₁₋₄ alkylene) is, independently, optionally substituted with one or more substituents as defined anywhere herein;
each R is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;
each R⁶ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚaryl optionally substituted with 1-5 R¹³, —(C₁₋₄ alkylene)ₚheterocyclyl optionally substituted with 1-10 R¹⁴, —C(═O)N(R⁵)₂, —NHC(═O)R¹⁶, —(C₁₋₄ alkylene)ₚN(R¹⁷)(R¹⁸), —SO₂R¹⁹, and —OR²⁰; wherein each —(C₁₋₄ alkylene) is, independently, optionally substituted with one or more substituents as defined anywhere herein;
each R⁷ is independently selected from the group consisting of H, unsubstituted —(C₁₋₆ alkyl), unsubstituted —(C₂₋₆ alkenyl), and unsubstituted —(C₂₋₆ alkynyl);
each R⁸ is independently selected from the group consisting of H, unsubstituted —(C₁₋₆ alkyl), unsubstituted —(C₂₋₆ alkenyl), unsubstituted —(C₂₋₆ alkynyl), and -heterocyclyl optionally substituted with 1-10 R²¹;
alternatively, R⁷ and R⁸ are taken together to form a -heterocyclyl ring optionally substituted with 1-10 R²¹;
each R⁹ is independently selected from the group consisting of —N(R²²)₂, -carbocyclyl optionally substituted with 1-12 R²³, -heterocyclyl optionally substituted with 1-10 R²¹, and -aryl optionally substituted with 1-5 R²⁴;
each R¹⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -heterocyclyl optionally substituted with 1-10 R²¹;
each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹² is independently selected from the group consisting of halide, —(C₁₋₄ alkylene)ₚOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;
each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁴ is independently selected from the group consisting of halide, —(C₁₋₄ alkylene)pOH, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); wherein —(C₁₋₄ alkylene) is optionally substituted with one or more substituents as defined anywhere herein;
each R¹⁵ is independently selected from the group consisting of H, unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;
alternatively, two adjacent R¹⁵ are taken together to form a -heterocyclyl ring optionally substituted with 1-10 R²¹;
each R¹⁶ is independently selected from the group consisting of unsubstituted —(C₁₋₆ alkyl), unsubstituted —(C₂₋₆ alkenyl), unsubstituted —(C₂₋₆ alkynyl), and -carbocyclyl optionally substituted with 1-12 R²³;
each R¹⁷ is independently selected from the group consisting of H, unsubstituted —(C₁₋₆ alkyl), unsubstituted —(C₂₋₆ alkenyl), and unsubstituted —(C₂₋₆ alkynyl);
each R¹⁸ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)NMe₂, and -heterocyclyl ring optionally substituted with 1-10 R²¹; wherein —(C₁₋₄ alkylene) is optionally substituted with one or more substituents as defined anywhere herein;
each R¹⁹ is independently selected from the group consisting of unsubstituted —(C₁₋₆ alkyl), unsubstituted —(C₂₋₆ alkenyl), and unsubstituted —(C₂₋₆ alkynyl).
each R²⁰ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —CH(CH₂OH)₂, —(C₁₋₄ alkylene)ₚheterocyclyl ring optionally substituted with 1-10 R²¹, and -aryl optionally substituted with 1-5 R²⁴; wherein —(C₁₋₄ alkylene) is optionally substituted with one or more substituents as defined anywhere herein;
each R²¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²² is independently selected from the group consisting of unsubstituted —(C₁₋₆ alkyl), unsubstituted —(C₂₋₆ alkenyl), and unsubstituted —(C₂₋₆ alkynyl);
each R²³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²⁴ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); and
each p is independently 0 or 1.

60. The method of any one of claims 1-55, wherein the CLK inhibitor is a compound of Formula V 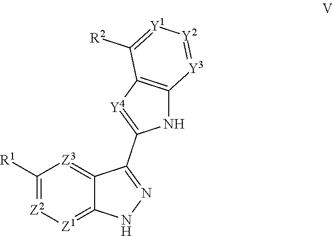
or a pharmaceutically acceptable salt or solvate thereof or a pharmaceutically acceptable salt or solvate thereof, wherein:
R¹ is a -heteroaryl optionally substituted with 1-2 R³;
R² is selected from the group consisting of H, halide, -aryl optionally substituted with 1-5 R⁴-heteroaryl optionally substituted with 1-4 R⁵, and -heterocyclyl ring optionally substituted with 1-10 R⁶;
each R³ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R⁷, —C(═O)N(R⁸)₂, —NHC(═O)R⁹, —(C₁₋₄ alkylene)ₚN(R¹⁰)(R¹¹), —(C₁₋₄ alkylene)ₚOR¹², and -carbocyclyl optionally substituted with 1-12 R¹³; wherein each —(C₁₋₄ alkylene) is, independently, optionally substituted with one or more substituents as defined anywhere herein;
each R⁴ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚNHSO₂R¹⁴, —NR⁵(C₁₋₄ alkylene)NR¹⁵R¹⁶, —(C₁₋₄ alkylene)ₚNR¹⁵R¹⁶, —OR¹⁷, and -heterocyclyl optionally substituted with 1-10 R¹⁹; wherein each —(C₁₋₄ alkylene) is, independently, optionally substituted with one or more substituents as defined anywhere herein;
each R⁵ is independently selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and —C(═O)R¹⁸;
each R⁶ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁷ is independently selected from the group consisting of halide, —NH₂, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁸ is independently selected from the group consisting of H, unsubstituted —(C₁₋₆ alkyl), unsubstituted —(C₂₋₆ alkenyl), unsubstituted —(C₂₋₆ alkynyl), -heterocyclyl optionally substituted with 1-10 R¹⁹, —(C₁₋₄ alkylene)ₚcarbocyclyl optionally substituted with 1-12 R²⁰; wherein —(C₁₋₄ alkylene) is optionally substituted with one or more substituents as defined anywhere herein;
each R⁹ is independently selected from the group consisting of unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²²)₂; wherein each —(C₁₋₄ alkylene) is, independently, optionally substituted with one or more substituents as defined anywhere herein;
each R¹⁰ is independently selected from the group consisting of H, unsubstituted —(C₁₋₆ alkyl), unsubstituted —(C₂₋₆ alkenyl), and unsubstituted —(C₂₋₆ alkynyl);
each R¹¹ is independently selected from the group consisting of H, unsubstituted —(C₁₋₆ alkyl), unsubstituted —(C₂₋₆ alkenyl), unsubstituted —(C₂₋₆ alkynyl), —(C₁₋₄ alkylene)ₚcarbocyclyl optionally substituted with 1-12 R²⁰; and —(C₁₋₄ alkylene)ₚaryl optionally substituted with 1-5 R²¹; wherein each —(C₁₋₄ alkylene) is, independently, optionally substituted with one or more substituents as defined anywhere herein;
each R¹² is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, —(C₁₋₄ alkylene)ₚcarbocyclyl optionally substituted with 1-12 R²⁰; —(C₁₋₄ alkylene)ₚaryl optionally substituted with 1-5 R²¹, —(C₁₋₄ alkylene)ₚN(R²)₂; wherein each —(C₁₋₄ alkylene) is, independently, optionally substituted with one or more substituents as defined anywhere herein;
each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁴ is independently selected from the group consisting of unsubstituted —(C₁₋₆ alkyl), unsubstituted —(C₂₋₆ alkenyl), and unsubstituted —(C₂₋₆ alkynyl);
each R¹⁵ is independently selected from the group consisting of H, unsubstituted —(C₁₋₆ alkyl), unsubstituted —(C₂₋₆ alkenyl), and unsubstituted —(C₂₋₆ alkynyl);
each R¹⁶ is independently selected from the group consisting of H, unsubstituted —(C₁₋₆ alkyl), unsubstituted —(C₂₋₆ alkenyl), and unsubstituted —(C₂₋₆ alkynyl);
each R¹⁷ is independently selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —(C₁₋₄ alkylene)ₚheterocyclyl optionally substituted with 1-10 R¹⁹, and, —(C₁₋₄ alkylene)ₚN(R²²)₂; wherein each —(C₁₋₄ alkylene) is, independently, optionally substituted with one or more substituents as defined anywhere herein;
each R¹⁸ is independently selected from the group consisting of unsubstituted —(C₁₋₆ alkyl), unsubstituted —(C₂₋₆ alkenyl), and unsubstituted —(C₂₋₆ alkynyl);
each R¹⁹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²⁰ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R²² is independently selected from the group consisting of H, unsubstituted —(C₁₋₆ alkyl), unsubstituted —(C₂₋₆ alkenyl), and unsubstituted —(C₂₋₆ alkynyl);
each R²³ is independently selected from the group consisting of H and halide;
Y¹, Y², and Y³ are independently selected from the group consisting of —CR²³═ and —N═;
Y⁴ is selected from the group of —CH═ and —N═;
Z¹, Z², and Z³ are independently selected from the group consisting of —CR²³═ and —N═; and
each p is independently 0 or 1.

61. The method of any one of claims 1-55, wherein the CLK inhibitor is a compound of Formula VI 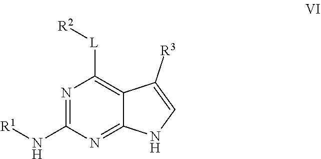
or a pharmaceutically acceptable salt or solvate thereof, wherein:
R¹ is selected from the group consisting of H, unsubstituted —(C₁₋₆ alkyl), unsubstituted —(C₂₋₆ alkenyl), unsubstituted —(C₂₋₆ alkynyl), and -heteroaryl optionally substituted with 1-4 R⁴, -aryl optionally substituted with 1-5 R⁵;
R² is selected from the group consisting of H, —(C₁₋₄ alkylene)ₚheteroaryl optionally substituted with 1-4 R⁶, —(C₁₋₄ alkylene)ₚheterocyclyl optionally substituted with 1-10 R⁷, and —(C₁₋₄ alkylene)ₚcarbocyclyl optionally substituted with 1-12 R⁸; wherein each —(C₁₋₄ alkylene) is, independently, optionally substituted with one or more substituents as defined anywhere herein;
R³ is selected from the group consisting of -heteroaryl optionally substituted with 1-4 R⁹ and -aryl optionally substituted with 1-5 R¹⁰;
each R⁴ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R¹⁴, and —(C₁₋₄ alkylene)ₚcarbocyclyl optionally substituted with 1-12 R⁵; wherein each —(C₁₋₄ alkylene) is, independently, optionally substituted with one or more substituents as defined anywhere herein;
each R⁵ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, —(C₁₋₄ alkylene)ₚheterocyclyl optionally substituted with 1-10 R¹³, —SO₂R¹⁴, and —(C₁₋₄ alkylene)ₚcarbocyclyl optionally substituted with 1-12 R⁵; wherein each —(C₁₋₄ alkylene) is, independently, optionally substituted with one or more substituents as defined anywhere herein;
each R⁶ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;
each R⁷ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁸ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R⁹ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;
each R¹⁰ is independently selected from the group consisting of halide, —CN, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —OR¹¹, —C(═O)N(R¹²)₂, and —SO₂R¹⁴;
each R¹¹ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹² is independently selected from the group consisting of H, halide, unsubstituted —(C₁₋₆ alkyl), unsubstituted —(C₂₋₆ alkenyl), and unsubstituted —(C₂₋₆ alkynyl);
each R¹³ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
each R¹⁴ is independently selected from the group consisting of halide, unsubstituted —(C₁₋₆ alkyl), unsubstituted —(C₂₋₆ alkenyl), and unsubstituted —(C₂₋₆ alkynyl);
each R¹⁵ is independently selected from the group consisting of halide, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
L is selected from the group consisting of a bond, —O—, and —NH—; and
each p is independently 0 or 1.

62. The method of any one of claims 1-55, wherein the CLK inhibitor is a compound of Formula VII 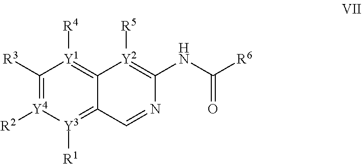
or a pharmaceutically acceptable salt or solvate thereof, wherein:
R¹, R², R⁴, and R⁵ are independently absent or selected from the group consisting of H and halide;
R³ is selected from the group of -heteroaryl optionally substituted with 1-4 R⁸ and -Xheterocyclyl optionally substituted with one or more halides or one or more unsubstituted —(C₁₋₅ alkyl);
R⁶ is selected from the group consisting of -aryl substituted with 1-5 R⁹, —(C₂₋₄ alkenylene)aryl substituted with 1-5 R⁹, —(C₁₋₄ alkylene)ₚheteroaryl optionally substituted with 1-6 R¹⁰; -heterocyclyl optionally substituted with 1-10 R¹¹, -carbocyclyl optionally substituted with 1-12 R¹², and —(C₂₋₉ alkynyl) optionally substituted with one or more halides; wherein each —(C₁₋₄ alkylene) is, independently, optionally substituted with one or more substituents as defined anywhere herein; wherein —(C₁₋₄ alkenylene) is, optionally substituted with one or more substituents as defined anywhere herein;
with the proviso that R⁶ is heterocyclyl only when R³ is a 6-membered heteroaryl;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —N(R¹⁵)(R¹⁸), —(C₁₋₄ alkylene)ₚXR¹⁹, —C(═O)N(R⁵)₂, —(C₁₋₄ alkylene)ₚheterocyclyl optionally substituted with 1-10 R²⁰, and -carbocyclyl optionally substituted with 1-12 R²¹; wherein each —(C₁₋₄ alkylene) is, independently, optionally substituted with one or more substituents as defined anywhere herein;
alternatively, two adjacent R⁸ are taken together to form a ring which is selected from the group consisting of -heterocyclyl optionally substituted with 1-10 R²² and -carbocyclyl optionally substituted with 1-12 R²¹;
each R⁹ is independently selected from the group consisting of D, halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —XR²³, —(C₁₋₄ alkylene)ₚN(R²⁴)₂, —(C₁₋₄ alkylene)ₚheterocyclyl optionally substituted with 1-10 R²²; wherein each —(C₁₋₄ alkylene) is, independently, optionally substituted with one or more substituents as defined anywhere herein;
each R¹⁰ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —CN, —XR²³, —C(═O)N(R¹⁵)₂, —(C₁₋₄ alkylene)ₚN(R²⁴)₂, -heterocyclyl optionally substituted with 1-10 R²², and -carbocyclyl optionally substituted with 1-12 R²¹; wherein —(C₁₋₄ alkylene) is optionally substituted with one or more substituents as defined anywhere herein;
each R¹¹ is independently selected from the group consisting of halide, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
each R¹² is independently selected from the group consisting of halide, —(C₁₋₄ alkylene)ₚOR¹⁹; wherein —(C₁₋₄ alkylene) is optionally substituted with one or more substituents as defined anywhere herein;
each R¹⁵ is selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
R¹⁸ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —(C₁₋₄ alkylene)ₚheterocyclyl optionally substituted with one or more halides or one or more unsubstituted —(C₁₋₅ alkyl); wherein —(C₁₋₄ alkylene) is, independently, optionally substituted with one or more substituents as defined anywhere herein;
each R¹⁹ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl); wherein —(C₁₋₄ alkylene) is optionally substituted with one or more substituents as defined anywhere herein;
each R²⁰ independently is selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —OH;
each R²¹ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), and —CN;
each R²² is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —OH, —N(R¹⁵)₂, —C(═O)R³⁴, and -carbocyclyl optionally substituted with 1-12 R²¹;
each R²³ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)N(R¹⁵)₂, -heterocyclyl optionally substituted with 1-10 R³¹, and -carbocyclyl optionally substituted with 1-12 R²¹; wherein —(C₁₋₄ alkylene) is optionally substituted with one or more substituents as defined anywhere herein;
each R²⁴ is independently selected from the group consisting of H, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —(C₁₋₄ alkylene)ₚheterocyclyl optionally substituted with one or more halides or one or more unsubstituted —(C₁₋₅ alkyl), and —(C₁₋₄ alkylene)N(R⁵)₂; wherein each —(C₁₋₄ alkylene) is, independently, optionally substituted with one or more substituents as defined anywhere herein;
each R³¹ is independently selected from the group consisting of halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
each R³⁴ is independently selected from the group consisting of —O(C₁₋₅ alkyl) and a heteroaryl optionally substituted with 1-6 R³⁵;
each R³⁵ is a -heterocyclyl optionally substituted with one or more halides or one or more unsubstituted —(C₁₋₅ alkyl);
each X is selected from the group consisting of O and S;
Y¹, Y², Y³, and Y⁴ are independently selected from the group consisting of carbon and nitrogen; wherein
if Y¹ is nitrogen then Y², Y³, and Y⁴ are carbon, and R⁴ is absent;
if Y² is nitrogen then Y¹, Y³, and Y⁴ are carbon, and R⁵ is absent;
if Y³ is nitrogen then Y¹, Y², and Y⁴ are carbon, and R¹ is absent;
if Y⁴ is nitrogen then Y¹, Y², and Y³ are carbon, and R² is absent; and
each p is independently 0 or 1.

63. The method of any one of claims 1-55, wherein the CLK inhibitor is a compound of Formula VIII 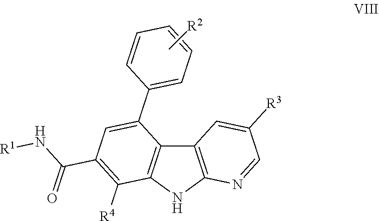
or a pharmaceutically acceptable salt or solvate thereof, wherein:
R¹ is selected from the group consisting of —(C₁₋₄ alkylene)N(R⁵)₂, —(C₁₋₄ alkylene)ₚheterocyclyl optionally substituted with 1-10 R⁶, and —(C₁₋₄ alkylene)ₚcarbocyclyl optionally substituted with 1-12 R⁷; wherein each —(C₁₋₄ alkylene) is, independently, optionally substituted with one or more substituents as defined anywhere herein;
R² is selected from the group consisting of halide, unsubstituted —(C₁₋₆ alkyl), unsubstituted —(C₂₋₆ alkenyl), unsubstituted —(C₂₋₆ alkynyl), unsubstituted —(C₁₋₆ haloalkyl), —CN, —OR, —C(═O)NHR⁹, —NHC(═O)(R¹⁰), —SO₂R¹⁰, —NHSO₂R¹⁰, and —SO₂NHR⁹;
R³ is selected from the group consisting of H, halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
R⁴ is selected from the group consisting of H, halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
each R⁵ is independently selected from the group consisting of H, unsubstituted —(C₁₋₅ alkyl), unsubstituted —(C₂₋₅ alkenyl), and unsubstituted —(C₂₋₅ alkynyl);
each R⁶ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —OH, and —CN;
each R⁷ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —OH, and —CN;
R⁸ is selected from the group consisting of H, unsubstituted —(C₁₋₆ alkyl), unsubstituted —(C₂₋₆ alkenyl), unsubstituted —(C₂₋₆ alkynyl), and —(C₁₋₄ alkylene)ₚheterocyclyl optionally substituted with 1-10 R⁶; wherein —(C₁₋₄ alkylene) is optionally substituted with one or more substituents as defined anywhere herein;
each R⁹ is independently selected from the group consisting of H, unsubstituted —(C₁₋₅ alkyl), unsubstituted —(C₂₋₆ alkenyl), and unsubstituted —(C₂₋₅ alkynyl), and —(C₁₋₄ alkylene)ₚheterocyclyl optionally substituted with 1-10 R⁶; wherein —(C₁₋₄ alkylene) is optionally substituted with one or more substituents as defined anywhere herein;
each R¹⁰ is independently selected from the group consisting of unsubstituted —(C₁₋₅ alkyl), unsubstituted —(C₂₋₅ alkenyl), and unsubstituted —(C₂₋₅ alkynyl), and —(C₁₋₄ alkylene)ₚheterocyclyl optionally substituted with 1-10 R⁶; wherein —(C₁₋₄ alkylene) is optionally substituted with one or more substituents as defined anywhere herein; and
each p is independently 0 or 1.

64. The method of any one of claims 1-55, wherein the CLK inhibitor is a compound of Formula IX 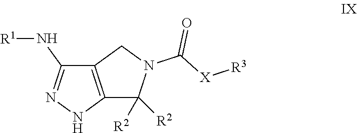
or a pharmaceutically acceptable salt or solvate thereof, wherein:
R¹ is -heteroaryl optionally substituted with 1-6 R⁴;
each R² is independently selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
R³ is —CH(R⁵)R⁶;
each R⁴ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OR⁷, -carbocyclyl optionally substituted with 1-12 R⁸;
R⁵ is -aryl optionally substituted with 1-5 R⁹;
R⁶ is —(C₁₋₄ alkylene)N(R¹⁰)₂; wherein —(C₁₋₄ alkylene) is optionally substituted with one or more substituents as defined anywhere herein;
each R⁷ is independently selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
each R⁹ is independently selected from the group consisting of halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
each R⁹ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, and —OR⁷;
each R¹⁰ is independently selected from the group consisting of H, unsubstituted —(C₁₋₅ alkyl), unsubstituted —(C₂₋₅ alkenyl), and unsubstituted —(C₂₋₅ alkynyl); and
X is selected from the group consisting of O, S, and NH.

65. The method of any one of claims 1-55, wherein the CLK inhibitor is a compound of Formula X 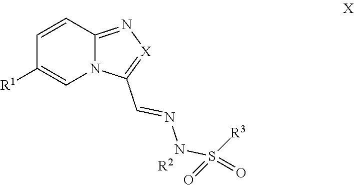
or a pharmaceutically acceptable salt or solvate thereof, wherein:
R¹ is selected from the group consisting of H, halide, unsubstituted —(C₁₋₅ alkyl), unsubstituted —(C₁₋₅ haloalkyl), and —CN;
R² is selected from the group consisting of H, unsubstituted —(C₁₋₅ alkyl), unsubstituted —(C₂₋₅ alkenyl), and unsubstituted —(C₂₋₅ alkynyl);
R³ is -aryl optionally substituted with 1-5 R⁴;
each R⁴ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —NO₂, —CN, and —OMe;
R⁵ is selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl); and
X is selected from the group consisting of N and CR⁵.

66. The method of any one of claims 1-55, wherein the CLK inhibitor is a compound of Formula XI 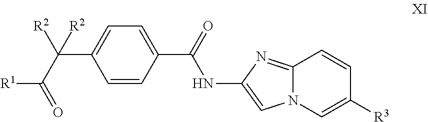
or a pharmaceutically acceptable salt or solvate thereof, wherein:
R¹ is —N(R⁴)₂;
R² is selected from the group consisting of H, unsubstituted —(C₁₋₅ alkyl), unsubstituted —(C₂₋₅ alkenyl), unsubstituted —(C₂₋₅ alkynyl), and unsubstituted —(C₁₋₅ haloalkyl);
R³ is -heteroaryl optionally substituted with 1-6 R⁵;
each R⁴ is independently selected from the group consisting of H, unsubstituted —(C₁₋₅ alkyl), unsubstituted —(C₂₋₅ alkenyl), unsubstituted —(C₂₋₅ alkynyl), and -heterocyclyl optionally substituted with 1-10 R⁶;
alternatively, two adjacent R⁴ are taken together to form a ring which is selected from the group consisting of -heterocyclyl optionally substituted with 1-10 R⁶;
each R⁵ is independently selected from the group consisting of halide, unsubstituted —(C₁₋₅ alkyl), unsubstituted —(C₂₋₅ alkenyl), unsubstituted —(C₂₋₅ alkynyl), unsubstituted —(C₁₋₅ haloalkyl), —CN, —OH, and —OMe; and
each R⁶ is independently selected from the group consisting of halide, unsubstituted —(C₁₋₅ alkyl), unsubstituted —(C₂₋₅ alkenyl), unsubstituted —(C₂₋₅ alkynyl), and unsubstituted —(C₁₋₅ haloalkyl).

67. The method of any one of claims 1-55, wherein the CLK inhibitor is a compound of Formula XII 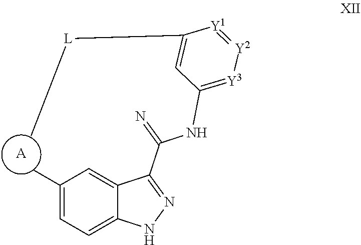
or a pharmaceutically acceptable salt or solvate thereof, wherein:
Ring A is a 5-6-membered heteroaryl optionally substituted with 1-3 R¹;
L is -L1-L²-L³-L⁴-
L¹ is selected from the group consisting of unsubstituted —(C₁₋₃ alkylene)-, —NR²—, —NR³(C═O)—, —(C═O)NR³—, and —O—;
L² is selected from the group consisting of unsubstituted —(C₁₋₆ alkylene)-, —NR²—, —NR³(C═O)—, and —(C═O)NR³—;
L³ is selected from the group consisting of unsubstituted —(C₁₋₆ alkylene)-, —O—, and carbocyclylene optionally substituted with one or more halides;
L⁴ is selected from the group consisting of unsubstituted —(C₁₋₆ alkylene)-, —O—, —NR²—, —NR³(C═O)—, —(C═O)NR³—, -arylene substituted with 1-5 R⁴, and -heteroarylene optionally substituted with 1-4 R⁵;
with the proviso that —NR²— and —O— are not adjacent to each other;
with the proviso that two —NR³(C═O)— and/or —(C═O)NR³—, are not adjacent to each other;
each R¹ is selected from the group consisting of halide, unsubstituted —(C₁₋₃ alkyl), unsubstituted —(C₁₋₃ haloalkyl), and —CN;
each R² is selected from the group consisting of H and unsubstituted —(C₁₋₆ alkyl);
each R³ is selected from the group consisting of H and unsubstituted —(C₁₋₆ alkyl);
each R⁴ is selected from the group consisting of halide, unsubstituted —(C₁₋₆ alkyl), unsubstituted —(C₁₋₆ haloalkyl), and —CN;
each R⁵ is selected from the group consisting of halide, unsubstituted —(C₁₋₆ alkyl), unsubstituted —(C₁₋₆ haloalkyl), and —CN;
Y¹, Y², and Y³ are independently selected from the group consisting of carbon and nitrogen; wherein
if Y¹ is nitrogen then Y² and Y³ are CH;
if Y² is nitrogen then Y¹ and Y³ are CH; and
if Y³ is nitrogen then Y¹ and Y² are CH.


SEQUENCE LISTING (34 sequences)

SEQ ID NO: 1 - protein, 484 aa, Homo sapiens
MRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRRCKPLKEFMLSQDVEHERLFDLIQKMLEYDPAKRITLREALKHPFFDLLKKSI

SEQ ID NO: 2 - DNA, 1455 bp, Homo sapiens
atgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatag

SEQ ID NO: 3 - protein, 526 aa, Homo sapiens
MAAGRRPASALWPERRGSPLRGDLLGFQNVREPSSCGETLSGMRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRRCKPLKEFMLSQDVEHERLFDLIQKMLEYDPAKRITLREALKHPFFDLLKKSI

SEQ ID NO: 4 - DNA, 1581 bp, Homo sapiens
atggcggctgggcggaggccggcttcggccctgtggccggaaaggcgaggctccccgttgaggggggatttgctggggttccagaatgtgcgtgagccaagcagctgtggggaaacgttgtctggaatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatag

SEQ ID NO: 5 - protein, 499 aa, Homo sapiens
MPHPRRYHSSERGSRGSYREHYRSRKHKRRRSRSWSSSSDRTRRRRREDSYHVRSRSSYDDRSSDRRVYDRRYCGSYRRNDYSRDRGDAYYDTDYRHSYEYQRENSSYRSQRSSRRKHRRRRRRSRTFSRSSSQHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 6 - DNA, 1500 bp, Homo sapiens
atgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcagcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 7 - protein, 498 aa, Homo sapiens
MPHPRRYHSSERGSRGSYREHYRSRKHKRRRSRSWSSSSDRTRRRRREDSYHVRSRSSYDDRSSDRRVYDRRYCGSYRRNDYSRDRGDAYYDTDYRHSYEYQRENSSYRSQRSSRRKHRRRRRRSRTFSRSSSHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 8 - DNA, 1497 bp, Homo sapiens
atgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagcagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 9 - protein, 271 aa, Homo sapiens
MFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 10 - DNA, 816 bp, Homo sapiens
atgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 11 - protein, 497 aa, Homo sapiens
MPHPRRYHSSERGSRGSYREHYRSRKHKRRRSRSWSSSSDRTRRRRREDSYHVRSRSYDDRSSDRRVYDRRYCGSYRRNDYSRDRGDAYYDTDYRHSYEYQRENSSYRSQRSSRRKHRRRRRRSRTFSRSSSHSSRRAKSVEDDAEGHLIYHVGDWLQERYEIVSTLGEGTFGRVVQCVDHRRGGARVALKIIKNVEKYKEAARLEINVLEKINEKDPDNKNLCVQMFDWFDYHGHMCISFELLGLSTFDFLKDNNYLPYPIHQVRHMAFQLCQAVKFLHDNKLTHTDLKPENILFVNSDYELTYNLEKKRDERSVKSTAVRVVDFGSATFDHEHHSTIVSTRHYRAPEVILELGWSQPCDVWSIGCIIFEYYVGFTLFQTHDNREHLAMMERILGPIPSRMIRKTRKQKYFYRGRLDWDENTSAGRYVRENCKPLRRYLTSEAEEHHQLFDLIESMLEYEPAKRLTLGEALQHPFFARLRAEPPNKLWDSSRDISR

SEQ ID NO: 12 - DNA, 1494 bp, Homo sapiens
atgccgcatcctcgaaggtaccactcctcagagcgaggcagccgggggagttaccgtgaacactatcggagccgaaagcataagcgacgaagaagtcgctcctggtcaagtagtagtgaccggacacgacggcgtcggcgagaggacagctaccatgtccgttctcgaagttatgatgatcgttcgtccgaccggagggtgtatgaccggcgatactgtggcagctacagacgcaacgattatagccgggatcggggagatgcctactatgacacagactatcggcattcctatgaatatcagcgggagaacagcagttaccgcagccagcgcagcagccggaggaagcacagacggcggaggaggcgcagccggacatttagccgctcatcttcgcacagcagccggagagccaagagtgtagaggacgacgctgagggccacctcatctaccacgtcggggactggctacaagagcgatatgaaatcgttagcaccttaggagaggggaccttcggccgagttgtacaatgtgttgaccatcgcaggggtggggctcgagttgccctgaagatcattaagaatgtggagaagtacaaggaagcagctcgacttgagatcaacgtgctagagaaaatcaatgagaaagaccctgacaacaagaacctctgtgtccagatgtttgactggtttgactaccatggccacatgtgtatctcctttgagcttctgggccttagcaccttcgatttcctcaaagacaacaactacctgccctaccccatccaccaagtgcgccacatggccttccagctgtgccaggctgtcaagttcctccatgataacaagctgacacatacagacctcaagcctgaaaatattctgtttgtgaattcagactatgagctcacctacaacctagagaagaagcgagatgagcgcagtgtgaagagcacagctgtgcgggtggtagactttggcagtgccacctttgaccatgagcaccatagcaccattgtctccactcgccattaccgagcaccagaagtcatccttgagttgggctggtcacagccttgtgatgtgtggagtataggctgcatcatctttgaatactatgtgggattcaccctcttccagacccatgacaacagagagcatctagccatgatggaaaggatcttgggtcctatcccttcccggatgatccgaaagacaagaaagcagaaatatttttaccggggtcgcctggattgggatgagaacacatcagctgggcgctatgttcgtgagaactgcaaaccgctgcggcggtatctgacctcagaggcagaggaacaccaccagctcttcgatctgattgaaagcatgctagagtatgaaccagctaagcggctgaccttgggtgaagcccttcagcatcctttcttcgcccgccttcgggctgagccgcccaacaagttgtgggactccagtcgggatatcagtcggtga

SEQ ID NO: 13 - protein, 638 aa, Homo sapiens
MPVLSARRRELADHAGSGRRSGPSPTARSGPHLSALRAQPARAAHLSGRGTYVRRDTAGGGPGQARPLGPPGTSLLGRGARRSGEGWCPGAFESGARAARPPSRVEPRLATAASREGAGLPRAEVAAGSGRGARSGEWGLAAAGAWETMHHCKRYRSPEPDPYLSYRWKRRRSYSREHEGRLRYPSRREPPPRRSRSRSHDRLPYQRRYRERRDSDTYRCEERSPSFGEDYYGPSRSRHRRRSRERGPYRTRKHAHHCHKRRTRSCSSASSRSQQSSKRSSRSVEDDKEGHLVCRIGDWLQERYEIVGNLGEGTFGKVVECLDHARGKSQVALKIIRNVGKYREAARLEINVLKKIKEKDKENKFLCVLMSDWFNFHGHMCIAFELLGKNTFEFLKENNFQPYPLPHVRHMAYQLCHALRFLHENQLTHTDLKPENILFVNSEFETLYNEHKSCEEKSVKNTSIRVADFGSATFDHEHHTTIVATRHYRPPEVILELGWAQPCDVWSIGCILFEYYRGFTLFQTHENREHLVMMEKILGPIPSHMIHRTRKQKYFYKGGLVWDENSSDGRYVKENCKPLKSYMLQDSLEHVQLFDLMRRMLEFDPAQRITLAEALLHPFFAGLTPEERSFHTSRNPSR

SEQ ID NO: 14 - DNA, 1918 bp, Homo sapiens
aatgcccgtcctctccgcgcgcaggagggagttggcggaccacgcggggtcggggcgacggagcgggcccagccccacggccaggtcggggccccacctctcggctctgagagcccagccggcccgggccgcgcacctgtcaggtcgggggacctacgtgcgccgcgacacggcgggaggcgggccgggccaggctcgtcccctcggccctcccggaactagtctcctaggccgcggcgcccgccggagcggagagggctggtgccccggagccttcgagtcgggggctagagcggccaggcctccgagccgggtcgagccgaggctggcgacggctgcgtcacgcgagggggcggggctgccacgggcggaggtcgcagccggaagcggaagaggcgctcggagcggggagtggggcctagctgcagccggagcctgggagacgatgcatcactgtaagcgataccgctcccctgaaccagacccgtacctgagctaccgatggaagaggaggaggtcctacagtcgggaacatgaagggagactgcgatacccgtcccgaagggagcctcccccacgaagatctcggtccagaagccatgaccgcctgccctaccagaggaggtaccgggagcgccgtgacagcgatacataccggtgtgaagagcggagcccatcctttggagaggactactatggaccttcacgttctcgtcatcgtcggcgatcgcgggagagggggccataccggacccgcaagcatgcccaccactgccacaaacgccgcaccaggtcttgtagcagcgcctcctcgagaagccaacagagcagtaagcgcagcagccggagtgtggaagatgacaaggagggtcacctggtgtgccggatcggcgattggctccaagagcgatatgagattgtggggaacctgggtgaaggcacctttggcaaggtggtggagtgcttggaccatgccagagggaagtctcaggttgccctgaagatcatccgcaacgtgggcaagtaccgggaggctgcccggctagaaatcaacgtgctcaaaaaaatcaaggagaaggacaaagaaaacaagttcctgtgtgtcttgatgtctgactggttcaacttccacggtcacatgtgcatcgcctttgagctcctgggcaagaacacctttgagttcctgaaggagaataacttccagccttaccccctaccacatgtccggcacatggcctaccagctctgccacgcccttagatttctgcatgagaatcagctgacccatacagacttgaaaccagagaacatcctgtttgtgaattctgagtttgaaaccctctacaatgagcacaagagctgtgaggagaagtcagtgaagaacaccagcatccgagtggctgactttggcagtgccacatttgaccatgagcaccacaccaccattgtggccacccgtcactatcgcccgcctgaggtgatccttgagctgggctgggcacagccctgtgacgtctggagcattggctgcattctctttgagtactaccggggcttcacactcttccagacccacgaaaaccgagagcacctggtgatgatggagaagatcctagggcccatcccatcacacatgatccaccgtaccaggaagcagaaatatttctacaaagggggcctagtttgggatgagaacagctctgacggccggtatgtgaaggagaactgcaaacctctgaagagttacatgctccaagactccctggagcacgtgcagctgtttgacctgatgaggaggatgttagaatttgaccctgcccagcgcatcacactggccgaggccctgctgcaccccttctttgctggcctgacccctgaggagcggtccttccacaccagccgcaacccaagcagatga

SEQ ID NO: 15 - protein, 490 aa, Homo sapiens
MHHCKRYRSPEPDPYLSYRWKRRRSYSREHEGRLRYPSRREPPPRRSRSRSHDRLPYQRRYRERRDSDTYRCEERSPSFGEDYYGPSRSRHRRRSRERGPYRTRKHAHHCHKRRTRSCSSASSRSQQSSKRSSRSVEDDKEGHLVCRIGDWLQERYEIVGNLGEGTFGKVVECLDHARGKSQVALKIIRNVGKYREAARLEINVLKKIKEKDKENKFLCVLMSDWFNFHGHMCIAFELLGKNTFEFLKENNFQPYPLPHVRHMAYQLCHALRFLHENQLTHTDLKPENILFVNSEFETLYNEHKSCEEKSVKNTSIRVADFGSATFDHEHHTTIVATRHYRPPEVILELGWAQPCDVWSIGCILFEYYRGFTLFQTHENREHLVMMEKILGPIPSHMIHRTRKQKYFYKGGLVWDENSSDGRYVKENCKPLKSYMLQDSLEHVQLFDLMRRMLEFDPAQRITLAEALLHPFFAGLTPEERSFHTSRNPSR

SEQ ID NO: 16 - DNA, 1473 bp, Homo sapiens
atgcatcactgtaagcgataccgctcccctgaaccagacccgtacctgagctaccgatggaagaggaggaggtcctacagtcgggaacatgaagggagactgcgatacccgtcccgaagggagcctcccccacgaagatctcggtccagaagccatgaccgcctgccctaccagaggaggtaccgggagcgccgtgacagcgatacataccggtgtgaagagcggagcccatcctttggagaggactactatggaccttcacgttctcgtcatcgtcggcgatcgcgggagagggggccataccggacccgcaagcatgcccaccactgccacaaacgccgcaccaggtcttgtagcagcgcctcctcgagaagccaacagagcagtaagcgcagcagccggagtgtggaagatgacaaggagggtcacctggtgtgccggatcggcgattggctccaagagcgatatgagattgtggggaacctgggtgaaggcacctttggcaaggtggtggagtgcttggaccatgccagagggaagtctcaggttgccctgaagatcatccgcaacgtgggcaagtaccgggaggctgcccggctagaaatcaacgtgctcaaaaaaatcaaggagaaggacaaagaaaacaagttcctgtgtgtcttgatgtctgactggttcaacttccacggtcacatgtgcatcgcctttgagctcctgggcaagaacacctttgagttcctgaaggagaataacttccagccttaccccctaccacatgtccggcacatggcctaccagctctgccacgcccttagatttctgcatgagaatcagctgacccatacagacttgaaaccagagaacatcctgtttgtgaattctgagtttgaaaccctctacaatgagcacaagagctgtgaggagaagtcagtgaagaacaccagcatccgagtggctgactttggcagtgccacatttgaccatgagcaccacaccaccattgtggccacccgtcactatcgcccgcctgaggtgatccttgagctgggctgggcacagccctgtgacgtctggagcattggctgcattctctttgagtactaccggggcttcacactcttccagacccacgaaaaccgagagcacctggtgatgatggagaagatcctagggcccatcccatcacacatgatccaccgtaccaggaagcagaaatatttctacaaagggggcctagtttgggatgagaacagctctgacggccggtatgtgaaggagaactgcaaacctctgaagagttacatgctccaagactccctggagcacgtgcagctgtttgacctgatgaggaggatgttagaatttgaccctgcccagcgcatcacactggccgaggccctgctgcaccccttctttgctggcctgacccctgaggagcggtccttccacaccagccgcaacccaagcagatga

SEQ ID NO: 17 - protein, 481 aa, Homo sapiens
MRHSKRTHCPDWDSRESWGHESYRGSHKRKRRSHSSTQENRHCKPHHQFKESDCHYLEARSLNERDYRDRRYVDEYRNDYCEGYVPRHYHRDIESGYRIHCSKSSVRSRRSSPKRKRNRHCSSHQSRSKSHRRKRSRSIEDDEEGHLICQSGDVLRARYEIVDTLGEGAFGKVVECIDHGMDGMHVAVKIVKNVGRYREAARSEIQVLEHLNSTDPNSVFRCVQMLEWFDHHGHVCIVFELLGLSTYDFIKENSFLPFQIDHIRQMAYQICQSINFLHHNKLTHTDLKPENILFVKSDYVVKYNSKMKRDERTLKNTDIKVVDFGSATYDDEHHSTLVSTRHYRAPEVILALGWSQPCDVWSIGCILIEYYLGFTVFQTHDSKEHLAMMERILGPIPQHMIQKTRKRKYFHHNQLDWDEHSSAGRYVRRRCKPLKEFMLCHDEEHEKLFDLVRRMLEYDPTQRITLDEALQHPFFDLLKKK

SEQ ID NO: 18 - DNA, 1446 bp, Homo sapiens
atgcggcattccaaaagaactcactgtcctgattgggatagcagagaaagctggggacatgaaagctatcgtggaagtcacaagcggaagaggagatctcatagtagcacacaagagaacaggcattgtaaaccacatcaccagtttaaagaatctgattgtcattatttagaagcaaggtccttgaatgagcgagattatcgggaccggagatacgttgacgaatacaggaatgactactgtgaaggatatgttcctagacattatcacagagacattgaaagcgggtatcgaatccactgcagtaaatcttcagtccgcagcaggagaagcagtcctaaaaggaagcgcaatagacactgttcaagtcatcagtcacgttcgaagagccaccgaaggaaaagatccaggagtatagaggatgatgaggagggtcacctgatctgtcaaagtggagacgttctaagagcaagatatgaaatcgtggacactttgggtgaaggagcctttggcaaagttgtagagtgcattgatcatggcatggatggcatgcatgtagcagtgaaaatcgtaaaaaatgtaggccgttaccgtgaagcagctcgttcagaaatccaagtattagagcacttaaatagtactgatcccaatagtgtcttccgatgtgtccagatgctagaatggtttgatcatcatggtcatgtttgtattgtgtttgaactactgggacttagtacttacgatttcattaaagaaaacagctttctgccatttcaaattgaccacatcaggcagatggcgtatcagatctgccagtcaataaattttttacatcataataaattaacccatacagatctgaagcctgaaaatattttgtttgtgaagtctgactatgtagtcaaatataattctaaaatgaaacgtgatgaacgcacactgaaaaacacagatatcaaagttgttgactttggaagtgcaacgtatgatgatgaacatcacagtactttggtgtctacccggcactacagagctcccgaggtcattttggctttaggttggtctcagccttgtgatgtttggagcataggttgcattcttattgaatattaccttggtttcacagtctttcagactcatgatagtaaagagcacctggcaatgatggaacgaatattaggacccataccacaacacatgattcagaaaacaagaaaacgcaagtattttcaccataaccagctagattgggatgaacacagttctgctggtagatatgttaggagacgctgcaaaccgttgaaggaatttatgctttgtcatgatgaagaacatgagaaactgtttgacctggttcgaagaatgttagaatatgatccaactcaaagaattaccttggatgaagcattgcagcatcctttctttgacttattaaaaaagaaatga

SEQ ID NO: 19 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 20 - protein, 221 aa, Homo sapiens
MSYGRPPPDVEGMTSLKVDNLTYRTSPDTLRRVFEKYGRVGDVYIPRDRYTKESRGFAFVRFHDKRDAEDAMDAMDGAVLDGRELRVQMARYGRPPDSHHSRRGPPPRRYGGGGYGRRSRSPRRRRRSRSRSRSRSRSRSRSRYSRSKSRSRTRSRSRSTSKSRSARRSKSKSSSVSRSRSRSRSRSRSRSPPPVSKRESKSRSRSKSPPKSPEEEGAVSS

SEQ ID NO: 21 - protein, 164 aa, Homo sapiens
MHRDSCPLDCKVYVGNLGNNGNKTELERAFGYYGPLRSVWVARNPPGFAFVEFEDPRDAADAVRELDGRTLCGCRVRVELSNGEKRSRNRGPPPSWGRRPRDDYRRRSPPPRRRSPRRRSFSRSRSRSLSRDRRRERSLSRERNHKPSRSFSRSRSRSRSNERK

SEQ ID NO: 22 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 23 - protein, 494 aa, Homo sapiens
MPRVYIGRLSYQARERDVERFFKGYGKILEVDLKNGYGFVEFDDLRDADDAVYELNGKDLCGERVIVEHARGPRRDGSYGSGRSGYGYRRSGRDKYGPPTRTEYRLIVENLSSRCSWQDLKDYMRQAGEVTYADAHKGRKNEGVIEFVSYSDMKRALEKLDGTEVNGRKIRLVEDKPGSRRRRSYSRSRSHSRSRSRSRHSRKSRSRSGSSKSSHSKSRSRSRSGSRSRSKSRSRSQSRSRSKKEKSRSPSKEKSRSRSHSAGKSRSKSKDQAEEKIQNNDNVGKPKSRSPSRHKSKSKSRSRSQERRVEEEKRGSVSRGRSQEKSLRQSRSRSRSKGGSRSRSRSRSKSKDKRKGRKRSREESRSRSRSRSKSERSRKRGSKRDSKAGSSKKKKKEDTDRSQSRSPSRSVSKEREHAKSESSQREGRGESENAGTNQETRSRSRSNSKSKPNLPSESRSRSKSASKTRSRSKSRSRSASRSPSRSRSRSHSRS

SEQ ID NO: 24 - protein, 272 aa, Homo sapiens
MSGCRVFIGRLNPAAREKDVERFFKGYGRIRDIDLKRGFGFVEFEDPRDADDAVYELDGKELCSERVTIEHARARSRGGRGRGRYSDRFSSRRPRNDRRNAPPVRTENRLIVENLSSRVSWQDLKDFMRQAGEVTFADAHRPKLNEGVVEFASYGDLKNAIEKLSGKEINGRKIKLIEGSKRHSRSRSRSRSRTRSSSRSRSRSRSRSRKSYSRSRSRSRSRSRSKSRSVSRSPVPEKSQKRGSSSRSKSPASVDRQRSRSRSRSRSVDSGN

SEQ ID NO: 25 - protein, 344 aa, Homo sapiens
MPRVYIGRLSYNVREKDIQRFFSGYGRLLEVDLKNGYGFVEFEDSRDADDAVYELNGKELCGERVIVEHARGPRRDRDGYSYGSRSGGGGYSSRRTSGRDKYGPPVRTEYRLIVENLSSRCSWQDLKDFMRQAGEVTYADAHKERTNEGVIEFRSYSDMKRALDKLDGTEINGRNIRLIEDKPRTSHRRSYSGSRSRSRSRRRSRSRSRRSSRSRSRSISKSRSRSRSRSKGRSRSRSKGRKSRSKSKSKPKSDRGSHSHSRSRSKDEYEKSRSRSRSRSPKENGKGDIKSKSRSRSQSRSNSPLPVPPSKARSVSPPPKRATSRSRSRSRSKSRSRSRSSSRD

SEQ ID NO: 26 - protein, 183 aa, Homo sapiens
MSRYLRPPNTSLFVRNVADDTRSEDLRREFGRYGPIVDVYVPLDFYTRRPRGFAYVQFEDVRDAEDALHNLDRKWICGRQIEIQFAQGDRKTPNQMKAKEGRNVYSSSRYDDYDRYRRSRSRSYERRRSRSRSFDYNYRRSYSPRNSRPTGRPRRSRSHSDNDRPNCSWNTQYSSAYYTSRKI

SEQ ID NO: 27 - protein, 240 aa, Homo sapiens
MAEYLASIFGTEKDKVNCSFYFKIGACRHGDRCSRLHNKPTFSQTILIQNIYRNPQNSAQTADGSHCAVSDVEMQEHYDEFFEEVFTEMEEKYGEVEEMNVCDNLGDHLVGNVYVKFRREEDAEKAVIDLNNRWFNGQPIHAELSPVTDFREACCRQYEMGECTRGGFCNFMHLKPISRELRRELYGRRRKKHRSRSRSRERRSRSRDRGRGGGGGGGGGGGGRERDRRRSRDRERSGRF

SEQ ID NO: 28 - protein, 482 aa, Homo sapiens
MAAPEKMTFPEKPSHKKYRAALKKEKRKKRRQELARLRDSGLSQKEEEEDTFIEEQQLEEEKLLERERQRLHEEWLLREQKAQEEFRIKKEKEEAAKKRQEEQERKLKEQWEEQQRKEREEEEQKRQEKKEKEEALQKMLDQAENELENGTTWQNPEPPVDFRVMEKDRANCPFYSKTGACRFGDRCSRKHNFPTSSPTLLIKSMFTTFGMEQCRRDDYDPDASLEYSEEETYQQFLDFYEDVLPEFKNVGKVIQFKVSCNLEPHLRGNVYVQYQSEEECQAALSLFNGRWYAGRQLQCEFCPVTRWKMAICGLFEIQQCPRGKHCNFLHVFRNPNNEFWEANRDIYLSPDRTGSSFGKNSERRERMGHHDDYYSRLRGRRNPSPDHSYKRNGESERKSSRHRGKKSHKRTSKSRERHNSRSRGRNRDRSRDRSRGRGSRSRSRSRSRRSRRSRSQSSSRSRSRGRRRSGNRDRTVQSPKSK

SEQ ID NO: 29 - DNA, 20 bp, Artificial
  Synthetic Sequence Forward primer
gctgccaaattgttggtttt

SEQ ID NO: 30 - DNA, 20 bp, Artificial
  Synthetic Sequence Reverse primer
caggctagaaaggggagctt

SEQ ID NO: 31 - DNA, 20 bp, Artificial
  Synthetic Sequence Forward Primer
acatcctggctcacactgaa

SEQ ID NO: 32 - DNA, 18 bp, Artificial
  Synthetic Sequence Reverse Primer
atgtctcggtcccaggtg

SEQ ID NO: 33 - DNA, 20 bp, Artificial
  Synthetic Sequence Target Sequence
gccgtgacagcgatacatac

SEQ ID NO: 34 - DNA, 20 bp, Artificial
  Synthetic Sequence
acccgtacctgagctaccga